SEQUENCE LISTING (251 sequences)

SEQ ID NO: 1 - RNA, 86 nt, Homo sapiens
  hsa-mir-323 precursor
uugguacuuggagagaggugguccguggcgcguucgcuuuauuuauggcgcacauuacacggucgaccucuuugcaguaucuaauc

SEQ ID NO: 2 - RNA, 99 nt, Homo sapiens
  hsa-mir-342 precursor
gaaacugggcucaaggugaggggugcuaucugugauugagggacaugguuaauggaauugucucacacagaaaucgcacccgucaccuuggccuacuua

SEQ ID NO: 3 - RNA, 87 nt, Homo sapiens
  hsa-mir-520f precursor
ucucaggcugugacccucuaaagggaagcgcuuucuguggucagaaagaaaagcaagugcuuccuuuuagaggguuaccguuuggga

SEQ ID NO: 4 - RNA, 85 nt, Homo sapiens
  hsa-miR-3157 precursor
gggaagggcuucagccaggcuagugcagucugcuuugugccaacacuggggugaugacugcccuagucuagcugaagcuuuuccc

SEQ ID NO: 5 - RNA, 88 nt, Homo sapiens
  hsa-miR-193a precursor
cgaggaugggagcugagggcugggucuuugcgggcgagaugagggugucggaucaacuggccuacaaagucccaguucucggcccccg

SEQ ID NO: 6 - RNA, 110 nt, Homo sapiens
  hsa-miR-7-1 precursor
uuggauguuggccuaguucuguguggaagacuagugauuuuguuguuuuuagauaacuaaaucgacaacaaaucacagucugccauauggcacaggccaugccucuacag

SEQ ID NO: 7 - RNA, 110 nt, Homo sapiens
  hsa-miR-7-2 precursor
cuggauacagaguggaccggcuggccccaucuggaagacuagugauuuuguuguugucuuacugcgcucaacaacaaaucccagucuaccuaauggugccagccaucgca

SEQ ID NO: 8 - RNA, 110 nt, Homo sapiens
  hsa-miR-7-3 precursor
agauuagaguggcuguggucuagugcuguguggaagacuagugauuuuguuguucugauguacuacgacaacaagucacagccggccucauagcgcagacucccuucgac

SEQ ID NO: 9 - DNA, 483 bp, Artificial sequence
  hsa-mir-323 DNA sequence screening
ttcctggtatttgaagatgcggttgaccatggtgtgtacgctttatttgtgacgtaggacacatggtctacttcttctcaatatcacatctcgccttggaagacttccaggaggtgatatcagctttgcggaagagccactgtcctggtgtcagtacggctgctgcttggtacttggagagaggtggtccgtggcgcgttcgctttttttatggcgcacattacacggtcgacctctttgcagtatctaatcccgccttgcaagctttcctggagctaacatcaactgcgggggtgggggccactaggtctgcgctcagtgcgacccagcggggtttgtgatgtgtctgtcttgtgtgtgacgataactcacgtgtggcagccctcttctcagcacactgctctggcttggcagcagggttaacttgcggacgaggagcgtggtgtcagcacgtgcctggatacatgagatggttgaccagag

SEQ ID NO: 10 - DNA, 488 bp, Artificial sequence
  hsa-mir-342 DNA sequence screening
cctgaagagagactgacacatcagaggtgtcyggtgactgaacaagctcccagcttgcgcccatgtcatattgtgtgcctctcatagcctggcacttcctgccattgcatccttctctgcagactaagatggagttcctgaaccaagaccgcttgctggccaacctgtgaaactgggctcaaggtgaggggtgctatctgtgattgagggacatggttaatggaattgtctcacacagaaatcgcacccgtcaccttggcctacttatcaccaccccaaacagaggaacacgccttctccagccacagcctatggaagggccttcagctgctgtggccccgaggtgtgcatactgtggaaggaacttcggacgtgaactcggatctggttccagtaccagctgtgccaggagtgcccttgggcatgtcactgacctaagactcagtttcgccatctgtgaaatggctgaatcagactcacctcacagg

SEQ ID NO: 11 - DNA, 214 bp, Artificial sequence
  hsa-mir-520f DNA sequence screening
tgtgtccatttaaacctggtcaaggaagattcccacaaaaaatccacggtgctggagcaagaggatctcaggctgtgaccctctaaagggaagcgctttctgtggtcagaaagaaaagcaagtgcttccttttagagggttaccgtttgggaaaagcaatgttgaagttgatgctgatcttggtaaaatatttgcagagcgtgcttatcatcag

SEQ ID NO: 12 - DNA, 240 bp, Artificial sequence
  hsa-miR-3157 DNA sequence screening
acaacttctcaatgagtctgccctcactgtccaacaattgagctgagaatataagaagggaagggcttcagccaggctagtgcagtctgctttgtgccaacactggggtgatgactgccctagtctagctgaagcttttcccttctttctacacccagctcaagtcccaggtccataaaacctttagaaactcttcagaaactctttagagcttcagaagctcttgagaattggaagatg

SEQ ID NO: 13 - DNA, 294 bp, Artificial sequence
  hsa-miR-193a DNA sequence screening
agggacacccagagcttcggcggagcggagcgcggtgcacagagccggcgaccggacccagccccgggaagcccgtcggggacgcaccccgaactccgaggatgggagctgagggctgggtctttgcgggcgagatgagggtgtcggatcaactggcctacaaagtcccagttctcggcccccgggaccagcgtcttctccccggtcctcgccccaggccggcttcctcccgggctggcgtgcgctccggccaggctgcctctcaggtccacgctggagaaggagtggtgaggt

SEQ ID NO: 14 - DNA, 255 bp, Artificial sequence
  hsa-miR-7-1 DNA sequence screening
gccttaaccaagcaaacttctcatttctctggtgaaaactgctgccaaaaccacttgttaaaaattgtacagagcctgtagaaaatatagaagattcattggatgttggcctagttctgtgtggaagactagtgattttgttgtttttagataactaaatcgacaacaaatcacagtctgccatatggcacaggccatgcctctacaggacaaatgattggtgctgtaaaatgcagcatttcacaccttactagc

SEQ ID NO: 15 - DNA, 239 bp, Artificial sequence
  hsa-miR-7-2 DNA sequence screening
tgaaggagcatccagaccgctgacctggtggcgaggggaggggggtggtcctcgaacgccttgcagaactggcctggatacagagtggaccggctggccccatctggaagactagtgattttgttgttgtcttactgcgctcaacaacaaatcccagtctacctaatggtgccagccatcgcagcggggtgcaggaaatgggggcagcccccctttttggctatccttccacgtgttct

SEQ ID NO: 16 - DNA, 282 bp, Artificial sequence
  hsa-miR-7-3 DNA sequence screening
tcatagcttggctcaggtgagaaggaggagctgggcaggggtctcagacatggggcagagggtggtgaagaagattagagtggctgtggtctagtgctgtgtggaagactagtgattttgttgttctgatgtactacgacaacaagtcacagccggcctcatagcgcagactcccttcgaccttcgccttcaatgggctggccagtgggggagaaccggggaggtcggggaagaatcgcttccactcggagtgggggggctggctcactccaggcgatacag

SEQ ID NO: 17 - RNA, 7 nt, Homo sapiens
  hsa-miR-323-5p seed
ggugguc

SEQ ID NO: 18 - RNA, 7 nt, Homo sapiens
  hsa-miR-342-5p seed
ggggugc

SEQ ID NO: 19 - RNA, 7 nt, Homo sapiens
  has-miR-520f-3p  seed
agugcuu

SEQ ID NO: 20 - RNA, 7 nt, Homo sapiens
  hsa-mir-520f-3p-i3 seed
aagugcu

SEQ ID NO: 21 - RNA, 7 nt, Homo sapiens
  hsa-miR-3157-5p seed
ucagcca

SEQ ID NO: 22 - RNA, 7 nt, Homo sapiens
  hsa-miR-193a-3p seed
acuggcc

SEQ ID NO: 23 - RNA, 7 nt, Homo sapiens
  hsa-miR-7-5p seed
ggaagac

SEQ ID NO: 24 - RNA, 7 nt, Homo sapiens
  hsa-miR-323-5p isomiR seed
cugcuug

SEQ ID NO: 25 - RNA, 7 nt, Homo sapiens
  hsa-miR-323-5p isomiR seed
ugcuugg

SEQ ID NO: 26 - RNA, 7 nt, Homo sapiens
  hsa-miR-323-5p isomiR seed
gcugcuu

SEQ ID NO: 27 - RNA, 7 nt, Homo sapiens
  hsa-miR-323-5p isomiR seed
agguggu

SEQ ID NO: 28 - RNA, 7 nt, Homo sapiens
  hsa-miR-323-5p isomiR seed
guggucc

SEQ ID NO: 29 - RNA, 7 nt, Homo sapiens
  hsa-miR-342-5p isomiR seed
gggugcu

SEQ ID NO: 30 - RNA, 7 nt, Homo sapiens
  hsa-miR-342-5p isomiR seed
gcuaucu

SEQ ID NO: 31 - RNA, 7 nt, Homo sapiens
  hsa-miR-342-5p isomiR seed
ggugcua

SEQ ID NO: 32 - RNA, 7 nt, Homo sapiens
  hsa-miR-342-5p isomiR seed
ugugaaa

SEQ ID NO: 33 - RNA, 7 nt, Homo sapiens
  hsa-miR-342-5p isomiR seed
gugcuau

SEQ ID NO: 34 - RNA, 7 nt, Homo sapiens
  hsa-miR-342-5p isomiR seed
ugaaacu

SEQ ID NO: 35 - RNA, 7 nt, Homo sapiens
  hsa-miR-342-5p isomiR seed
cugugaa

SEQ ID NO: 36 - RNA, 7 nt, Homo sapiens
  hsa-miR-342-5p isomiR seed
cuaucug

SEQ ID NO: 37 - RNA, 7 nt, Homo sapiens
  hsa-miR-342-5p isomiR seed
ugcuauc

SEQ ID NO: 38 - RNA, 7 nt, Homo sapiens
  hsa-miR-342-5p isomiR seed
augguua

SEQ ID NO: 39 - RNA, 7 nt, Homo sapiens
  hsa-miR-342-5p isomiR seed
aucugug

SEQ ID NO: 40 - RNA, 7 nt, Homo sapiens
  hsa-miR-342-5p isomiR seed
gaaacug

SEQ ID NO: 41 - RNA, 7 nt, Homo sapiens
  hsa-miR-342-5p isomiR seed
uaucugu

SEQ ID NO: 42 - RNA, 7 nt, Homo sapiens
  hsa-miR-342-5p isomiR seed
gugaaac

SEQ ID NO: 43 - RNA, 7 nt, Homo sapiens
  has-miR-520f-3p  isomiR seed
agugcuu

SEQ ID NO: 44 - RNA, 7 nt, Homo sapiens
  has-miR-520f-3p  isomiR seed
aagugcu

SEQ ID NO: 45 - RNA, 7 nt, Homo sapiens
  hsa-miR-3157-5p isomiR seed
ucagcca

SEQ ID NO: 46 - RNA, 7 nt, Homo sapiens
  hsa-miR-3157-5p isomiR seed
uucagcc

SEQ ID NO: 47 - RNA, 7 nt, Homo sapiens
  hsa-miR-3157-5p isomiR seed
cagccag

SEQ ID NO: 48 - RNA, 7 nt, Homo sapiens
  hsa-miR-3157-5p isomiR seed
uucagcc

SEQ ID NO: 49 - RNA, 7 nt, Homo sapiens
  hsa-miR-193a-3p isomiR seed
acuggcc

SEQ ID NO: 50 - RNA, 7 nt, Homo sapiens
  hsa-miR-7-5p isomiR seed
ggaagac

SEQ ID NO: 51 - RNA, 22 nt, Homo sapiens
  hsa-miR-323-5p mature miRNA
aggugguccguggcgcguucgc

SEQ ID NO: 52 - RNA, 21 nt, Homo sapiens
  hsa-miR-342-5p mature miRNA
aggggugcuaucugugauuga

SEQ ID NO: 53 - RNA, 22 nt, Homo sapiens
  has-miR-520f-3p  mature miRNA
aagugcuuccuuuuagaggguu

SEQ ID NO: 54 - RNA, 23 nt, Homo sapiens
  hsa-mir-520f-3p-i3 mature miRNA
caagugcuuccuuuuagaggguu

SEQ ID NO: 55 - RNA, 22 nt, Homo sapiens
  hsa-miR-3157-5p mature miRNA
uucagccaggcuagugcagucu

SEQ ID NO: 56 - RNA, 22 nt, Homo sapiens
  hsa-miR-193a-3p mature miRNA
aacuggccuacaaagucccagu

SEQ ID NO: 57 - RNA, 23 nt, Homo sapiens
  hsa-miR-7-5p mature miRNA
uggaagacuagugauuuuguugu

SEQ ID NO: 58 - RNA, 20 nt, Homo sapiens
  hsa-miR-323-5p isomiR
aggugguccguggcgcguuc

SEQ ID NO: 59 - RNA, 21 nt, Homo sapiens
  hsa-miR-323-5p isomiR
aggugguccguggcgcguucg

SEQ ID NO: 60 - RNA, 20 nt, Homo sapiens
  hsa-miR-323-5p isomiR
gcugcuugguacuuggagag

SEQ ID NO: 61 - RNA, 19 nt, Homo sapiens
  hsa-miR-323-5p isomiR
aggugguccguggcgcguu

SEQ ID NO: 62 - RNA, 19 nt, Homo sapiens
  hsa-miR-323-5p isomiR
cugcuugguacuuggagag

SEQ ID NO: 63 - RNA, 21 nt, Homo sapiens
  hsa-miR-323-5p isomiR
ugcugcuugguacuuggagag

SEQ ID NO: 64 - RNA, 21 nt, Homo sapiens
  hsa-miR-323-5p isomiR
gaggugguccguggcgcguuc

SEQ ID NO: 65 - RNA, 23 nt, Homo sapiens
  hsa-miR-323-5p isomiR
aggugguccguggcgcguucgcu

SEQ ID NO: 66 - RNA, 18 nt, Homo sapiens
  hsa-miR-323-5p isomiR
ggugguccguggcgcguu

SEQ ID NO: 67 - RNA, 18 nt, Homo sapiens
  hsa-miR-323-5p isomiR
aggugguccguggcgcgu

SEQ ID NO: 68 - RNA, 20 nt, Homo sapiens
  hsa-miR-323-5p isomiR
gaggugguccguggcgcguu

SEQ ID NO: 69 - RNA, 26 nt, Homo sapiens
  hsa-miR-342-5p isomiR
ggggugcuaucugugauugagggaca

SEQ ID NO: 70 - RNA, 25 nt, Homo sapiens
  hsa-miR-342-5p isomiR
ggggugcuaucugugauugagggac

SEQ ID NO: 71 - RNA, 24 nt, Homo sapiens
  hsa-miR-342-5p isomiR
ggggugcuaucugugauugaggga

SEQ ID NO: 72 - RNA, 22 nt, Homo sapiens
  hsa-miR-342-5p isomiR
ggggugcuaucugugauugagg

SEQ ID NO: 73 - RNA, 22 nt, Homo sapiens
  hsa-miR-342-5p isomiR
ugcuaucugugauugagggaca

SEQ ID NO: 74 - RNA, 23 nt, Homo sapiens
  hsa-miR-342-5p isomiR
aggggugcuaucugugauugagg

SEQ ID NO: 75 - RNA, 23 nt, Homo sapiens
  hsa-miR-342-5p isomiR
ggggugcuaucugugauugaggg

SEQ ID NO: 76 - RNA, 27 nt, Homo sapiens
  hsa-miR-342-5p isomiR
aggggugcuaucugugauugagggaca

SEQ ID NO: 77 - RNA, 25 nt, Homo sapiens
  hsa-miR-342-5p isomiR
aggggugcuaucugugauugaggga

SEQ ID NO: 78 - RNA, 20 nt, Homo sapiens
  hsa-miR-342-5p isomiR
ggggugcuaucugugauuga

SEQ ID NO: 79 - RNA, 21 nt, Homo sapiens
  hsa-miR-342-5p isomiR
ugcuaucugugauugagggac

SEQ ID NO: 80 - RNA, 23 nt, Homo sapiens
  hsa-miR-342-5p isomiR
gggugcuaucugugauugaggga

SEQ ID NO: 81 - RNA, 24 nt, Homo sapiens
  hsa-miR-342-5p isomiR
aggggugcuaucugugauugaggg

SEQ ID NO: 82 - RNA, 24 nt, Homo sapiens
  hsa-miR-342-5p isomiR
gggugcuaucugugauugagggac

SEQ ID NO: 83 - RNA, 22 nt, Homo sapiens
  hsa-miR-342-5p isomiR
aggggugcuaucugugauugag

SEQ ID NO: 84 - RNA, 22 nt, Homo sapiens
  hsa-miR-342-5p isomiR
gggugcuaucugugauugaggg

SEQ ID NO: 85 - RNA, 21 nt, Homo sapiens
  hsa-miR-342-5p isomiR
gggugcuaucugugauugagg

SEQ ID NO: 86 - RNA, 20 nt, Homo sapiens
  hsa-miR-342-5p isomiR
ugcuaucugugauugaggga

SEQ ID NO: 87 - RNA, 25 nt, Homo sapiens
  hsa-miR-342-5p isomiR
gggugcuaucugugauugagggaca

SEQ ID NO: 88 - RNA, 26 nt, Homo sapiens
  hsa-miR-342-5p isomiR
aggggugcuaucugugauugagggac

SEQ ID NO: 89 - RNA, 22 nt, Homo sapiens
  hsa-miR-342-5p isomiR
cugugaaacugggcucaaggug

SEQ ID NO: 90 - RNA, 20 nt, Homo sapiens
  hsa-miR-342-5p isomiR
aggggugcuaucugugauug

SEQ ID NO: 91 - RNA, 21 nt, Homo sapiens
  hsa-miR-342-5p isomiR
ggggugcuaucugugauugag

SEQ ID NO: 92 - RNA, 23 nt, Homo sapiens
  hsa-miR-342-5p isomiR
ugcuaucugugauugagggacau

SEQ ID NO: 93 - RNA, 23 nt, Homo sapiens
  hsa-miR-342-5p isomiR
cugugaaacugggcucaagguga

SEQ ID NO: 94 - RNA, 23 nt, Homo sapiens
  hsa-miR-342-5p isomiR
ggugcuaucugugauugagggac

SEQ ID NO: 95 - RNA, 20 nt, Homo sapiens
  hsa-miR-342-5p isomiR
gugaaacugggcucaaggug

SEQ ID NO: 96 - RNA, 20 nt, Homo sapiens
  hsa-miR-342-5p isomiR
gggugcuaucugugauugag

SEQ ID NO: 97 - RNA, 19 nt, Homo sapiens
  hsa-miR-342-5p isomiR
ggggugcuaucugugauug

SEQ ID NO: 98 - RNA, 21 nt, Homo sapiens
  hsa-miR-342-5p isomiR
gcuaucugugauugagggaca

SEQ ID NO: 99 - RNA, 23 nt, Homo sapiens
  hsa-miR-342-5p isomiR
ccugugaaacugggcucaaggug

SEQ ID NO: 100 - RNA, 22 nt, Homo sapiens
  hsa-miR-342-5p isomiR
gugcuaucugugauugagggac

SEQ ID NO: 101 - RNA, 28 nt, Homo sapiens
  hsa-miR-342-5p isomiR
aggggugcuaucugugauugagggacau

SEQ ID NO: 102 - RNA, 19 nt, Homo sapiens
  hsa-miR-342-5p isomiR
ugcuaucugugauugaggg

SEQ ID NO: 103 - RNA, 18 nt, Homo sapiens
  hsa-miR-342-5p isomiR
gggugcuaucugugauug

SEQ ID NO: 104 - RNA, 19 nt, Homo sapiens
  hsa-miR-342-5p isomiR
caugguuaauggaauuguc

SEQ ID NO: 105 - RNA, 27 nt, Homo sapiens
  hsa-miR-342-5p isomiR
ggggugcuaucugugauugagggacau

SEQ ID NO: 106 - RNA, 19 nt, Homo sapiens
  hsa-miR-342-5p isomiR
gggugcuaucugugauuga

SEQ ID NO: 107 - RNA, 19 nt, Homo sapiens
  hsa-miR-342-5p isomiR
uaucugugauugagggaca

SEQ ID NO: 108 - RNA, 21 nt, Homo sapiens
  hsa-miR-342-5p isomiR
gugaaacugggcucaagguga

SEQ ID NO: 109 - RNA, 24 nt, Homo sapiens
  hsa-miR-342-5p isomiR
ccugugaaacugggcucaagguga

SEQ ID NO: 110 - RNA, 20 nt, Homo sapiens
  hsa-miR-342-5p isomiR
ggugcuaucugugauugagg

SEQ ID NO: 111 - RNA, 20 nt, Homo sapiens
  hsa-miR-342-5p isomiR
cuaucugugauugagggaca

SEQ ID NO: 112 - RNA, 19 nt, Homo sapiens
  hsa-miR-342-5p isomiR
ugaaacugggcucaaggug

SEQ ID NO: 113 - RNA, 22 nt, Homo sapiens
  hsa-miR-342-5p isomiR
ugugaaacugggcucaagguga

SEQ ID NO: 114 - RNA, 21 nt, Homo sapiens
  hsa-mir-520f-3p isomiR
aagugcuuccuuuuagagggu

SEQ ID NO: 115 - RNA, 22 nt, Homo sapiens
  hsa-mir-520f-3p isomiR
caagugcuuccuuuuagagggu

SEQ ID NO: 116 - RNA, 21 nt, Homo sapiens
  hsa-miR-3157-5p isomiR
uucagccaggcuagugcaguc

SEQ ID NO: 117 - RNA, 22 nt, Homo sapiens
  hsa-miR-3157-5p isomiR
cuucagccaggcuagugcaguc

SEQ ID NO: 118 - RNA, 21 nt, Homo sapiens
  hsa-miR-3157-5p isomiR
ucagccaggcuagugcagucu

SEQ ID NO: 119 - RNA, 20 nt, Homo sapiens
  hsa-miR-3157-5p isomiR
uucagccaggcuagugcagu

SEQ ID NO: 120 - RNA, 24 nt, Homo sapiens
  hsa-miR-3157-5p isomiR
cuucagccaggcuagugcagucug

SEQ ID NO: 121 - RNA, 20 nt, Homo sapiens
  hsa-miR-193a-3p isomiR
aacuggccuacaaaguccca

SEQ ID NO: 122 - RNA, 21 nt, Homo sapiens
  hsa-miR-193a-3p isomiR
aacuggccuacaaagucccag

SEQ ID NO: 123 - RNA, 24 nt, Homo sapiens
  hsa-miR-7-5p isomiR
uggaagacuagugauuuuguuguu

SEQ ID NO: 124 - RNA, 22 nt, Homo sapiens
  hsa-miR-7-5p isomiR
uggaagacuagugauuuuguug

SEQ ID NO: 125 - RNA, 25 nt, Homo sapiens
  hsa-miR-7-5p isomiR
uggaagacuagugauuuuguuguuc

SEQ ID NO: 126 - RNA, 22 nt, Homo sapiens
  hsa-miR-323-5p mature miRNA sense
gcgaacgcgccacggaccaccu

SEQ ID NO: 127 - RNA, 21 nt, Homo sapiens
  hsa-miR-342-5p mature miRNA sense
ucaaucacagauagcaccccu

SEQ ID NO: 128 - RNA, 22 nt, Homo sapiens
  has-miR-520f-3p  mature miRNA sense
aacccucuaaaaggaagcacuu

SEQ ID NO: 129 - RNA, 23 nt, Homo sapiens
  hsa-mir-520f-3p-i3 mature miRNA sense
aacccucuaaaaggaagcacuug

SEQ ID NO: 130 - RNA, 22 nt, Homo sapiens
  hsa-miR-3157-5p mature miRNA sense
agacugcacuagccuggcugaa

SEQ ID NO: 131 - RNA, 22 nt, Homo sapiens
  hsa-miR-193a-3p mature miRNA sense
acugggacuuuguaggccaguu

SEQ ID NO: 132 - RNA, 23 nt, Homo sapiens
  hsa-miR-7-5p mature miRNA sense
acaacaaaaucacuagucuucca

SEQ ID NO: 133 - RNA, 20 nt, Homo sapiens
  hsa-miR-323-5p isomiR sense
  misc_feature (19)..(20): n is a, c, g, or u
acgcgccacggaccaccunn

SEQ ID NO: 134 - RNA, 21 nt, Homo sapiens
  hsa-miR-323-5p isomiR sense
  misc_feature (20)..(21): n is a, c, g, or u
aacgcgccacggaccaccunn

SEQ ID NO: 135 - RNA, 20 nt, Homo sapiens
  hsa-miR-323-5p isomiR sense
  misc_feature (19)..(20): n is a, c, g, or u
cuccaaguaccaagcagcnn

SEQ ID NO: 136 - RNA, 19 nt, Homo sapiens
  hsa-miR-323-5p isomiR sense
  misc_feature (18)..(19): n is a, c, g, or u
cgcgccacggaccaccunn

SEQ ID NO: 137 - RNA, 19 nt, Homo sapiens
  hsa-miR-323-5p isomiR sense
  misc_feature (18)..(19): n is a, c, g, or u
cuccaaguaccaagcagnn

SEQ ID NO: 138 - RNA, 21 nt, Homo sapiens
  hsa-miR-323-5p isomiR sense
  misc_feature (20)..(21): n is a, c, g, or u
cuccaaguaccaagcagcann

SEQ ID NO: 139 - RNA, 21 nt, Homo sapiens
  hsa-miR-323-5p isomiR sense
  misc_feature (20)..(21): n is a, c, g, or u
acgcgccacggaccaccucnn

SEQ ID NO: 140 - RNA, 23 nt, Homo sapiens
  hsa-miR-323-5p isomiR sense
  misc_feature (22)..(23): n is a, c, g, or u
cgaacgcgccacggaccaccunn

SEQ ID NO: 141 - RNA, 18 nt, Homo sapiens
  hsa-miR-323-5p isomiR sense
  misc_feature (17)..(18): n is a, c, g, or u
cgcgccacggaccaccnn

SEQ ID NO: 142 - RNA, 18 nt, Homo sapiens
  hsa-miR-323-5p isomiR sense
  misc_feature (17)..(18): n is a, c, g, or u
gcgccacggaccaccunn

SEQ ID NO: 143 - RNA, 20 nt, Homo sapiens
  hsa-miR-323-5p isomiR sense
  misc_feature (19)..(20): n is a, c, g, or u
cgcgccacggaccaccucnn

SEQ ID NO: 144 - RNA, 26 nt, Homo sapiens
  hsa-miR-342-5p isomiR sense
  misc_feature (25)..(26): n is a, c, g, or u
ucccucaaucacagauagcaccccnn

SEQ ID NO: 145 - RNA, 25 nt, Homo sapiens
  hsa-miR-342-5p isomiR sense
  misc_feature (24)..(25): n is a, c, g, or u
cccucaaucacagauagcaccccnn

SEQ ID NO: 146 - RNA, 24 nt, Homo sapiens
  hsa-miR-342-5p isomiR sense
  misc_feature (23)..(24): n is a, c, g, or u
ccucaaucacagauagcaccccnn

SEQ ID NO: 147 - RNA, 22 nt, Homo sapiens
  hsa-miR-342-5p isomiR sense
  misc_feature (21)..(22): n is a, c, g, or u
ucaaucacagauagcaccccnn

SEQ ID NO: 148 - RNA, 22 nt, Homo sapiens
  hsa-miR-342-5p isomiR sense
  misc_feature (21)..(22): n is a, c, g, or u
ucccucaaucacagauagcann

SEQ ID NO: 149 - RNA, 23 nt, Homo sapiens
  hsa-miR-342-5p isomiR sense
  misc_feature (22)..(23): n is a, c, g, or u
ucaaucacagauagcaccccunn

SEQ ID NO: 150 - RNA, 23 nt, Homo sapiens
  hsa-miR-342-5p isomiR sense
  misc_feature (22)..(23): n is a, c, g, or u
cucaaucacagauagcaccccnn

SEQ ID NO: 151 - RNA, 27 nt, Homo sapiens
  hsa-miR-342-5p isomiR sense
  misc_feature (26)..(27): n is a, c, g, or u
ucccucaaucacagauagcaccccunn

SEQ ID NO: 152 - RNA, 25 nt, Homo sapiens
  hsa-miR-342-5p isomiR sense
  misc_feature (24)..(25): n is a, c, g, or u
ccucaaucacagauagcaccccunn

SEQ ID NO: 153 - RNA, 20 nt, Homo sapiens
  hsa-miR-342-5p isomiR sense
  misc_feature (19)..(20): n is a, c, g, or u
aaucacagauagcaccccnn

SEQ ID NO: 154 - RNA, 21 nt, Homo sapiens
  hsa-miR-342-5p isomiR sense
  misc_feature (20)..(21): n is a, c, g, or u
cccucaaucacagauagcann

SEQ ID NO: 155 - RNA, 23 nt, Homo sapiens
  hsa-miR-342-5p isomiR sense
  misc_feature (22)..(23): n is a, c, g, or u
ccucaaucacagauagcacccnn

SEQ ID NO: 156 - RNA, 24 nt, Homo sapiens
  hsa-miR-342-5p isomiR sense
  misc_feature (23)..(24): n is a, c, g, or u
cucaaucacagauagcaccccunn

SEQ ID NO: 157 - RNA, 24 nt, Homo sapiens
  hsa-miR-342-5p isomiR sense
  misc_feature (23)..(24): n is a, c, g, or u
cccucaaucacagauagcacccnn

SEQ ID NO: 158 - RNA, 22 nt, Homo sapiens
  hsa-miR-342-5p isomiR sense
  misc_feature (21)..(22): n is a, c, g, or u
caaucacagauagcaccccunn

SEQ ID NO: 159 - RNA, 22 nt, Homo sapiens
  hsa-miR-342-5p isomiR sense
  misc_feature (21)..(22): n is a, c, g, or u
cucaaucacagauagcacccnn

SEQ ID NO: 160 - RNA, 21 nt, Homo sapiens
  hsa-miR-342-5p isomiR sense
  misc_feature (20)..(21): n is a, c, g, or u
ucaaucacagauagcacccnn

SEQ ID NO: 161 - RNA, 20 nt, Homo sapiens
  hsa-miR-342-5p isomiR sense
  misc_feature (19)..(20): n is a, c, g, or u
ccucaaucacagauagcann

SEQ ID NO: 162 - RNA, 25 nt, Homo sapiens
  hsa-miR-342-5p isomiR sense
  misc_feature (24)..(25): n is a, c, g, or u
ucccucaaucacagauagcacccnn

SEQ ID NO: 163 - RNA, 26 nt, Homo sapiens
  hsa-miR-342-5p isomiR sense
  misc_feature (25)..(26): n is a, c, g, or u
cccucaaucacagauagcaccccunn

SEQ ID NO: 164 - RNA, 22 nt, Homo sapiens
  hsa-miR-342-5p isomiR sense
  misc_feature (21)..(22): n is a, c, g, or u
ccuugagcccaguuucacagnn

SEQ ID NO: 165 - RNA, 20 nt, Homo sapiens
  hsa-miR-342-5p isomiR sense
  misc_feature (19)..(20): n is a, c, g, or u
aucacagauagcaccccunn

SEQ ID NO: 166 - RNA, 21 nt, Homo sapiens
  hsa-miR-342-5p isomiR sense
  misc_feature (20)..(21): n is a, c, g, or u
caaucacagauagcaccccnn

SEQ ID NO: 167 - RNA, 23 nt, Homo sapiens
  hsa-miR-342-5p isomiR sense
  misc_feature (22)..(23): n is a, c, g, or u
gucccucaaucacagauagcann

SEQ ID NO: 168 - RNA, 23 nt, Homo sapiens
  hsa-miR-342-5p isomiR sense
  misc_feature (22)..(23): n is a, c, g, or u
accuugagcccaguuucacagnn

SEQ ID NO: 169 - RNA, 23 nt, Homo sapiens
  hsa-miR-342-5p isomiR sense
  misc_feature (22)..(23): n is a, c, g, or u
cccucaaucacagauagcaccnn

SEQ ID NO: 170 - RNA, 20 nt, Homo sapiens
  hsa-miR-342-5p isomiR sense
  misc_feature (19)..(20): n is a, c, g, or u
ccuugagcccaguuucacnn

SEQ ID NO: 171 - RNA, 20 nt, Homo sapiens
  hsa-miR-342-5p isomiR sense
  misc_feature (19)..(20): n is a, c, g, or u
caaucacagauagcacccnn

SEQ ID NO: 172 - RNA, 19 nt, Homo sapiens
  hsa-miR-342-5p isomiR sense
  misc_feature (18)..(19): n is a, c, g, or u
aucacagauagcaccccnn

SEQ ID NO: 173 - RNA, 21 nt, Homo sapiens
  hsa-miR-342-5p isomiR sense
  misc_feature (20)..(21): n is a, c, g, or u
ucccucaaucacagauagcnn

SEQ ID NO: 174 - RNA, 23 nt, Homo sapiens
  hsa-miR-342-5p isomiR sense
  misc_feature (22)..(23): n is a, c, g, or u
ccuugagcccaguuucacaggnn

SEQ ID NO: 175 - RNA, 22 nt, Homo sapiens
  hsa-miR-342-5p isomiR sense
  misc_feature (21)..(22): n is a, c, g, or u
cccucaaucacagauagcacnn

SEQ ID NO: 176 - RNA, 28 nt, Homo sapiens
  hsa-miR-342-5p isomiR sense
  misc_feature (27)..(28): n is a, c, g, or u
gucccucaaucacagauagcaccccunn

SEQ ID NO: 177 - RNA, 19 nt, Homo sapiens
  hsa-miR-342-5p isomiR sense
  misc_feature (18)..(19): n is a, c, g, or u
cucaaucacagauagcann

SEQ ID NO: 178 - RNA, 18 nt, Homo sapiens
  hsa-miR-342-5p isomiR sense
  misc_feature (17)..(18): n is a, c, g, or u
aucacagauagcacccnn

SEQ ID NO: 179 - RNA, 19 nt, Homo sapiens
  hsa-miR-342-5p isomiR sense
  misc_feature (18)..(19): n is a, c, g, or u
caauuccauuaaccaugnn

SEQ ID NO: 180 - RNA, 27 nt, Homo sapiens
  hsa-miR-342-5p isomiR sense
  misc_feature (26)..(27): n is a, c, g, or u
gucccucaaucacagauagcaccccnn

SEQ ID NO: 181 - RNA, 19 nt, Homo sapiens
  hsa-miR-342-5p isomiR sense
  misc_feature (18)..(19): n is a, c, g, or u
aaucacagauagcacccnn

SEQ ID NO: 182 - RNA, 19 nt, Homo sapiens
  hsa-miR-342-5p isomiR sense
  misc_feature (18)..(19): n is a, c, g, or u
ucccucaaucacagauann

SEQ ID NO: 183 - RNA, 21 nt, Homo sapiens
  hsa-miR-342-5p isomiR sense
  misc_feature (20)..(21): n is a, c, g, or u
accuugagcccaguuucacnn

SEQ ID NO: 184 - RNA, 24 nt, Homo sapiens
  hsa-miR-342-5p isomiR sense
  misc_feature (23)..(24): n is a, c, g, or u
accuugagcccaguuucacaggnn

SEQ ID NO: 185 - RNA, 20 nt, Homo sapiens
  hsa-miR-342-5p isomiR sense
  misc_feature (19)..(20): n is a, c, g, or u
ucaaucacagauagcaccnn

SEQ ID NO: 186 - RNA, 20 nt, Homo sapiens
  hsa-miR-342-5p isomiR sense
  misc_feature (19)..(20): n is a, c, g, or u
ucccucaaucacagauagnn

SEQ ID NO: 187 - RNA, 19 nt, Homo sapiens
  hsa-miR-342-5p isomiR sense
  misc_feature (18)..(19): n is a, c, g, or u
ccuugagcccaguuucann

SEQ ID NO: 188 - RNA, 22 nt, Homo sapiens
  hsa-miR-342-5p isomiR sense
  misc_feature (21)..(22): n is a, c, g, or u
accuugagcccaguuucacann

SEQ ID NO: 189 - RNA, 21 nt, Homo sapiens
  hsa-mir-520f-3p isomiR sense
  misc_feature (20)..(21): n is a, c, g, or u
ccucuaaaaggaagcacuunn

SEQ ID NO: 190 - RNA, 23 nt, Homo sapiens
  hsa-mir-520f-3p isomiR sense
  misc_feature (22)..(23): n is a, c, g, or u
cccucuaaaaggaagcacuugnn

SEQ ID NO: 191 - RNA, 21 nt, Homo sapiens
  hsa-miR-3157-5p isomiR sense
  misc_feature (20)..(21): n is a, c, g, or u
cugcacuagccuggcugaann

SEQ ID NO: 192 - RNA, 22 nt, Homo sapiens
  hsa-miR-3157-5p isomiR sense
  misc_feature (21)..(22): n is a, c, g, or u
cugcacuagccuggcugaagnn

SEQ ID NO: 193 - RNA, 21 nt, Homo sapiens
  hsa-miR-3157-5p isomiR sense
  misc_feature (20)..(21): n is a, c, g, or u
acugcacuagccuggcugann

SEQ ID NO: 194 - RNA, 20 nt, Homo sapiens
  hsa-miR-3157-5p isomiR sense
  misc_feature (19)..(20): n is a, c, g, or u
ugcacuagccuggcugaann

SEQ ID NO: 195 - RNA, 24 nt, Homo sapiens
  hsa-miR-3157-5p isomiR sense
  misc_feature (23)..(24): n is a, c, g, or u
gacugcacuagccuggcugaagnn

SEQ ID NO: 196 - RNA, 20 nt, Homo sapiens
  hsa-miR-193a-3p isomiR sense
  misc_feature (19)..(20): n is a, c, g, or u
ggacuuuguaggccaguunn

SEQ ID NO: 197 - RNA, 21 nt, Homo sapiens
  hsa-miR-193a-3p isomiR sense
  misc_feature (20)..(21): n is a, c, g, or u
gggacuuuguaggccaguunn

SEQ ID NO: 198 - RNA, 24 nt, Homo sapiens
  hsa-miR-7-5p isomiR sense
  misc_feature (23)..(24): n is a, c, g, or u
caacaaaaucacuagucuuccann

SEQ ID NO: 199 - RNA, 22 nt, Homo sapiens
  hsa-miR-7-5p isomiR sense
  misc_feature (21)..(22): n is a, c, g, or u
acaaaaucacuagucuuccann

SEQ ID NO: 200 - RNA, 25 nt, Homo sapiens
  hsa-miR-7-5p isomiR sense
  misc_feature (24)..(25): n is a, c, g, or u
acaacaaaaucacuagucuuccann

SEQ ID NO: 201 - RNA, 22 nt, Homo sapiens
  hsa-miR-323-5p mimic sense
gaacgcgccacggaccaccuuu

SEQ ID NO: 202 - RNA, 21 nt, Homo sapiens
  hsa-miR-342-5p mimic sense
aaucacagauagcaccccuuu

SEQ ID NO: 203 - RNA, 20 nt, Homo sapiens
  has-miR-520f-3p  mimic sense
cccucuaaaaggaagcacuu

SEQ ID NO: 204 - RNA, 21 nt, Homo sapiens
  hsa-mir-520f-3p-i3 mimic sense
cccucuaaaaggaagcacuug

SEQ ID NO: 205 - RNA, 22 nt, Homo sapiens
  hsa-miR-3157-5p mimic sense
agacugcacuagccuggcugaa

SEQ ID NO: 206 - RNA, 20 nt, Homo sapiens
  hsa-miR-193a-3p mimic sense
ugggacuuuguaggccaguu

SEQ ID NO: 207 - RNA, 21 nt, Homo sapiens
  hsa-miR-7-5p mimic sense
aacaaaaucacuagucuucca

SEQ ID NO: 208 - RNA, 22 nt, Homo sapiens
  hsa-miR-323-5p mimic sense
  2′-O-methylnucleoside (1)..(2)
gaacgcgccacggaccaccuuu

SEQ ID NO: 209 - RNA, 22 nt, Homo sapiens
  hsa-miR-323-5p mimic antisense
aggugguccguggcgcguucgc

SEQ ID NO: 210 - RNA, 21 nt, Homo sapiens
  hsa-miR-342-5p mimic sense
  2′-O-methylnucleoside (1)..(2)
aaucacagauagcaccccuuu

SEQ ID NO: 211 - RNA, 21 nt, Homo sapiens
  hsa-miR-342-5p mimic antisense
aggggugcuaucugugauuga

SEQ ID NO: 212 - DNA, 22 bp, Homo sapiens
  hsa-miR-520f-3p mimic sense
  RNA (1)..(20)
  2′-O-methylnucleoside (1)..(2)
  2′-O-methylnucleoside (19)..(20)
  DNA (21)..(22)
cccucuaaaaggaagcacuutt

SEQ ID NO: 213 - RNA, 22 nt, Homo sapiens
  hsa-miR-520f-3p mimic antisense
aagugcuuccuuuuagaggguu

SEQ ID NO: 214 - DNA, 23 bp, Homo sapiens
  hsa-miR-520-i3-3p mimic sense
  RNA (1)..(21)
  2′-O-methylnucleoside (1)..(2)
  2′-O-methylnucleoside (20)..(21)
  DNA (22)..(23)
cccucuaaaaggaagcacuugtt

SEQ ID NO: 215 - RNA, 23 nt, Homo sapiens
  hsa-miR-520-i3-3p mimic antisense
caagugcuuccuuuuagaggguu

SEQ ID NO: 216 - RNA, 22 nt, Homo sapiens
  hsa-miR-3157-5p mimic sense
  2′-O-methylnucleoside (1)..(1)
agacugcacuagccuggcugaa

SEQ ID NO: 217 - RNA, 24 nt, Homo sapiens
  hsa-miR-3157-5p mimic antisense
  2′-O-methylnucleoside (22)..(24)
uucagccaggcuagugcagucuua

SEQ ID NO: 218 - DNA, 22 bp, Homo sapiens
  hsa-miR-193a-3p mimic sense
  RNA (1)..(20)
  2′-O-methylnucleoside (1)..(2)
  2′-O-methylnucleoside (19)..(20)
  DNA (21)..(22)
ugggacuuuguaggccaguutt

SEQ ID NO: 219 - RNA, 22 nt, Homo sapiens
  hsa-miR-193a-3p mimic antisense
aacuggccuacaaagucccagu

SEQ ID NO: 220 - DNA, 23 bp, Homo sapiens
  hsa-miR-7-5p mimic sense
  RNA (1)..(21)
  2′-O-methylnucleoside (1)..(2)
  2′-O-methylnucleoside (20)..(21)
  DNA (22)..(23)
aacaaaaucacuagucuuccatt

SEQ ID NO: 221 - RNA, 23 nt, Homo sapiens
  hsa-miR-7-5p mimic antisense
uggaagacuagugauuuuguugu

SEQ ID NO: 222 - DNA, 21 bp, Artificial sequence
  HPRT1 forward primer
tccaaagatggtcaaggtcgc

SEQ ID NO: 223 - DNA, 23 bp, Artificial sequence
  HPRT1 reverse primer
cacgaagatctgcattgtcaagt

SEQ ID NO: 224 - DNA, 18 bp, Artificial sequence
  UBS forward primer
cagccgggatttgggtcg

SEQ ID NO: 225 - DNA, 23 bp, Artificial sequence
  UBS reverse primer
cacgaagatctgcattgtcaagt

SEQ ID NO: 226 - DNA, 20 bp, Artificial sequence
  GUSB forward primer
tgcgtagggacaagaaccac

SEQ ID NO: 227 - DNA, 20 bp, Artificial sequence
  GUSB reverse primer
gggaggggtccaaggatttg

SEQ ID NO: 228 - DNA, 20 bp, Artificial sequence
  mPPIH forward primer
aatcgagctctttgcagacg

SEQ ID NO: 229 - DNA, 20 bp, Artificial sequence
  mPPIH reverse primer
tatcctatcggaacgccatc

SEQ ID NO: 230 - DNA, 20 bp, Artificial sequence
  mSDHA forward primer
gaggaagcacaccctctcat

SEQ ID NO: 231 - DNA, 20 bp, Artificial sequence
  mSDHA reverse primer
ggagcggatagcaggaggta

SEQ ID NO: 232 - DNA, 20 bp, Artificial sequence
  mMCL-1 forward primer
taaggacgaaacgggactgg

SEQ ID NO: 233 - DNA, 20 bp, Artificial sequence
  mMCL-1 reverse primer
cgccttctaggtcctgtacg

SEQ ID NO: 234 - DNA, 20 bp, Artificial sequence
  mENTPD1 forward primer
gccgaatgcatggaactgtc

SEQ ID NO: 235 - DNA, 20 bp, Artificial sequence
  mENTPD1 reverse primer
ctgccgattgttcgctttcc

SEQ ID NO: 236 - DNA, 22 bp, Artificial sequence
  mKRAS forward primer
gtggatgagtatgaccctacga

SEQ ID NO: 237 - DNA, 20 bp, Artificial sequence
  mKRAS reverse primer
ctcctcttgacctgctgtgt

SEQ ID NO: 238 - DNA, 20 bp, Artificial sequence
  mTIM3 forward primer
gcaggatacagttccctggt

SEQ ID NO: 239 - DNA, 20 bp, Artificial sequence
  mTIM3 reverse primer
tctgagctggagtgaccttg

SEQ ID NO: 240 - DNA, 20 bp, Artificial sequence
  hMpp2 fwd primer
ccaggatgatgccaactggt

SEQ ID NO: 241 - DNA, 20 bp, Artificial sequence
  hMpp2 rev primer
atgctttccgcttctcctcc

SEQ ID NO: 242 - DNA, 20 bp, Artificial sequence
  hSTMN1 fwd primer
ccagaattccccctttcccc

SEQ ID NO: 243 - DNA, 20 bp, Artificial sequence
  hSTMN1 rev primer
ccagctgcttcaagacctca

SEQ ID NO: 244 - DNA, 25 bp, Artificial sequence
  hYWHAZ fwd primer
agaaaattgagacggagctaagaga

SEQ ID NO: 245 - DNA, 24 bp, Artificial sequence
  hYWHAZ rev primer
agaagactttgctctctgcttgtg

SEQ ID NO: 246 - DNA, 20 bp, Artificial sequence
  hCCNA2 fwd primer
cggtactgaagtccgggaac

SEQ ID NO: 247 - DNA, 20 bp, Artificial sequence
  hCCNA2 rev primer
tgctttccaaggaggaacgg

SEQ ID NO: 248 - DNA, 20 bp, Artificial sequence
  hNT5E fwd primer
aacaacctgagacacacgga

SEQ ID NO: 249 - DNA, 21 bp, Artificial sequence
  hNT5E rev primer
tggattccattgttgcgttca

SEQ ID NO: 250 - DNA, 22 bp, Artificial sequence
  hENTPD1 fwd primer
gcttcttgtgctatgggaagga

SEQ ID NO: 251 - DNA, 21 bp, Artificial sequenceh
  hENTPD1 rev primer
gatgaaagcatgggtccctga

(19) United States
(12) Patent Application Publication
(10) Pub. No.: US 2022/0275368 A1
(43) Pub. Date: Sep. 1, 2022

(54) NEW TREATMENTS INVOLVING MIRNA-193A

(71) Applicant: INTERNA TECHNOLOGIES B.V., Nijmegen (NL)

(72) Inventors: Sanaz YAHYANEJAD, Rotterdam (NL); Bryony Jane TELFORD, Utrecht (NL); Marion Tina Jolien VAN DEN BOSCH, Zeist (NL); Mir Farshid ALEMDEHY, Nootdorp (NL); Matheus Maria DE GUNST, Woudenberg (NL); Laurens Adrianus Hendricus VAN PINXTEREN, Den Haag (NL); Roeland Quirinus Jozef SCHAAPVELD, Veenendaal (NL); Michel JANICOT, Brussels (BE)

(73) Assignee: INTERNA TECHNOLOGIES B.V., Nijmegen (NL)

(21) Appl. No.: 17/630,457

(22) PCT Filed: Mar. 6, 2020
(86) PCT No.: PCT/EP2020/055965

§ 371 Date: Jan. 26, 2022

(30) Foreign Application Priority Data
Aug. 12, 2019 (EP) 19191222.9
Feb. 28, 2020 (EP) 20160235.6

(51) Int. Cl. C12N 15/113; A61P 35/00; A61K 45/06

(52) U.S. Cl. CPC C12N 15/113; A61P 35/00; A61K 45/06; C12N 2310/141; C12N 2320/31

(57) ABSTRACT

The invention relates to the use of miRNA-193a for regulating gene expression, particularly it relates to the use of miRNA-193a as a PTEN agonist. This allows the advantageous treatment of PTEN-deficient cancers. The invention further relates to compositions comprising the miRNA for use as a PTEN agonist.

### FIELD OF THE INVENTION

The invention relates to the use of miRNA-193a for regulating gene expression, particularly it relates to the use of miRNA-193a as a PTEN agonist. This allows the advantageous treatment of PTEN-deficient conditions such as various cancers. The invention further relates to compositions comprising the miRNA for use as a PTEN agonist.

### BACKGROUND ART

MicroRNAs (miRNAs) are naturally occurring single-stranded, non-coding small RNA molecules that control gene expression by binding to complementary sequences in their target mRNAs, thereby inhibiting translation or inducing mRNA degradation. miRNAs have recently emerged as key regulators of gene expression during development and are frequently misexpressed in human disease states, for example in cancer. In fact, miRNAs can be used to silence specific cancer genes. Several miRNAs are reported to be effective modulators of cancer. For example, miRNA-193a has been described as effective in treating melanoma (WO2012005572).

Phosphatase and tensin homolog (PTEN) is 47-kDa protein and was first identified as a candidate tumour suppressor gene in 1997 after its positional cloning from a region of chromosome 10q23 known to exhibit loss in a wide spectrum of tumour types. Since then, mutations of PTEN have been detected in a variety of human cancers including breast, thyroid, glioblastoma, endometrial, and prostate cancer, and melanoma. Inherited mutations in this gene also predispose carriers to develop Cowden's disease, a heritable cancer risk syndrome, and several related conditions. PTEN is classified as a tumour suppressor because in various cancers its activity is lost by deletion, mutation, or through epigenetic changes. Molecular mechanistic studies of PTEN have provided insight into the basis for its involvement in tumour suppression. The PTEN protein has both protein phosphatase and lipid phosphatase activity. Although the tumour suppressive function of PTEN has mainly been attributed to its lipid phosphatase activity, a role for PTEN protein phosphatase activity in cell-cycle regulation and inhibition of cell invasion in vitro has been suggested as well. Loss of PTEN function seems to be responsible for many of the phenotypic features of PTEN-deficient melanoma, thus PTEN may serve as a potential target for drug development. Even when mutation of PTEN has minimal effects, it frequently contributes to tumorigenesis in the context of other genetic alterations (Aguissa-Toure et al., Cellular and Molecular Life Sciences 69: 1475-1491 (2012)).

PTEN agonists are known in the art, and their use in treating cancer has been described (WO2009126842). Their activity can stem from inhibition of mTOR. Known PTEN agonists include rapamycin (sirolimus) and its chemical analogues such as CCI-779 (temsirolimus), and RAD-001 (everolimus). Many PTEN agonists are small molecules (i.e., a compound having relatively low molecular weight, most often less than 500 or 600 kDa, or about 1000 kDa in the case of a macrolide such as rapamycin). Other agonists include monoclonal antibodies, and zinc finger proteins or nucleic acids encoding the same, engineered to bind to and activate transcription of PTEN (see WO 00/00388). Other PTEN agonists are described in US20070280918. Exemplary sequences for human PTEN and mTOR(FRAPI) are assigned UniProtKB/Swiss-Prot accession numbers P60484 and P42345. A disadvantage of PTEN agonists is that they are associated with several adverse effects. For example, the PTEN agonist sirolimus is commonly (over 30% occurrence) associated with effects as diverse as peripheral edema, hypercholesterolemia, abdominal pain, headache, nausea, diarrhea, pain, constipation, hypertriglyceridemia, hypertension, increased creatinine, fever, urinary tract infection, anemia, arthralgia, and thrombocytopenia, in addition to diabetes-like symptoms, and even an increased risk for contracting skin cancers from exposure to UV radiation (see ““Rapamune Prescribing Information”, United States Food and Drug Administration, Wyeth Pharmaceuticals, Inc. May 2015). The PTEN agonist temsirolimus is associated with fatigue, skin rash, mucositis, decreased haemoglobin, and decreased lymphocytes (Bellmunt et al., Annals of Oncology, 2008 DOI: 10.1093/annonc/mdn066).

Accordingly, there is an ongoing need for alternative and improved PTEN agonists. There is an ongoing need for improved microRNA therapies for tumours, as well as an ongoing need for deeper mechanistic insight into microRNA treatment of tumours, which can open up new strategies for treatment.

### SUMMARY OF THE INVENTION

The invention provides a miRNA-193a or a source thereof, for use in treating a condition associated with PTEN-deficiency. Preferably the miRNA-193a is a PTEN agonist. Preferably the miRNA-193a is a miRNA-193a molecule, an isomiR, or a mimic thereof, wherein it is preferably an oligonucleotide with a seed sequence comprising at least 6 of the 7 nucleotides of the seed sequence represented by SEQ ID NO: 22. Preferably the source of a miRNA is a precursor of a miRNA and is a nucleic acid of at least 50 nucleotides in length. Preferably, said miRNA shares at least 70% sequence identity with any one of SEQ ID NOs: 56, 121, or 122, and/or said miRNA is from 15-30 nucleotides in length, and/or said source of a miRNA is a precursor of said miRNA and shares at least 70% sequence identity with any one of SEQ ID NOs: 5 or 13. Preferably, the condition associated with PTEN deficiency is a PTEN-deficient cancer. Preferably, the PTEN-deficient cancer is a PTEN-deficient sarcoma, brain cancer, head and neck cancer, breast cancer, lung cancer, kidney cancer, liver cancer, colon cancer, ovarian cancer, melanoma, pancreatic cancer, thyroid cancer, hamartoma, tumour of the haematopoietic and lymphoid malignancy, or prostate cancer. Preferably, the miRNA-193a modulates expression of a gene selected from the group consisting of RPS6KB2, KRAS, PDGFRB, SOS2, TGFBR3, CASP9, INPPL1, PIK3R1, PTK2, CBL, PDPK1, CCND1, BCAR1, MAG13, MDM2, YWHAZ, and MCL1, preferably from the group consisting of RPS6KB2, KRAS, PDGFRB, CASP9, INPPL1, PIK3R1, PTK2, CBL, PDPK1, CCND1, BCAR1, MAG13, MDM2, YWHAZ, MCL1, more preferably selected from PDPK1 or INPPL1.

The invention further provides a composition comprising a miRNA-193a or a source thereof as defined above, for use as defined above. Preferably the composition further comprises a further miRNA or precursor thereof, wherein the further miRNA is selected from the group consisting of miRNA-323, miRNA-342, miRNA-520f, miRNA-520f-i3, miRNA-3157, and miRNA-7, or an isomiR thereof, or a mimic thereof. It preferably further comprises an additional pharmaceutically active compound, preferably selected from the group consisting of a PP2A methylating agent, an inhibitor of hepatocyte growth factor (HGF), an antibody, a PI3K inhibitor, an Akt inhibitor, an mTOR inhibitor, a binder of a T cell co-stimulatory molecule such as a binder of OX40, and a chemotherapeutic agent.

The invention further provides a nanoparticle composition, for use as defined above, the nanoparticle comprising a diamino lipid and a miRNA-193a or a source thereof as defined in any one of claims 1-8, wherein the diamino lipid is of general formula (I)

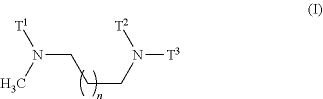

wherein
n is 0, 1, or 2, and
   T¹, T², and T³ are each independently a C₁₀-C₁₈ chain with optional unsaturations and with zero, one, two, three, or four substitutions, wherein the substitutions are selected from the group consisting of C₁-C₄ alkyl, C₁-C₄ alkenyl, and C₁-C₄ alkoxy.

Preferably the nanoparticles comprise 20-60 mol % of diamino lipid, and 0-40 mol % of a phospholipid, and 30-70 mol % of a sterol, and 0-10 mol % of a conjugate of a water soluble polymer and a lipophilic anchor.

The invention also provides an in vivo, in vitro, or ex vivo method for agonising PTEN, the method comprising the step of contacting a cell with a miRNA as defined above, or with a composition as defined above.

The invention also provides a method for treating a PTEN-deficient cancer, the method comprising the step of administering to a subject a miRNA-193a as defined above, or a composition as defined above.

### DESCRIPTION OF EMBODIMENTS

Surprisingly, the inventors identified miRNA-193a as a PTEN agonist, allowing the use of miRNA-193a for treating diseases or conditions associated with PTEN-deficiency, particularly PTEN-deficient tumours. Accordingly, the invention provides a miRNA-193a or a source thereof, for use in treating a condition associated with PTEN-deficiency. Such a miRNA-193a or a source thereof is referred to hereinafter as a miRNA for use according to the invention, or a miRNA-193a for use according to the invention. Preferably, the miRNA for use according to the invention is a PTEN agonist.

As used herein, an “agonist of PTEN” or “PTEN agonist” refers to an agent that stimulates the production of PTEN mRNA in a cell, or stimulates expression of PTEN protein in a cell, or stimulates the activity of PTEN protein, or which can provide one or more of the functions of PTEN, e.g., in regulating the PTEN pathway or the PI3K/Akt/mTOR pathway. For example, PTEN is able to indirectly reduce the activity of mTOR (mammalian target of rapamycin) by downregulating the activity of Akt. An inhibitor of mTOR directly reproduces this particular role of PTEN—reduction of mTOR activity—so such an inhibitor is considered herein to be a PTEN agonist. This type of PTEN agonist will replace some but not necessarily all the functions of the tumour suppressor PTEN in a tumour cell with mutated, deleted, or dysfuctional PTEN, and may therefore cause the cell to revert to a more normal, less malignant phenotype. Insofar as a protein has more than one known form in a species due to natural allelic variation between individuals, an inhibitor can bind to and inhibit any, or all, of such known allelic forms, and preferably binds to and inhibits the wildtype, most common or first published allelic form.

miRNA, isomiR, Mimic, or a Source Thereof

MicroRNAs (miRNAs) are small RNAs of 17-25 nucleotides, which function as regulators of gene expression in eukaryotes. miRNAs are initially expressed in the nucleus as part of long primary transcripts called primary miRNAs (pri-miRNAs). Inside the nucleus, pri-miRNAs are partially digested by the enzyme Drosha, to form 65-120 nucleotide-long hairpin precursor miRNAs (pre-miRNAs) that are exported to the cytoplasm for further processing by Dicer into shorter, mature miRNAs, which are the active molecules. In animals, these short RNAs comprise a 5′ proximal “seed” region (generally nucleotides 2 to 8) which appears to be the primary determinant of the pairing specificity of the miRNA to the 3′ untranslated region (3′-UTR) of a target mRNA.

Each of the definitions given below concerning a miRNA molecule, a miRNA mimic or a miRNA isomiR or a source of any of those is to be used for each of the identified miRNAs, molecules or mimics or isomiRs or sources thereof mentioned in this application: miRNA-193a, miRNA-323, miRNA-342, miRNA-520f, miRNA-520f-i3, miRNA-3157, and miRNA-7, or isomiRs or mimics or sources thereof. Preferred mature sequences (SEQ ID NOs: 51-57), seed sequences (SEQ ID NOs: 17-50, where SEQ ID NOs: 17-23 are seed sequences for canonical miRNAs and SEQ ID NOs: 24-50 are seed sequences for isomiRs), isomiR sequences (SEQ ID NOs: 58-125), or source sequences (RNA precursor as SEQ ID NOs: 1-8, or DNA encoding a RNA precursor as SEQ ID NOs: 9-16) of said miRNA molecule or mimic or isomiR thereof respectively are identified in the sequence listing.

In the context of this invention, a miRNA-193a refers to a miRNA-193a molecule (that is to the canonical oligonucleotide) or to an isomiR thereof or to a mimic thereof. Preferably, miRNA-193a is a miRNA-193a-3p, more preferably a miRNA-193a-3p molecule, isomiR, or mimic thereof, and comprises at least 6 of the 7 nucleotides present in the seed sequence of SEQ ID NO: 22 and more preferably has a length of at least 6, 7, 8, 9, 10, 11, 12, 13, 14, 15, 16, 17, 18, 19, 20, 21, 22, 23, 24, 25, 26, 27, 28, 29, 30 nucleotides or more. For a miRNA-193a molecules (that is for the canonical miRNA) the preferred seed sequence is SEQ ID NO: 22. For an isomiR of miRNA-193a a preferred seed sequence is SEQ ID NO: 22.

A preferred mimic of miRNA-193a has a sense strand and an antisense strand, wherein the antisense strand comprises at least 6 of the 7 nucleotides present in the seed sequence of SEQ ID NO: 22 and wherein the antisense strand preferably has a length of at least 6, 7, 8, 9, 10, 11, 12, 13, 14, 15, 16, 17, 18, 19, 20, 21, 22, 23, 24, 25, 26, 27, 28, 29, 30 nucleotides or more, and wherein the antisense strand preferably has at least 70%, 75%, 80%, 85%, 90%, 95%, 97%, 98%, 99% or 100% identity over SEQ ID NOs: 56, 121, 122, or 219, preferably 56 or 219, more preferably 219, and wherein the sense strand preferably has at least 70%, 75%, 80%, 85%, 90%, 95%, 97%, 98%, 99% or 100% identity over SEQ ID NOs: 131, 196, 197, 206, or 218, more preferably 218, and wherein the sense strand preferably has a length of at least 6, 7, 8, 9, 10, 11, 12, 13, 14, 15, 16, 17, 18, 19, 20, 21, 22, 23, 24, 25, 26, 27, 28, 29, 30 nucleotides or more.

A mimic is a molecule which has a similar or identical activity with a miRNA molecule. In this context a similar activity is given the same meaning as an acceptable level of an activity. A mimic is, in a functional determination, opposed to an antagomir. Preferred mimics are synthetic oligonucleotides, preferably comprising one or more nucleotide analogues such as locked nucleic acid monomers, and/or nucleotides comprising scaffold modifications and/or nucleotides comprising base modifications. A mimic can be a mimic for a miRNA or for an isomiR. Preferred mimics are mimics for a miRNA or for an isomiR. Preferred mimics are double stranded mimics.

Preferred mimics are double stranded oligonucleotides comprising a sense strand (also referred to as a passenger strand) and an antisense strand (also referred to as a guide strand). The canonical miRNA as it naturally occurs is defined herein as having an antisense sequence, because it is complementary to the sense sequence of naturally occurring targets. It follows that in a double stranded mimic as is a preferred mimic for use according to the invention, there are two strands, one of which is designated as a sense strand, and one of which is designated as an antisense strand. The antisense strand can have the same sequence as a miRNA, or as a precursor of a miRNA, or as an isomiR, or it can have the same sequence as a fragment thereof, or comprise the same sequence, or comprise the same sequence as a fragment thereof. The sense strand is at least partially reverse complementary to the antisense strand, to allow formation of the double stranded mimic. The sense strand is not necessarily biologically active per se, one of its important functions is to stabilize the antisense strand or to prevent its degradation or to facilitate its delivery. An examples of a sense strand for a mature miRNA is SEQ ID NO: 131. Examples of sense strands for isomiRs are SEQ ID NOs: 196 or 197.

A preferred mimic of miRNA-193a has a sense strand and an antisense strand, wherein the antisense strand comprises at least 6 of the 7 nucleotides present in the seed sequence of SEQ ID NO: 22 and wherein the antisense strand preferably has a length of at least 6, 7, 8, 9, 10, 11, 12, 13, 14, 15, 16, 17, 18, 19, 20, 21, 22, 23, 24, 25, 26, 27, 28, 29, 30 nucleotides or more, and wherein the antisense strand preferably has at least 70%, 75%, 80%, 85%, 90%, 95%, 97%, 98%, 99% or 100% identity over SEQ ID NOs: 56, 121, 122, or 219, preferably 56, more preferably 219, and wherein the sense strand preferably has at least 70%, 75%, 80%, 85%, 90%, 95%, 97%, 98%, 99% or 100% identity over SEQ ID NOs: 131, 196, 197, 206, or 218, more preferably 218, and wherein the sense strand preferably has a length of at least 6, 7, 8, 9, 10, 11, 12, 13, 14, 15, 16, 17, 18, 19, 20, 21, 22, 23, 24, 25, 26, 27, 28, 29, 30 nucleotides or more.

In preferred embodiments an antisense strand comprises at least one modified nucleoside, preferably selected from the group consisting of a bridged nucleic acid nucleoside such as a locked nucleic acid (LNA) nucleoside, a 2′-O-alkylnucleoside such as a 2′-O-methylnucleoside, a 2′-fluoronucleoside, and a 2′-azidonucleoside, preferably a 2′-O-alkylnucleoside such as a 2′-O-methylnucleoside. It is preferred that such an at least one modified nucleoside replaces the first or the last RNA nucleoside, or replaces the second or second-to-last RNA nucleoside. In preferred embodiments at least two modified nucleosides replace the first two or the last two RNA nucleosides. More preferably both the first and the last RNA nucleosides are replaced, even more preferably both the first two and the last two. It is to be understood that the replacing modified nucleoside has the same pairing capacity as the nucleoside it replaces, preferably it has the same nucleobase. Preferably an antisense strand does not comprise modified nucleosides outside of the first two or the last two RNA nucleosides. In preferred embodiments, the last base of an antisense strand is a DNA nucleoside; more preferably the last two bases of an antisense strand are DNA nucleosides. Preferably the last one or two residues of an antisense strand form an overhang when the antisense strand forms a pair with the sense strand; more preferably the last two residues of an antisense strand form such an overhang. Preferably an antisense sense does not comprise DNA nucleosides outside of the last two nucleosides, or outside of an overhang. Preferably a sense strand comprises only RNA nucleosides.

In preferred embodiments a sense strand comprises at least one modified nucleoside, preferably selected from the group consisting of a bridged nucleic acid nucleoside such as a locked nucleic acid (LNA) nucleoside, a 2′-O-alkylnucleoside such as a 2′-O-methylnucleoside, a 2′-fluoronucleoside, and a 2′-azidonucleoside, preferably a 2′-O-alkylnucleoside such as a 2′-O-methylnucleoside. It is preferred that such an at least one modified nucleoside replaces the first or the last RNA nucleoside, or replaces the second or second-to-last RNA nucleoside. In preferred embodiments at least two modified nucleosides replace the first two or the last two RNA nucleosides. More preferably both the first and the last RNA nucleosides are replaced, even more preferably both the first two and the last two. It is to be understood that the replacing modified nucleoside has the same pairing capacity as the nucleoside it replaces, preferably it has the same nucleobase. Preferably a sense strand does not comprise modified nucleosides outside of the first two or the last two RNA nucleosides. In preferred embodiments, the 3′ prime end of the sense strand is elongated by a DNA nucleoside; more preferably the last two bases of a sense strand are DNA nucleosides, even more preferably the DNA nucleoside is deoxythymidine. Preferably the last one or two residues of a sense strand form an overhang when the sense strand forms a pair with the antisense strand; more preferably the last two residues of a sense strand form such an overhang. Preferably a sense strand does not comprise DNA nucleosides outside of the last two nucleosides, or outside of an overhang. In particularly preferred embodiments a mimic comprises an antisense strand that comprises only RNA nucleosides and a sense strand that comprises modifications as described above.

Preferably, the sense strand and the antisense strand do not fully overlap, having one, two, three, or four additional bases at their 3′-end, preferably having two additional bases at their 3′-end, forming a sticky end. Accordingly, in the corresponding antisense strand, the 3′-end one, two, three, or four bases preferably do not have a reverse complementary base in the sense strand, also forming a sticky end; more preferably the first two bases of a sense strand form a sticky end, not having complementary bases in the antisense strand. The sense strand is not necessarily biologically active, it serves primarily to increase the stability of the antisense strand. Examples of preferred sequences for sense/antisense pairs for mimics are SEQ ID NOs: 206 and 218 for sense strands, more preferably SEQ ID NO: 218 for sense strands, and SEQ ID NO: 219 for antisense strands. A preferred pair is SEQ ID NOs: 206 or 218 and SEQ ID NO: 219, more preferably SEQ ID NO: 218 and SEQ ID NO: 219.

In preferred embodiments, a mimic is a double stranded oligonucleotide comprising a sense strand and an antisense strand, wherein both strands have a length of 15 to 30 nucleotides, preferably of 17 to 27 nucleotides, wherein the antisense strand has 70, 75, 80, 85, 90, 95, 96, 97, 98, 99, or 100% sequence identity with any one of SEQ ID NOs: 56, 121, or 122, wherein the sense strand optionally has 70, 75, 80, 85, 90, 95, 96, 97, 98, 99, or 100% sequence identity with any one of SEQ ID NOs: 131, 196 or 197, preferably 131 or 196, wherein the sense strand and the antisense strand preferably can anneal to form said double stranded oligonucleotide, wherein optionally one or both ends of the oligonucleotide are sticky ends having an overlap of one, two, three, or four, preferably of two nucleotides, wherein the sense strand optionally comprises chemically modified nucleotides. Preferably, the two strands of a double stranded mimic have the same length, or differ by one, two, three, four, five, or six nucleotides in length.

Within the whole text of the application unless otherwise indicated, a miRNA may also be named a miRNA molecule, a miR, an isomiR, or a mimic, or a source or a precursor thereof. Each sequence identified herein may be identified as being SEQ ID NO as used in the text of the application or as corresponding SEQ ID NO in the sequence listing. A SEQ ID NO as identified in this application may refer to the base sequence of said miRNA, isomiR, mimic, or source thereof such as a precursor. For all SEQ ID NOs, a skilled person knows that some bases can be interchanged. For example, each instance of T can be individually substituted by U, and vice versa. An RNA sequence provided for a mature miRNA can for example be synthesized as a DNA oligonucleotide using DNA nucleotides instead of RNA nucleotides. In such a case, thymine bases can be used instead of uracil bases. Alternately, thymine bases on deoxyribose scaffolds can be used. A skilled person understands that the base pairing behaviour is more important than the exact sequence, and that T and U are generally interchangeable for such purposes. Accordingly, a mimic can be either a DNA or an RNA molecule, or a further modified oligonucleotide as defined later herein.

In the context of the invention, a miRNA molecule or a mimic or an isomiR may be a synthetic or natural or recombinant or mature or part of a mature miRNA or a human miRNA or derived from a human miRNA as further defined in the part dedicated to the general definitions. A human miRNA molecule is a miRNA molecule which is found in a human cell, tissue, organ or body fluids (i.e. endogenous human miRNA molecule). A human miRNA molecule may also be a human miRNA molecule derived from an endogenous human miRNA molecule by substitution, deletion and/or addition of a nucleotide. A miRNA molecule or a mimic or an isomiR may be a single stranded or double stranded RNA molecule.

Preferably a miRNA molecule or a mimic or an isomiR thereof is from 6 to 30 nucleotides in length, preferably 12 to 30 nucleotides in length, preferably 15 to 28 nucleotides in length, more preferably said molecule has a length of at least 6, 7, 8, 9, 10, 11, 12, 13, 14, 15, 16, 17, 18, 19, 20, 21, 22, 23, 24, 25, 26, 27, 28, 29, 30 nucleotides or more.

In a preferred embodiment, a miRNA molecule or a mimic or isomiR comprises at least 6 of the 7 nucleotides present in the seed sequence of said miRNA molecule or a mimic or isomiR thereof (SEQ ID NOs: 17-50). Preferably in this embodiment, a miRNA molecule or a mimic or isomiR is from 6 to 30 nucleotides in length and more preferably comprises at least 6 of the 7 nucleotides present in the seed sequence of said miRNA molecule or mimic or isomiR. Even more preferably a miRNA molecule or a mimic or isomiR is from 15 to 28 nucleotides in length and more preferably comprises at least 6 of the 7 nucleotides present in the seed sequence, even more preferably a miRNA molecule has a length of at least 6, 7, 8, 9, 10, 11, 12, 13, 14, 15, 16, 17, 18, 19, 20, 21, 22, 23, 24, 25, 26, 27, 28, 29, 30 nucleotides or more.

In this context, to comprise at least 6 of the 7 nucleotides present in a seed sequence is intended to refer to a continuous stretch of 7 nucleotides that differs from the seed sequence in at most one position. Alternately, this can refer to a continuous stretch of 6 nucleotides that differs from the seed sequence only through omission of a single nucleotide. Throughout the application, more preferred miRNA molecules, isomiRs, mimics, or precursors thereof comprise all 7 of the 7 nucleotides present in an indicated seed sequence, or in other words have 100% sequence identity with said seed sequences. Preferably, when comprised in a miRNA, isomiR, or mimic, a seed sequence starts at nucleotide number 1, 2, or 3, and ends at nucleotide number 7, 8, 9, 10, or 11; most preferably such a seed sequence starts at nucleotide number 2 and ends at nucleotide number 8.

The miRNA-193a for use according to the invention can be combined with a further miRNA selected from the group consisting of miRNA-323, miRNA-342, miRNA-520f, miRNA-520f-i3, miRNA-3157, and miRNA-7, or an isomiR thereof, or a mimic thereof.

A preferred miRNA-323 is a miRNA-323-5p molecule, isomiR, or mimic thereof and comprises at least 6 of the 7 nucleotides present in the seed sequence of SEQ ID NOs: 17 or 24-28 and more preferably has a length of at least 6, 7, 8, 9, 10, 11, 12, 13, 14, 15, 16, 17, 18, 19, 20, 21, 22, 23, 24, 25, 26, 27, 28, 29, 30 nucleotides or more.

A preferred mimic of miRNA-323 has a sense strand and an antisense strand, wherein the antisense strand comprises at least 6 of the 7 nucleotides present in the seed sequence of SEQ ID NOs: 17 or 24-28 and wherein the antisense strand preferably has a length of at least 6, 7, 8, 9, 10, 11, 12, 13, 14, 15, 16, 17, 18, 19, 20, 21, 22, 23, 24, 25, 26, 27, 28, 29, 30 nucleotides or more, and wherein the antisense strand preferably has at least 70%, 75%, 80%, 85%, 90%, 95%, 97%, 98%, 99% or 100% identity over SEQ ID NOs: 51, 58-68, or 209 and wherein the sense strand preferably has at least 70%, 75%, 80%, 85%, 90%, 95%, 97%, 98%, 99% or 100% identity over SEQ ID NOs: 126,133-143, 201, or 208 and wherein the sense strand preferably has a length of at least 6, 7, 8, 9, 10, 11, 12, 13, 14, 15, 16, 17, 18, 19, 20, 21, 22, 23, 24, 25, 26, 27, 28, 29, 30 nucleotides or more.

A preferred miRNA-342 is a miRNA-342-5p molecule, isomiR, or mimic thereof and comprises at least 6 of the 7 nucleotides present in the seed sequence of SEQ ID NOs: 18 or 29-42 and more preferably has a length of at least 6, 7, 8, 9, 10, 11, 12, 13, 14, 15, 16, 17, 18, 19, 20, 21, 22, 23, 24, 25, 26, 27, 28, 29, 30 nucleotides or more.

A preferred mimic of miRNA-342 has a sense strand and an antisense strand, wherein the antisense strand comprises at least 6 of the 7 nucleotides present in the seed sequence of SEQ ID NOs: 18 or 29-42 and wherein the antisense strand preferably has a length of at least 6, 7, 8, 9, 10, 11, 12, 13, 14, 15, 16, 17, 18, 19, 20, 21, 22, 23, 24, 25, 26, 27, 28, 29, 30 nucleotides or more, and wherein the antisense strand preferably has at least 70%, 75%, 80%, 85%, 90%, 95%, 97%, 98%, 99% or 100% identity over SEQ ID NOs: 52, 69-113, or 211 and wherein the sense strand preferably has at least 70%, 75%, 80%, 85%, 90%, 95%, 97%, 98%, 99% or 100% identity over SEQ ID NOs: 127,144-188, 202, or 210 and wherein the sense strand preferably has a length of at least 6, 7, 8, 9, 10, 11, 12, 13, 14, 15, 16, 17, 18, 19, 20, 21, 22, 23, 24, 25, 26, 27, 28, 29, 30 nucleotides or more.

A preferred miRNA-520f is a miRNA-520f-3p molecule, isomiR, or mimic thereof and comprises at least 6 of the 7 nucleotides present in the seed sequence of SEQ ID NOs: 19 or 43-44 and more preferably has a length of at least 6, 7, 8, 9, 10, 11, 12, 13, 14, 15, 16, 17, 18, 19, 20, 21, 22, 23, 24, 25, 26, 27, 28, 29, 30 nucleotides or more.

A preferred mimic of miRNA-520f has a sense strand and an antisense strand, wherein the antisense strand comprises at least 6 of the 7 nucleotides present in the seed sequence of SEQ ID NOs: 19 or 43-44 and wherein the antisense strand preferably has a length of at least 6, 7, 8, 9, 10, 11, 12, 13, 14, 15, 16, 17, 18, 19, 20, 21, 22, 23, 24, 25, 26, 27, 28, 29, 30 nucleotides or more, and wherein the antisense strand preferably has at least 70%, 75%, 80%, 85%, 90%, 95%, 97%, 98%, 99% or 100% identity over SEQ ID NOs: 53, 114, 115, or 213 and wherein the sense strand preferably has at least 70%, 75%, 80%, 85%, 90%, 95%, 97%, 98%, 99% or 100% identity over SEQ ID NOs: 128, 189, 190, 203, or 212, and wherein the sense strand preferably has a length of at least 6, 7, 8, 9, 10, 11, 12, 13, 14, 15, 16, 17, 18, 19, 20, 21, 22, 23, 24, 25, 26, 27, 28, 29, 30 nucleotides or more.

A further preferred miRNA-520f is a miRNA-520f-3p-i3 molecule or mimic thereof comprises at least 6 of the 7 nucleotides present in the seed sequence of SEQ ID NO: 20 and more preferably has a length of at least 6, 7, 8, 9, 10, 11, 12, 13, 14, 15, 16, 17, 18, 19, 20, 21, 22, 23, 24, 25, 26, 27, 28, 29, 30 nucleotides or more.

A preferred mimic of miRNA-520f-3p-i3 has a sense strand and an antisense strand, wherein the antisense strand comprises at least 6 of the 7 nucleotides present in the seed sequence of SEQ ID NO: 20 and wherein the antisense strand preferably has a length of at least 6, 7, 8, 9, 10, 11, 12, 13, 14, 15, 16, 17, 18, 19, 20, 21, 22, 23, 24, 25, 26, 27, 28, 29, 30 nucleotides or more, and wherein the antisense strand preferably has at least 70%, 75%, 80%, 85%, 90%, 95%, 97%, 98%, 99% or 100% identity over SEQ ID NOs: 54 or 215, and wherein the sense strand preferably has at least 70%, 75%, 80%, 85%, 90%, 95%, 97%, 98%, 99% or 100% identity over SEQ ID NOs: 129, 204, or 214 and wherein the sense strand preferably has a length of at least 6, 7, 8, 9, 10, 11, 12, 13, 14, 15, 16, 17, 18, 19, 20, 21, 22, 23, 24, 25, 26, 27, 28, 29, 30 nucleotides or more.

A preferred miRNA-3157 is a miRNA-3157-5p molecule, isomiR, or mimic thereof and comprises at least 6 of the 7 nucleotides present in the seed sequence of SEQ ID NOs: 21 or 45-48 and more preferably has a length of at least 6, 7, 8, 9, 10, 11, 12, 13, 14, 15, 16, 17, 18, 19, 20, 21, 22, 23, 24, 25, 26, 27, 28, 29, 30 nucleotides or more.

A preferred mimic of miRNA-3157 has a sense strand and an antisense strand, wherein the antisense strand comprises at least 6 of the 7 nucleotides present in the seed sequence of SEQ ID NOs: 21 or 45-48 and wherein the antisense strand preferably has a length of at least 6, 7, 8, 9, 10, 11, 12, 13, 14, 15, 16, 17, 18, 19, 20, 21, 22, 23, 24, 25, 26, 27, 28, 29, 30 nucleotides or more, and wherein the antisense strand preferably has at least 70%, 75%, 80%, 85%, 90%, 95%, 97%, 98%, 99% or 100% identity over SEQ ID NOs: 55, 116-120, or 217, and wherein the sense strand preferably has at least 70%, 75%, 80%, 85%, 90%, 95%, 97%, 98%, 99% or 100% identity over SEQ ID NOs: 130, 191-195, 205, or 216, and wherein the sense strand preferably has a length of at least 6, 7, 8, 9, 10, 11, 12, 13, 14, 15, 16, 17, 18, 19, 20, 21, 22, 23, 24, 25, 26, 27, 28, 29, 30 nucleotides or more.

A preferred miRNA-7 is a miRNA-7-5p molecule, isomiR, or mimic thereof and comprises at least 6 of the 7 nucleotides present in the seed sequence of SEQ ID NOs: 23 or 50 and more preferably has a length of at least 6, 7, 8, 9, 10, 11, 12, 13, 14, 15, 16, 17, 18, 19, 20, 21, 22, 23, 24, 25, 26, 27, 28, 29, 30 nucleotides or more.

A preferred mimic of miRNA-7 has a sense strand and an antisense strand, wherein the antisense strand comprises at least 6 of the 7 nucleotides present in the seed sequence of SEQ ID NOs: 23 or 50 and wherein the antisense strand preferably has a length of at least 6, 7, 8, 9, 10, 11, 12, 13, 14, 15, 16, 17, 18, 19, 20, 21, 22, 23, 24, 25, 26, 27, 28, 29, 30 nucleotides or more, and wherein the antisense strand preferably has at least 70%, 75%, 80%, 85%, 90%, 95%, 97%, 98%, 99% or 100% identity over SEQ ID NOs: 57, 123-125, or 221, and wherein the sense strand preferably has at least 70%, 75%, 80%, 85%, 90%, 95%, 97%, 98%, 99% or 100% identity over SEQ ID NOs: 132, 198-200, 207, or 220, and wherein the sense strand preferably has a length of at least 6, 7, 8, 9, 10, 11, 12, 13, 14, 15, 16, 17, 18, 19, 20, 21, 22, 23, 24, 25, 26, 27, 28, 29, 30 nucleotides or more.

Preferably, a miRNA molecule, isomiR, or mimic thereof has a length of at least 6, 7, 8, 9, 10, 11, 12, 13, 14, 15, 16, 17, 18, 19, 20, 21, 22, 23, 24, 25, 26, 27, 28, 29, 30 nucleotides or more, comprises at least 6 of the 7 nucleotides present in a given seed sequence of any one of SEQ ID NOs: 17-50 and has at least 70% identity over the whole mature sequence of any one of SEQ ID NOs: 51-125. Preferably, identity is at least 75%, 80%, 85%, 90%, 95%, 97%, 98%, 99% or 100%.

Alternatively, preferably, a miRNA molecule, isomiR, or mimic thereof has a length of not more than 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 nucleotides, comprises at least 6 of the 7 nucleotides present in a given seed sequence of any one of SEQ ID NOs: 17-50 and has at least 70% identity over the whole mature sequence of any one of SEQ ID NOs: 51-125. Preferably, identity is at least 75%, 80%, 85%, 90%, 95%, 97%, 98%, 99% or 100%.

In another preferred embodiment, an isomiR of a miRNA molecule has at least 70% identity over the whole isomiR sequence of any one of SEQ ID NOs: 58-125. Preferably, identity is at least 75%, 80%, 85%, 90%, 95% or higher. Preferably in this embodiment, an isomiR of a miRNA molecule or a mimic thereof has a length of at least 6, 7, 8, 9, 10, 11, 12, 13, 14, 15, 16, 17, 18, 19, 20, 21, 22, 23, 24, 25, 26, 27, 28, 29, 30 nucleotides or more.

Accordingly a preferred miRNA-323 molecule, isomiR, or mimic thereof is a miRNA-323-5p molecule, isomiR, or mimic thereof and comprises at least 6 of the 7 nucleotides present in the seed sequence identified as SEQ ID NOs: 17, 24-28 and/or has at least 70%, 75%, 80%, 85%, 90%, 95%, 97%, 98%, 99% or 100% identity over SEQ ID NOs: 51, 58-68 and/or has a length of at least 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 nucleotides or more.

Accordingly a preferred miRNA-323 molecule, isomiR, or mimic thereof is a miRNA-323-5p molecule, isomiR, or mimic thereof and comprises at least 6 of the 7 nucleotides present in the seed sequence identified as SEQ ID NOs: 17, 24-28 and/or has at least 70%, 75%, 80%, 85%, 90%, 95%, 97%, 98%, 99% or 100% identity over SEQ ID NOs: 51, 58-68 and/or has a length of at least 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 nucleotides or more.

Accordingly a preferred miRNA-342 molecule, isomiR, or mimic thereof is a miRNA-342-5p molecule, isomiR, or mimic thereof and comprises at least 6 of the 7 nucleotides present in the seed sequence identified as SEQ ID NOs: 18, 29-42 and/or has at least 70%, 75%, 80%, 85%, 90%, 95%, 97%, 98%, 99% or 100% identity over SEQ ID NOs: 52, 69-113 and/or has a length of at least 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 nucleotides or more.

Accordingly a preferred miRNA-520f molecule, isomiR, or mimic thereof is a miRNA-520f-3p molecule, isomiR, or mimic thereof and comprises at least 6 of the 7 nucleotides present in the seed sequence identified as SEQ ID NOs: 19, 43-44 and/or has at least 70%, 75%, 80%, 85%, 90%, 95%, 97%, 98%, 99% or 100% identity over SEQ ID NOs: 53, 114-115 and/or has a length of at least 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 nucleotides or more. A further preferred miRNA 520f molecule, isomiR, or mimic thereof is a miRNA-520f-3p-i3 molecule or a mimic thereof and comprises at least 6 of the 7 nucleotides present in the seed sequence identified as SEQ ID NO: 20 and/or has at least 70%, 75%, 80%, 85%, 90%, 95%, 97%, 98%, 99% or 100% identity over SEQ ID NO: 54 and/or has a length of at least 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 nucleotides or more.

Accordingly a preferred miRNA-3157 molecule, isomiR, or mimic thereof is a miRNA-3157-5p molecule, isomiR, or mimic thereof and comprises at least 6 of the 7 nucleotides present in the seed sequence identified as SEQ ID NOs: 21, 45-48 and/or has at least 70%, 75%, 80%, 85%, 90%, 95%, 97%, 98%, 99% or 100% identity over SEQ ID NOs: 55, 116-120 and/or has a length of at least 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 nucleotides or more.

Accordingly a preferred miRNA-193a molecule, isomiR, or mimic thereof is a miRNA-193a-3p molecule, isomiR, or mimic thereof and comprises at least 6 of the 7 nucleotides present in the seed sequence identified as SEQ ID NO: 22 and/or has at least 70%, 75%, 80%, 85%, 90%, 95%, 97%, 98%, 99% or 100% identity over SEQ ID NOs: 56, 121-122 and/or has a length of at least 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 nucleotides or more.

Accordingly a preferred miRNA-7 molecule, isomiR, or mimic thereof is a miRNA-7-5p molecule, isomiR, or mimic thereof and comprises at least 6 of the 7 nucleotides present in the seed sequence identified as SEQ ID NOs: 23 or 50 and/or has at least 70%, 75%, 80%, 85%, 90%, 95%, 97%, 98%, 99% or 100% identity over SEQ ID NOs: 57, 123-125 and/or has a length of at least 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 nucleotides or more.

Another preferred miRNA molecule, isomiR, or mimic thereof has at least 60% identity with a seed sequence of any one of SEQ ID NOs: 17-50, or with a mature sequence of any one of SEQ ID NOs: 51-57, or with a precursor sequence of any one of SEQ ID NOs: 1-16, preferably of any one of SEQ ID NOs: 1-8, or with a DNA encoding an RNA precursor of any one of SEQ ID NOs: 9-16, or with an isomiR sequence of any one of SEQ ID NOs: 58-125. Identity may be at least 65%, 70%, 75%, 80%, 85%, 90%, 95%, 99% or 100%. Identity is preferably assessed on the whole SEQ ID NO as identified in a given SEQ ID NO. However, identity may also be assessed on part of a given SEQ ID NO. Part may mean at least 50% of the length of the SEQ ID NO, at least 60%, 70%, 80%, 90% or 100%.

A precursor sequence may result in more than one isomiR sequences depending on the maturation process—see for example miRNA-323 (mature sequence SEQ ID NO: 51) where in certain tissues multiple isomiRs have been identified (SEQ ID NOs: 58-68). IsomiRs of a miRNA molecule stem from the same precursor, and conversely a precursor can lead to multiple miRNA molecules, one of which is referred to as the canonical miRNA (such as miRNA-323-5p, SEQ ID NO: 51) and others being referred to as isomiRs (such as the oligonucleotide represented by SEQ ID NOs: 58-68). The difference between a canonical miRNA and its isomiRs can be said lie only in their prevalence—generally, the most prevalent molecule is called the canonical miRNA, while the others are isomiRs. Dependent on the type, environment, position in its life cycle, or pathological state of a cell, individual isomiRs or miRNAs can be expressed at different levels; expression can even differ between population groups or gender (Loher et al., Oncotarget (2014) DOI: 10.18632/oncotarget.2405).

The chemical structure of the nucleotides of a miRNA molecule or mimics or sources thereof, or of a sense strand or an antisense strand in a mimic of a miRNA or of an isomiR, may be modified to increase stability, binding affinity and/or specificity. Said sense strand or antisense strand may comprise or consists of a RNA molecule or preferably a modified RNA molecule. A preferred modified RNA molecule comprises a modified sugar. One example of such modification is the introduction of a 2′-O-methyl or 2′-O-methoxyethyl group or 2′ fluoride group on the nucleic acid to improve nuclease resistance and binding affinity to RNA. Another example of such modification is the introduction of a methylene bridge connecting the 2′-0 atom and the 4′-C atom of the nucleic acid to lock the conformation (Locked Nucleic Acid (LNA)) to improve affinity towards complementary single-stranded RNA. A third example is the introduction of a phosphorothioate group as linker between nucleic acid in the RNA-strand to improve stability against a nuclease attack. A fourth modification is conjugation of a lipophilic moiety on the 3′ end of the molecule, such as cholesterol to improve stability and cellular delivery.

In a preferred embodiment, the first two bases of a sense strand of a mimic have modified sugars, preferably 2′-O-methyl modifications. In a preferred embodiment, the first two of the last four bases of a sense strand of a mimic have modified sugars, preferably 2′-O-methyl modifications. In a preferred embodiment, the first two bases and the first two of the last four bases of a sense strand of a mimic have modified sugars, preferably 2′-O-methyl modifications. In a preferred embodiment, the last two bases of a sense strand of a mimic have modified sugars, preferably 2′-O-methyl modifications. In a preferred embodiment, the first two and the last two bases of a sense strand of a mimic have modified sugars, preferably 2′-O-methyl modifications. In a preferred embodiment, the last two bases of a sense strand of a mimic are DNA bases. In a preferred embodiment, the first two bases and the first two of the last four bases of a sense strand of a mimic have modified sugars, preferably 2′-O-methyl modifications, and the last two bases of said sense strand are DNA bases. In a preferred embodiment, the first two bases of a sense strand of a mimic have modified sugars, preferably 2′-O-methyl modifications, and the last two bases of said sense strand are DNA bases. In a preferred embodiment, the first two of the last four bases of a sense strand of a mimic have modified sugars, preferably 2′-O-methyl modifications, and the last two bases of said sense strand are DNA bases.

A source of a miRNA molecule or a source of a mimic or an isomiR may be any molecule which is able to induce the production of a miRNA molecule or of a mimic or isomiR as identified herein and which preferably comprises a hairpin-like structure and/or a double stranded nucleic acid molecule. The presence of a hairpin-like structure may be assessed using the RNAshapes program (Steffen P. et al 2006) using sliding windows of 80, 100 and 120 nt or more. The hairpin-like structure is usually present in a natural or endogenous source of a miRNA molecule whereas a double-stranded nucleic acid molecule is usually present in a recombinant or synthetic source of a miRNA molecule or of an isomiR or mimic thereof.

A source of a miRNA molecule or of a mimic or an isomiR may be a single stranded, a double stranded RNA or a partially double stranded RNA or may comprise three strands, an example of which is described in WO2008/10558. As used herein partially double stranded refers to double stranded structures that also comprise single stranded structures at the 5′ and/or at the 3′ end. It may occur when each strand of a miRNA molecule does not have the same length. In general, such partial double stranded miRNA molecule may have less than 75% double stranded structure and more than 25% single stranded structure, or less than 50% double stranded structure and more than 50% single stranded structure, or more preferably less than 25%, 20% or 15% double stranded structure and more than 75%, 80%, 85% single stranded structure.

Alternatively, a source of a miRNA molecule or of a mimic or an isomiR thereof is a DNA molecule encoding a precursor of a miRNA molecule or a mimic or an isomiR thereof. Preferred DNA molecules in this context are SEQ ID NOs: 9-16. For the miRNA for use according to the invention, SEQ ID NO: 13 is preferred. The invention encompasses the use of a DNA molecule encoding a precursor of a miRNA molecule that has at least 70% identity with said SEQ ID NO: 13. Preferably, the identity is at least 75%, 80%, 85%, 90%, 95%, 97%, 98%, 99% or 100%. Preferably in this embodiment, a DNA molecule has a length of at least 50, 55, 60, 70, 75, 80, 85, 90, 95, 100, 130, 150, 200, 250, 300, 350, 400 nucleotides or more and has at least 70% identity with a DNA sequence of SEQ ID NOs: 13.

The induction of the production of a given miRNA molecule or of a mimic or an isomiR is preferably obtained when said source is introduced into a cell using one assay as defined below. Cells encompassed by the present invention are later on defined.

A preferred source of a miRNA molecule or of a mimic or an isomiR thereof is a precursor thereof, more preferably a nucleic acid encoding said miRNA molecule or a mimic or an isomiR thereof. A preferred precursor is a naturally-occurring precursor. A precursor may be a synthetic or recombinant precursor. A synthetic or recombinant precursor may be a vector that can express a naturally-occurring precursor. In preferred embodiments, this aspect provides the miRNA for use according to the invention, wherein a source of a miRNA is a precursor of a miRNA and is a nucleic acid of at least 50 nucleotides in length. In preferred embodiments is provided the miRNA-193a or a source thereof for use according to the invention, wherein said miRNA shares at least 70% sequence identity with any one of SEQ ID NOs: 56, 121, or 122, and/or wherein said miRNA is from 15-30 nucleotides in length, and/or wherein said source of a miRNA is a precursor of said miRNA and shares at least 70% sequence identity with any one of SEQ ID NOs: 5 or 13. More preferably the miRNA-193a for use according to the invention shares at least 70% sequence identity with any one of SEQ ID NOs: 56, 121, or 122, and is from 15-30 nucleotides in length; more preferably said source of a miRNA-193a is a precursor of said miRNA-193a and shares at least 70% sequence identity with any one of SEQ ID NOs: 5 or 13.

A preferred precursor of a given miRNA molecule has a sequence represented by any one of SEQ ID NOs: 1-16. The invention encompasses the use of a precursor of a miRNA molecule or of an isomiR or mimic thereof that has at least 70% identity with said sequence. Preferably, identity is at least 75%, 80%, 85%, 90%, 95%, 97%, 98%, 99% or 100%. Preferably in this embodiment, a DNA molecule has a length of at least 50, 55, 60, 70, 75, 80, 85, 90, 95, 100, 130, 150, 200, 250, 300, 350, 400 nucleotides or more and has at least 70% identity with a sequence represented by any one of SEQ ID NOs: 1-16. Preferably, in this embodiment, a precursor comprises a seed sequence that shares at least 6 of the 7 nucleotides with a seed sequence selected from the group represented by SEQ ID NOs: 17-50. More preferably, a precursor comprises a seed sequence selected from the group represented by SEQ ID NOs: 17-50. A more preferred precursor of a given miRNA molecule has a sequence represented by any one of SEQ ID NOs: 1-8. The invention encompasses the use of a precursor of a miRNA molecule or of an isomiR or mimic thereof that has at least 70% identity with said sequence. Preferably, identity is at least 75%, 80%, 85%, 90%, 95%, 97%, 98%, 99% or 100%. Preferably in this embodiment, a DNA molecule has a length of at least 50, 55, 60, 70, 75, 80, 85, 90, 95, 100, 130, 150, 200, 250, 300, 350, 400 nucleotides or more and has at least 70% identity with a sequence represented by any one of SEQ ID NOs: 1-8. Preferably, in this embodiment, a precursor comprises a seed sequence that shares at least 6 of the 7 nucleotides with a seed sequence selected from the group represented by SEQ ID NOs: 17-50. More preferably, a precursor comprises a seed sequence selected from the group represented by SEQ ID NOs: 17-50.

Accordingly, a preferred source of a miRNA-323 molecule has at least 70%, 75%, 80%, 85%, 90%, 95%, 97%, 98%, 99% or 100% identity with SEQ ID NOs: 1 or 9, preferably SEQ ID NO: 1, and optionally has a length of at least 50, 55, 60, 70, 75, 80, 85, 90, 95, 100, 130, 150, 200, 250, 300, 350, 400 nucleotides or more and optionally comprises a seed sequence that shares at least 6 of the 7 nucleotides of any one of SEQ ID NOs: 17 or 24-28. Such a source is a precursor of a miRNA-323 molecule and of miRNA-323 isomiRs.

Accordingly, a preferred source of a miRNA-342 molecule has at least 70%, 75%, 80%, 85%, 90%, 95%, 97%, 98%, 99% or 100% identity with SEQ ID NOs: 2 or 10, preferably SEQ ID NO: 2, and optionally has a length of at least 50, 55, 60, 70, 75, 80, 85, 90, 95, 100, 130, 150, 200, 250, 300, 350, 400 nucleotides or more and optionally comprises a seed sequence that shares at least 6 of the 7 nucleotides of any one of SEQ ID NOs: 18 or 29-42. Such a source is a precursor of a miRNA-342 molecule and of miRNA-342 isomiRs.

Accordingly, a preferred source of a miRNA-520f molecule has at least 70%, 75%, 80%, 85%, 90%, 95%, 97%, 98%, 99% or 100% identity with SEQ ID NOs: 3 or 11, preferably SEQ ID NO: 3, and optionally has a length of at least 50, 55, 60, 70, 75, 80, 85, 90, 95, 100, 130, 150, 200, 250, 300, 350, 400 nucleotides or more and optionally comprises a seed sequence that shares at least 6 of the 7 nucleotides of any one of SEQ ID NOs: 19, 20, 43, or 44. Such a source is a precursor of a miRNA-520f molecule and of miRNA-520f isomiRs such as miRNA-520f-3p-i3.

Accordingly, a preferred source of a miRNA-3157 molecule has at least 70%, 75%, 80%, 85%, 90%, 95%, 97%, 98%, 99% or 100% identity with SEQ ID NOs: 4 or 12, preferably SEQ ID NO: 4, and optionally has a length of at least 50, 55, 60, 70, 75, 80, 85, 90, 95, 100, 130, 150, 200, 250, 300, 350, 400 nucleotides or more and optionally comprises a seed sequence that shares at least 6 of the 7 nucleotides of any one of SEQ ID NOs: 21 or 45-48. Such a source is a precursor of a miRNA-3157 molecule and of miRNA-3157 isomiRs.

Accordingly, a preferred source of a miRNA-193a molecule has at least 70%, 75%, 80%, 85%, 90%, 95%, 97%, 98%, 99% or 100% identity with SEQ ID NOs: 5 or 13, preferably SEQ ID NO: 5, and optionally has a length of at least 50, 55, 60, 70, 75, 80, 85, 90, 95, 100, 130, 150, 200, 250, 300, 350, 400 nucleotides or more and optionally comprises a seed sequence that shares at least 6 of the 7 nucleotides of any one of SEQ ID NO: 22. Such a source is a precursor of a miRNA-193a molecule and of miRNA-193a isomiRs.

Accordingly, a preferred source of a miRNA-7 molecule has at least 70%, 75%, 80%, 85%, 90%, 95%, 97%, 98%, 99% or 100% identity with SEQ ID NOs: 6-8 or 14-16, preferably SEQ ID NOs: 6-8, and optionally has a length of at least 50, 55, 60, 70, 75, 80, 85, 90, 95, 100, 130, 150, 200, 250, 300, 350, 400 nucleotides or more and optionally comprises a seed sequence that shares at least 6 of the 7 nucleotides of any one of SEQ ID NOs: 23 or 50. Such a source is a precursor of a miRNA-7 molecule and of miRNA-7 isomiRs.

In this context, it is pointed that several precursors of a given mature miRNA molecule may lead to an identical miRNA molecule. For example, miRNA-7 may originate from precursor miRNA-7-1 or miRNA-7-2 or miRNA-7-3 (preferably identified as being SEQ ID NOs: 6, 8, or 8, respectively). Also in this context, it is pointed that several isomirs of a given mature miRNA molecule may lead to miRNA molecules with identical seed sequences. For example, mature miRNA-323-5p (SEQ ID NO: 51) and at least isomirs with SEQ ID NOs: 58 or 59 all share the same seed sequence (preferably identified as being SEQ ID NO: 17).

Preferred sources or precursors have been defined elsewhere herein. A preferred source includes or comprises an expression construct comprising a nucleic acid, i.e. DNA encoding said precursor of said miRNA, more preferably said expression construct is a viral gene therapy vector selected from gene therapy vectors based on an adenovirus, an adeno-associated virus (AAV), a herpes virus, a pox virus and a retrovirus. A preferred viral gene therapy vector is an AAV or Lentiviral vector. Other preferred vectors are oncolytic viral vectors. Such vectors are further described herein below. Alternatively, a source may be a synthetic miRNA molecule or a chemical mimic as further defined in the part dedicated to general definitions.

Conditions Associated with PTEN-Deficiency

The use according to the invention is use in treating a condition associated with PTEN-deficiency. Such a condition, or disease, is referred to herein as a PTEN-deficient condition. The invention provides this new medical use of miRNA-193a. This use can also be the use of the composition or miRNA in the manufacture of a medicament. Compositions are defined in a later section. Treatment preferably refers to preventing, ameliorating, reverting, curing and/or delaying a condition. When the PTEN-deficient condition is a PTEN-deficient tumour, preferred treatment can be obtaining an anti-tumour effect.

By the term “treating” and derivatives thereof as used herein, is meant therapeutic therapy. In reference to a particular condition, treating means: (1) to ameliorate the condition or one or more of the biological manifestations of the condition; (2) to interfere with (a) one or more points in the biological cascade that leads to or is responsible for the condition or (b) one or more of the biological manifestations of the condition; (3) to alleviate one or more of the symptoms, effects or side effects associated with the condition or one or more of the symptoms, effects or side effects associated with the condition or treatment thereof; (4) to slow the progression of the condition or one or more of the biological manifestations of the condition and/or (5) to cure said condition or one or more of the biological manifestations of the condition by eliminating or reducing (preferably to undetectable levels) one or more of the biological manifestations of the condition for a period of time considered to be a state of remission for that manifestation without additional treatment over the period of remission. One skilled in the art will understand the duration of time considered to be remission for a particular disease or condition. Prophylactic therapy is also contemplated. The skilled artisan will appreciate that “prevention” is not always an absolute term. In medicine, “prevention” is understood to refer to the prophylactic administration of a drug to substantially diminish the likelihood or severity of a condition or biological manifestation thereof, or to delay the onset of such condition or biological manifestation thereof. Prophylactic therapy is appropriate, for example, when a subject is considered at high risk for developing cancer, such as when a subject has a strong family history of cancer or when a subject has been exposed to a carcinogen, or when PTEN-deficiency is diagnosed in a patient.

T cell-mediated immunotherapies are promising cancer treatments. However, many patients still fail to respond to these therapies. The molecular determinants of immune resistance are poorly understood. Loss of PTEN in tumour cells in preclinical models of melanoma inhibits T cell-mediated tumour killing and decreases T-cell trafficking into tumours. In patients (e.g., subjects), PTEN loss correlates with decreased T-cell infiltration at tumour sites, reduced likelihood of successful T-cell expansion from resected tumours, and inferior outcomes with PD-1 inhibitor therapy. PTEN loss in tumour cells increased the expression of immunosuppressive cytokines, resulting in decreased T-cell infiltration in tumours, and inhibited autophagy, which decreased T cell-mediated cell death. Treatment with a selective R13Kb (PI3Kb) inhibitor can improve the efficacy of both anti-PD-1 and anti-CTLA-4 antibodies in murine models. These findings demonstrate that PTEN loss promotes immune resistance and support the rationale to explore combinations of immunotherapies and PI3K-AKT pathway inhibitors. See Peng et al., Cancer Discovery 6:202-216 (2016).

The PI3K pathway plays a critical role in cancer by regulating several critical cellular processes, including proliferation and survival. One of the most common ways that this pathway is activated in cancer is by loss of expression of the tumour suppressor PTEN, which is a lipid phosphatase that dampens the activity of PI3K signalling. Loss of PTEN corresponds with increased activation of the PI3K-AKT pathway in multiple tumour types. Loss of PTEN is not universal in cancer—for example, it occurs in up to 30% of melanomas.

As used herein, “PTEN deficient” or “PTEN deficiency” preferably refers to a condition caused by or exacerbated by a deficiency of the tumour suppressor function of PTEN, e.g., loss of expression of the PTEN tumour suppressor. Such deficiency preferably includes mutation in the PTEN gene, reduction or absence of PTEN protein when compared to PTEN wild-type, or mutation or absence of other genes that cause suppression of PTEN function. It more preferably includes PTEN activity or expression lost by deletion, mutation, or through epigenetic changes. Multiple mechanisms exist for the regulation of PTEN, including transcription, mRNA stability, miRNA targeting, translation, and protein stability. PTEN is transcriptionally silenced by promoter methylation in PTEN-deficient endometrial, gastric, lung, thyroid, breast and ovarian tumours, as well as glioblastoma. Mutations resulting in the loss of function or reduced levels of PTEN, as well as PTEN deletions or alteration are found in several sporadic tumours. See Aguissa-Toure et al., Cellular and Molecular Life Sciences 69: 1475-1491 (2012). A skilled person knows how to determine whether a condition such as a cancer is PTEN deficient. PTEN deficiency can be determined by methods such as Q-PCR or ELISA or immunohistochemistry. Human PTEN qPCR primer pairs are commercially available, e.g., from Sino Biological and Genecopoeia. A PTEN (Human) ELISA kit is commercially available, e.g., from BioVision and Abeam. An immunohistochemistry protocol is provided, e.g., in Sakr et al., Appl. Immunohistochem. Mol. Morphol. 18:371-374 (2010). PTEN antibodies are commercially available, e.g., from Abeam and Sino Biological. For reference, the human PTEN mRNA sequence is NCBI Accession No. NM_000314.4; the protein sequence is NCBI Accession No. AAH05821.1.

PTEN-deficient conditions are known in the art, and as described above the PTEN-deficient nature of a condition can be readily established using routine assays. Examples of conditions of which PTEN-deficient variants exist are cancer, autism, macrocephaly, benign tumours, and non-cancerous neoplasia. Preferred conditions of which PTEN-deficient variants exist are cancer, benign tumours, and non-cancerous neoplasia, which are herein collectively referred to as PTEN-deficient tumours. Examples of non-cancerous neoplasia are hamartoma such as those occurring in Bannayan-Zonana syndrome, Bannayan-Riley-Ruvalcaba syndrome, Proteus syndrome, Proteus-like syndrome, Cowden disease, PTEN hamartoma tumour syndrome (PHTS), and Lhermitte-Duclos disease. A most highly preferred PTEN-deficient tumour is a PTEN-deficient cancer.

A preferred PTEN-deficient condition is a tumour, in other words a preferred use according to the invention is in treating a PTEN-deficient tumour, more preferably a PTEN-deficient cancer. Generally, as used herein, reference to treatment of cancer is intended to refer to treatment of PTEN-deficient cancer. Unless otherwise indicated, an anti-tumour effect is preferably assessed or detected before treatment and after at least one week, two weeks, three weeks, four weeks, one month, two months, three months, four months, five months, six months or more in a treated subject. An anti-tumour effect is preferably identified in a subject as:
an inhibition of proliferation or a detectable decrease of proliferation of tumour cells or a decrease in cell viability of tumour cells or melanocytes, and/or
an increase in the capacity of differentiation of tumour cells, and/or
an increase in tumour cell death, which is equivalent to a decrease in tumour cell survival, and/or
a delay in occurrence of metastases and/or of tumour cell migration, and/or
an inhibition or prevention or delay of the increase of a tumour weight or growth, and/or
a prolongation of patient survival of at least one month, several months or more (compared to those not treated or treated with a control or compared with the subject at the onset of the treatment), and/or
a decrease in tumour size or volume.

In the context of the invention, a patient may survive and may be considered as being disease free. Alternatively, the disease or condition may have been stopped or delayed or regressed. An inhibition of the proliferation of tumour cells may be at least 20%, 30%, 40%, 50%, 55%, 60%, 65%, 70% or 75% or more. Proliferation of cells may be assessed using known techniques. An decrease in cell viability of tumour cells or melanocytes may be a decrease of at least 20%, 30%, 40%, 50%, 55%, 60%, 65%, 70% or 75% or more. Such decrease may be assessed 4 days after transfection with a given miRNA molecule, equivalent or source thereof. Cell viability may be assessed via known techniques such as the MTS assay.

Treatment of tumour or cancer can be the reduction of tumour volume or a decrease of tumour cell viability. Reduction of tumour volume can be assessed using a calliper. A decrease of tumour volume or cell viability or survival may be at least a decrease of at least 1%, 5%, 10%, 15%, 20%, 25%, 30%, 40%, 50%, 55%, 60%, 65%, 70% or 75%, or more. An induction of apoptosis in tumour cells or an induction of tumour cell death may be at least 1%, 5%, 10%, 15%, 20%, 25%, 30%, 40%, 50%, 55%, 60%, 65%, 70% or 75%, or more. Tumour cell viability or survival or death may be assessed using techniques known to the skilled person. Tumour cell viability and death may be assessed using routine imaging methods such as MRI, CT or PET, and derivatives thereof, or in biopsies. Tumour cell viability may be assessed by visualising the extension of the lesion at several time points. A decrease of 10%, 15%, 20%, 25%, 30%, 40%, 50%, 55%, 60%, 65%, 70% or 75%, or more of the lesion observed at least once will be seen as a decrease of tumour cell viability.

An inhibition of the proliferation of tumour cells may be at least 1%, 5%, 10%, 15%, 20%, 30%, 40%, 50%, 55%, 60%, 65%, 70% or 75%, or more. Proliferation of cells may be assessed using known techniques as a standard proliferation assay. Such a proliferation assay may use of vital stains such as Cell Titer Blue (Promega). This includes a substrate molecule that is converted into a fluorescent molecule by metabolic enzymes. The level of fluorescence then reflects the number of living and metabolically active cells. Alternatively, such proliferation assay may determine the mitotic index. The mitotic index is based on the number of tumour cells under proliferation stage compared to the number of total tumour cells. The labelling of proliferative cells can be performed by using the antibody Ki-67 and immunohistochemistry staining. An inhibition of the proliferation of tumours cells may be seen when the mitotic index is reduced by at least 20%, at least 30%, at least 50% or more (as described in Kearsley J. H., et al, 1990, PMID: 2372483).

A delay in occurrence of metastases and/or of tumour cell migration may be a delay of at least one week, one month, several months, one year or longer. The presence of metastases may be assessed using MRI, CT or Echography or techniques allowing the detection of circulating tumour cells (CTC). Examples of the latter tests are CellSearch CTC test (Veridex), an EpCam-based magnetic sorting of CTCs from peripheral blood.

In certain embodiments, an inhibition or a decrease of a tumour weight or a delayed tumour growth or an inhibition of a tumour growth may be of at least 1%, 5%, 10%, 15%, 20%, 30%, 40%, 50%, 55%, 60%, 65%, 70% or 75%, or more. Tumour weight or volume tumour growth may be assessed using techniques known to the skilled person. The detection of tumour growth or the detection of the proliferation of tumour cells may be assessed in vivo by measuring changes in glucose utilization by positron emission tomography with the glucose analogue 2-[¹³F]-fluor-2-deoxy-D-glucose (FDG-PET) or [¹⁸F]-3′-fluoro-3′-deoxy-L-thymidine PET. An ex vivo alternative may be staining of a tumour biopsy with Ki67. An increase in the capacity of differentiation of tumour cells may be assessed using a specific differentiation marker and following the presence of such marker on cells treated. Preferred markers or parameters are p16, Trp-1 and PLZF, c-Kit, MITF, Tyrosinase, and Melanin. This may be done using RT-PCR, western blotting or immunohistochemistry. An increase of the capacity of differentiation may be at least a detectable increase after at least one week of treatment using any of the identified techniques. Preferably, the increase is of 1%, 5%, 10%, 15%, 20%, 25%, or more, which means that the number of differentiated cells within a given sample will increase accordingly. In certain embodiments, tumour growth may be delayed at least one week, one month, two months or more. In a certain embodiment, an occurrence of metastases is delayed at least one week, two weeks, three weeks, four weeks, one months, two months, three months, four months, five months, six months or more.

In preferred embodiments, the PTEN-deficient tumour is a PTEN-deficient sarcoma, brain cancer, head and neck cancer, breast cancer, lung cancer, kidney cancer, liver cancer, colon cancer, ovarian cancer, melanoma, pancreatic cancer, thyroid cancer, hamartoma, tumour of the haematopoietic and lymphoid malignancy, or prostate cancer. In other more preferred embodiments, the PTEN-deficient tumour is a PTEN-deficient sarcoma, brain cancer, head and neck cancer, breast cancer, lung cancer, kidney cancer, liver cancer, colon cancer, ovarian cancer, pancreatic cancer, thyroid cancer, hamartoma, tumour of the haematopoietic and lymphoid malignancy, or prostate cancer. In other more preferred embodiments, the PTEN-deficient tumour is a PTEN-deficient sarcoma, brain cancer, head and neck cancer, ovarian cancer, thyroid cancer, or hamartoma. In other more preferred embodiments, the PTEN-deficient tumour is a PTEN-deficient lung cancer (preferably non small cell lung cancer), liver cancer (preferably hepatocellular carcinoma), breast cancer (preferably triple-negative breast cancer), and melanoma (preferably melanoma with an activating BRAF mutation). In other more preferred embodiments, the PTEN-deficient tumour is a PTEN-deficient lung cancer (preferably non small cell lung cancer), liver cancer (preferably hepatocellular carcinoma), or breast cancer (preferably triple-negative breast cancer).

Further examples of cancers that are suitable for treatment according to the invention include, but are not limited to, both primary and metastatic forms of head and neck, breast, lung, colon, ovary, and prostate cancers. Preferably the cancer is selected from: brain (gliomas), glioblastomas, astrocytomas, glioblastoma multiforme, Bannayan-Zonana syndrome, Cowden disease, Lhermitte-Duclos disease, breast, inflammatory breast cancer, Wilm's tumour, Ewing's sarcoma, Rhabdomyosarcoma, ependymoma, medulloblastoma, colon, head and neck, kidney, lung, liver, melanoma, ovarian, pancreatic, prostate, sarcoma, osteosarcoma, giant cell tumour of bone, thyroid, lymphoblastic T cell leukemia, Chronic myelogenous leukemia, Chronic lymphocytic leukemia, Hairy-cell leukemia, acute lymphoblastic leukemia, acute myelogenous leukemia, AML, Chronic neutrophilic leukemia, Acute lymphoblastic T cell leukemia, plasmacytoma, Immunoblastic large cell leukemia, Mantle cell leukemia, Multiple myeloma Megakaryoblastic leukemia, multiple myeloma, acute megakaryocytic leukemia, promyelocytic leukemia, Erythroleukemia, malignant lymphoma, hodgkins lymphoma, non-hodgkins lymphoma, lymphoblastic T cell lymphoma, Burkitt's lymphoma, follicular lymphoma, neuroblastoma, bladder cancer, urothelial cancer, lung cancer, vulval cancer, cervical cancer, endometrial cancer, renal cancer, mesothelioma, esophageal cancer, salivary gland cancer, hepatocellular cancer, gastric cancer, nasopharangeal cancer, buccal cancer, cancer of the mouth, GIST (gastrointestinal stromal tumour) and testicular cancer. Preferred hamartoma are Bannayan-Zonana syndrome, Bannayan-Riley-Ruvalcaba syndrome, Proteus syndrome, Proteus-like syndrome, Cowden disease, PTEN hamartoma tumour syndrome (PHTS), and Lhermitte-Duclos disease.

Additionally, examples of a cancer to be treated (when PTEN-deficient) include Barret's adenocarcinoma; billiary tract carcinomas; breast cancer; cervical cancer; cholangiocarcinoma; central nervous system tumours including primary CNS tumours such as glioblastomas, astrocytomas (e.g., glioblastoma multiforme) and ependymomas, and secondary CNS tumours (i.e., metastases to the central nervous system of tumours originating outside of the central nervous system); colorectal cancer including large intestinal colon carcinoma; gastric cancer; carcinoma of the head and neck including squamous cell carcinoma of the head and neck; hematologic cancers including leukemias and lymphomas such as acute lymphoblastic leukemia, acute myelogenous leukemia (AML), myelodysplastic syndromes, chronic myelogenous leukemia, Hodgkin's lymphoma, non-Hodgkin's lymphoma, megakaryoblastic leukemia, multiple myeloma and erythroleukemia; hepatocellular carcinoma; lung cancer including small cell lung cancer and non-small cell lung cancer; ovarian cancer; endometrial cancer; pancreatic cancer; pituitary adenoma; prostate cancer; renal cancer; sarcoma; skin cancers including melanomas; and thyroid cancers.

In preferred embodiments the cancer is selected from the group consisting of: brain (gliomas), glioblastomas, astrocytomas, glioblastoma multiforme, Bannayan-Zonana syndrome, Cowden disease, Lhermitte-Duclos disease, breast cancer, colon cancer, head and neck cancer, kidney cancer, lung cancer, liver cancer, melanoma, ovarian cancer, pancreatic cancer, prostate cancer, sarcoma and thyroid cancer. In other preferred embodiments the cancer is selected from the group consisting of: ovarian, breast cancer, pancreatic cancer and prostate cancer. In other preferred embodiments the cancer is non-small cell lung carcinoma (NSCLC), small cell lung cancer (SCLC), bladder cancer or metastatic hormone-refractory prostate cancer. In other preferred embodiments the cancer is breast cancer, thyroid cancer, glioblastoma, endometrial cancer, prostate cancer, or melanoma. In other preferred embodiments the cancer is breast cancer, thyroid cancer, glioblastoma, endometrial cancer, or prostate cancer.

In preferred embodiments, the miRNA for use according to the invention is for use in the treatment of chemotherapy-resistant cancer such as sorafenib-resistant cancer.

In preferred embodiments, the miRNA for use according to the invention is for use in the treatment of carcinoma. More preferably, the miRNA for use according to the invention is for use in the treatment of chemotherapy-resistant carcinoma such as sorafenib-resistant carcinoma.

In preferred embodiments, the miRNA for use according to the invention is for use in the treatment of hepatocellular carcinoma (HCC). More preferably, the miRNA for use according to the invention is for use in the treatment of chemotherapy-resistant HCC such as hepatocellular carcinoma (HCC) that is resistant to receptor tyrosine kinase inhibitors such as VEGF receptor inhibitors, for example axitinib, cediranib, lenvatinib, nintedanib, pazopanib, regorafenib, semaxanib, sorafenib, sunitinib, tivozanib, toceranib, or vandetanib, preferably sorafenib.

In preferred embodiments, the miRNA for use according to the invention is for use in the treatment of non-small-cell lung carcinoma (NSCLC). More preferably, the miRNA for use according to the invention is for use in the treatment of chemotherapy-resistant NSCLC such as NSCLC that is resistant to platinum-based cell-cycle nonspecific antineoplastic agents (for example carboplatin, cisplatin, dicycloplatin, nedaplatin, oxaliplatin, or satraplatin, preferably cisplatin or carboplatin), or that is resistant to taxanes (for example cabazitaxel, docetaxel, larotaxel, ortataxel, paclitaxel, or tesetaxel, preferably paclitaxel or docetaxel, more preferably paclitaxel), or that is resistant to pyrimidine-based antimetabolites (for example fluorouracil, capecitabine, doxifluridine, tegafur, carmofur, floxuridine, cytarabine, gemcitabine, azacitidine, or decitabine, preferably gemcitabine), or that is resistant to vinca alkaloids (for example vinblastine, vincristine, vinflunine, vindesine, or vinorelbine, preferably vinorelbine), or that is resistant to folic acid antimetabolites (aminopterin, methotrexate, pemetrexed, pralatrexate, or raltitrexed, preferably pemetrexed).

In preferred embodiments, the miRNA for use according to the invention is for use in the treatment of triple-negative breast cancer (TNBC). More preferably, the miRNA for use according to the invention is for use in the treatment of chemotherapy-resistant TNBC such as anthracyclin-resistant TNBC, for example TNBC resistant to aclarubicin, daunorubicin, doxorubicin, epirubicin, idarubicin, amrubicin, pirarubicin, valrubicin, or zorubicin, preferably to doxorubicin.

In preferred embodiments, the miRNA for use according to the invention is for use in the treatment of melanoma. More preferably, the miRNA for use according to the invention is for use in the treatment of chemotherapy-resistant melanoma such as melanoma that is resistant to nonclassical cell-cycle nonspecific antineoplastic agents (for example procarbazine, dacarbazine, temozolomide, altretamine, mitobronitol, or pipobroman, preferably dacarbazine or temozolomide), or that is resitant to taxanes (for example cabazitaxel, docetaxel, larotaxel, ortataxel, paclitaxel, or tesetaxel, preferably paclitaxel such as albumin-bound paclitaxel), or that is resistant to platinum-based cell-cycle nonspecific antineoplastic agents (for example carboplatin, cisplatin, dicycloplatin, nedaplatin, oxaliplatin, or satraplatin, preferably cisplatin or carboplatin), or that is resistant to vinca alkaloids (for example vinblastine, vincristine, vinflunine, vindesine, or vinorelbine, preferably vinblastine). In other preferred embodiments, the miRNA for use according to the invention is not for use in the treatment of melanoma.

In preferred embodiments, the miRNA for use according to the invention is for use in the treatment of pancreas cancer. More preferably, the miRNA for use according to the invention is for use in the treatment of chemotherapy-resistant pancreas cancer such as pancreas cancer that is resitant to taxanes (for example cabazitaxel, docetaxel, larotaxel, ortataxel, paclitaxel, or tesetaxel, preferably paclitaxel such as albumin-bound paclitaxel), or that is resistant to pyrimidine-based antimetabolites (for example fluorouracil, capecitabine, doxifluridine, tegafur, carmofur, floxuridine, cytarabine, gemcitabine, azacitidine, or decitabine, preferably fluorouracil or gemcitabine), or that is resistant to topoisomerase inhibitors (for example camptothecin, cositecan, belotecan, gimatecan, exatecan irinotecan, lurtotecan, silatecan, topotecan, rubitecan, preferably irinotecan).

In preferred embodiments, the miRNA for use according to the invention is for use in the treatment of colon cancer. More preferably, the miRNA for use according to the invention is for use in the treatment of chemotherapy-resistant colon cancer such as colon cancer that is resistant to pyrimidine-based antimetabolites (for example fluorouracil, capecitabine, doxifluridine, tegafur, carmofur, floxuridine, cytarabine, gemcitabine, azacitidine, or decitabine, preferably fluorouracil or capecitabine), or that is resistant to topoisomerase inhibitors (for example camptothecin, cositecan, belotecan, gimatecan, exatecan irinotecan, lurtotecan, silatecan, topotecan, rubitecan, preferably irinotecan), or that is resistant to platinum-based cell-cycle nonspecific antineoplastic agents (for example carboplatin, cisplatin, dicycloplatin, nedaplatin, oxaliplatin, or satraplatin, preferably oxaliplatin), or that is resistant to trifluridine or tipiracil, or a combination of trifluridine and tipiracil.

In preferred embodiments, the miRNA for use according to the invention is for use in the treatment of renal cell cancer (RCC). More preferably, the miRNA for use according to the invention is for use in the treatment of chemotherapy-resistant RCC such as RCC that is resistant to receptor tyrosine kinase inhibitors such as VEGF receptor inhibitors, for example axitinib, cediranib, lenvatinib, nintedanib, pazopanib, regorafenib, semaxanib, sorafenib, sunitinib, tivozanib, toceranib, or vandetanib, preferably suntinib, sorafenib, or pazopanib, more preferably sorafenib.

In preferred embodiments, the miRNA for use according to the invention is for use in the treatment of head and neck cancer (HNSCC). More preferably, the miRNA for use according to the invention is for use in the treatment of chemotherapy-resistant HNSCC such as HNSCC that is resistant to taxanes (for example cabazitaxel, docetaxel, larotaxel, ortataxel, paclitaxel, or tesetaxel, preferably paclitaxel or docetaxel), or that is resistant to pyrimidine-based antimetabolites (for example fluorouracil, capecitabine, doxifluridine, tegafur, carmofur, floxuridine, cytarabine, gemcitabine, azacitidine, or decitabine, preferably fluorouracil), or that is resistant to folic acid antimetabolites (aminopterin, methotrexate, pemetrexed, pralatrexate, or raltitrexed, preferably methotrexate), or that is resistant to platinum-based cell-cycle nonspecific antineoplastic agents (for example carboplatin, cisplatin, dicycloplatin, nedaplatin, oxaliplatin, or satraplatin, preferably cisplatin), or that is resistant to anthracyclins (for example aclarubicin, daunorubicin, doxorubicin, epirubicin, idarubicin, amrubicin, pirarubicin, valrubicin, or zorubicin, preferably doxorubicin), or that is resistant to intercalating crosslinking agents (for example actinomycin, bleomycin, mitomycins, plicamycin, preferably bleomycin or mitomycin).

In preferred embodiments, the miRNA for use according to the invention is for use in the treatment of prostate cancer. More preferably, the miRNA for use according to the invention is for use in the treatment of chemotherapy-resistant prostate cancer such as prostate cancer that is resistant to taxanes (for example cabazitaxel, docetaxel, larotaxel, ortataxel, paclitaxel, or tesetaxel, preferably docetaxel), or that is resistant to anthracenediones (for example mitoxantrone or pixantrone, preferably mitoxantrone), or that is resistant to alkylating antineoplastic agents (for example estrogen-based alkylating antineoplastic agents such as alestramustine, atrimustine, cytestrol acetate, estradiol mustard, estramustine, estromustine, stilbostat; or phenestrol, preferably estramustine).

In preferred embodiments, the miRNA for use according to the invention is for use in the treatment of tumours of the haematopoietic and lymphoid malignancies. More preferably, the miRNA for use according to the invention is for use in the treatment of chemotherapy-resistant tumours of the haematopoietic and lymphoid malignancies such as myeloma that is resistant to bortezomib, or that is resistant to lenalidomide, or such as lymphoma that is resistant to CHOP or to rituximab, such as resistance to cyclophosphamide or to anthracyclines such as hydroxydaunorubicin or to oncovin or to prednisone, or such as leukemia resistant to vincristine, anthracyclines such as doxorubicine, L-asparaginase, cyclophosphamide, methotrexate, 6-mercaptopurine, chlorambucil, cyclophosphamide, corticosteroids such as prednisone or prednisolone, fludarabine, pentostatin, or cladribine. Treatment of chemotherapy-resistant cancer such as sorafenib-resitant cancer as described herein can be as second line treatment when chemotherapy such as sorafenib treatment has been found to be ineffective, or to be less effective than anticipated or desired.

Solid tumours are often epithelial in origin (i.e. carcinomas). A loss of epithelial cell markers (e.g. E-cadherin) and gain of mesenchymal cell markers (e.g. N-cadherin and Vimentin) is known for patient tumour samples, including prostate cancer. Cancer cells can dedifferentiate through this so-called Epithelial to Mesenchymal Transition (EMT). During EMT, intercellular cell junctions are broken down, thereby giving tumour cells the ability to migrate and invade into the surrounding tissue or through blood vessel walls. Such phenotypic changes play a major role in dissemination of the disease and ultimately lead to disease progression, which is often associated with poor prognosis for the patients.

Loss of E-cadherin expression is considered as a molecular hallmark of EMT. EMT in tumour cells results from a transcriptional reprogramming of the cell. In particular the transcriptional repression of the E-cadherin (CDH1) gene promoter has been shown to trigger the EMT phenotype. The E-cadherin protein is one of the most important cadherin molecules mediating cell-cell contacts in epithelial cells/tissues. CDH1 is repressed by binding of the transcriptional repressors, SNAI1, SNAI2, TCF3, TWIST, ZEB1, ZEB2 or KLF8, to three so-called E-boxes in the CDH1 proximal promoter region. Inhibiting the binding of these repressors to the CDH1 promoter can revert EMT, also called mesenchymal to epithelial transition (MET), and inhibits tumour cell invasion and tumour progression.

In preferred embodiments, the miRNA for use according to the invention is for use in treatment, prevention, delay, or amelioration of a disease or a condition associated with EMT, when such a disease or condition is associated with PTEN-deficiency. Herein the miRNA is preferably combined with a miRNA-518b molecule, miRNA-520f molecule, or a miRNA-524 molecule; or an isomiR or mimic thereof, or a precursor thereof. The disease or condition associated with EMT is preferably a cancer, more preferably a bladder or prostate cancer. This use is preferably by inducing a mesenchymal to epithelial transition.

In preferred embodiments, the composition for use according to the invention (compositions for use according to the invention are defined later herein) or the miRNA for use according to the invention is for use in treatment, prevention, delay, or amelioration of cancer by downregulating the immunosuppressive tumour microenvironment. In related preferred embodiments, the composition for use according to the invention or the miRNA for use according to the invention is for use in treatment, prevention, delay, or amelioration of cancer by preventing or reducing evasion of host immunity by a tumour. Such use is preferably for preventing, inhibiting, or reducing adenosine generation, for example by inhibiting or reducing activity of cell surface ectoenzymes such as those that dephosphorylate ATP to produce adenosine. Such use is more preferably for reducing NT5E expression and/or reducing ENTPD1 expression and/or inhibiting adenosine generation. More preferably, the composition for use according to the invention or the miRNA for use according to the invention is for reducing NT5E expression. More preferably, this composition for use according to the invention or this miRNA for use according to the invention is for reducing ENTPD1 expression. More preferably, this composition for use according to the invention or this miRNA for use according to the invention is for inhibiting adenosine generation. In even more preferred embodiments, this composition for use according to the invention or this miRNA for use according to the invention is for reducing cancer cell migration, preferably for reducing adenosine-induced cancer cell migration, most preferably for reducing adenosine-induced cancer cell migration associated with NT5E expression. Reduction of NT5E or ENTPD1 expression is preferably assessed by luciferase assay or by RT-PCR. Reduction of cancer cell migration is preferably assessed by in vitro transwell assays.

In preferred embodiments, the composition for use according to the invention or the miRNA for use according to the invention is for use in treatment, prevention, delay, or amelioration of cancer by promoting or increasing G2/M arrest in cancer cells, preferably in liver cancer cells, in lung cancer cells, in pancreatic cancer cells, in carcinoma cells, or in melanoma cells, more preferably in liver cancer cells, in carcinoma cells, or in melanoma cells, even more preferably in hepatocellular carcinoma cells or in melanoma cells. Such use is preferably for reducing the expression or activity of factors that regulate cell division and/or proliferation by associating with the cytoskeleton, such as MPP2 and/or STMN1. Such use is preferably for promoting or increasing factors that bind and/or sequester cyclin-dependent kinases, such as YWHAZ and/or CCNA2. Preferably, the composition for use according to the invention or the miRNA for use according to the invention is for use in treatment, prevention, delay, or amelioration of cancer by reducing the expression or activity of at least one of MPP2, STMN1, YWHAZ, and CCNA2, more preferably by reducing the expression or activity of at least YWHAZ or STMN1, even more preferably of at least YWHAZ, most preferably of each of MPP2, STMN1, YWHAZ, and CCNA2. Increase in G2/M arrest is preferably an increase as compared to untreated cells, and is preferably an increase of at least 5, 10, 15, 20, 25, 30, 35, 40, 45, or 50% or more. It is preferably assessed by DNA staining followed by microscopy imaging to determine nucleus intensity based on DNA content. Reduction of the expression or activity of at least one of MPP2, STMN1, YWHAZ, and CCNA2 is preferably assessed using RT-PCR.

In preferred embodiments, the composition for use according to the invention or the miRNA for use according to the invention is for use in treatment, prevention, delay, or amelioration of cancer by decreasing or reducing cancer cell migration, cancer cell adhesion, or cancer cell proliferation, or by increasing or promoting cancer cell apoptosis. These cancer cells are preferably lung cancer cells, liver cancer cells, breast cancer cells, melanoma cells, or carcinoma cells, more preferably lung cancer cells, liver cancer cells, breast cancer cells, or melanoma cells, even more preferably lung cancer cells such as A549 and H460, liver cancer cells such as Hep3B and Huh7, breast cancer cells such as BT549, and skin cancer cells such as A2058. In more preferred embodiments this use in treatment, prevention, delay, or amelioration of cancer is by decreasing expression or activity of at least one gene selected from the group consisting of FOXRED2, ERMP1, NT5E, SHMT2, HYOU1, TWISTNB, AP2M1, CLSTN1, TNFRSF21, DAZAP2, C1QBP, STARD7, ATP5SL, DCAF7, DHCR24, DPY19L1, AGPAT1, SLC30A7, AIMP2, UBP1, RUSC1, DCTN5, ATP5F1, CCDC28A, SLC35D2, WSB2, SEC61A1, MPP2, FAM60A, PITPNB, and POLE3, even more preferably from the group consisting of NT5E and TNFRSF21; preferably the use as described above for apoptosis, cell migration, adhesion, and proliferation is use for apoptosis, cell migration, adhesion, and/or proliferation associated with at least one of these genes. Expression is preferably assessed by RT-PCR.

In preferred embodiments, the composition for use according to the invention or the miRNA for use according to the invention is for use in treatment, prevention, delay, or amelioration of cancer by increasing or promoting apoptosis of cancer cells, preferably by increasing or promoting apoptosis associated with at least one gene selected from the group consisting of KCNMA1, NOTCH2, TNFRSF21, YWHAZ, CADM1, NOTCH1, CRYAA, ETS1, AIMP2, SQSTM1, ZMAT3, TGM2, CECR2, PDE3A, STRADB, NIPA1, MAPK8, TP53INP1, PRNP, PRT1, GCH1, DHCR24, TGFB2, NET1, PHLDA2, and TPP1, more preferably from the group consisting of NOTCH2, TNFRSF21, YWHAZ, ETS1, TGFB2, and MAPK8. Expression or activity of the gene is preferably reduced by the composition for use according to the invention or by the miRNA for use according to the invention.

In preferred embodiments, the composition for use according to the invention or the miRNA for use according to the invention is for use in treatment, prevention, delay, or amelioration of cancer by decreasing or inhibiting angiogenesis, preferably angiogenesis associated with cancer cells, more preferably by decreasing or inhibiting angiogenesis associated with at least one gene selected from the group consisting of CRKL, CTGF, ZMIZ1, TGM2, ELK3, LOX, UBP1, PLAU, CYR61, and TGFB2, even more preferably CRKL, TGFB2 or PLAU, most preferably PLAU. Expression or activity of the gene is preferably reduced by the composition for use according to the invention or by the miRNA for use according to the invention.

In preferred embodiments, the composition for use according to the invention or the miRNA for use according to the invention is for use in treatment, prevention, delay, or amelioration of cancer by modulating the unfolded protein response in cancer cells, more preferably by modulating the unfolded protein response associated with at least one gene selected from the group consisting of ERMP1, NCEH1, SEC31A, CLSTN1, FOXRED2, SEPN1, EXTL2, HYOU1, SLC35D1, SULF2, PTPLB, HHAT, ERAP2, FAF2, DPM3, PDZD2, SEC61A1, DHCR24, IDS, MOSPD2, DPM, PRNP, and AGPAT1. Expression or activity of the gene is preferably reduced by the composition for use according to the invention or by the miRNA for use according to the invention. Modulation of the unfolded protein response is preferably an inhibition or reduction of the unfolded protein response.

In preferred embodiments, the composition for use according to the invention or the miRNA for use according to the invention is for use in treatment, prevention, delay, or amelioration of cancer by decreasing or inhibiting chemotaxis of cancer cells, more preferably by decreasing or inhibiting chemotaxis associated with at least one gene selected from the group consisting of CXCL1, RAC2, CXCL5, CYR61, PLAUR, KCNMA1, ABI2, and HPRT1, most preferably PLAUR. Expression or activity of the gene is preferably reduced by the composition for use according to the invention or by the miRNA for use according to the invention.

In preferred embodiments, the composition for use according to the invention or the miRNA for use according to the invention is for use in treatment, prevention, delay, or amelioration of cancer by decreasing or inhibiting protein transport in cancer cells, more preferably by decreasing or inhibiting protein transport associated with at least one gene selected from the group consisting of STON2, RAB11FIP5, SRP54, YWHAZ, SYNRG, GCH1, THBS4, SRP54, TOMM20, SEC31A, TPP1, SLC30A7, TGFB2, AKAP12, AP2M1, ITGB3, GNAI3, SORL1, KRAS, SLC15A1, SEC61A1, APPL1, LRP4, PLEKHA8, STRADB, SCAMP4, HFE, CADM1, ZMAT3, ARF3, VAMP8, NUP50, DHCR24, RAB11FIP5, ATP6V1B2, SQSTM1, and WNK4, even more preferably YWHAZ, TGFB2, or KRAS, most preferably YWHAZ. Expression or activity of the gene is preferably reduced by the composition for use according to the invention or by the miRNA for use according to the invention.

In preferred embodiments, the composition for use according to the invention or the miRNA for use according to the invention is for use in treatment, prevention, delay, or amelioration of cancer by decreasing or inhibiting nucleoside metabolism in cancer cells, more preferably by decreasing or inhibiting nucleoside metabolism associated with at least one gene selected from the group consisting of NUDT3, NUDT15, NUDT21, DERA, NT5E, GCH1, and HPRT1, most preferably NT5E. Expression or activity of the gene is preferably reduced by the composition for use according to the invention or by the miRNA for use according to the invention.

In preferred embodiments, the composition for use according to the invention or the miRNA for use according to the invention is for use in treatment, prevention, delay, or amelioration of cancer by decreasing or inhibiting glycosylation of cancer cells, more preferably by decreasing or inhibiting glycosylation associated with at least one gene selected from the group consisting of SLC35D1, ST3GAL5, SULF2, LAT2, GALNT1, NCEH1, ST3GAL4, CHST14, B3GNT3, DPM3, GALNT13, DHCR24, NUDT15, IDH2, PPTC7, HPRT1, EXTL2, SEC61A1, ERAP2, and GALNT14. Expression or activity of the gene is preferably reduced by the composition for use according to the invention or by the miRNA for use according to the invention.

In preferred embodiments, the composition for use according to the invention or the miRNA for use according to the invention is for use in treatment, prevention, delay, or amelioration of cancer by decreasing or inhibiting oncogenesis, more preferably by decreasing or inhibiting oncogenesis associated with at least one gene selected from the group consisting of CCND1, CBL, CXCL1, CRKL, MAX, KCNMA1, TBL1XR1, GNAI3, YWHAZ, RAC2, ETS1, PTCH1, MAPK8, LAMC2, PIK3R1, CDK6, CBL, APPL1, GNAI3, PDE3A, TGFB2, ABI2, MAX, ITGB3, LOX, CXCL5, ARPC5, PPARGC1A, and THBS4, even more preferably selected from CRKL, TGFB2, YWHAZ, ETS1, MAPK8, and CDK6, most preferably from YWHAZ, ETS1, MAPK8, and CDK6. Expression or activity of the gene is preferably reduced by the composition for use according to the invention or by the miRNA for use according to the invention.

In preferred embodiments, the composition for use according to the invention or the miRNA for use according to the invention is for use in treatment, prevention, delay, or amelioration of cancer by decreasing or inhibiting dysfunctional wound healing, more preferably by decreasing or inhibiting dysfunctional wound healing associated with at least one gene selected from the group consisting of NOTCH2, KCNMA1, CXCL1, ITGB3, PLAU, CCND1, ZMIZ1, ELK3, YWHAZ, I11, PLAUR, LOX, CTGF, and TGFB2, even more preferably selected from TGFB2, NOTCH2, PLAU, YWHAZ, and PLAUR, most preferably from NOTCH2, PLAU, YWHAZ, and optionally PLAUR. Expression or activity of the gene is preferably reduced by the composition for use according to the invention or by the miRNA for use according to the invention.

In preferred embodiments, the composition for use according to the invention or the miRNA for use according to the invention is for use in treatment, prevention, delay, or amelioration of cancer by increasing or promoting immune activation, preferably immune activation associated with an immune response against cancer, more preferably by increasing or promoting immune activation associated with at least one gene selected from the group consisting of NOTCH2, LAT2, CRKL, LRRC8A, YWHAZ, PIK3R1, IRF1, TGFB2, IL111, UNG, CDK6, and HPRT1, even more preferably selected from CRKL, TGFB2, NOTCH2, YWHAZ, and CDK6, most preferably from NOTCH2, YWHAZ, and CDK6. Expression or activity of the gene is preferably reduced by the composition for use according to the invention or by the miRNA for use according to the invention.

The invention also provides a T-cell obtained from a subject treated with a miRNA for use according to the invention or with a composition for use according to the invention. Such a T-cell can be for use in the treatment of cancer as described elsewhere herein. In its use, the T-cell is preferably previously obtained from a subject treated with a miRNA for use according to the invention or with a composition for use according to the invention. The T-cell is preferably from a human subject. It is preferably for use as a vaccine, or for preventing recurrence or metastasis of cancer.

In preferred embodiments, the composition for use according to the invention or the miRNA for use according to the invention is for use in treatment, prevention, delay, or amelioration of a cancer associated with at least one gene selected from the group consisting of CDK6, EIF4B, ETS1, IL17RD, MCL1, MAPK8, NOTCH2, NT5E, PLAU, PLAUR, TNFRSF21, and YWHAZ, more preferably selected from NOTCH2, NT5E, PLAU, PLAUR, and YWHAZ.

In preferred embodiments, the composition for use according to the invention or the miRNA for use according to the invention is for use in treatment, prevention, delay, or amelioration of a cancer associated with at least one gene selected from the group consisting of CDK4, CDK6, CRKL, NT5E, HMGB1, IL17RD, KRAS, KIT, HDAC3, RTK2, TGFB2, TNFRSF21, PLAU, NOTCH1, NOTCH2, and YAP1. These genes have known involvement in anti-tumour immunity.

In preferred embodiments, the composition for use according to the invention or the miRNA for use according to the invention is for use in treatment, prevention, delay, or amelioration of a cancer associated with at least one gene selected from the group consisting of ETS1, YWHAZ, MPP2, PLAU, CDK4, CDK6, EIF4B, RAD51, CCNA2, STMN1, and DCAF7. These genes are involved in regulation of the cell cycle.

In preferred embodiments, the composition for use according to the invention or the miRNA for use according to the invention is for use in treatment, prevention, delay, or amelioration of cancer, wherein a preferred cancer is a cancer selected from the group consisting of colon cancer such as colon carcinoma, lung cancer such as lung carcinoma, melanoma, lymphoma such as reticulum cell sarcoma, pancreas cancer such as pancreatic adenocarcinoma, liver cancer such as hepatocarcinoma or hepatoma, breast cancer such as breast carcinoma, prostate cancer, kidney cancer such as renal adenocarcinoma, carcinoma such as adenocarcinoma or colon, lung, liver, pancreas, kidney, or breast carcinoma, and adenocarcinoma such as pancreatic or renal adenocarcinoma. A more preferred cancer is a cancer selected from the group consisting of colon cancer such as colon carcinoma, lung cancer such as lung carcinoma, melanoma, lymphoma such as reticulum cell sarcoma, pancreas cancer such as pancreatic adenocarcinoma, liver cancer such as hepatocarcinoma, breast cancer such as breast carcinoma, prostate cancer, carcinoma such as adenocarcinoma or colon, lung, liver, pancreas, or breast carcinoma, and adenocarcinoma such as pancreatic adenocarcinoma. An even more preferred cancer is a cancer selected from the group consisting of colon cancer such as colon carcinoma, lung cancer such as lung carcinoma, melanoma, lymphoma such as reticulum cell sarcoma, and carcinoma such as colon or lung carcinoma.

In further preferred embodiments, the miRNA for use according to the invention is for use in the treatment of cancer wherein the composition is combined with a further chemotherapeutic agent such as sorafenib. This is referred to hereinafter as a combination according to the invention. A combination according to the invention is preferably for use as described above for the composition for use according to the invention.

A combination according to the invention is a combination comprising a composition for use according to the invention or the miRNA for use according to the invention and comprising a chemotherapeutic agent such as a kinase inhibitor drug suitable for the treatment of cancer, for example such as a combination comprising a composition for use according to the invention and comprising sorafenib, or for example comprising a miRNA for use according to the invention and comprising sorafenib.

Suitable chemotherapeutic agents are kinase inhibitor drugs such as sorafenib or B-raf inhibitors or MEK inhibitors or RNR inhibitors or AURKB inhibitors. A preferred B-raf inhibitor is vemurafenib and/or dabrafenib. A preferred MEK inhibitor is trametinib and/or selumetinib. A preferred RNR inhibitor is selected from the group consisting of gemcitabine, hydroxyurea, clolar clofarabine and triapine

B-raf inhibitors are compounds that specifically inhibit the B-raf protein, for which a mutated form of the BRAF gene encodes. Several mutations of the BRAF gene are known to cause melanoma, and specific compounds have been developed which inhibit the mutated form of the B-raf protein. B-raf inhibitors are known in the art and include, but are not limited to vemurafenib, dabrafenib, trametinib, GDC-0879, PLX-4720, sorafenib, SB590885, PLX4720, XL281 and RAF265. B-raf inhibitors are e.g. described in Wong K. K., et al. One B-raf inhibitor may be used or together with other B-raf inhibitors in a combination according to the invention. Preferred B-raf inhibitors to be used in the present invention are vemurafenib, dabrafenib or a mixture of vemurafenib and dabrafenib. Vemurafenib is also known as RG7204 or N-(3-{[5-(4-chlorophenyl)-1H-pyrrolo[2,3-b]pyridin-3-yl]carbonyl}-2,4-difluorophenyl)propane-1-sulfonamide, and marketed as Zelboraf. Dabrafenib is also known as N-{3-[5-(2-aminopyrimidin-4-yl)-2-(1,1-dimethylethyl)thiazol-4-yl]-2-fluorophenyl}-2,6-difluorobenzenesulfonamide.

MEK inhibitors are compounds that specifically inhibit a MEK protein. Several MEK inhibitors are known in the art and include, but are not limited to trametinib (GSK1120212), selumetinib (AZD-6244), XL518, CI-1040, PD035901. Trametinib is also known as N-(3-(3-cyclopropyl-5-(2-fluoro-4-iodophenylamino)-6,8-dimethyl-2,4,7-trioxo-3,4,6,7-tetrahydropyrido[4,3-d]pyrimidin-1(2H)-yl)phenyl)acetamide. Selumetinib is also known as: 6-(4-bromo-2-chlorophenylamino)-7-fluoro-N-(2-hydroxyethoxy)-3-methyl-3Hbenzo[d]imidazole-5-carboxamide. MEK inhibitors are e.g. described in Wong, K. K. (PMID: 19149686). One MEK inhibitor may be used or together with other MEK inhibitors in a combination according to the invention. Several MEK inhibitors is synonymous with several distinct MEK inhibitors. Preferred MEK inhibitors to be used in the present invention are trametinib and/or selumetinib.

RNR and/or AURKB inhibitors are compounds that specifically inhibit RNR and/or AURKB proteins. RNR is a ribonucleotide reductase (RNR) and as such is the only enzyme responsible for the de novo conversion of ribonucleoside diphosphate (NDP) to deoxyribonucleoside diphosphate (dNDP) (Zhou et al. 2013). RNR is the key regulator of intracellular dNTP supply. Maintenance of a balanced dNTP pool is a fundamental cellular function because the consequences of imbalance in the substrates for DNA synthesis and repair include mutagenesis and cell death. Human RNR is composed of a subunits (RRM1) that contain the catalytic site and two binding sites for enzyme regulators and b subunits (RRM2) with a binuclear iron cofactor that generates the stable tyrosyl radical necessary for catalysis. An inhibitor of RNR may inhibit RRM1 and/or RRM2. Preferred RNR inhibitors are selected from the group consisting of gemcitabine, hydroxyurea, clolar clofarabine and triapine.

AURKB (Aurora B kinase) is a protein that functions in the attachment of the mitotic spindle to the centromere. Chromosomal segregation during mitosis as well as meiosis is regulated by kinases and phosphatases. The Aurora kinases associate with microtubules during chromosome movement and segregation. In cancerous cells, over-expression of these enzymes causes unequal distribution of genetic information, creating aneuploid cells, a hallmark of cancer.

A chemotherapeutic agent is a drug that is able to induce or promote an anti-cancer effect as defined herein. A preferred chemotherapeutic agent is a kinase inhibitor or an RNR inhibitor or an AURKB inhibitor. Examples of such inhibitors are compounds that specifically inhibit the RNR and/or the AURKB proteins. To evaluate the ability of a therapeutic compound to inhibit RNR and/or AURKB proteins, one can perform western blotting with RNR (RRM1 and/or RRM2) or AURKB protein as read-out. Cells are plated in 6-well plates and treated for 72 hours at 0.01, 0.1 and 1 μM of said compound. After treatment cells are scraped into a lysis buffer as a RIPA lysis buffer. Equal amounts of protein extracts are separated by using 10% SDS PAGE, and then transferred to a polyvinylidene difluoride membrane. After blocking for 1 hour in a Tris-buffered saline containing 0.1% Tween 20 and 5% nonfat milk, the membrane is probed with a RNR (i.e. RRM1 and/or RRM2) and/or a AURKB primary antibody, followed by a secondary antibody conjugated to horseradish peroxidase for chemiluminescent detection on film. Tubulin is used as loading control. A preferred RRM2 antibody used is from Santa Cruz (product #sc-10846) and/or a preferred AURKB antibody is from Cell Signalling (product #3094). The evaluation of the therapeutic ability of said RNR and/or AURKB inhibitor may also be assessed at the RNA level by carrying out a Nothern blot or by PCR.

Preferred combinations according to the invention comprise:
i) a composition for use according to the invention or a miRNA for use according to the invention, and
ii) at least one chemotherapeutic agent selected from the group consisting of
   a. receptor tyrosine kinase inhibitors such as VEGF receptor inhibitors, for example axitinib, cediranib, lenvatinib, nintedanib, pazopanib, regorafenib, semaxanib, sorafenib, sunitinib, tivozanib, toceranib, or vandetanib, preferably suntinib, sorafenib, or pazopanib, more preferably sorafenib;
   b. platinum-based cell-cycle nonspecific antineoplastic agents, for example carboplatin, cisplatin, dicycloplatin, nedaplatin, oxaliplatin, or satraplatin, preferably cisplatin or carboplatin or oxaliplatin;
   c. taxanes, for example cabazitaxel, docetaxel, larotaxel, ortataxel, paclitaxel, or tesetaxel, preferably paclitaxel or docetaxel, more preferably paclitaxel or docetaxel;
   d. pyrimidine-based antimetabolites, for example fluorouracil, capecitabine, doxifluridine, tegafur, carmofur, floxuridine, cytarabine, gemcitabine, azacitidine, or decitabine, preferably fluorouracil or gemcitabine or capecitabine;
   e. vinca alkaloids, for example vinblastine, vincristine, vinflunine, vindesine, or vinorelbine, preferably vinorelbine or vinblastine;
   f. folic acid antimetabolites, aminopterin, methotrexate, pemetrexed, pralatrexate, or raltitrexed, preferably pemetrexed or methotrexate;
   g. anthracyclins, for example aclarubicin, daunorubicin, doxorubicin, epirubicin, idarubicin, amrubicin, pirarubicin, valrubicin, or zorubicin, preferably to doxorubicin;
   h. nonclassical cell-cycle nonspecific antineoplastic agents, for example procarbazine, dacarbazine, temozolomide, altretamine, mitobronitol, or pipobroman, preferably dacarbazine or temozolomide;
   i. taxanes, for example cabazitaxel, docetaxel, larotaxel, ortataxel, paclitaxel, or tesetaxel, preferably paclitaxel such as albumin-bound paclitaxel;
   j. topoisomerase inhibitors, for example camptothecin, cositecan, belotecan, gimatecan, exatecan irinotecan, lurtotecan, silatecan, topotecan, rubitecan, preferably irinotecan;
   k. trifluridine or tipiracil, or a combination of trifluridine and tipiracil;
   l. intercalating crosslinking agents, for example actinomycin, bleomycin, mitomycins, plicamycin, preferably bleomycin or mitomycin;
   m. anthracenediones, for example mitoxantrone or pixantrone, preferably mitoxantrone; and
   n. alkylating antineoplastic agents, for example estrogen-based alkylating antineoplastic agents such as alestramustine, atrimustine, cytestrol acetate, estradiol mustard, estramustine, estromustine, stilbostat; or phenestrol, preferably estramustine.

In preferred embodiments, a composition for use according to the invention or a miRNA for use according to the invention is for use in the treatment of cancer, wherein the composition increases the immune response to cancer cells. This may mean that it initiates an immune response in cases where no immune response was present.

In more preferred embodiments for increasing immune response, the composition for use according to the invention or a miRNA for use according to the invention is for increasing the production of immune system activating cytokines, such as IL-2. Preferably, cytokine production is increased by 1%, 5%, 10%, 15%, 20%, 25%, 30%, 40%, 50%, 55%, 60%, 65%, 70% or 75%, or more, and is preferably detected through FACS. Immune system activating cytokines are increased in a 4T1 mouse model for triple negative breast cancer (TNBC) after one week of treatment. The increase in cytokines leads to increased immune suppression of cancers, and can lead to immune suppression or partial immune suppression of cancers that would otherwise not be susceptible to immune suppression. In preferred embodiments, the composition for use according to the invention or a miRNA for use according to the invention is for increasing T-cell function, such as increasing production of IFNγ and IL-2.

In more preferred embodiments for increasing immune response, the composition for use according to the invention or a miRNA for use according to the invention is for decreasing regulatory T cell population. Regulatory T cells (Tregs) are immunosuppressive T regulatory cells, and decreasing Tregs increases the immune response to a cancer. Preferably, Tregs are decreased by 1%, 5%, 10%, 15%, 20%, 25%, 30%, 40%, 50%, 55%, 60%, 65%, 70% or 75%, or more. Decrease of Tregs can be determined via the determination of FOXP3 or LAG3. This effect is preferably in parallel with increased cytokine production as described above.

Recruitment of CD8+ T effector cells is increased in a 4T1 mouse model for triple negative breast cancer (TNBC) after two weeks of treatment, and T-cell function is induced, while Treg population is decreased. Accordingly, in preferred embodiments for increasing immune response, the composition for use according to the invention or a miRNA for use according to the invention is for increasing T-cell frequency. Preferably, such an increase is by 1%, 5%, 10%, 15%, 20%, 25%, 30%, 40%, 50%, 55%, 60%, 65%, 70% or 75%, or more. Such an increase can be determined by measuring CD8. In preferred embodiments for increasing immune response, the composition for use according to the invention or a miRNA for use according to the invention is for inducing T-cell function, preferably for inducing T-cell function by inducing IFNγ production. Most preferably, the composition for use according to the invention or a miRNA for use according to the invention is for increasing T-cell frequency and simultaneously inducing T-cell function, preferably while simultaneously decreasing regulatory T cell population. Tumours with decreased Tregs and with increased CD8+ T effector cells are referred to as ‘hot’ tumours, which are tumours that do not have an immunosuppressed microenvironment. Conversely, tumours in an immunosuppressed microenvironment are referred to as ‘cold’ tumours.

Additionally, compositions according to the invention can reduce expression of immune suppressive target genes such as ENTPD1 (CD39) or TIM-3. Such a reduction is preferably by 1%, 5%, 10%, 15%, 20%, 25%, 30%, 40%, 50%, 55%, 60%, 65%, 70% or 75%, or more. TIM-3 or ENTPD1 expression can be determined via qPCR. ENTPD1 is an ectonucleotidase that catalyses the hydrolysis of γ- and β-phosphate residues of triphospho- and diphosphonucleosides to the monophosphonucleoside derivative. It has an immune suppressive role through its generation of high amounts of adenosine. Reduction of ENTPD1 expression increases the immune response to tumour cells. TIM-3 is also known as hepatitis A virus cellular receptor 2 (HAVCR2), and is an immune checkpoint, an inhibitory receptor acting as an immune-suppressive marker. TIM-3 is mainly expressed on activated CD8+ T cells and suppresses macrophage activation. Reduction of TIM-3 expression increases the immune response to tumour cells. In preferred embodiments, the composition for use according to the invention or a miRNA for use according to the invention is for reducing expression of ENTPD1 or of TIM-3 or for reducing expression of ENTPD1 and TIM-3.

The positive effect of compositions according to the invention and miRNA for use according to the invention on the immune system as it relates to tumour cells and cancer cells leads to the invention being suitable for preventing the growth of new tumours, preventing metastasis, or reducing the growth of tumours that have been removed in size, for example through surgery. For example treatment with a composition for use according to the invention reduces the regrowth of surgically excised tumours, and reduces metastasis of such tumours, increasing survival in affected subjects. A tumour from which metastases derive is referred to as a primary tumour. Moreover, subjects with a particular tumour type that had been treated with a composition for use according to the invention or with a miRNA for use according to the invention show limited tumour take when re-challenged with new tumour cells of the same type that had already been treated. After the limited tumour take, the tumour fully regresses. When challenged with a different tumour type, the tumour fully takes, but also subsequently regresses entirely.

Accordingly, in preferred embodiments the compositions according to the invention and miRNA for use according to the invention are for use as a medicament for preventing, reducing, or delaying cancer or metastatic cancer. In this context, preferred cancers are breast cancer, carcinoma, and liver cancer, more preferably breast cancer and liver cancer.

Accordingly, in preferred embodiments the compositions according to the invention and miRNA for use according to the invention are for use as a cancer vaccine, preferably for use as a cancer vaccine for the prevention or treatment of cancer. Such vaccines are preferably for preventing or reducing regrowth or recurrence of primary tumours. Preferably, regrowth is reduced by 1%, 5%, 10%, 15%, 20%, 25%, 30%, 40%, 50%, 55%, 60%, 65%, 70% or 75%, or more. In another use, such vaccines are preferably for reducing or treating metastatic cancer. Preferably, metastatic cancer is reduced by 1%, 5%, 10%, 15%, 20%, 25%, 30%, 40%, 50%, 55%, 60%, 65%, 70% or 75%, or more, or motility of cancer cells is reduced by 1%, 5%, 10%, 15%, 20%, 25%, 30%, 40%, 50%, 55%, 60%, 65%, 70% or 75%, or more. In this context, preferred cancers are breast cancer, carcinoma, and liver cancer, more preferably breast cancer and liver cancer.

Accordingly, in preferred embodiments the compositions according to the invention and miRNA for use according to the invention are for use as a medicament, wherein the medicament is for the prevention, reduction, or treatment of metastatic cancer, preferably wherein the primary tumour has been surgically excised or has regressed, more preferably wherein the primary tumour has been surgically excised. Preferably, metastatic cancer is reduced by by 1%, 5%, 10%, 15%, 20%, 25%, 30%, 40%, 50%, 55%, 60%, 65%, 70% or 75%, or more. In this context, preferred cancers are breast cancer, carcinoma, and liver cancer, more preferably breast cancer and liver cancer.

Accordingly, in preferred embodiments the compositions according to the invention and miRNA for use according to the invention are for use as a medicament, wherein the medicament is for the prevention, reduction, or treatment of regrowth or recurrence of a cancer after surgical excision. Preferably, regrowth or recurrence is reduced by 1%, 5%, 10%, 15%, 20%, 25%, 30%, 40%, 50%, 55%, 60%, 65%, 70% or 75%, or more. In this context, preferred cancers are breast cancer, carcinoma, and liver cancer, more preferably breast cancer and liver cancer.

Accordingly, in preferred embodiments the compositions according to the invention and miRNA for use according to the invention are for use as a medicament, wherein the medicament is for the prevention, reduction, or treatment of regrowth or recurrence of a cancer after said cancer has regressed or has been successfully treated. Preferably, regrowth or recurrence is reduced by 1%, 5%, 10%, 15%, 20%, 25%, 30%, 40%, 50%, 55%, 60%, 65%, 70% or 75%, or more. In this context, preferred cancers are breast cancer, carcinoma, and liver cancer, more preferably breast cancer and liver cancer.

In preferred embodiments, the composition for use according to the invention or a miRNA for use according to the invention is for inhibiting proliferation of tumour cells. Compositions according to the invention can reduce K-RAS and MCL1 expression, leading to a reduced proliferation of tumour cells. K-RAS, also known as KRAS, K-ras, Ki-ras, is a proto-oncogene known in the art. MCL1 is also known as induced myeloid leukaemia cell differentiation protein Mcl-1. It can enhance cancer cell survival by inhibiting apoptosis. Both K-RAS and MCL1 enhance proliferation of cancer cells. In preferred embodiments, the composition for use according to the invention or a miRNA for use according to the invention is for reducing expression of K-RAS or of MCL1 or for reducing expression of K-RAS and MCL1. In preferred embodiments, the composition for use according to the invention or a miRNA for use according to the invention is for reducing expression of K-RAS and MCL1 and ENTPD1 and TIM-3.

Inhibition of proliferation is preferably via induction of apoptosis. Compositions according to the invention induce apoptosis in cancer cells through caspase activation and PARP inactivation through PARP cleavage. Preferred caspase activation is activation of caspase 3/7. PARP is also known as poly (ADP-ribose) polymerase and refers to a family of proteins involved in programmed cell death. It is cleaved in vivo by caspase 3 and by caspase 7, which triggers apoptosis. Cleavage of PARP can be determined through blotting techniques, and caspase activation can be assayed by determining PARP cleavage through blotting, or by qPCR. In preferred embodiments, the composition for use according to the invention or a miRNA for use according to the invention is for inducing apoptosis in cancer cells. In preferred embodiments, the composition for use according to the invention or a miRNA for use according to the invention is for activating caspase 3 and caspase 7. In preferred embodiments, the composition for use according to the invention or a miRNA for use according to the invention is for inactivating PARP. Preferably, PARP is inactivated by 1%, 5%, 10%, 15%, 20%, 25%, 30%, 40%, 50%, 55%, 60%, 65%, 70% or 75%, or more. Inactivation of PARP can be monitored by blotting techniques, detecting the smaller fragments of the uncleaved enzyme. Preferably, caspase activity is increased by 1%, 5%, 10%, 15%, 20%, 25%, 30%, 40%, 50%, 55%, 60%, 65%, 70% or 75%, or more.

In further preferred embodiments, the composition for use according to the invention or a miRNA for use according to the invention is for reducing expression of at least one of the genes selected from the group consisting of K-RAS, MCL1, ENTPD1, TIM-3, c-Kit, CyclinD1, and CD73. c-Kit is a proto-oncogene also known as tyrosine-protein kinase Kit or CD117, and codes for a receptor tyrosine kinase protein. Cyclin D1 overexpression correlates with early cancer onset and tumour progression. CD73 is also known as 5′-nucleotidase (5′-NT), and as ecto-5′-nucleotidase. The enzyme encoded by CD73 is ecto-5-prime-nucleotidase (5-prime-ribonucleotide phosphohydrolase; EC 3.1.3.5) and catalyzes the conversion at neutral pH of purine 5-prime mononucleotides to nucleosides, the preferred substrate being AMP. Expression of such genes is preferably reduced by 1%, 5%, 10%, 15%, 20%, 25%, 30%, 40%, 50%, 55%, 60%, 65%, 70% or 75%, or more, which can for example be determined via qPCR techniques.

In preferred embodiments, the composition for use according to the invention or a miRNA for use according to the invention is for regulating the adenosine A2A receptor pathway. The adenosine A2A receptor, also known as ADORA2A, is an adenosine receptor that can suppress immune cells. The activity of compositions according to the invention in reducing expression of CD73 and/or of ENTPD1, as described above, interferes with the A2A receptor pathway, reducing immune suppression. This leads to an anti-tumour effect because tumour cells ability to escape immune surveillance is reduced. In preferred embodiments, the composition for use according to the invention or a miRNA for use according to the invention is for increasing the susceptibility of tumour cells to immune surveillance. Such an increase preferably leads to a reduction of tumour volume of 1%, 5%, 10%, 15%, 20%, 25%, 30%, 40%, 50%, 55%, 60%, 65%, 70% or 75%, or more. In more preferred embodiments, the composition for use according to the invention or a miRNA for use according to the invention is for increasing the susceptibility of tumour cells to immune surveillance, while increasing recruitment of CD8+ T effector cells, preferably while decreasing Tregs, such as through reducing expression of LAG3 or of FoxP3, or of both. Increased susceptibility to immune surveillance preferably leads to reduced tumour volume.

The inventors have found that miRNA-193a modulates several pathways and genes. This activity of miRNA-193a can be used for treating conditions associated with those pathways or genes. Accordingly, in preferred embodiments is provided the miRNA-193a or a source thereof for use according to the invention, wherein the miRNA-193a modulates expression of a gene selected from the group consisting of RPS6KB2, KRAS, PDGFRB, SOS2, TGFBR3, CASP9, INPPL1, PIK3R1, PTK2, CBL, PDPK1, CCND1, BCAR1, MAGI3, MDM2, YWHAZ, and MCL1, preferably from the group consisting of RPS6KB2, KRAS, PDGFRB, CASP9, INPPL1, PIK3R1, PTK2, CBL, PDPK1, CCND1, BCAR1, MAGI3, MDM2, YWHAZ, MCL1, more preferably selected from PDPK1 or INPPL1. This modulation is preferably downregulation. In preferred embodiments PDPK1 is modulated, preferably downregulated by the miRNA. In preferred embodiments INPPL1 is modulated, preferably downregulated by the miRNA.

Modulation is defined elsewhere herein. Upregulation refers to an increased expression, which can refer to an increased transcription, production of mRNA, translation, production of gene product, and/or activity of gene product. Downregulation refers to a decreased expression, can refer to a decreased transcription, production of mRNA, translation, production of gene product, and/or activity of gene product. Preferably, upregulation and downregulation refer to transcription of production of mRNA. In other preferred embodiments upregulation and downregulation refer to activity of gene product. Upregulation and downregulation are preferably as compared to a reference, such as to a healthy cell, or such as to an untreated cell, when cultivated under otherwise identical conditions. For example, when miRNA-193a is used for downregulation of INPPL1 in a cell, then miRNA-193a preferably decreases INPPL1 expression in that cell as compared to a cell (of the same type) that has not been contacted with miRNA-193a. The change in expression is preferably at least 1, 2, 3, 4, 5, 6, 7, 8, 9, 10, 15, 20, 25, 30, 35, 40, 45, 50, 55, 60, 65, 70, 75, 80, 85, 90, 95, 96, 97, 98, 99, 100, 125, 150, 200, 250% or more, more preferably at least 50% or more, even more preferably at least 100% or more. In case of downregulation, optionally there is no longer any detectable expression after downregulation.

The invention provides a miRNA-193a molecule, isomiR, mimic, or source thereof as described herein, or a miRNA-193a for use in treating a condition associated with PTEN-deficiency such as for use as a PTEN agonist, wherein the miRNA-193a molecule, isomiR, mimic, or source thereof is for upregulation of a gene selected from the group consisting of STAT3, TMEM2, PEG10, GCC2, RFX5, CPEB2, UNKL, RNF44, PGM2L1, NACC2, TDG, IFT81, CAMK2N1, BDNF, KANK1, CPS1, HDHD1, THBD, SEMA4G, SAMD4A, RP11-438N16.1, C2orf69, TPRG1L, CHIC1, HOXC13, DYRK1B, RASA2, CELSR3, ADM, KLHDC3, ABCC5, PNKD, MOK, PBXIP1, NUAK2, CLDN2, PHLPP2, CPOX, ZNF76, MBLAC2, VTN, CDH1, RMND5A, SCML1, LMBR1L, TGFBR2, ENTPD7, LZTFL1, C7orf60, ZNF558, CTNNBIP1, SNN, IFT140, RALGAPA1, WIPI1, C8orf58, PDCD4, RPL26, HOXA10, RP11-443N24.1, EIF4EBP2, RECK, ZNF614, HBP1, PRICKLE4, WDR25, DNMT3B, GNAZ, PER2, LMLN, SARM1, C17orf97, AC011841.1, CCNG1, ANKRD46, SYNGR3, TPPP3, KIAA0513, UBE2Q2P6, FAM227A, NEK11, KLHDC7A, ARID3A, CROT, PER1, F3, FAM217B, AK7, FRAT2, FOS, STX1B, APH1B, FER1L4, SAMD15, C3orf67, SKIDA1, SESN3, CTD-2366F13.1, SESN1, PADI1, C9orf9, ZBTB3, CCAT1, LATS2, RP11-553L6.5, JHDM1D-AS1, ZNF438, CEBPD, CTB-171A8.1, TEF, CYP1A1, LIN28B, DPY19L1P1, ZNF138, LINC00886, KLF9, LRRIQ1, LRRC46, C5orf55, YOD1, RP11-339B21.14, RP11-504P24.8, ZNF77, GABPB1, SLC25A42, NPR1, LIPT1, TMEM91, ROPN1L, VIL1, TJP3, SDCBP2-AS1, CLEC2B, RPS6KL1, LRRC6, DFNB59, USH1G, GPR157, C7orf63, CTD-3065J16.9, MIR210HG, ADRB1, GXYLT2, SLC2A3, WDR65, TSNAXIP1, ATP2A1, PHYHIP, OSCP1, C12orf55, RP11-706015.7, RGS9BP, MSH5, RP11-498C9.12, LRRC29, MPZL3, TTC40, RP11-195F19.9, R3HDML, 43347, SNORA5C, RP11-214N9.1, COL6A3, MAOB, RP11-86H7.7, KCNJ16, DHRS9, ALOX5AP, ISM2, LYPD3, NPHS1, DBNDD2, GLT8D2, SELPLG, RP11-263K19.6, RP13-131K19.2, SERPINA5, TCP10L, AC018816.3, RP11-318A15.2, ZMYND10, RP11-576C2.1, GPR132, CTD-2292M16.8, HOXD11, CDH16, CCDC148, ZNF404, ZMIZ1-AS1, RP13-631K18.3, FAR2P1, C10orf107, RP1-121G13.3, RP5-1157M23.2, C2orf62, CSPG4P8, RP11-108K14.4, EEF1DP2, RP11-12A20.7, PSG1, ARMC12, CDH8, RP11-309L24.9, ALS2CR12, POU5F1P3, DNM1P35, TAF7L, RAPSN, RFPL3S, RNF223, AURKC, TRBV200R9-2, C6orf165, RP11-699L21.1, CXorf58, DNAAF1, RP11-167N4.2, XAGE1B, TMEM88, TMEM229B, COL5A1, RRM2, MAZ, LTV1, KLHDC3, RNF44, C9orf69, LMNB1, GRPEL2, DICER1, ZMYND19, ERGIC2, H2AFX, LATS2, SLC35E1, MORC2, PUS7, DDX19A, C2orf69, WDR37, RFC4, TMUB1, GSG2, CTXN1, CPA4, FBXO10, EIF4EBP2, EIF5A2, GMNN, HDHD1, E2F8, ARID3A, MZF1, SPATA33, GNB1L, C6orf120, CEBPD, TMC7, ZNRF2, MAPK15, INTU, ASB13, ZNF107, SCML2, LMLN, AK4, ZNF367, HIC2, SKP2, DPF1, KPNA5, KRT10, ZNF724P, C14orf79, CIPC, C17orf97, SUV420H2, AC012146.7, SCAI, SFXN5, TOR2A, LHPP, ZNF680, RP11-296110.6, CDK5R1, LINC00116, CDC25A, TTLL7, FAM227A, RP11-313J2.1, NKIRAS1, EIF4A1, KIF17, FER1L4, HMGA2, BOP1, THRB, FAM86B1, NRARP, KIAA1407, CTD-2583A14.11, GPR157, C17orf89, EPB41 L4B, SLC2A3, ZNF257, RP11-263K19.4, ZNF487, ZNF846, RP11-305E6.4, DYNC2L11, NEURL2, MORN1, DUSP2, PBX4, NCALD, ZNF2, YOD1, AC006115.3, RP4-717123.3, RP4-635E18.8, RP11-384K6.2, RP11-242D8.1, C20orf24, RORA, C4orf48, NKX2-5, IRAK1BP1, ZNF695, RP4-758J24.5, GPR75, DENND2C, NR6A1, MIR210HG, AC005224.2, LIPE, NPIPA1, GPS2, LPAR2, AOC3, HS6ST1P1, HOXC-AS2, NIM1K, MRPL45P2, RP11-254B13.1, RP11-524H19.2, PDF, DTX2P1, TAS2R14, CBWD3, ZNF19, FGF14-AS2, CTC-251D13.1, NKD2, TTC18, AC007551.3, SNAP91, IP04, C9orf163, FSIP1, CTC-546K23.1, DRP2, AP006216.11, RND1, CTC-462L7.1, RP11-517B11.2, BAIAP3, RMRP, RP11-795F19.5, DIO2, RP11-69E11.4, RP1-63G5.5, SCNN1A, PCDH17, ZNF595, EFCAB6, SENP3-EIF4A1, ZEB2-AS1, C5orf66, LRGUK, AP001468.1, RP5-940J5.6, SLC05A1, CATSPER3, COL5A2, SLC6A6, RHOB, GOLGA4, DCAF6, CPS1, TRIML2, RFX5, LATS2, CDKN1A, POMT2, DCAF4, HMOX1, SUCO, OPN3, MNT, DICER1, PDCD4, ATXN7L3B, C9orf69, YOD1, CLIP4, KDM5B, TSPYL2, RABL5, CASP7, ELOVL4, C3orf49, THNSL1, TCTN2, CYBRD1, WDR19, UBXN11, TIGD1, ZNF33B, FBXW9, SPATA2, SNN, CCDC113, LZTFL1, DYNLL1-AS1, AMDHD1, TLDC1, FRAT2, CALCOCO1, PIK3R3, CROT, IFT81, PLD1, IFT140, LINC01139, EXOC6B, HOXC13, BTG2, RABL2A, GABPB1, SOX6, HDHD1, TPPP, NDRG4, TRANK1, NPHP1, ZNF287, C6orf120, ODF2L, TEF, LMBR1L, APH1B, AC006273.5, SESN3, YPEL5, ANKRD46, CCNG2, DUSP18, ABCA1, FAM229A, MXD4, MPZL3, SUV420H2, RNF44, HIST1H2AC, LDHD, IQCG, RP11-280G9.1, RP11-356J5.12, SLC15A5, ZNF354C, CTD-2270L9.4, USP49, RAB36, GRK5, CTXN1, RP11-420G6.4, DYNC2L11, TTC30A, ARL17B, DNAJB5, CREBRF, RP11-115D19.1, AP001372.2, LINC01021, DENND2C, CHIC1, ZBTB3, THRB, BBS12, SPEF2, RBKS, ZNF385A, AKAP6, RAD51-AS1, RPS17, LINC00319, SRP14-AS1, SAMD15, DNAJC3-AS1, RIBC1, NEK10, FBXO10, RP4-717123.3, RP11-108K14.4, RP11-253M7.1, FAM227A, HIST1H2BC, KLF9, CPEB3, RP11-545E17.3, CASC2, PIH1D2, C17orf97, SLC2A3, NME5, C7orf63, SEC31B, HIST1H4H, LMLN, SLC46A3, KLHDC9, OSCP1, C9orf9, ZNF599, C2orf70, C4orf48, ZNF606, HOGA1, FGGY, MDH1B, LRRC46, RP11-235E17.6, FAM160A1, RP11-299G20.2, HLA-DMA, RP11-144N1.1, HBP1, LINC00668, LRRC48, KCNH3, ZNF347, RGCC, YPEL2, RP11-504P24.2, AC016700.5, CFHR3, RP11-175K6.1, AL773604.8, RN7SKP97, CPT1C, CDS1, ZNF876P, RP11-43F13.3, RP11-31506.1, RPL9P8, FANK1, RP5-1092A3.4, RP4-740C4.6, HYDIN, PTCHD4, SLC9A9, HIC1, C5, CCDC30, LRRIQ1, CETN4P, KLHDC1, STK32A, CCDC146, RBM44, CTB-51J22.1, RSPH1, FBXO15, RP11-159N11.4, AK8, RP11-467H10.1, CTC-203F4.2, PIK3IP1, RP11-465N4.4, TLL2, ZNF410, RP11-526A4.1, KIAA2022, RP11-417E7.1, RP11-290L1.2, PRKXP1, ZMYND10, THBS4, CCL26, ENO4, RN7SL441P, RP11-1000B6.5, CDKL2, TPRXL, AC007551.3, WDR63, RP5-940J5.6, ARMC3, MT-C03, DPP9, NCAPD2, CLPTM1, KPNA2, TMEM245, ACSL4, RNF4, ASNA1, MINK1, PON2, RACGAP1, TMEM194A, ELK4, CEP192, ABCA1, TGFBR2, SRPR, HBP1, PNKD, TNFRSF10B, TRIM71, NRBP1, TRMT61A, ENTPD7, WDR90, KPNA5, ZNF542, CI orf109, M6PR, TSEN2, PHF1, LRRC14, MVK, PIGW, CASP7, ZNF680, TBC1 D17, LIN28B, MRPL24, GNS, NR6A1, CTXN1, C2CD2, GOLGA1, TPRG1L, NHLRC3, NAB2, YOD1, AP15P1, CREBL2, GABPB1, KLHL17, RAB23, DYNC211, BRMS1L, ATP8B1, FAM206A, ZNF837, POLD3, ZNF470, ZNF138, COG6, UNKL, TCTN2, DCAF6, SPATA2, PIGL, WDR5B, DNAH1, ZNF626, SCN1B, CROT, TTC30B, DCAF4, C2orf69, RP11-500C11.3, TMEM160, TECPR2, RRAS, CDS1, C20orf24, CHIC1, ZNF862, TPGS1, VAMP1, BANF1P3, CDK5R1, CHD9, C14orf79, MTND2P28, RPL4P5, TEF, KCTD13, SENP8, DNAH5, SFR1, IQCC, NR4A2, PFKFB4, DYNLL1-AS1, ZNF808, GREB1L, AC133528.2, DNAH6, KCNK1, FDXACB1, RP11-620J15.3, RNF32, C17orf82, GPX2, RP11-29402.2, DUSP19, NIPSNAP3B, GNAS-AS1, RP11-66N24.3, C2orf27A, LINC01003, ZNF396, GEMIN8P4, RHOH, LINC00476, CDKL2, BEGAIN, RP11-566J3.4, RPS17L, RP11-111M22.2, PDLIM3, CPEB1, SPNS1, RP5-890E16.2, SH3RF3-AS1, RP11-411B10.4, RP11-254B113.1, CTC-360G5.9, DIO3OS, RP13-766D20.2, FRZB, MIR4519, GFRA3, RP11-347C18.3, SOCS2-AS1, AC105760.2, RBPMS-AS1, LCA5L, ANKRD20A5P, RP11-336K24.12, RP11-37C7.3, IL9RP3, BCO2, RN7SL832P, CTD-3203P2.1, C11orf94, RP11-545E17.3, RP3-329A5.8, C3orf18, DDX11-AS1, ALX3, RN7SKP97, SPG200S, CYP2C8, TMEM150C, RP11-417L119.4, RP11-152F13.7, RP11-196G11.4, CYP2W1, ATF3, HERPUD1, FAM127A, SEMA4B, JUND, TBC1D17, LZTFL1, TPRG1L, PNRC1, STX4, PNPLA6, PLXNA3, SYNGR2, SESN3, YPEL2, APH1B, BTG2, SLC39A13, CPEB2, SLC2A14, LZTS3, CELSR3, LMBR1L, PPFIA3, C1orf216, ARRDC3, PDCD4, ZNF493, SOCS2, MTMR9, ZNF117, PCSK1N, MT1M, C9orf69, PHLPP2, SPATA2, CROT, ITGB8, NR6A1, ZNF616, MT1F, LMLN, ZNF449, ADCK1, TCTN2, DET1, DUSP8, MPP3, DNAJB5, FAM211B, MT1X, RABL2B, C17orf107, SPA17, CXCL12, BAIAP2-AS1, LINC00847, DYNC211, CAPN12, DZANK1, THBS3, C12orf36, DCAF4, TMEM27, OCA2, ZNF699, RP11-284F21.10, C14orf79, MXD4, CCDC176, NRBP2, C19orf54, ZFP41, HSD17B3, PCSK4, RP11-235E17.6, GPC2, ARMCX1, CTSK, ZNF596, CLIP3, TUBA1A, CSPG4P11, RP11-284F21.7, DNAH1, AC005154.6, LIN37, RP11-923111.7, RFTN2, GS1-124K5.11, PCDHA4, ANKRD42, CTHRC1, ZNF25, DYRK1B, ATL1, LINC00884, ZNF23, AQPEP, CTC-429P9.5, CARD9, CCT6P3, SNORA33, C14orf28, C17orf97, RNA28S5, RDH5, SPEF2, CCDC40, NEK10, RP5-1112D6.4, RP11-498D10.6, B3GNT9, LINC00886, RNF112, CCDC113, AC079922.3, RP11-522120.3, ZNF793, TINCR, LIN28A, ZNF404, CTD-2540F13.2, MAP3K14-AS1, SLC35E2, MST1R, RP11-390F4.6, TTLL3, H1FX-AS1, ZIC4, METTL25, PFKP, ZNF490, RBFADN, TSNAXIP1, TRIM29, EIF3CL, NEK11, RP11-73K9.2, ALKBH6, PHYHIP, ZNF214, HM13-IT1, PRRX1, RPS17, AC074212.5, ARMC3, DNAAF3, RP11-554A11.9, LINC00865, EFCAB13, ZNF701, RP11-552M11.8, PCDHGC4, PNPLA7, SPACA6P, RPS17L, RP11-48B3.4, AC005076.5, AC008174.3, PCDHB6, CCDC81, SH2D4B, CASP16, SRD5A2, RP11-254B113.1, RP11-1105G2.3, CTB-152G17.6, SAMD15, PRKG2, NUGGC, NOXO1, EPS8L1, LINC00086, RP11-1391J7.1, TTC18, MYLK2, ODF3L2, SOCS2-AS1, ASIC3, AC117395.1, ABCA9, ACOT12, ST6GALNAC1, FAM65B, CPNE7, RGS16, RP4-798A10.2, LINC00894, AC012442.6, PRKG1-AS1, CTAGE8, CTD-2015H6.3, CCDC71L, C9orf163, TBC1D3F, CTD-2083E4.4, LINC00176, USP3-AS1, and NRG3. This use is preferably for treatment of cancer, such as for treatment of PTEN-deficient cancer, wherein the cancer is preferably associated with lowered or insufficient expression of said gene. These genes were found to be upregulated by miRNA-193a in Example 1.2.1.

In more preferred embodiments, it is for upregulation of a gene selected from the group consisting of STAT3, TMEM2, PEG10, GCC2, RFX5, CPEB2, UNKL, RNF44, PGM2L1, NACC2, TDG, IFT81, CAMK2N1, BDNF, KANK1, CPS1, HDHD1, THBD, SEMA4G, SAMD4A, RP11-438N16.1, C2orf69, TPRG1L, CHIC1, HOXC13, DYRK1B, RASA2, CELSR3, ADM, KLHDC3, ABCC5, PNKD, MOK, PBXIP1, NUAK2, CLDN2, PHLPP2, CPOX, ZNF76, MBLAC2, VTN, CDH1, RMND5A, SCML1, LMBR1L, TGFBR2, ENTPD7, LZTFL1, C7orf60, ZNF558, CTNNBIP1, SNN, IFT140, RALGAPA1, WIPI1, C8orf58, PDCD4, RPL26, HOXA10, RP11-443N24.1, EIF4EBP2, RECK, ZNF614, HBP1, PRICKLE4, WDR25, DNMT3B, GNAZ, PER2, LMLN, SARM1, C17orf97, AC011841.1, CCNG1, ANKRD46, SYNGR3, TPPP3, KIAA0513, UBE2Q2P6, FAM227A, NEK11, KLHDC7A, ARID3A, CROT, PER1, F3, FAM217B, AK7, FRAT2, FOS, STX1B, APH1B, FER1L4, SAMD15, C3orf67, SKIDA1, SESN3, CTD-2366F13.1, SESN1, PADI1, C9orf9, ZBTB3, CCAT1, LATS2, RP11-553L6.5, JHDM1D-AS1, ZNF438, CEBPD, CTB-171A8.1, TEF, CYP1A1, LIN28B, DPY19L1P1, ZNF138, LINC00886, KLF9, LRRIQ1, LRRC46, C5orf55, YOD1, RP11-339B21.14, RP11-504P24.8, ZNF77, GABPB1, SLC25A42, NPR1, LIPT1, TMEM91, ROPN1L, VIL1, TJP3, SDCBP2-AS1, CLEC2B, RPS6KL1, LRRC6, DFNB59, USH1G, GPR157, C7orf63, CTD-3065J16.9, MIR210HG, ADRB1, GXYLT2, SLC2A3, WDR65, TSNAXIP1, ATP2A1, PHYHIP, OSCP1, C12orf55, RP11-706015.7, RGS9BP, MSH5, RP11-498C9.12, LRRC29, MPZL3, TTC40, RP11-195F19.9, R3HDML, 43347, SNORA5C, RP11-214N9.1, COL6A3, MAOB, RP11-86H7.7, KCNJ16, DHRS9, ALOX5AP, ISM2, LYPD3, NPHS1, DBNDD2, GLT8D2, SELPLG, RP11-263K19.6, RP13-131K19.2, SERPINA5, TCP10L, AC018816.3, RP11-318A15.2, ZMYND10, RP11-576C2.1, GPR132, CTD-2292M16.8, HOXD11, CDH16, CCDC148, ZNF404, ZMIZ1-AS1, RP13-631K18.3, FAR2P1, C10orf107, RP1-121 G13.3, RP5-1157M23.2, C2orf62, CSPG4P8, RP11-108K14.4, EEF1DP2, RP11-12A20.7, PSG1, ARMC12, CDH8, RP11-309L24.9, ALS2CR12, POU5F1P3, DNM1P35, TAF7L, RAPSN, RFPL3S, RNF223, AURKC, TRBV200R9-2, C6orf165, RP11-699L21.1, CXorf58, DNAAF1, RP11-167N4.2, XAGE1B, TMEM88, and TMEM229B, even more preferably it is for upregulation of all of these genes. This use is preferably for treatment of lung cancer, such as for treatment of PTEN-deficient lung cancer, wherein the lung cancer is preferably associated with lowered or insufficient expression of said gene. These genes were found to be upregulated by miRNA-193a in A549 cells (Example 1.2.1).

In other more preferred embodiments, it is for upregulation of a gene selected from the group consisting of COL5A1, RRM2, MAZ, LTV1, KLHDC3, RNF44, C9orf69, LMNB1, GRPEL2, DICER1, ZMYND19, ERGIC2, H2AFX, LATS2, SLC35E1, MORC2, PUS7, DDX19A, C2orf69, WDR37, RFC4, TMUB1, GSG2, CTXN1, CPA4, FBXO10, EIF4EBP2, EIF5A2, GMNN, HDHD1, E2F8, ARID3A, MZF1, SPATA33, GNB1L, C6orf120, CEBPD, TMC7, ZNRF2, MAPK15, INTU, ASB13, ZNF107, SCML2, LMLN, AK4, ZNF367, HIC2, SKP2, DPF1, KPNA5, KRT10, ZNF724P, C14orf79, CIPC, C17orf97, SUV420H2, AC012146.7, SCAI, SFXN5, TOR2A, LHPP, ZNF680, RP11-296110.6, CDK5R1, LINC00116, CDC25A, TTLL7, FAM227A, RP11-313J2.1, NKIRAS1, EIF4A1, KIF17, FER1L4, HMGA2, BOP1, THRB, FAM86B1, NRARP, KIAA1407, CTD-2583A14.11, GPR157, C17orf89, EPB41L4B, SLC2A3, ZNF257, RP11-263K19.4, ZNF487, ZNF846, RP11-305E6.4, DYNC2LI1, NEURL2, MORN1, DUSP2, PBX4, NCALD, ZNF2, YOD1, AC006115.3, RP4-717123.3, RP4-635E18.8, RP11-384K6.2, RP11-242D8.1, C20orf24, RORA, C4orf48, NKX2-5, IRAK1BP1, ZNF695, RP4-758J24.5, GPR75, DENND2C, NR6A1, MIR210HG, AC005224.2, LIPE, NPIPA1, GPS2, LPAR2, AOC3, HS6ST1P1, HOXC-AS2, NIM1K, MRPL45P2, RP11-254B13.1, RP11-524H19.2, PDF, DTX2P1, TAS2R14, CBWD3, ZNF19, FGF14-AS2, CTC-251 D13.1, NKD2, TTC18, AC007551.3, SNAP91, IPO4, C9orf163, FSIP1, CTC-546K23.1, DRP2, AP006216.11, RND1, CTC-462L7.1, RP11-517B11.2, BAIAP3, RMRP, RP11-795F19.5, DIO2, RP11-69E11.4, RP1-63G5.5, SCNN1A, PCDH17, ZNF595, EFCAB6, SENP3-EIF4A1, ZEB2-AS1, C5orf66, LRGUK, AP001468.1, RP5-940J5.6, SLCO5A1, and CATSPER3, even more preferably it is for upregulation of all of these genes. This use is preferably for treatment of breast cancer, more preferably of triple negative breast cancer, wherein the breast cancer is preferably associated with lowered or insufficient expression of said gene. These genes were found to be upregulated by miRNA-193a in BT549 cells (Example 1.2.1).

In other more preferred embodiments, it is for upregulation of a gene selected from the group consisting of COL5A2, SLC6A6, RHOB, GOLGA4, DCAF6, CPS1, TRIML2, RFX5, LATS2, CDKN1A, POMT2, DCAF4, HMOX1, SUCO, OPN3, MNT, DICER1, PDCD4, ATXN7L3B, C9orf69, YOD1, CLIP4, KDM5B, TSPYL2, RABL5, CASP7, ELOVL4, C3orf49, THNSL1, TCTN2, CYBRD1, WDR19, UBXN11, TIGD1, ZNF33B, FBXW9, SPATA2, SNN, CCDC113, LZTFL1, DYNLL1-AS1, AMDHD1, TLDC1, FRAT2, CALCOCO1, PIK3R3, CROT, IFT81, PLD1, IFT140, LINC01139, EXOC6B, HOXC13, BTG2, RABL2A, GABPB1, SOX6, HDHD1, TPPP, NDRG4, TRANK1, NPHP1, ZNF287, C6orf120, ODF2L, TEF, LMBR1L, APH1B, AC006273.5, SESN3, YPEL5, ANKRD46, CCNG2, DUSP18, ABCA1, FAM229A, MXD4, MPZL3, SUV420H2, RNF44, HIST1H2AC, LDHD, IQCG, RP11-280G9.1, RP11-356J5.12, SLC15A5, ZNF354C, CTD-2270L9.4, USP49, RAB36, GRK5, CTXN1, RP11-420G6.4, DYNC2L1I1, TTC30A, ARL17B, DNAJB5, CREBRF, RP11-115D19.1, AP001372.2, LINC01021, DENND2C, CHIC1, ZBTB3, THRB, BBS12, SPEF2, RBKS, ZNF385A, AKAP6, RAD51-AS1, RPS17, LINC00319, SRP14-AS1, SAMD15, DNAJC3-AS1, RIBC1, NEK10, FBXO10, RP4-717123.3, RP11-108K14.4, RP11-253M7.1, FAM227A, HIST1H2BC, KLF9, CPEB3, RP11-545E17.3, CASC2, PIH1D2, C17orf97, SLC2A3, NME5, C7orf63, SEC31B, HIST1H4H, LMLN, SLC46A3, KLHDC9, OSCP1, C9orf9, ZNF599, C2orf70, C4orf48, ZNF606, HOGA1, FGGY, MDH1B, LRRC46, RP11-235E17.6, FAM160A1, RP11-299G20.2, HLA-DMA, RP11-144N1.1, HBP1, LINC00668, LRRC48, KCNH3, ZNF347, RGCC, YPEL2, RP11-504P24.2, AC016700.5, CFHR3, RP11-175K6.1, AL773604.8, RN7SKP97, CPT1C, CDS1, ZNF876P, RP11-43F13.3, RP11-31506.1, RPL9P8, FANK1, RP5-1092A3.4, RP4-740C4.6, HYDIN, PTCHD4, SLC9A9, HIC1, C5, CCDC30, LRRIQ1, CETN4P, KLHDC1, STK32A, CCDC146, RBM44, CTB-51J22.1, RSPH1, FBXO15, RP11-159N11.4, AK8, RP11-467H10.1, CTC-203F4.2, PIK31P1, RP11-465N4.4, TLL2, ZNF410, RP11-526A4.1, KIAA2022, RP11-417E7.1, RP11-290L1.2, PRKXP1, ZMYND10, THBS4, CCL26, ENO4, RN7SL441P, RP11-1000B6.5, CDKL2, TPRXL, AC007551.3, WDR63, RP5-940J5.6, and ARMC3, even more preferably it is for upregulation of all of these genes. This use is preferably for treatment of lung cancer, such as for treatment of PTEN-deficient lung cancer, wherein the lung cancer is preferably associated with lowered or insufficient expression of said gene. These genes were found to be upregulated by miRNA-193a in H460 cells (Example 1.2.1).

In other more preferred embodiments, it is for upregulation of a gene selected from the group consisting of MT-C03, DPP9, NCAPD2, CLPTM1, KPNA2, TMEM245, ACSL4, RNF4, ASNA1, MINK1, PON2, RACGAP1, TMEM194A, ELK4, CEP192, ABCA1, TGFBR2, SRPR, HBP1, PNKD, TNFRSF10B, TRIM71, NRBP1, TRMT61A, ENTPD7, WDR90, KPNA5, ZNF542, C1orf109, M6PR, TSEN2, PHF1, LRRC14, MVK, PIGW, CASP7, ZNF680, TBC1 D17, LIN28B, MRPL24, GNS, NR6A1, CTXN1, C2CD2, GOLGA1, TPRG1L, NHLRC3, NAB2, YOD1, API5P1, CREBL2, GABPB1, KLHL17, RAB23, DYNC2LI1, BRMS1L, ATP8B1, FAM206A, ZNF837, POLD3, ZNF470, ZNF138, COG6, UNKL, TCTN2, DCAF6, SPATA2, PIGL, WDR5B, DNAH1, ZNF626, SCN1B, CROT, TTC30B, DCAF4, C2orf69, RP11-500C1.3, TMEM160, TECPR2, RRAS, CDS1, C20orf24, CHIC1, ZNF862, TPGS1, VAMP1, BANF1P3, CDK5R1, CHD9, C14orf79, MTND2P28, RPL4P5, TEF, KCTD13, SENP8, DNAH5, SFR1, IQCC, NR4A2, PFKFB4, DYNLL1-AS1, ZNF808, GREB1L, AC133528.2, DNAH6, KCNK1, FDXACB1, RP11-620J15.3, RNF32, C17orf82, GPX2, RP11-29402.2, DUSP19, NIPSNAP3B, GNAS-AS1, RP11-66N24.3, C2orf27A, LINC01003, ZNF396, GEMIN8P4, RHOH, LINC00476, CDKL2, BEGAIN, RP11-566J3.4, RPS17L, RP11-111M22.2, PDLIM3, CPEB1, SPNS1, RP5-890E16.2, SH3RF3-AS1, RP11-411B10.4, RP11-254B113.1, CTC-360G5.9, DIO3OS, RP13-766D20.2, FRZB, MIR4519, GFRA3, RP11-347C18.3, SOCS2-AS1, AC105760.2, RBPMS-AS1, LCA5L, ANKRD20A5P, RP11-336K24.12, RP11-37C7.3, IL9RP3, BCO2, RN7SL832P, CTD-3203P2.1, C11 orf94, RP11-545E17.3, RP3-329A5.8, C3orf18, DDX11-AS1, ALX3, RN7SKP97, SPG200S, CYP2C8, TMEM150C, RP11-417L19.4, RP11-152F13.7, RP11-196G11.4, and CYP2W1, even more preferably it is for upregulation of all of these genes. This use is preferably for treatment of liver cancer, such as for treatment of PTEN-deficient liver cancer, wherein the liver cancer is preferably associated with lowered or insufficient expression of said gene. These genes were found to be upregulated by miRNA-193a in HEP38 cells (Example 1.2.1).

In other more preferred embodiments, it is for upregulation of a gene selected from the group consisting of ATF3, HERPUD1, FAM127A, SEMA4B, JUND, TBC1 D17, LZTFL1, TPRG1L, PNRC1, STX4, PNPLA6, PLXNA3, SYNGR2, SESN3, YPEL2, APH1B, BTG2, SLC39A13, CPEB2, SLC2A14, LZTS3, CELSR3, LMBR1L, PPFIA3, C1 orf216, ARRDC3, PDCD4, ZNF493, SOCS2, MTMR9, ZNF117, PCSK1N, MT1M, C9orf69, PHLPP2, SPATA2, CROT, ITGB8, NR6A1, ZNF616, MT1F, LMLN, ZNF449, ADCK1, TCTN2, DET1, DUSP8, MPP3, DNAJB5, FAM211B, MT1X, RABL2B, C17orf107, SPA17, CXCL12, BAIAP2-AS1, LINC00847, DYNC2LI1, CAPN12, DZANK1, THBS3, C12orf36, DCAF4, TMEM27, OCA2, ZNF699, RP11-284F21.10, C14orf79, MXD4, CCDC176, NRBP2, C19orf54, ZFP41, HSD17B3, PCSK4, RP11-235E17.6, GPC2, ARMCX1, CTSK, ZNF596, CLIP3, TUBA1A, CSPG4P11, RP11-284F21.7, DNAH1, AC005154.6, LIN37, RP11-923111.7, RFTN2, GS1-124K5.11, PCDHA4, ANKRD42, CTHRC1, ZNF25, DYRK1B, ATL1, LINC00884, ZNF23, AQPEP, CTC-429P9.5, CARD9, CCT6P3, SNORA33, C14orf28, C17orf97, RNA28S5, RDH5, SPEF2, CCDC40, NEK10, RP5-1112D6.4, RP11-498D10.6, B3GNT9, LINC00886, RNF112, CCDC113, AC079922.3, RP11-522120.3, ZNF793, TINCR, LIN28A, ZNF404, CTD-2540F13.2, MAP3K14-AS1, SLC35E2, MST1R, RP11-390F4.6, TTLL3, H1FX-AS1, ZIC4, METTL25, PFKP, ZNF490, RBFADN, TSNAXIP1, TRIM29, EIF3CL, NEK11, RP11-73K9.2, ALKBH6, PHYHIP, ZNF214, HM13-IT1, PRRX1, RPS17, AC074212.5, ARMC3, DNAAF3, RP11-554A11.9, LINC00865, EFCAB13, ZNF701, RP11-552M11.8, PCDHGC4, PNPLA7, SPACA6P, RPS17L, RP11-48B3.4, AC005076.5, AC008174.3, PCDHB6, CCDC81, SH2D4B, CASP16, SRD5A2, RP11-254B13.1, RP11-1105G2.3, CTB-152G17.6, SAMD15, PRKG2, NUGGC, NOXO1, EPS811, LINC00086, RP11-1391J7.1, TTC18, MYLK2, ODF3L2, SOCS2-AS1, ASIC3, AC117395.1, ABCA9, ACOT12, ST6GALNAC1, FAM65B, CPNE7, RGS16, RP4-798A10.2, LINC00894, AC012442.6, PRKG1-AS1, CTAGE8, CTD-2015H6.3, CCDC71 L, C9orf163, TBC1 D3F, CTD-2083E4.4, LINC00176, USP3-AS1, and NRG3, even more preferably it is for upregulation of all of these genes. This use is preferably for treatment of liver cancer, such as for treatment of PTEN-deficient liver cancer, wherein the liver cancer is preferably associated with lowered or insufficient expression of said gene. These genes were found to be upregulated by miRNA-193a in HUH7 cells (Example 1.2.1).

The invention provides a miRNA-193a molecule, isomiR, mimic, or source thereof as described herein, or a miRNA-193a for use in treating a condition associated with PTEN-deficiency such as for use as a PTEN agonist, wherein the miRNA-193a molecule, isomiR, mimic, or source thereof is for downregulation of a gene selected from the group consisting of RPS17L, GPR137C, EEF1A1P19, NEFH, KRT14, RP5-973M2.2, OVOL2, RP11-873E20.1, RP5-968P14.2, MYB, AC000068.5, NOTUM, RP11-209D14.2, RP11-326K13.4, RP11-339B21.10, IRF8, HIST1H4C, DPF3, RP11-276H7.3, RP4-694A7.4, RP11-17M16.2, KB-226F1.2, SHBG, LAT2, SNORA33, SNORD12, AC005592.2, RP11-796E2.4, RP11-280G9.1, NOG, LINC00035, 7SK, GJB2, MYH11, BHLHE41, RP11-211N8.2, IL12A, EPB41L3, ROR2, UNC5CL, NINJ2, SUCNR1, CTD-2369P2.2, MYLK4, SLC35D2, SHMT2, ERMP1, TEX15, COL20A1, SORL1, PHLDA2, C10orf91, TWISTNB, HPD, PLAU, IL17RD, RNF182, KRAS, LL22NC03-N14H11.1, AC004158.2, ANKRD44, STMN1, CSPG5, PARD6A, FOXRED2, TMPPE, TNFRSF21, DNASE1L2, HHAT, TOX3, STARD7, MPP2, B3GNT3, ZDHHC18, RP11-173M1.8, PITPNB, RP11-34016.6, ETV1, ATP5SL, EPHB3, AIMP2, PI3, DHCR24, DAZAP2, C9orf47, ZNF365, RP11-204C16.4, SLC30A7, KIAA1875, NYAP1, CCND1, CHD5, SHOX2, ST3GAL4, SEPN1, KLRG2, VAMP8, AP2M1, FAM60A, SULF2, ZSWIM5, MED21, RP11-24B19.3, Z83851.4, DUSP7, C1QBP, NCEH1, TBX20, UBP1, RP11-421N8.1, LY6K, GPR146, ST3GAL5, ATP5F1, OSMR, CBL, CCDC28A, YWHAZP2, YWHAZ, DPY19L1, EXTL2, NAP1 L5, CTC-428G20.3, ETS1, UBE2L6, GREB1, FAM168B, CLDN4, YWHAZP3, ALX1, CRKL, RPS17, HFE, TRIM62, MSANTD3, ZMAT3, ENDOD1, KIAA1644, MOSPD2, FIBCD1, ATP8B2, PRNP, DLEU2, SLC2A12, WSB2, VGF, SERINC2, DCTN5, XK, NUP50, SMPD1, CNOT6, RP11-395A13.2, GABPA, CLSTN1, POLE3, BOD1, LRRC8A, SLC35G1, TP53INP1, AGPAT1, HPRT1, RUSC1, SLC23A2, PDE3A, EBF1, FANCE, WDYHV1, MAX, WDFY2, MCL1, MORN4, FAM72A, CDCA7L, TPP1, AREL1, COPRS, NT5E, TMEM121, 43346, SLC30A1, PRRG4, RBBP5, LAMC2, SLC35D1, C18orf54, GLYCTK, DPM3, HACE1, CDK6, GPATCH11, IDH2, PTPLB, EMILIN2, CCNA2, FAM60CP, TK2, FAM20B, GNPDA1, TIMP4, NR2F6, TMEM180, TRAF1, DERA, ATP8B3, XYLT1, SLC39A10, KLHL2, PIK3AP1, IDS, CADM1, TBL1XR1, KCNMA1, FAM101B, PPTC7, SLC29A3, AP5M1, PEX11B, MEX3C, ARF3, PLD6, INO80C, SNX10, NUDT15, CCDC149, SLC26A2, GNAI3, DCAF7, APOL6, ADCY9, NUDT21, PRR5L, HYOU1, BCYRN1, CCNDBP1, DSEL, PAFAH2, FAF2, SLC25A15, NEIL2, USP39, GCH1, FCHSD2, TXLNA, TMEM135, RN7SL2, RAB11FIP5, NAGA, ACOT8, WDR82, PCDHAC2, VAV3, SRP54, RN7SL1, FAM72D, SEC31A, TYW3, ZNF512B, HCG11, ARHGAP29, SAPCD2, PALM3, MIR17HG, DGKH, CASP9, LINC00657, TMEM30A, SLC30A4, CHEK1, RTKN2, IGSF3, NBL1, TGFBR3, RP11-67L2.2, STRADB, APPL1, ARPC5, NIPA1, ZNF710, CA12, NET1, CTD-2196E14.9, ABI2, R3HDM1, SFXN1, MESDC1, MTA2, DOCK10, PHACTR2, KBTBD11, ELK3, PSRC1, MSANTD2, RASGRF2, ATP6V1B2, ALDH9A1, LARS2, CDKL5, RNF216P1, LRRC40, LUZP1, MORC4, MYLK, PLAUR, CCSER2, RP11-73M18.7, ERBB2IP, ACSS2, NOL9, DLEU1, C17orf58, PITHD1, SEC61A1, ARHGAP19, CDC42EP2, HNRNPUL2, BCAT2, RP11-329A14.1, ST5, NUDT3, TNFSF9, PADI2, EMC6, IRF1, PLXNA2, COPS3, CCP110, ABCA12, MPST, CYTH1, PLEKHB2, MED19, GALNT14, MLKL, TOR4A, SYNRG, AFAP1 L1, TRAK2, SGMS1, MARCKSL1, IL6R, PIP4K2C, JADE3, CBX1, HELZ, NSF, IF127L2, TMEM216, SDE2, RTN4RL2, SSRP1, TRPA1, GDF11, SPECC1L, RUFY2, DNAJC3, KIAA1191, AC007560.1, HSBP1, IL11, UNG, HEG1, MAP4K5, PPM1F, GLO1, ANKRD13A, KIF1B, KIAA1147, TRAFD1, PAK4, FAM114A2, DIO2, POPDC3, PLEK2, DNAJC16, NT5DC3, RAB27B, TWF1, CLN6, KDM7A, R3HDM4, ZNF618, LRP4, ITPKB, PDPK1, ALKBH5, C11orf24, DSTYK, AAED1, CEP41, MAP3K3, KLHL15, PTPLA, AFMID, LMNB2, MLLT11, DESI1, WDR5, B4GALT6, CCNJ, SENP1, ZBTB14, SIPA1 L2, PHF19, TP53BP2, ASF1B, USP43, SS18, CHCHD5, BCL2L2, CAPN15, ADNP2, RCAN3, RNF2, TAP2, SOS2, HDAC3, AP1 S2, GFPT1, ABR, FOCAD, ERRF11, RC3H2, EML4, PTPN9, AFF4, CD97, RABEPK, SQSTM1, PRR14L, HP1BP3, GPRC5B, CBFB, ARPC2, GPC6, TCF7L2, GALNT2, TRIP6, PIK3R1, DDAH1, RHOU, UBE2Z, DYNC2H1, ENOX2, IER31P1, ZC3H7B, ZNF324, SPOPL, FBXW2, ORAI1, MUC5AC, C4orf46, KIDINS220, MYADM, SLC3A2, PM20D2, PSEN1, RPS6KB2, TPCN2, GALNT1, RFWD3, GALNT13, FOXN2, TWF1P1, FKBP9, CCL2, RNF168, GINS3, MRPL42, LYRM2, PTTG1IP, NRIP1, SSX21P, DEF8, WDR48, TLR6, EXOSC3, SCP2, FILIP1, INPPL1, TTLL4, TCEB3, SEC22C, IWS1, GBE1, GNL3L, GOSR2, LGR4, SAAL1, UHRF1 BP1, SLC29A1, WDR6, VPS37B, HSPA13, TOMM20, PCBD1, CHML, SLC7A5, TP53RK, RUSC2, UTP18, STARD3, C2orf49, BRPF3, PODXL, TUBA1B, PDE8A, DYNLL2, CAPN10, HMGB1, IL4R, SYT1, TUFM, PCBP1, TMBIM1, KCTD5, POM121C, WHSC1, CTDSP2, AGAP2-AS1, KDM5C, PTK2, CPNE3, KIAA0430, CAMKK1, TPCN1, KLHL9, TRIM25, CAPRIN1, UBFD1, MED14, TMEM164, ELMO2, KANK2, ABCB10, CNBP, ITPRIPL2, SOGA1, QDPR, B4GALNT1, FBXW5, TROVE2, FGD6, SUDS3, MTHFD1, KIF14, MAP3K2, AKAP12, OSER1, ACTR3, KIAA0141, ABCE1, HELLS, MRPL19, EIF2AK1, EPHB2, XPNPEP1, YAP1, RBFOX2, CDCA5, ENTPD4, ATP2B4, RBM10, LPCAT1, TPD52, CDK2, AGFG1, WWC3, LBR, PPP2R4, EIF4B, EXTL3, BTBD11, POM121, RIPK2, SFN, MCM2, TMEM230, CMTM4, GSR, TUBA4A, EDEM1, KIRREL, GOLPH3, NF1, TGFB2, PPP3R1, AKR1C3, NOTCH2, CCDC88A, KIAA1522, CTCF, BCAR1, SREBF2, GBF1, WWTR1, PDE4D, CDK4, PGRMC1, AKR1C2, MAP7D1, SET, NCOA3, SERINC3, ARHGAP11A, DEK, PRKCA, MLEC, SYNM, GNB1, PLS3, DDB1, F2RL1, GPC1, SERPINE1, KRT79, HMGCLL1, LINC00920, BTBD11, RP11-390F4.8, NEURL3, RP11-423P10.2, PAX5, KCNIP1, CD93, PLCB2, RP11-290F20.2, PDGFRB, MEDAG, CRISPLD1, RP5-1086K13.1, DLL1, AL139099.1, AC007383.3, AC046143.3, DNM3, AC111200.7, C11orf35, RP5-1157M23.2, PDE5A, CSF2, CMAHP, C6orf58, ITPKA, SLC22A14, SLC29A3, FOXRED2, ACTG2, SULF2, FAM211A, AC011043.1, CYS1, CTD-2313J17.5, AKNAD1, RP11-456K23.1, APOBEC3F, ZMYND15, RP11-588K22.2, CYP2D7P, ERMP1, ADAM22, ABCA9, GRB7, LL22NC03-86G7.1, HSPB7, FAM196B, SOX9-AS1, FAM227B, BEST3, TRAM1 L1, SGIP1, ADCY7, PCED1B, SEPN1, APOBEC3G, CCDC28A, NGFR, MPP2, IL17RD, PLAU, TMEM173, IFT27, CTD-2292P10.4, ZNRF2P2, NT5E, DGKB, TWISTNB, STMN1, RTN4RL2, SLC25A34, HFE, S100A16, RP11-807H7.1, KRT15, ITGB3, CIB2, SHMT2, GAB2, CMTM8, GALNT13, CCDC149, GALNT14, SLC35D2, CCND1, SYNPO2, ATP5SL, ETV1, TMEM216, TNFRSF1B, USP18, BCAT2, ACOT8, HYOU1, AP2M1, HTR7, PALM3, RP4-760C5.5, OTUB2, PLEKHA5, MIR621, TMPPE, RGS2, TNFRSF21, ERAP2, DCAF7, SFXN2, KRAS, DAZAP2, CLSTN1, ARHGDIB, FAM114A2, TP531NP1, TCEAL8, ST6GALNAC3, CERS1, PTPRE, PDE3A, CTSO, SLC30A4, ENDOD1, SLC23A2, C1QBP, UBE2L6, CNRIP1, ST3GAL5, ENPP4, PARD3B, PLD6, DPY19L1, ABCA8, MORN4, MYB, SLC26A2, NSF, FAT4, TPP1, SLC30A7, ZNF512B, ACPL2, RP11-2711.4, EDNRA, A4GALT, ZNF836, RNF146, PLCD1, STARD7, PEX11B, UPRT, CEP41, PTPRZ1, AGPAT1, ARHGAP19, XAF1, DHCR24, OSMR, AGAP2, MAST4, ACSS2, FBXL16, RHOU, RP11-18114.10, GBP3, POU2F2, AC009948.5, FAT3, PLCD3, LRRC8C, PGPEP1, SEC31A, SLC18B1, ISLR2, LINC00669, ZMAT3, IL1B, AIMP2, CCNJ, MOSPD2, GLYCTK, ST3GAL4, LRRC8A, TNIP3, MSANTD3, ANKRD13A, PCBD1, DERA, ARHGAP27, GLDCP1, GABPA, DGKA, ATP8B2, RUSC1, ZNF362, PRPF40B, SAMD9L, STS, RAB5B, CCL20, PCYOX1L, NFE2L3, USP27X, KDM7A, CDC42EP2, MMP1, FAM72B, GPR146, WNK4, MEF2BNB, MYOZ3, PAD12, CDKL5, HTR7P1, PTCH1, OAS2, ZNF365, OBSCN, PDE4D, WSB2, CYTH1, NCEH1, KIF5C, PRNP, MTSS1, FAM60A, LINC00657, GPD1L, FOCAD, DCTN5, PIK3R1, UBP1, RP11-34016.6, ZDHHC18, LOX, PIGA, CA12, APOLD1, PGM5, AKAP12, MCL1, PHLDA2, ZNF608, HACE1, BMF, IGSF3, PITPNB, ZSWIM3, ERBB2IP, NUDT18, PTPN9, ZCCHC10, ITGA2, PIP4K2C, TRAK2, LGR4, AP5M1, EBF1, DOCK4, AL390877.1, MED21, ELMO3, AC108676.1, GPRASP2, NAGA, CNOT6, ATP5F1, ZNF710, EPM2A, OSBPL5, COPRS, FCHSD2, TRIB2, TK2, TBX20, RUFY2, SREK1IP1, GNA13, RP11-421N8.1, IL8, FAM132B, YWHAZ, TAF9B, WDFY2, YWHAZP3, MARCKSL1, ETS1, TRIM62, HK2P1, ALDH9A1, OSBP2, TMEM180, GNPDA1, SAMD9, BTN3A2, YWHAZP2, PTK2, PNRC2, RAD17, IQCD, DNAJB9, ARHGEF9, POLE3, ARMCX2, DPM3, KANK2, DOK3, PLAUR, INPPL1, NT5DC3, DNMBP, LRRC40, ARHGEF40, SYNRG, GPATCH11, IWS1, RGL1, SEC61A1, PHACTR2, CDC14B, ZNF181, KLHL2, CBX7, IDS, PAK4, FAM72A, MPST, WBP5, ARF3, ACSL5, UBE2Q2P6, DDAH1, ASAP3, TRO, GAS1, PTPLB, ST5, SCP2, DOCK10, PXK, ARHGAP29, CXCL2, HECW1, LAMC2, R3HDM4, MAP3K3, MLLT11, GBE1, HYAL2, RAB11FIP5, GRAMD4, C11orf95, ADAMTS18, APBB2, CCSER2, WDR48, FAF2, STC1, IDH1, NUDT3, PARP14, NET1, AKR1C3, CHCHD5, HEMK1, TUFM, ELK3, DGKQ, CDK6, LPAR1, GDF15, CTDSP2, GULP1, MMP14, SIX4, LARS2, CD38, LRP5, CRKL, SMPD1, DUSP16, JAK2, B3GNT1, KIAA1147, FAM214B, PARD6A, SLC12A9, SS18L1, DGKH, PSEN1, ENOX2, PAX6, UFL1, FAM210B, TPCN1, SMG6, MAG13, PALMD, NEIL2, PDK4, APAF1, AGFG1, SLC35D1, SLC25A15, RNF215, GALNT1, HEG1, TRAF1, SRP54, PDGFD, HNRNPUL2, MDM2, TMEM30A, RSPO2, GPC6, PLEKHA2, CACNG4, CASZ1, PAG1, EXTL2, IFIT3, KANK1, RNF2, TNIK, PTPN21, ENTPD4, QDPR, PTPN3, SYNJ2, TMEM164, KITLG, FBXL15, PGAP1, DENND4C, GSDMD, TRAPPC9, ALKBH5, TRAFD1, DAB2, JADE3, PDPK1, COPS3, ABL1, EVA1C, EML4, SFXN1, LRP3, DDX60, EIF4BP3, DNAJC3, TGFBR3, DAK, CTTNBP2NL, GNA11, STARD3, TGM2, SLC9A3, IRF1, HK2, PLEKHB2, MAGEF1, PPTC7, RPS6KB2, ADAMTS15, EIF4BP7, SCAMP4, ADAMTSL1, NDFIP2, EIF4B, GPR176, MORC4, ERBB2, FAM20B, AREL1, GNL3L, USP39, SLC39A10, BOD1, ATP6V1B2, ARHGAP18, KIAA0430, GFPT1, EIF4BP6, SREBF2, FBXL17, MAX, CBFB, NECAP2, GEM, CDC42EP3, KIAA1522, KLHL9, CBL, KIAA1644, RCAN1, SUSD5, JADE2, GRHL3, SMARCA1, USP40, SQSTM1, KIF1B, LUZP1, SMIM14, MEX3C, ARHGEF1, NUP50, HELZ, CCDC90B, PPM1H, BCAR1, RAB27B, PSMB8, ANTXR1, SENP1, F2RL1, ARPC5, SIPA1L2, LNPEP, UBALD2, ZC3H7B, NUDT21, YAP1, FAM65A, LRBA, BMPR2, FRMD6, APPL1, AMIGO2, SCAMP1, AES, LPHN2, ZNF395, WDR82, HPRT1, PRKCA, TDO2, TCF4, TRIM8, SFT2D2, SLC20A2, ADAMTS1, SEC23B, RSF1, CPNE3, MAMLD1, DYRK2, LLGL1, NR2F2, TRIP6, SOS2, ARPC2, ERRF11, IDO1, PLSCR1, RNF182, BNC2, STAM, MX1, TCTN3, CHML, ELMO2, PITPNA, GALNT2, KLF3, RIPK2, PPM1F, LPCAT1, TBX18, MRPS18B, KIRREL, HSPA13, MAP4K5, LRRC8D, MAGED2, NCOA3, BACH1, IL7R, CCNA2, KDM5C, SLC30A1, CCNY, PIP4K2A, DDB1, RND3, DAPK1, GOLPH3, SSRP1, INTS3, FAM168B, TMCC3, CDK4, ZMIZ1, TM4SF1, NSD1, MTA2, SNHG5, GIT1, PPP2R4, KIAA1191, TXLNA, RC3H2, TMBIM1, TNFAIP8, HELZ2, UHMK1, CREBBP, WIP12, FRMD8, PLIN2, NOTCH2, LIF, ANGPTL4, DUSP4, SLC7A5, LAMC1, PLS3, SNX9, GPRC5B, RP11-30P6.6, LEF1, RGS17P1, CTC-428G20.6, CAMKV, RP11-440D17.3, RASA4, OXCT2, GRAP, CTA-217C2.2, ADAMTS16, AC119673.1, MPP2, CAMK2B, FGFR2, MIR103A2, LINC00460, RP11-540B6.3, AC005789.11, RP11-196016.1, TCERG1L, TNFRSF1B, ARMCX4, STON2, PARD6A, FAM156A, AGAP1-IT1, AC010525.6, MYRF, FBXL16, MAPK13, RLTPR, EXOC3L4, CCDC28A, HMX3, NDN, TP73, CTA-445C9.15, EXPH5, PHLDA2, RASSF5, ST3GAL5, 03-Sep, STMN1, INSRR, SHMT2, N4BP3, TWISTNB, CACNG6, PLAU, ERMP1, FOXRED2, SEPN1, KALRN, LRP4, IL1RL1, AC009061.1, PDE9A, TGM2, IGSF9B, PTGER2, DAZAP2, PITPNB, FAM132B, FKBP9L, ATP5SL, STARD7, HOXD13, RHOV, WDFY2, GNA15, HYOU1, DDAH1, INO80C, UBE2L6, ATP8B2, PRKCH, AP2M1, DHCR24, TOR4A, TMEM121, SRRM3, ARHGAP19, SLC39A10, RP11-82L18.2, AGPAT1, DND1, NT5E, GJB2, SLC30A7, F2RL1, FAM105A, ELK3, GCH1, GRTP1, NID1, SLC30A1, IRF1, PTK7, SERINC2, TMEM173, MARCKSL1, CCND1, FIBCD1, KIAA1644, COPRS, P2RX5, ZNF365, HHAT, TNFRSF21, VAMP8, SLC35D2, RP11-34016.6, KRAS, ZDHHC18, WNT9A, IGSF3, DPM3, ALDH1A3, PRDM8, SLC26A2, ROR1, ACSS2, C11orf95, GALNT14, STC1, IL8, NPIPB4, UBP1, NR2F6, PRNP, USP39, DUSP7, FAM101B, FAM60A, ST3GAL4, OSMR, SH2B2, FAM168B, STRADB, ZNF703, TRIM62, SOX18, YWHAZ, CDK6, GNAI3, RP11-204C16.4, FOXL1, ACPL2, GNPDA1, LRRC8A, GREB1, SLC30A4, SORL1, TBC1D5, RP3-425P12.4, ALX4, NCEH1, CHST14, MCL1, VASH2, SLC45A3, AIMP2, RUSC1, RASL11A, ICAM1, RP11-329A14.1, HEG1, PIK3R1, ETS1, KIAA1147, ANKLE1, CXCL3, PTX3, EFNB2, FAM20B, DGKH, YWHAZP2, DPY19L1, YWHAZP3, DCAF7, BCAT2, SCAMP4, DCTN5, RAB11FIP5, BOD1, ZMAT3, C12orf39, CCNJ, WSB2, GPR161, POLE3, NEFH, GPR3, RBM24, SUCNR1, B4GALNT3, IDH2, TEX15, TERT, RP11-101E13.5, SFXN1, C16orf59, C1QBP, TGFBR3, ZCCHC10, KLHL2, IL6R, AP5M1, GALNT13, PSRC1, PDGFRB, LMNB2, COL5A3, MYEOV, TMEM164, SERPINE1, CXCL2, STAMBPL1, GFPT1, DERA, WDYHV1, PDPK1, SIPA1L2, CCNDBP1, MAX, KANK2, PITHD1, IL4R, NT5DC3, ATP5F1, FAM60CP, PFDN1, CA12, PMAIP1, NPTX1, CLSTN1, MOSPD2, NUDT21, SLC35D1, GABPA, TRAFD1, RP11-22P6.3, UBE2Q2P6, NUDT15, SLC7A5, MESDC1, ADCY1, TMEM216, PDE3A, ENDOD1, CRKL, LOXL1, NHS, NES, TBX2, DMTN, EGR1, GPATCH11, CNOT6, TEAD3, UNG, AREL1, PLSCR1, HPRT1, RNF138, TAF4B, RFWD3, MAMLD1, ARHGAP26, AKAP12, PAK4, TXLNA, MPST, TNFAIP8, RAB5B, SMPD1, PM20D2, MSANTD3, CXCL1, SOX7, PAPPA2, CMTM3, CAPN15, RP11-421N8.1, DOCK10, SEC61A1, KCNK5, NAGA, LINC00941, CXCL5, TBL1XR1, FAM72D, ZG16B, TMOD1, PNRC2, GDF11, SEC31A, PLCD3, PTPLB, PLEKHB2, FOSB, NRIP3, HSD11B2, GPR27, WDR5, ARF3, RNF216P1, ZNF35, CASP9, SLC29A3, ST8SIA4, SCP2, FCHSD2, ABR, ARHGEF40, KLHL15, PPM1F, KCTD12, APLN, DTL, CCNA2, SRP54, SLC16A6, LRRC40, MED21, EML4, TNFRSF8, IL1RAP, HFE, FOXN2, ALKBH5, CCDC85C, SLC23A2, ARPC4, GLO1, SYNRG, ORAI1, ZNF678, NOTCH2, ST5, LUZP1, KIF1B, KCTD5, DLX1, RGS2, TANGO2, FAM72B, CASP2, UBE2Z, SSH3, FAF2, ADCY9, C18orf54, MAFF, MAP3K3, RBBP5, KLHL23, JADE3, ZNF618, BAI2, CBX1, PLXNA2, CDK2, CBFB, CBL, NUP50, GLI2, MMP1, CMTM4, BMP6, PSEN1, JAG2, LINC00657, ARHGAP29, ACSS3, ARPC5, TUBG1, FOCAD, TUFM, ZC3H7B, KIF26A, TP73-AS1, PAG1, RC3H2, SENP1, MTA2, CDCA7, SLC29A1, TRAK2, RNF2, POM121C, RNF146, TONSL, TEAD4, ELMO2, ENTPD4, BRPF3, PGRMC1, CLN6, OSBPL10, ERRFI1, PODXL, AMIGO2, LRRC8C, ANKRD13A, GALNT1, ASAP3, NUDT4, OSBPL8, CDC42EP2, SLC19A2, IL18R1, SMOX, EFNB1, TMEM30A, POM121, SLC16A9, UNC119B, ARPC2, INPPL1, KIRREL, CNKSR2, BCL2L2, TOMM20, SPRY4, SDC1, AFF4, FOS, SH2B3, KIAA1191, RNF215, SLC18B1, CTDSP2, PXK, TCEB3, SREBF2, C12orf49, KLHL8, APOL6, UBALD2, HK2, NET1, RUFY2, C17orf58, C11 orf24, CDCA7L, SAMD8, MAPK8, NOTCH1, PEX11B, HSPA13, PPTC7, DMRTA2, NEIL2, COPS3, TPD52, HNRNPUL2, FKBP9, EXOSC3, CCP110, PLAUR, GATA2, ABI2, SSRP1, SYNJ2, CBX6, CHCHD5, WDR82, PPP2R4, HSF1, ERBB2IP, PCBD1, SREK1IP1, MAP4K5, FRMD8, CRLF3, DDA1, EIF4B, FERMT3, CSRNP1, IWS1, LARS2, ID1, R3HDM1, ENOX2, WNT5A, FBXW2, PTK2, MTFR1, WNK2, SCAMP1, QDPR, PPAT, HELZ2, TK2, LPHN2, FZD8, TMBIM1, ALDH9A1, ELF4, BHLHE40, NUDT3, ASF1B, STS, WDR6, JAG1, PSMB8, PIP4K2C, CYP51A1P2, RNH1, THRA, MAP7D1, MFN2, PHF19, RNF168, ETS2, ANTXR2, SLC35G1, MEX3C, UTP18, PPP4R1, MDC1, HELLS, ATP6VOA2, DYNLL2, GOLPH3, SQSTM1, PATZ1, DESI1, GALNT2, HIP1, LINC00152, SAPCD2, FAM210B, PLXNA1, R3HDM4, REXO4, TYW3, CCDC14, SPECC1L, STARD4, ABCB10, NSF, ALG2, MAGEA1, KRT80, ZBED4, DEF8, SH3PXD2B, LSM14B, DUSP5, PAQR4, HSPB8, TRIB3, FBXW5, RBM10, SFT2D2, PDE4D, WHSC1, UPP1, FAM115C, EPDR1, RASA3, XPNPEP1, CDC45, MYADM, HN1L, BCOR, PRKAA2, RAPH1, CCSER2, CHEK1, NAB1, SLCO4A1, ADRBK1, PXN, B4GALNT1, TSPAN14, RIN1, TCOF1, SMG5, HP1BP3, RP11-1055B8.7, HSBP1, SKA2, OGFRL1, CDT1, SGMS1, MCM10, APPL1, ATP6V1B2, TROVE2, CD97, TRIP13, SS18, PHLDA1, TRIM25, FOSL1, ID3, PPP1R26, PPP3R1, RFC3, MRPS18B, GPC1, SET, IDS, MED14, IER2, TFPI2, UBFD1, CDCA4, OGFR, CNBP, PAPOLA, MRPL19, TNFAIP2, AKR1C3, TOMM34, FGFRL1, MCM2, KIAA0141, ADNP, LPCAT1, CDC6, MLEC, KIDINS220, AGFG1, HMGB1, LIF, IDH3A, UHMK1, TRIP6, RBM8A, FARSA, URB1, PITPNA, GNB1, WWTR1, SETP14, RPS21, CAPRIN1, TGOLN2, STC2, OSGIN1, NOTCH3, IDH1, BAZ1B, DDB1, TNPO1, LASP1, PCBP1, FASN, TUBB, ACTB, RP11-313P13.5, CTB-31N19.3, LINC00607, LRRC15, RGS17P1, NPAS3, CTD-3203P2.2, CSTF3-AS1, CTD-2342J14.6, CTD-2537I9.5, MYEOV, ANKRD31, CIDEC, MYO1G, SRRM3, LINC01132, ENDOD1, TSGA10IP, ADH1A, I11, RP11-572C15.6, CD207, RP11-274H2.5, TFF3, UXT-AS1, RPS19P3, RP11-305K5.1, CTD-2192J16.20, LLOXNC01-250H12.3, ZSCAN23, LINC01096, RPSAP52, CDC42EP3, AK4P3, GALNT16, ETS1, SEC14L2, CHST6, RP11-255H23.2, LINC01057, ULK2, FAM162B, RP11-1017G21.5, CTD-2161E19.1, MFSD4, ASAP3, AC026150.5, AC005077.12, LINC00312, TRIM62, CCDC28A, ROM1, TRPV3, RP11-73M18.9, HHAT, B3GNT3, TMEM30B, CRYAA, TFAP2A-AS1, TMEM151A, DACT3, SLC35D2, CCDC17, TGFBR3, TIMP4, MPP2, MYCN, TWISTNB, C19orf77, CCND1, NT5E, PTX3, RP11-116D2.1, SOCS3, SHMT2, PRR15L, DOK7, NAPA-AS1, SPP1, ERMP1, UBE2L6, NACAD, SLC6A12, KCNG3, CHCHD2P6, ERBB4, ANGPTL2, FAM150A, LOX, ANKLE1, ACOT8, ST3GAL5, AMPD3, SLC15A1, IL17RD, MYADML2, C8A, FOXRED2, GRIN2D, STMN1, DCAF7, TRIM17, HR, C1QBP, LINC00657, HFE, RP11-469M7.1, ATP5SL, FAM60CP, RP11-421N8.1, MPZ, SLC29A3, PNRC2, ARHGAP19, CDC42EP4, FAM168B, GTF2H2B, SORL1, PPARGC1A, P2RY1, KRAS, PHLDA2, DPY19L1, CCDC149, CCNJ, TNFRSF21, RUSC1, UBP1, IDS, DCTN5, SYNE3, LAMC2, KDM7A, LEF1, GABPA, PCBD1, PCYOX1L, TNFSF9, PDGFRB, MAT1A, VAMP8, ARID3B, CXXC4, MYB, ATP5F1, RP11-204C16.4, RP11-973N13.2, FAM101B, ENPP4, KRT80, HYOU1, PITPNB, WSB2, CRKL, UPK1A-AS1, AP2M1, FAM60A, PSRC1, DUSP7, DDAH1, ANKRD1, FAM203A, CNOT6, CER1, RP11-613M10.6, ATP8B2, IL6R, AAED1, ESRRG, INO80C, HSPB8, SLC23A2, SOX5, PDE3A, HFE2, NPTX1, ADCY9, MAX, NPPB, SLC30A7, RAB11FIP5, ZCCHC10, PAG1, MAPK8, FOXC1, UNC5CL, STARD7, LRRC8A, HRK, MISP, AIMP2, PIK3R1, PLAG1, POLE3, ACPL2, NCEH1, SCNN1A, AP5M1, CLSTN1, AC005077.14, HEG1, SLC39A5, MCL1, MED21, INPPL1, DAZAP2, ELFN1, CDK6, ST3GAL4, TGFB2, PRICKLE2, CYTH1, PLEKHA8, TAF4B, RP11-34016.6, EFNB1, RP11-91J19.4, CTD-2196E14.9, B4GALT4, RASA3, GREB1, ZDHHC18, KRT17, ELMO2, MSANTD3, AGPAT1, YWHAZP3, CGNL1, CUX2, SH3BP5, ZMAT3, SCP2, PEX11B, PPM1K, KIAA1191, GFPT1, GALNT13, C18orf54, CXCR4, ETV1, RNF146, RGL1, HPRT1, EIF4B, CDC42EP2, CTD-2369P2.2, ABI2, BFSP1, SLC6A15, PELI2, TRAK2, DERA, TPP1, EFEMP1, FCHSD2, RSF1, TP53INP1, TK2, PPTC7, HACE1, BOD1, DLX1, YWHAZ, FAM114A2, EFNB2, SMPD1, UBE2F, GPR176, GRB7, ZBTB5, ATOH8, TWSG1, PRNP, OLFML2A, ARPC5, RP11-342K6.1, GCH1, NOTUM, LUZP1, SPECC1L, NUDT21, APPL1, SFXN1, FAT4, UNC119B, ZMIZ1, SCLT1, CD97, AREL1, NYNRIN, ADAMTS1, SEC61A1, SIX4, PNMA3, FAF2, INPP5B, CTGF, OSTF1, TYW3, RAB5B, CBX1, TBC1D12, USP39, ACSS3, CYR61, PCDH17, LMCD1, KIAA1147, EEF1A1P13, KIF3A, EDN1, RP11-53019.3, SMARCA2, EFNA4, GPR133, RBBP5, NCF2, NUP50, OGFRL1, WDFY2, DHCR24, SOS2, YWHAZP2, KLHL2, IGSF3, CCDC80, THSD7A, ZNF618, ACSS2, PDPK1, BCAT2, PHACTR2, GLIS2, MARCKSL1, C11 orf24, CBL, CCNDBP1, NDUFC1, ERBB2IP, IRF1, EIF4BP6, PAFAH2, ALDH9A1, SIPA1 L2, HSBP1, HNRNPUL2, NRP2, NUDT4P1, SDE2, SGMS1, SLC30A1, FOCAD, SERINC2, DESI1, TBL1XR1, TMEM30A, PLEKHB2, DYNLL2, HELZ, TSPAN14, PHGDH, RP1-203P18.1, C17orf103, TRAFD1, DUSP16, NUDT4, FLNC, RGS2, EIF4BP3, DUSP1, TMEM59L, GADD45B, WWC3, RP11-329A14.1, MRPL42, ELOVL7, BACH2, MAP4K5, GNPDA1, NET1, PBX1, BCL2L2, ATP6V1B2, RNF2, SYNRG, SMIM13, COPS3, ARPC2, MDM2, VGLL3, MIR22HG, TRIM10, HSPA13, PTK2, PCDH9, ZC3H7B, LARS2, PITHD1, C1orf106, MAGI3, TNIK, CHSY1, KANK2, TXLNA, TUFM, GMEB1, IWS1, ZNF710, TSC22D3, MVB12A, TMEM216, TAF8, SREK1IP1, IDH2, KIF1B, SLC39A10, STRADB, SLC7A5, PAK4, PTPN9, TGM2, R3HDM1, UGT2B7, CHCHD5, TMEM164, RUSC2, MESDC1, COPRS, EIF4BP7, NOTCH1, USP53, MTA2, NUDT15, DGKH, PLCD3, LPHN2, SLC6A19, KIRREL, IRGQ, RPS6KB2, PSEN1, ANKRD13A, MOCOS, SLC34A2, AMZ1, GBA2, EML4, LINC00511, TEAD4, CA12, KDM5C, CABLES1, NINJ1, WDR82, MAST4, IGFBP4, LPCAT1, CBX6, ZNF512B, ARF3, TMEM135, PDE4D, LSM14B, AFF4, DYRK2, SS18, PTTG1 IP, GLYR1, LUM, NEDD9, JADE3, SEPN1, GGCX, MEX3C, ARHGAP29, MECP2, AMOTL2, PPP1R26, MAGEF1, GABARAPL1, GLIS3, IDH1, SEC22C, NR2F6, PHF10, KATNB1, R3HDM4, AES, WDR48, GNAI3, MYLK, DDA1, HK2, CAPRIN1, CADM4, UNG, SENP5, ARFIP1, KIF14, SLC35D1, NSF, FBXW2, RND3, PLS3, TLE4, CBFB, ALKBH5, CDC14B, GRAMD4, SLC19A2, ELF4, EPHA2, SCAMP4, ARHGEF12, PITPNM1, GGA3, FAM20B, GLO1, MTMR12, DLC1, TAPT1, MPST, UBE2J1, ID3, PRKAA2, PDXK, PIP4K2C, TRIP6, CASP2, NECAP2, TUBB4A, MRPL19, GALNT1, CD2AP, RBFOX2, GOLPH3, PITPNA, TMEM230, KIAA0430, RP11-427H3.3, PPP2R4, AJUBA, KLHL9, EEF1A2, MYADM, RBM8A, PRR14L, AKAP12, NUS1, YAP1, CTDSP2, CHML, PTPLB, DNAJA1, CLN6, DLG1, C12orf49, ZBED6CL, CAB39, ZNF629, FILIP1L, ETNK1, LRRFIP1, NUFIP2, SFT2D2, RAB21, SMAD3, NF1, RPL27A, LARP4B, FKBP9, EP300, TOMM20, CREBBP, SSRP1, SEC31A, BRPF3, SERPINE1, SERINC3, S100A14, CDCA7L, PIP5K1A, GSR, SQSTM1, BAZ2A, SLC20A2, SON, TMBIM1, LAMC1, LGR4, APOH, IGF2BP1, ARFGAP2, BCAR1, FZD5, GDF15, RP11-475C16.1, WDR6, ACTB, NRG4, RAC2, HMGA1P3, SAMD5, RP11-168L7.1, TMEFF2, CTA-14H9.5, AP001059.5, TMEM130, B3GNT4, NPHP3-AS1, HIST1H1E, SLC25A21, RP11-3P17.4, RP11-820L6.1, CTD-2555O16.2, RN7SL381P, RP11-274H2.3, KCNQ4, AC007292.3, RP3-330M21.5, FSIP1, HIST1H2BF, BRSK2, ARHGAP22, CREG2, KCNH2, CENPCP1, CCDC13, CTC-428G20.6, TMEM52B, NEFH, RP11-401P9.4, MYB, RP11-35G9.3, PRL, SYNPO2L, RASL10A, GOLGA7B, RP11-10017.1, SMTNL2, LINC00337, CTD-3092A11.1, CTD-2589H19.6, PLAU, TRPA1, RP11-326K13.4, MPP2, FAM101B, C1QBP, ZNF33B, LEF1, SHMT2, CDC45, CSPG4, PSMB8, UBE2L6, STMN1, RP11-424C20.2, ARHGAP19, UHRF1, FOXRED2, FAM111B, TWISTNB, VAMP8, TMEM30B, SORL1, RP11-296014.3, SLC35D2, E2F8, SLC30A2, KRT23, CCDC28A, ERMP1, RRM2, FCRLB, FAM72A, EVA1A, EXO1, PSRC1, DCAF7, MCM10, FAM72D, PNRC2, DPY19L1, GPR137C, CDT1, ST3GAL4, TGFBR3, ST3GAL5, FAM168B, SPC24, ZDHHC18, C8orf37, PDGFRB, IFIT3, PAX6, ABI2, FBXO5, KDM4D, ATP5F1, LPPR1, NT5E, RP11-386G11.10, RBL1, RP11-421N8.1, CDCA7, C12orf39, ATP5SL, TCF19, E2F2, SULF2, NRGN, ACOT8, POLE3, VASH1, GABPA, HIST4H4, STARD7, CHEK1, CASP9, COLGALT2, ETV1, DDAH1, INO80C, RP11-411B10.4, PCNA, WDR76, COPRS, TPBG, SLC15A1, CCNA2, PHLDA2, SLC23A2, TYMS, HPRT1, SLC29A3, TRPM6, TNFRSF21, NCEH1, DTL, DHCR24, PITPNB, WSB2, DCTN5, ADCY9, GINS2, CDCA5, THSD7A, ASF1B, E2F7, SPC25, CCND1, RUSC1, DAZAP2, TICRR, CLSTN1, RP5-837124.1, CDCA7L, FAM60CP, LRRC34, PPP2R2B, CRISPLD1, DSCC1, RP5-1033H22.2, SLC30A7, ENPP4, UNG, MCM5, KRAS, MCM2, TRHDE, AIMP2, AP2M1, SEPN1, ARID3B, CHAF1A, CDKN2C, PIF1, FAM72B, HACE1, BRCA1, CCNE2, STEAP2, RAD17, AGPAT1, PAQR4, CLSPN, TIMP4, PRPS2, KIF14, CADM1, USP39, CCNJ, GALNT13, MCL1, CCP110, RTN4RL2, FAM64A, UBP1, FAM60A, HYOU1, CXorf57, ARHGAP11A, MOSPD2, PKMYT1, KIAA0101, CKAP2L, PP13439, IL22RA1, CDK2, PLAUR, CNOT6, MND1, BAAT, DUSP7, SFXN2, AL390877.1, MED21, EFNA4, GPATCH11, FAM111A, MOCOS, DHFRP1, SAMD9, BHMT, RP11-253E3.3, NUDT21, CNKSR2, ACSS3, SREK1IP1, CTD-2196E14.9, LRMP, BRIP1, CRKL, RP11-34016.6, MGAT4A, PCYOX1L, MYCN, ZMAT3, LPAR3, NUDT15, CDK1, MCM6, TRAK2, NSF, ROR1, MYBL2, R3HDM1, RGS10, RILPL2, KANK2, PRIM1, PHF19, CA12, CLN6, MK167, SFXN1, SLC45A3, RMI2, HELLS, GAS2, KIF2C, IL17RD, STAMBPL1, NUP50, SPATA5, KLHL2, RGL1, HNRNPUL2, B4GALNT2, CENPM, COPS3, CCNF, CCDC149, RNF168, ORAI1, DERA, APOL6, AUNIP, PPIL3, GBA2, RP11-613M10.6, MAP3K3, ZBTB5, CDC6, POLA2, PCBD1, GNPDA1, SKA3, ORC1, ACOT11, PHACTR2, KIF18B, NCAPG, PLD6, FCHSD2, ACPL2, ERBB2IP, CXCR4, MELK, PTGDR2, CDK6, PIGA, RP1-249H11.4, TK1, IL6R, KIAA1147, C17orf58, CHST14, CEP41, UBR7, MASTL, ARPC5, TONSL, RP11-67L2.2, SAPCD2, UTP18, OSMR, AURKB, IQCC, ITPRIPL1, MSANTD3, SLC26A2, C14orf80, RAD51, LMNB1, UBE2T, SLC25A15, FANCE, PAK4, ZNF512B, DHFR, WDFY2, ZNF618, INCENP, IGF2BP1, ESPL1, PODXL, SS18L1, NR2F6, CHRNA5, MAPK8, KIFC1, MMS22L, BCAT2, OAS3, CDCA4, NAGA, TMPPE, CAPN15, RGS2, RNF146, AP5M1, SYNRG, SCP2, RP1-60019.1, LRRC8A, SGK2, DGKG, NUSAP1, IDH3A, KIF4A, MRPS18B, MBL2, GRB7, FAM20B, CDC25A, PDE4D, TIPIN, TMEM216, RAD51AP1, RP11-21L23.2, BLM, SAAL1, YWHAZP3, TXLNA, KIAA1191, ETS1, KIF15, FANCG, MAX, AREL1, KANK4, CDCA3, NIPA1, FARSA, RFWD3, CGNL1, ATP8B2, MAB21L2, EFHD1, FOCAD, ARMCX4, H2AFX, DEK, WDR62, PPTC7, KIRREL, PRR14L, SSRP1, UBALD2, LINC00657, HJURP, FGF19, GREB1, KNTC1, MCM4, SLC35G1, MARCKSL1, HEG1, MGME1, TAPT1, TPP1, FAF2, RP5-1024G6.8, ATAD5, LARS2, PLK1, ANKRD13A, ARHGEF34P, ATAD2, PAFAH2, TUFM, SLFN13, SKA2, UNC119B, SEC61A1, FEN1, ARF3, SPECC1L, CABLES2, MCM3, SMIM13, BRCA2, GINS1, HMGB1, TMEM30A, ALDH9A1, E2F1, PAG1, LMNB2, CECR2, SYTL5, TMEM194B, WHSC1, IWS1, JADE3, EIF4B, MYLK, SMPD1, PLEKHB2, CENPE, RP11-121L10.3, MPC1, CENPF, TUBA1B, PTPN9, ZWINT, ENTPD5, DSN1, DEPDC1B, SLC43A3, FOXM1, IDS, MORC4, BUB1B, MDM2, GALNT1, NROB2, KIF11, HELZ, C16orf59, MTHFD1, CDC20P1, TP53INP1, XRCC2, RCAN3, ITPKA, PLEKHA8, NDC80, TOP2A, DOLPP1, CASP2, GNL3L, ZCCHC10, GINS3, ABCB10, RAB11FIP5, TRIP13, SLC39A5, FAM83D, WDR82, TBL1XR1, DUT, ZNF395, RECQL4, TCF4, CHAF1B, TFAP4, USP1, ASPM, REXO4, LRRC40, SLC7A5, LIG1, SPP1, PIP4K2C, PDPK1, DNA2, ESCO2, LSM14B, GTSE1, HP1BP3, C10orf12, MCM7, PDE3A, ARHGEF39, CTDSP2, TWF1P1, RFC3, SP4, ACD, PLSCR1, MAD2L1, DKK1, CBX1, AKR1C2, TUBB4B, ZNF346, PLEKHA6, KIF18A, GXYLT1, SLC30A1, MAP7D3, ZNF710, YWHAZP2, RAD54L, WDR5, ARPC2, CBFB, EZH2, CASP8AP2, TFDP1, GLYCTK, SOS2, MXD3, TPRN, GLO1, RRP7A, EML4, MTA2, STIL, PLXNC1, MAGI3, QDPR, PARP14, CDC20, SIPA1 L2, MSH2, RRM1, ELMO2, IQGAP3, KIAA0430, TACC3, PTPLB, NOL9, SEC22C, PBX1, UBE2C, POLQ, HK2, RFC2, TUBA4A, EXOSC3, SS18, WDHD1, CTDSPL2, HSBP1, YWHAZ, NCAPH, RBM8A, XPNPEP1, IGSF3, POLE, C11 orf82, RP11-475C16.1, SLC19A2, ADRBK2, PPAT, TWF1, FST, SGMS1, KIF22, SLC20A2, MRPL19, MKL2, TRAFD1, ALKBH5, NUCKS1, DNMT1, ACSS2, INPPL1, PRR11, RAB5B, EIF4BP7, PRTG, ARHGEF5, DESI1, TMEM135, TUBG1, EIF4BP6, LIMD1, MBNL3, PLK4, CMTM4, SLC30A9, POLR1E, BRI3BP, PITPNA, HMGB2, BOD1, NASP, SLC35D1, ELOVL2, SCAMP4, SMG6, ARHGEF12, POLR2D, FANCD2, LPHN2, SMC4, WDR48, POLA1, KIF20A, DLGAP5, RSF1, SRP54, PIP4K2A, NET1, CDCA8, SYNM, MPST, PNP, SLC18B1, IDH2, OSGIN1, NUP210, RBM10, MDC1, C11 orf24, RPL27A, CDCA2, KIF1B, DYNLL2, PTPN3, TCOF1, LBR, RPS21, KIDINS220, LGR4, KIF23, TOMM20, LAMC1, GLYR1, RPS6KB2, RCC1, TMPO, PTK2, TPX2, SPAG5, CAPRIN1, GTF3C5, SLBP, HMGB1P5, CCNB1, AFF4, ANLN, SEC31A, GSR, H2AFZ, PTTG1IP, and SQSTM1. More preferably it is for downregulating all of these genes. This use is preferably for treatment of cancer, such as for treatment of PTEN-deficient cancer, wherein the cancer is preferably associated with increased or exacerbating expression of said gene. These genes were found to be downregulated by miRNA-193a in Example 1.2.1.

In more preferred embodiments, it is for downregulation of a gene selected from the group consisting of RPS17L, GPR137C, EEF1A1P19, NEFH, KRT14, RP5-973M2.2, OVOL2, RP11-873E20.1, RP5-968P14.2, MYB, AC000068.5, NOTUM, RP11-209D14.2, RP11-326K13.4, RP11-339B21.10, IRF8, HIST1H4C, DPF3, RP11-276H7.3, RP4-694A7.4, RP11-17M16.2, KB-226F1.2, SHBG, LAT2, SNORA33, SNORD12, AC005592.2, RP11-796E2.4, RP11-280G9.1, NOG, LINC00035, 7SK, GJB2, MYH11, BHLHE41, RP11-211N8.2, IL12A, EPB41L3, ROR2, UNC5CL, NINJ2, SUCNR1, CTD-2369P2.2, MYLK4, SLC35D2, SHMT2, ERMP1, TEX15, COL20A1, SORL1, PHLDA2, C10orf91, TWISTNB, HPD, PLAU, IL17RD, RNF182, KRAS, LL22NC03-N14H11.1, AC004158.2, ANKRD44, STMN1, CSPG5, PARD6A, FOXRED2, TMPPE, TNFRSF21, DNASE1 L2, HHAT, TOX3, STARD7, MPP2, B3GNT3, ZDHHC18, RP11-173M1.8, PITPNB, RP11-34016.6, ETV1, ATP5SL, EPHB3, AIMP2, PI3, DHCR24, DAZAP2, C9orf47, ZNF365, RP11-204C16.4, SLC30A7, KIAA1875, NYAP1, CCND1, CHD5, SHOX2, ST3GAL4, SEPN1, KLRG2, VAMP8, AP2M1, FAM60A, SULF2, ZSWIM5, MED21, RP11-24B119.3, Z83851.4, DUSP7, C1QBP, NCEH1, TBX20, UBP1, RP11-421N8.1, LY6K, GPR146, ST3GAL5, ATP5F1, OSMR, CBL, CCDC28A, YWHAZP2, YWHAZ, DPY19L1, EXTL2, NAP1 L5, CTC-428G20.3, ETS1, UBE2L6, GREB1, FAM168B, CLDN4, YWHAZP3, ALX1, CRKL, RPS17, HFE, TRIM62, MSANTD3, ZMAT3, ENDOD1, KIAA1644, MOSPD2, FIBCD1, ATP8B2, PRNP, DLEU2, SLC2A12, WSB2, VGF, SERINC2, DCTN5, XK, NUP50, SMPD1, CNOT6, RP11-395A13.2, GABPA, CLSTN1, POLE3, BOD1, LRRC8A, SLC35G1, TP53INP1, AGPAT1, HPRT1, RUSC1, SLC23A2, PDE3A, EBF1, FANCE, WDYHV1, MAX, WDFY2, MCL1, MORN4, FAM72A, CDCA7L, TPP1, AREL1, COPRS, NT5E, TMEM121, 43346, SLC30A1, PRRG4, RBBP5, LAMC2, SLC35D1, C18orf54, GLYCTK, DPM3, HACE1, CDK6, GPATCH11, IDH2, PTPLB, EMILIN2, CCNA2, FAM60CP, TK2, FAM20B, GNPDA1, TIMP4, NR2F6, TMEM180, TRAF1, DERA, ATP8B3, XYLT1, SLC39A10, KLHL2, PIK3AP1, IDS, CADM1, TBL1XR1, KCNMA1, FAM101B, PPTC7, SLC29A3, AP5M1, PEX11B, MEX3C, ARF3, PLD6, INO80C, SNX10, NUDT15, CCDC149, SLC26A2, GNAI3, DCAF7, APOL6, ADCY9, NUDT21, PRR5L, HYOU1, BCYRN1, CCNDBP1, DSEL, PAFAH2, FAF2, SLC25A15, NEIL2, USP39, GCH1, FCHSD2, TXLNA, TMEM135, RN7SL2, RAB11 FIP5, NAGA, ACOT8, WDR82, PCDHAC2, VAV3, SRP54, RN7SL1, FAM72D, SEC31A, TYW3, ZNF512B, HCG11, ARHGAP29, SAPCD2, PALM3, MIR17HG, DGKH, CASP9, LINC00657, TMEM30A, SLC30A4, CHEK1, RTKN2, IGSF3, NBL1, TGFBR3, RP11-67L2.2, STRADB, APPL1, ARPC5, NIPA1, ZNF710, CA12, NET1, CTD-2196E14.9, ABI2, R3HDM1, SFXN1, MESDC1, MTA2, DOCK10, PHACTR2, KBTBD11, ELK3, PSRC1, MSANTD2, RASGRF2, ATP6V1B2, ALDH9A1, LARS2, CDKL5, RNF216P1, LRRC40, LUZP1, MORC4, MYLK, PLAUR, CCSER2, RP11-73M18.7, ERBB2IP, ACSS2, NOL9, DLEU1, C17orf58, PITHD1, SEC61A1, ARHGAP19, CDC42EP2, HNRNPUL2, BCAT2, RP11-329A14.1, ST5, NUDT3, TNFSF9, PADI2, EMC6, IRF1, PLXNA2, COPS3, CCP110, ABCA12, MPST, CYTH1, PLEKHB2, MED19, GALNT14, MLKL, TOR4A, SYNRG, AFAP1 L1, TRAK2, SGMS1, MARCKSL1, IL6R, PIP4K2C, JADE3, CBX1, HELZ, NSF, IF127L2, TMEM216, SDE2, RTN4RL2, SSRP1, TRPA1, GDF11, SPECC1L, RUFY2, DNAJC3, KIAA1191, AC007560.1, HSBP1, I11, UNG, HEG1, MAP4K5, PPM1F, GLO11, ANKRD13A, KIF1B, KIAA1147, TRAFD1, PAK4, FAM114A2, DIO2, POPDC3, PLEK2, DNAJC16, NT5DC3, RAB27B, TWF1, CLN6, KDM7A, R3HDM4, ZNF618, LRP4, ITPKB, PDPK1, ALKBH5, C11orf24, DSTYK, AAED1, CEP41, MAP3K3, KLHL15, PTPLA, AFMID, LMNB2, MLLT11, DESI1, WDR5, B4GALT6, CCNJ, SENP1, ZBTB14, SIPA1L2, PHF19, TP53BP2, ASF1B, USP43, SS18, CHCHD5, BCL2L2, CAPN15, ADNP2, RCAN3, RNF2, TAP2, SOS2, HDAC3, AP1S2, GFPT1, ABR, FOCAD, ERRF11, RC3H2, EML4, PTPN9, AFF4, CD97, RABEPK, SQSTM1, PRR14L, HP1BP3, GPRC5B, CBFB, ARPC2, GPC6, TCF7L2, GALNT2, TRIP6, PIK3R1, DDAH1, RHOU, UBE2Z, DYNC2H1, ENOX2, IER31P1, ZC3H7B, ZNF324, SPOPL, FBXW2, ORAI1, MUC5AC, C4orf46, KIDINS220, MYADM, SLC3A2, PM20D2, PSEN1, RPS6KB2, TPCN2, GALNT1, RFWD3, GALNT13, FOXN2, TWF1IP1, FKBP9, CCL2, RNF168, GINS3, MRPL42, LYRM2, PTTG1 IP, NRIP1, SSX2IP, DEF8, WDR48, TLR6, EXOSC3, SCP2, FILIP1, INPPL1, TTLL4, TCEB3, SEC22C, IWS1, GBE1, GNL3L, GOSR2, LGR4, SAAL1, UHRF1BP1, SLC29A1, WDR6, VPS37B, HSPA13, TOMM20, PCBD1, CHML, SLC7A5, TP53RK, RUSC2, UTP18, STARD3, C2orf49, BRPF3, PODXL, TUBA1B, PDE8A, DYNLL2, CAPN10, HMGB1, IL4R, SYT1, TUFM, PCBP1, TMBIM1, KCTD5, POM121C, WHSC1, CTDSP2, AGAP2-AS1, KDM5C, PTK2, CPNE3, KIAA0430, CAMKK1, TPCN1, KLHL9, TRIM25, CAPRIN1, UBFD1, MED14, TMEM164, ELMO2, KANK2, ABCB10, CNBP, ITPRIPL2, SOGA1, QDPR, B4GALNT1, FBXW5, TROVE2, FGD6, SUDS3, MTHFD1, KIF14, MAP3K2, AKAP12, OSER1, ACTR3, KIAA0141, ABCE1, HELLS, MRPL19, EIF2AK1, EPHB2, XPNPEP1, YAP1, RBFOX2, CDCA5, ENTPD4, ATP2B4, RBM10, LPCAT1, TPD52, CDK2, AGFG1, WWC3, LBR, PPP2R4, EIF4B, EXTL3, BTBD11, POM121, RIPK2, SFN, MCM2, TMEM230, CMTM4, GSR, TUBA4A, EDEM1, KIRREL, GOLPH3, NF1, TGFB2, PPP3R1, AKR1C3, NOTCH2, CCDC88A, KIAA1522, CTCF, BCAR1, SREBF2, GBF1, WWTR1, PDE4D, CDK4, PGRMC1, AKR1C2, MAP7D1, SET, NCOA3, SERINC3, ARHGAP11A, DEK, PRKCA, MLEC, SYNM, GNB1, PLS3, DDB1, F2RL1, GPC1, and SERPINE1, even more preferably it is for downregulation of all of these genes. This use is preferably for treatment of lung cancer, such as for treatment of PTEN-deficient lung cancer, wherein the cancer is preferably associated with increased or exacerbating expression of said gene. These genes were found to be downregulated by miRNA-193a in A549 cells (Example 1.2.1).

In other more preferred embodiments, it is for downregulation of a gene selected from the group consisting of KRT79, HMGCLL1, LINC00920, BTBD11, RP11-390F4.8, NEURL3, RP11-423P10.2, PAX5, KCNIP1, CD93, PLCB2, RP11-290F20.2, PDGFRB, MEDAG, CRISPLD1, RP5-1086K13.1, DLL1, AL139099.1, AC007383.3, AC046143.3, DNM3, AC111200.7, C11 orf35, RP5-1157M23.2, PDE5A, CSF2, CMAHP, C6orf58, ITPKA, SLC22A14, SLC29A3, FOXRED2, ACTG2, SULF2, FAM211A, AC011043.1, CYS1, CTD-2313J17.5, AKNAD1, RP11-456K23.1, APOBEC3F, ZMYND15, RP11-588K22.2, CYP2D7P, ERMP1, ADAM22, ABCA9, GRB7, LL22NC03-86G7.1, HSPB7, FAM196B, SOX9-AS1, FAM227B, BEST3, TRAM1 L1, SGIP1, ADCY7, PCED1B, SEPN1, APOBEC3G, CCDC28A, NGFR, MPP2, IL17RD, PLAU, TMEM173, IFT27, CTD-2292P10.4, ZNRF2P2, NT5E, DGKB, TWISTNB, STMN1, RTN4RL2, SLC25A34, HFE, S100A16, RP11-807H7.1, KRT15, ITGB3, CIB2, SHMT2, GAB2, CMTM8, GALNT13, CCDC149, GALNT14, SLC35D2, CCND1, SYNPO2, ATP5SL, ETV1, TMEM216, TNFRSF1B, USP18, BCAT2, ACOT8, HYOU1, AP2M1, HTR7, PALM3, RP4-760C5.5, OTUB2, PLEKHA5, MIR621, TMPPE, RGS2, TNFRSF21, ERAP2, DCAF7, SFXN2, KRAS, DAZAP2, CLSTN1, ARHGDIB, FAM114A2, TP531NP1, TCEAL8, ST6GALNAC3, CERS1, PTPRE, PDE3A, CTSO, SLC30A4, ENDOD1, SLC23A2, C1QBP, UBE2L6, CNRIP1, ST3GAL5, ENPP4, PARD3B, PLD6, DPY19L1, ABCA8, MORN4, MYB, SLC26A2, NSF, FAT4, TPP1, SLC30A7, ZNF512B, ACPL2, RP11-2711.4, EDNRA, A4GALT, ZNF836, RNF146, PLCD1, STARD7, PEX11B, UPRT, CEP41, PTPRZ1, AGPAT1, ARHGAP19, XAF1, DHCR24, OSMR, AGAP2, MAST4, ACSS2, FBXL16, RHOU, RP11-18114.10, GBP3, POU2F2, AC009948.5, FAT3, PLCD3, LRRC8C, PGPEP1, SEC31A, SLC18B1, ISLR2, LINC00669, ZMAT3, IL1B, AIMP2, CCNJ, MOSPD2, GLYCTK, ST3GAL4, LRRC8A, TNIP3, MSANTD3, ANKRD13A, PCBD1, DERA, ARHGAP27, GLDCP1, GABPA, DGKA, ATP8B2, RUSC1, ZNF362, PRPF40B, SAMD9L, STS, RAB5B, CCL20, PCYOX1L, NFE2L3, USP27X, KDM7A, CDC42EP2, MMP1, FAM72B, GPR146, WNK4, MEF2BNB, MYOZ3, PAD12, CDKL5, HTR7P1, PTCH1, OAS2, ZNF365, OBSCN, PDE4D, WSB2, CYTH1, NCEH1, KIF5C, PRNP, MTSS1, FAM60A, LINC00657, GPD1L, FOCAD, DCTN5, PIK3R1, UBP1, RP11-34016.6, ZDHHC18, LOX, PIGA, CA12, APOLD1, PGM5, AKAP12, MCL1, PHLDA2, ZNF608, HACE1, BMF, IGSF3, PITPNB, ZSWIM3, ERBB21P, NUDT18, PTPN9, ZCCHC10, ITGA2, PIP4K2C, TRAK2, LGR4, AP5M1, EBF1, DOCK4, AL390877.1, MED21, ELMO3, AC108676.1, GPRASP2, NAGA, CNOT6, ATP5F1, ZNF710, EPM2A, OSBPL5, COPRS, FCHSD2, TRIB2, TK2, TBX20, RUFY2, SREK1IP1, GNA13, RP11-421N8.1, IL8, FAM132B, YWHAZ, TAF9B, WDFY2, YWHAZP3, MARCKSL1, ETS1, TRIM62, HK2P1, ALDH9A1, OSBP2, TMEM180, GNPDA1, SAMD9, BTN3A2, YWHAZP2, PTK2, PNRC2, RAD17, IQCD, DNAJB9, ARHGEF9, POLE3, ARMCX2, DPM3, KANK2, DOK3, PLAUR, INPPL1, NT5DC3, DNMBP, LRRC40, ARHGEF40, SYNRG, GPATCH11, IWS1, RGL1, SEC61A1, PHACTR2, CDC14B, ZNF181, KLHL2, CBX7, IDS, PAK4, FAM72A, MPST, WBP5, ARF3, ACSL5, UBE2Q2P6, DDAH1, ASAP3, TRO, GAS1, PTPLB, ST5, SCP2, DOCK10, PXK, ARHGAP29, CXCL2, HECW1, LAMC2, R3HDM4, MAP3K3, MLLT11, GBE1, HYAL2, RAB11FIP5, GRAMD4, C11orf95, ADAMTS18, APBB2, CCSER2, WDR48, FAF2, STC1, IDH1, NUDT3, PARP14, NET1, AKR1C3, CHCHD5, HEMK1, TUFM, ELK3, DGKQ, CDK6, LPAR1, GDF15, CTDSP2, GULP1, MMP14, SIX4, LARS2, CD38, LRP5, CRKL, SMPD1, DUSP16, JAK2, B3GNT1, KIAA1147, FAM214B, PARD6A, SLC12A9, SS18L1, DGKH, PSEN1, ENOX2, PAX6, UFL1, FAM210B, TPCN1, SMG6, MAG13, PALMD, NEIL2, PDK4, APAF1, AGFG1, SLC35D1, SLC25A15, RNF215, GALNT1, HEG1, TRAF1, SRP54, PDGFD, HNRNPUL2, MDM2, TMEM30A, RSPO2, GPC6, PLEKHA2, CACNG4, CASZ1, PAG1, EXTL2, IFIT3, KANK1, RNF2, TNIK, PTPN21, ENTPD4, QDPR, PTPN3, SYNJ2, TMEM164, KITLG, FBXL15, PGAP1, DENND4C, GSDMD, TRAPPC9, ALKBH5, TRAFD1, DAB2, JADE3, PDPK1, COPS3, ABL1, EVA1C, EML4, SFXN1, LRP3, DDX60, EIF4BP3, DNAJC3, TGFBR3, DAK, CTTNBP2NL, GNA11, STARD3, TGM2, SLC9A3, IRF1, HK2, PLEKHB2, MAGEF1, PPTC7, RPS6KB2, ADAMTS15, EIF4BP7, SCAMP4, ADAMTSL1, NDFIP2, EIF4B, GPR176, MORC4, ERBB2, FAM20B, AREL1, GNL3L, USP39, SLC39A10, BOD1, ATP6V1B2, ARHGAP18, KIAA0430, GFPT1, EIF4BP6, SREBF2, FBXL17, MAX, CBFB, NECAP2, GEM, CDC42EP3, KIAA1522, KLHL9, CBL, KIAA1644, RCAN1, SUSD5, JADE2, GRHL3, SMARCA1, USP40, SQSTM1, KIF1B, LUZP1, SMIM14, MEX3C, ARHGEF1, NUP50, HELZ, CCDC90B, PPM1H, BCAR1, RAB27B, PSMB8, ANTXR1, SENP1, F2RL1, ARPC5, SIPA1 L2, LNPEP, UBALD2, ZC3H7B, NUDT21, YAP1, FAM65A, LRBA, BMPR2, FRMD6, APPL1, AMIGO2, SCAMP1, AES, LPHN2, ZNF395, WDR82, HPRT1, PRKCA, TDO2, TCF4, TRIM8, SFT2D2, SLC20A2, ADAMTS1, SEC23B, RSF1, CPNE3, MAMLD1, DYRK2, LLGL1, NR2F2, TRIP6, SOS2, ARPC2, ERRF11, IDO1, PLSCR1, RNF182, BNC2, STAM, MX1, TCTN3, CHML, ELMO2, PITPNA, GALNT2, KLF3, RIPK2, PPM1F, LPCAT1, TBX18, MRPS18B, KIRREL, HSPA13, MAP4K5, LRRC8D, MAGED2, NCOA3, BACH1, IL7R, CCNA2, KDM5C, SLC30A1, CCNY, PIP4K2A, DDB1, RND3, DAPK1, GOLPH3, SSRP1, INTS3, FAM168B, TMCC3, CDK4, ZMIZ1, TM4SF1, NSD1, MTA2, SNHG5, GIT1, PPP2R4, KIAA1191, TXLNA, RC3H2, TMBIM1, TNFAIP8, HELZ2, UHMK1, CREBBP, WIPI2, FRMD8, PLIN2, NOTCH2, LIF, ANGPTL4, DUSP4, SLC7A5, LAMC1, PLS3, and SNX9, even more preferably it is for downregulation of all of these genes. This use is preferably for treatment of breast cancer, more preferably of triple negative breast cancer, wherein the cancer is preferably associated with increased or exacerbating expression of said gene. These genes were found to be downregulated by miRNA-193a in BT549 cells (Example 1.2.1).

In other more preferred embodiments, it is for downregulation of a gene selected from the group consisting of GPRC5B, RP11-30P6.6, LEF1, RGS17P1, CTC-428G20.6, CAMKV, RP11-440D17.3, RASA4, OXCT2, GRAP, CTA-217C2.2, ADAMTS16, AC119673.1, MPP2, CAMK2B, FGFR2, MIR103A2, LINC00460, RP11-540B6.3, AC005789.11, RP11-196016.1, TCERG1L, TNFRSF1B, ARMCX4, STON2, PARD6A, FAM156A, AGAP1-IT1, AC010525.6, MYRF, FBXL16, MAPK13, RLTPR, EXOC3L4, CCDC28A, HMX3, NDN, TP73, CTA-445C9.15, EXPH5, PHLDA2, RASSF5, ST3GAL5, 03-Sep, STMN1, INSRR, SHMT2, N4BP3, TWISTNB, CACNG6, PLAU, ERMP1, FOXRED2, SEPN1, KALRN, LRP4, I1 RL1, AC009061.1, PDE9A, TGM2, IGSF9B, PTGER2, DAZAP2, PITPNB, FAM132B, FKBP9L, ATP5SL, STARD7, HOXD13, RHOV, WDFY2, GNA15, HYOU1, DDAH1, INO80C, UBE2L6, ATP8B2, PRKCH, AP2M1, DHCR24, TOR4A, TMEM121, SRRM3, ARHGAP19, SLC39A10, RP11-82L118.2, AGPAT1, DND1, NT5E, GJB2, SLC30A7, F2RL1, FAM105A, ELK3, GCH1, GRTP1, NID1, SLC30A1, IRF1, PTK7, SERINC2, TMEM173, MARCKSL1, CCND1, FIBCD1, KIAA1644, COPRS, P2RX5, ZNF365, HHAT, TNFRSF21, VAMP8, SLC35D2, RP11-34016.6, KRAS, ZDHHC18, WNT9A, IGSF3, DPM3, ALDH1A3, PRDM8, SLC26A2, ROR1, ACSS2, C11 orf95, GALNT14, STC1, IL8, NPIPB4, UBP1, NR2F6, PRNP, USP39, DUSP7, FAM101B, FAM60A, ST3GAL4, OSMR, SH2B2, FAM168B, STRADB, ZNF703, TRIM62, SOX18, YWHAZ, CDK6, GNAI3, RP11-204C16.4, FOXL1, ACPL2, GNPDA1, LRRC8A, GREB1, SLC30A4, SORL1, TBC1D5, RP3-425P12.4, ALX4, NCEH1, CHST14, MCL1, VASH2, SLC45A3, AIMP2, RUSC1, RASL11A, ICAM1, RP11-329A14.1, HEG1, PIK3R1, ETS1, KIAA1147, ANKLE1, CXCL3, PTX3, EFNB2, FAM20B, DGKH, YWHAZP2, DPY19L1, YWHAZP3, DCAF7, BCAT2, SCAMP4, DCTN5, RAB11FIP5, BOD1, ZMAT3, C12orf39, CCNJ, WSB2, GPR161, POLE3, NEFH, GPR3, RBM24, SUCNR1, B4GALNT3, IDH2, TEX15, TERT, RP11-101E13.5, SFXN1, C16orf59, C1QBP, TGFBR3, ZCCHC10, KLHL2, IL6R, AP5M1, GALNT13, PSRC1, PDGFRB, LMNB2, COL5A3, MYEOV, TMEM164, SERPINE1, CXCL2, STAMBPL1, GFPT1, DERA, WDYHV1, PDPK1, SIPA1L2, CCNDBP1, MAX, KANK2, PITHD1, IL4R, NT5DC3, ATP5F1, FAM60CP, PFDN1, CA12, PMAIP1, NPTX1, CLSTN1, MOSPD2, NUDT21, SLC35D1, GABPA, TRAFD1, RP11-22P6.3, UBE2Q2P6, NUDT15, SLC7A5, MESDC1, ADCY1, TMEM216, PDE3A, ENDOD1, CRKL, LOXL1, NHS, NES, TBX2, DMTN, EGR1, GPATCH11, CNOT6, TEAD3, UNG, AREL1, PLSCR1, HPRT1, RNF138, TAF4B, RFWD3, MAMLD1, ARHGAP26, AKAP12, PAK4, TXLNA, MPST, TNFAIP8, RAB5B, SMPD1, PM20D2, MSANTD3, CXCL1, SOX7, PAPPA2, CMTM3, CAPN15, RP11-421N8.1, DOCK10, SEC61A1, KCNK5, NAGA, LINC00941, CXCL5, TBL1XR1, FAM72D, ZG16B, TMOD1, PNRC2, GDF11, SEC31A, PLCD3, PTPLB, PLEKHB2, FOSB, NRIP3, HSD11B2, GPR27, WDR5, ARF3, RNF216P1, ZNF35, CASP9, SLC29A3, ST8SIA4, SCP2, FCHSD2, ABR, ARHGEF40, KLHL15, PPM1F, KCTD12, APLN, DTL, CCNA2, SRP54, SLC16A6, LRRC40, MED21, EML4, TNFRSF8, IL1 RAP, HFE, FOXN2, ALKBH5, CCDC85C, SLC23A2, ARPC4, GLO1, SYNRG, ORAI1, ZNF678, NOTCH2, ST5, LUZP1, KIF1B, KCTD5, DLX1, RGS2, TANGO2, FAM72B, CASP2, UBE2Z, SSH3, FAF2, ADCY9, C18orf54, MAFF, MAP3K3, RBBP5, KLHL23, JADE3, ZNF618, BAI2, CBX1, PLXNA2, CDK2, CBFB, CBL, NUP50, GLI2, MMP1, CMTM4, BMP6, PSEN1, JAG2, LINC00657, ARHGAP29, ACSS3, ARPC5, TUBG1, FOCAD, TUFM, ZC3H7B, KIF26A, TP73-AS1, PAG1, RC3H2, SENP1, MTA2, CDCA7, SLC29A1, TRAK2, RNF2, POM121C, RNF146, TONSL, TEAD4, ELMO2, ENTPD4, BRPF3, PGRMC1, CLN6, OSBPL10, ERRF11, PODXL, AMIGO2, LRRC8C, ANKRD13A, GALNT1, ASAP3, NUDT4, OSBPL8, CDC42EP2, SLC19A2, IL18R1, SMOX, EFNB1, TMEM30A, POM121, SLC16A9, UNC119B, ARPC2, INPPL1, KIRREL, CNKSR2, BCL2L2, TOMM20, SPRY4, SDC1, AFF4, FOS, SH2B3, KIAA1191, RNF215, SLC18B1, CTDSP2, PXK, TCEB3, SREBF2, C12orf49, KLHL8, APOL6, UBALD2, HK2, NET1, RUFY2, C17orf58, C11orf24, CDCA7L, SAMD8, MAPK8, NOTCH1, PEX11B, HSPA13, PPTC7, DMRTA2, NEIL2, COPS3, TPD52, HNRNPUL2, FKBP9, EXOSC3, CCP110, PLAUR, GATA2, AB12, SSRP1, SYNJ2, CBX6, CHCHD5, WDR82, PPP2R4, HSF1, ERBB21P, PCBD1, SREK1IP1, MAP4K5, FRMD8, CRLF3, DDA1, EIF4B, FERMT3, CSRNP1, IWS1, LARS2, ID1, R3HDM1, ENOX2, WNT5A, FBXW2, PTK2, MTFR1, WNK2, SCAMP1, QDPR, PPAT, HELZ2, TK2, LPHN2, FZD8, TMBIM1, ALDH9A1, ELF4, BHLHE40, NUDT3, ASF1B, STS, WDR6, JAG1, PSMB8, PIP4K2C, CYP51A1P2, RNH1, THRA, MAP7D1, MFN2, PHF19, RNF168, ETS2, ANTXR2, SLC35G1, MEX3C, UTP18, PPP4R1, MDC1, HELLS, ATP6VOA2, DYNLL2, GOLPH3, SQSTM1, PATZ1, DESI1, GALNT2, HIP1, LINC00152, SAPCD2, FAM210B, PLXNA1, R3HDM4, REXO4, TYW3, CCDC14, SPECC1L, STARD4, ABCB10, NSF, ALG2, MAGEA1, KRT80, ZBED4, DEF8, SH3PXD2B, LSM14B, DUSP5, PAQR4, HSPB8, TRIB3, FBXW5, RBM10, SFT2D2, PDE4D, WHSC1, UPP1, FAM115C, EPDR1, RASA3, XPNPEP1, CDC45, MYADM, HN1L, BCOR, PRKAA2, RAPH1, CCSER2, CHEK1, NAB1, SLCO4A1, ADRBK1, PXN, B4GALNT1, TSPAN14, RIN1, TCOF1, SMG5, HP1BP3, RP11-1055B8.7, HSBP1, SKA2, OGFRL1, CDT1, SGMS1, MCM10, APPL1, ATP6V1B2, TROVE2, CD97, TRIP13, SS18, PHLDA1, TRIM25, FOSL1, ID3, PPP1R26, PPP3R1, RFC3, MRPS18B, GPC1, SET, IDS, MED14, IER2, TFPI2, UBFD1, CDCA4, OGFR, CNBP, PAPOLA, MRPL19, TNFAIP2, AKR1C3, TOMM34, FGFRL1, MCM2, KIAA0141, ADNP, LPCAT1, CDC6, MLEC, KIDINS220, AGFG1, HMGB1, LIF, IDH3A, UHMK1, TRIP6, RBM8A, FARSA, URB1, PITPNA, GNB1, WWTR1, SETP14, RPS21, CAPRIN1, TGOLN2, STC2, OSGIN1, NOTCH3, IDH1, BAZ1B, DDB1, TNPO1, LASP1, PCBP1, FASN, and TUBB, ACTB, even more preferably it is for downregulation of all of these genes. This use is preferably for treatment of lung cancer, such as for treatment of PTEN-deficient lung cancer, wherein the cancer is preferably associated with increased or exacerbating expression of said gene. These genes were found to be downregulated by miRNA-193a in H460 cells (Example 1.2.1).

In other more preferred embodiments, it is for downregulation of a gene selected from the group consisting of RP11-313P13.5, CTB-31N19.3, LINC00607, LRRC15, RGS17P1, NPAS3, CTD-3203P2.2, CSTF3-AS1, CTD-2342J14.6, CTD-2537I9.5, MYEOV, ANKRD31, CIDEC, MYO1G, SRRM3, LINC01132, ENDOD1, TSGA101P, ADH1A, IL11, RP11-572C15.6, CD207, RP11-274H2.5, TFF3, UXT-AS1, RPS19P3, RP11-305K5.1, CTD-2192J16.20, LLOXNC01-250H12.3, ZSCAN23, LINC01096, RPSAP52, CDC42EP3, AK4P3, GALNT16, ETS1, SEC14L2, CHST6, RP11-255H23.2, LINC01057, ULK2, FAM162B, RP11-1017G21.5, CTD-2161E19.1, MFSD4, ASAP3, AC026150.5, AC005077.12, LINC00312, TRIM62, CCDC28A, ROM1, TRPV3, RP11-73M18.9, HHAT, B3GNT3, TMEM30B, CRYAA, TFAP2A-AS1, TMEM151A, DACT3, SLC35D2, CCDC17, TGFBR3, TIMP4, MPP2, MYCN, TWISTNB, C19orf77, CCND1, NT5E, PTX3, RP11-116D2.1, SOCS3, SHMT2, PRR15L, DOK7, NAPA-AS1, SPP1, ERMP1, UBE2L6, NACAD, SLC6A12, KCNG3, CHCHD2P6, ERBB4, ANGPTL2, FAM150A, LOX, ANKLE1, ACOT8, ST3GAL5, AMPD3, SLC15A1, IL17RD, MYADML2, C8A, FOXRED2, GRIN2D, STMN1, DCAF7, TRIM17, HR, C1QBP, LINC00657, HFE, RP11-469M7.1, ATP5SL, FAM60CP, RP11-421N8.1, MPZ, SLC29A3, PNRC2, ARHGAP19, CDC42EP4, FAM168B, GTF2H2B, SORL1, PPARGC1A, P2RY1, KRAS, PHLDA2, DPY19L1, CCDC149, CCNJ, TNFRSF21, RUSC1, UBP1, IDS, DCTN5, SYNE3, LAMC2, KDM7A, LEF1, GABPA, PCBD1, PCYOX1L, TNFSF9, PDGFRB, MAT1A, VAMP8, ARID3B, CXXC4, MYB, ATP5F1, RP11-204C16.4, RP11-973N13.2, FAM101B, ENPP4, KRT80, HYOU1, PITPNB, WSB2, CRKL, UPK1A-AS1, AP2M1, FAM60A, PSRC1, DUSP7, DDAH1, ANKRD1, FAM203A, CNOT6, CER1, RP11-613M10.6, ATP8B2, IL6R, AAED1, ESRRG, INO80C, HSPB8, SLC23A2, SOX5, PDE3A, HFE2, NPTX1, ADCY9, MAX, NPPB, SLC30A7, RAB11FIP5, ZCCHC10, PAG1, MAPK8, FOXC1, UNC5CL, STARD7, LRRC8A, HRK, MISP, AIMP2, PIK3R1, PLAG1, POLE3, ACPL2, NCEH1, SCNN1A, AP5M1, CLSTN1, AC005077.14, HEG1, SLC39A5, MCL1, MED21, INPPL1, DAZAP2, ELFN1, CDK6, ST3GAL4, TGFB2, PRICKLE2, CYTH1, PLEKHA8, TAF4B, RP11-34016.6, EFNB1, RP11-91J19.4, CTD-2196E14.9, B4GALT4, RASA3, GREB1, ZDHHC18, KRT17, ELMO2, MSANTD3, AGPAT1, YWHAZP3, CGNL1, CUX2, SH3BP5, ZMAT3, SCP2, PEX11B, PPM1K, KIAA1191, GFPT1, GALNT13, C18orf54, CXCR4, ETV1, RNF146, RGL1, HPRT1, EIF4B, CDC42EP2, CTD-2369P2.2, ABI2, BFSP1, SLC6A15, PELI2, TRAK2, DERA, TPP1, EFEMP1, FCHSD2, RSF1, TP53INP1, TK2, PPTC7, HACE1, BOD1, DLX1, YWHAZ, FAM114A2, EFNB2, SMPD1, UBE2F, GPR176, GRB7, ZBTB5, ATOH8, TWSG1, PRNP, OLFML2A, ARPC5, RP11-342K6.1, GCH1, NOTUM, LUZP1, SPECC1L, NUDT21, APPL1, SFXN1, FAT4, UNC119B, ZMIZ1, SCLT1, CD97, AREL1, NYNRIN, ADAMTS1, SEC61A1, SIX4, PNMA3, FAF2, INPP5B, CTGF, OSTF1, TYW3, RAB5B, CBX1, TBC1D12, USP39, ACSS3, CYR61, PCDH17, LMCD1, KIAA1147, EEF1A1P13, KIF3A, EDN1, RP11-53019.3, SMARCA2, EFNA4, GPR133, RBBP5, NCF2, NUP50, OGFRL1, WDFY2, DHCR24, SOS2, YWHAZP2, KLHL2, IGSF3, CCDC80, THSD7A, ZNF618, ACSS2, PDPK1, BCAT2, PHACTR2, GLIS2, MARCKSL1, C11orf24, CBL, CCNDBP1, NDUFC1, ERBB2IP, IRF1, EIF4BP6, PAFAH2, ALDH9A1, SIPA1L2, HSBP1, HNRNPUL2, NRP2, NUDT4P1, SDE2, SGMS1, SLC30A1, FOCAD, SERINC2, DESI1, TBL1XR1, TMEM30A, PLEKHB2, DYNLL2, HELZ, TSPAN14, PHGDH, RP1-203P18.1, C17orf103, TRAFD1, DUSP16, NUDT4, FLNC, RGS2, EIF4BP3, DUSP1, TMEM59L, GADD45B, WWC3, RP11-329A14.1, MRPL42, ELOVL7, BACH2, MAP4K5, GNPDA1, NET1, PBX1, BCL2L2, ATP6V1B2, RNF2, SYNRG, SMIM13, COPS3, ARPC2, MDM2, VGLL3, MIR22HG, TRIM10, HSPA13, PTK2, PCDH9, ZC3H7B, LARS2, PITHD1, C1 orf106, MAGI3, TNIK, CHSY1, KANK2, TXLNA, TUFM, GMEB1, IWS1, ZNF710, TSC22D3, MVB12A, TMEM216, TAF8, SREK1IP1, IDH2, KIF1B, SLC39A10, STRADB, SLC7A5, PAK4, PTPN9, TGM2, R3HDM1, UGT2B7, CHCHD5, TMEM164, RUSC2, MESDC1, COPRS, EIF4BP7, NOTCH1, USP53, MTA2, NUDT15, DGKH, PLCD3, LPHN2, SLC6A19, KIRREL, IRGQ, RPS6KB2, PSEN1, ANKRD13A, MOCOS, SLC34A2, AMZ1, GBA2, EML4, LINC00511, TEAD4, CA12, KDM5C, CABLES1, NINJ1, WDR82, MAST4, IGFBP4, LPCAT1, CBX6, ZNF512B, ARF3, TMEM135, PDE4D, LSM14B, AFF4, DYRK2, SS18, PTTG1 IP, GLYR1, LUM, NEDD9, JADE3, SEPN1, GGCX, MEX3C, ARHGAP29, MECP2, AMOTL2, PPP1R26, MAGEF1, GABARAPL1, GLIS3, IDH1, SEC22C, NR2F6, PHF10, KATNB1, R3HDM4, AES, WDR48, GNAI3, MYLK, DDA1, HK2, CAPRIN1, CADM4, UNG, SENP5, ARFIP1, KIF14, SLC35D1, NSF, FBXW2, RND3, PLS3, TLE4, CBFB, ALKBH5, CDC14B, GRAMD4, SLC19A2, ELF4, EPHA2, SCAMP4, ARHGEF12, PITPNM1, GGA3, FAM20B, GLO1, MTMR12, DLC1, TAPT1, MPST, UBE2J1, ID3, PRKAA2, PDXK, PIP4K2C, TRIP6, CASP2, NECAP2, TUBB4A, MRPL19, GALNT1, CD2AP, RBFOX2, GOLPH3, PITPNA, TMEM230, KIAA0430, RP11-427H3.3, PPP2R4, AJUBA, KLHL9, EEF1A2, MYADM, RBM8A, PRR14L, AKAP12, NUS1, YAP1, CTDSP2, CHML, PTPLB, DNAJA1, CLN6, DLG1, C12orf49, ZBED6CL, CAB39, ZNF629, FILIP1L, ETNK1, LRRFIP1, NUFIP2, SFT2D2, RAB21, SMAD3, NF1, RPL27A, LARP4B, FKBP9, EP300, TOMM20, CREBBP, SSRP1, SEC31A, BRPF3, SERPINE1, SERINC3, S100A14, CDCA7L, PIP5K1A, GSR, SQSTM1, BAZ2A, SLC20A2, SON, TMBIM1, LAMC1, LGR4, APOH, IGF2BP1, ARFGAP2, BCAR1, FZD5, GDF15, RP11-475C16.1, WDR6, and ACTB, even more preferably it is for downregulation of all of these genes. This use is preferably for treatment of liver cancer, such as for treatment of PTEN-deficient liver cancer, wherein the cancer is preferably associated with increased or exacerbating expression of said gene. These genes were found to be downregulated by miRNA-193a in HEP3B cells (Example 1.2.1).

In other more preferred embodiments, it is for downregulation of a gene selected from the group consisting of NRG4, RAC2, HMGA1P3, SAMD5, RP11-168L7.1, TMEFF2, CTA-14H9.5, AP001059.5, TMEM130, B3GNT4, NPHP3-AS1, HIST1H1E, SLC25A21, RP11-3P17.4, RP11-820L6.1, CTD-2555O16.2, RN7SL381P, RP11-274H2.3, KCNQ4, AC007292.3, RP3-330M21.5, FSIP1, HIST1H2BF, BRSK2, ARHGAP22, CREG2, KCNH2, CENPCP1, CCDC13, CTC-428G20.6, TMEM52B, NEFH, RP11-401P9.4, MYB, RP11-35G9.3, PRL, SYNPO2L, RASL10A, GOLGA7B, RP11-10017.1, SMTNL2, LINC00337, CTD-3092A11.1, CTD-2589H119.6, PLAU, TRPA1, RP11-326K13.4, MPP2, FAM101B, C1QBP, ZNF33B, LEF1, SHMT2, CDC45, CSPG4, PSMB8, UBE2L6, STMN1, RP11-424C20.2, ARHGAP19, UHRF1, FOXRED2, FAM111B, TWISTNB, VAMP8, TMEM30B, SORL1, RP11-296014.3, SLC35D2, E2F8, SLC30A2, KRT23, CCDC28A, ERMP1, RRM2, FCRLB, FAM72A, EVA1A, EXO1, PSRC1, DCAF7, MCM10, FAM72D, PNRC2, DPY19L1, GPR137C, CDT1, ST3GAL4, TGFBR3, ST3GAL5, FAM168B, SPC24, ZDHHC18, C8orf37, PDGFRB, IFIT3, PAX6, ABI2, FBXO5, KDM4D, ATP5F1, LPPR1, NT5E, RP11-386G11.10, RBL1, RP11-421N8.1, CDCA7, C12orf39, ATP5SL, TCF19, E2F2, SULF2, NRGN, ACOT8, POLE3, VASH1, GABPA, HIST4H4, STARD7, CHEK1, CASP9, COLGALT2, ETV1, DDAH1, INO80C, RP11-411B10.4, PCNA, WDR76, COPRS, TPBG, SLC15A1, CCNA2, PHLDA2, SLC23A2, TYMS, HPRT1, SLC29A3, TRPM6, TNFRSF21, NCEH1, DTL, DHCR24, PITPNB, WSB2, DCTN5, ADCY9, GINS2, CDCA5, THSD7A, ASF1B, E2F7, SPC25, CCND1, RUSC1, DAZAP2, TICRR, CLSTN1, RP5-837124.1, CDCA7L, FAM60CP, LRRC34, PPP2R2B, CRISPLD1, DSCC1, RP5-1033H22.2, SLC30A7, ENPP4, UNG, MCM5, KRAS, MCM2, TRHDE, AIMP2, AP2M1, SEPN1, ARID3B, CHAF1A, CDKN2C, PIF1, FAM72B, HACE1, BRCA1, CCNE2, STEAP2, RAD17, AGPAT1, PAQR4, CLSPN, TIMP4, PRPS2, KIF14, CADM1, USP39, CCNJ, GALNT13, MCL1, CCP110, RTN4RL2, FAM64A, UBP1, FAM60A, HYOU1, CXorf57, ARHGAP11A, MOSPD2, PKMYT1, KIAA0101, CKAP2L, PP13439, IL22RA1, CDK2, PLAUR, CNOT6, MND1, BAAT, DUSP7, SFXN2, AL390877.1, MED21, EFNA4, GPATCH11, FAM111A, MOCOS, DHFRP1, SAMD9, BHMT, RP11-253E3.3, NUDT21, CNKSR2, ACSS3, SREK1IP1, CTD-2196E14.9, LRMP, BRIP1, CRKL, RP11-34016.6, MGAT4A, PCYOX1L, MYCN, ZMAT3, LPAR3, NUDT15, CDK1, MCM6, TRAK2, NSF, ROR1, MYBL2, R3HDM1, RGS10, RILPL2, KANK2, PRIM1, PHF19, CA12, CLN6, MK167, SFXN1, SLC45A3, RMI2, HELLS, GAS2, KIF2C, IL17RD, STAMBPL1, NUP50, SPATA5, KLHL2, RGL1, HNRNPUL2, B4GALNT2, CENPM, COPS3, CCNF, CCDC149, RNF168, ORAI1, DERA, APOL6, AUNIP, PPIL3, GBA2, RP11-613M10.6, MAP3K3, ZBTB5, CDC6, POLA2, PCBD1, GNPDA1, SKA3, ORC1, ACOT11, PHACTR2, KIF18B, NCAPG, PLD6, FCHSD2, ACPL2, ERBB2IP, CXCR4, MELK, PTGDR2, CDK6, PIGA, RP1-249H11.4, TK1, IL6R, KIAA1147, C17orf58, CHST14, CEP41, UBR7, MASTL, ARPC5, TONSL, RP11-67L2.2, SAPCD2, UTP18, OSMR, AURKB, IQCC, ITPRIPL1, MSANTD3, SLC26A2, C14orf80, RAD51, LMNB1, UBE2T, SLC25A15, FANCE, PAK4, ZNF512B, DHFR, WDFY2, ZNF618, INCENP, IGF2BP1, ESPL1, PODXL, SS18L1, NR2F6, CHRNA5, MAPK8, KIFC1, MMS22L, BCAT2, OAS3, CDCA4, NAGA, TMPPE, CAPN15, RGS2, RNF146, AP5M1, SYNRG, SCP2, RP1-60019.1, LRRC8A, SGK2, DGKG, NUSAP1, IDH3A, KIF4A, MRPS18B, MBL2, GRB7, FAM20B, CDC25A, PDE4D, TIPIN, TMEM216, RAD51AP1, RP11-21L23.2, BLM, SAAL1, YWHAZP3, TXLNA, KIAA1191, ETS1, KIF15, FANCG, MAX, AREL1, KANK4, CDCA3, NIPA1, FARSA, RFWD3, CGNL1, ATP8B2, MAB21L2, EFHD1, FOCAD, ARMCX4, H2AFX, DEK, WDR62, PPTC7, KIRREL, PRR14L, SSRP1, UBALD2, LINC00657, HJURP, FGF19, GREB1, KNTC1, MCM4, SLC35G1, MARCKSL1, HEG1, MGME1, TAPT1, TPP1, FAF2, RP5-1024G6.8, ATAD5, LARS2, PLK1, ANKRD13A, ARHGEF34P, ATAD2, PAFAH2, TUFM, SLFN13, SKA2, UNC119B, SEC61A1, FEN1, ARF3, SPECC1L, CABLES2, MCM3, SMIM13, BRCA2, GINS1, HMGB1, TMEM30A, ALDH9A1, E2F1, PAG1, LMNB2, CECR2, SYTL5, TMEM194B, WHSC1, IWS1, JADE3, EIF4B, MYLK, SMPD1, PLEKHB2, CENPE, RP11-121L10.3, MPC1, CENPF, TUBA1B, PTPN9, ZWINT, ENTPD5, DSN1, DEPDC1B, SLC43A3, FOXM1, IDS, MORC4, BUB1B, MDM2, GALNT1, NROB2, KIF11, HELZ, C16orf59, MTHFD1, CDC20P1, TP53INP1, XRCC2, RCAN3, ITPKA, PLEKHA8, NDC80, TOP2A, DOLPP1, CASP2, GNL3L, ZCCHC10, GINS3, ABCB10, RAB11FIP5, TRIP13, SLC39A5, FAM83D, WDR82, TBL1XR1, DUT, ZNF395, RECQL4, TCF4, CHAF1B, TFAP4, USP1, ASPM, REXO4, LRRC40, SLC7A5, LIG1, SPP1, PIP4K2C, PDPK1, DNA2, ESCO2, LSM14B, GTSE1, HP1BP3, C10orf12, MCM7, PDE3A, ARHGEF39, CTDSP2, TWF1P1, RFC3, SP4, ACD, PLSCR1, MAD2L1, DKK1, CBX1, AKR1C2, TUBB4B, ZNF346, PLEKHA6, KIF18A, GXYLT1, SLC30A1, MAP7D3, ZNF710, YWHAZP2, RAD54L, WDR5, ARPC2, CBFB, EZH2, CASP8AP2, TFDP1, GLYCTK, SOS2, MXD3, TPRN, GLO1, RRP7A, EML4, MTA2, STIL, PLXNC1, MAGI3, QDPR, PARP14, CDC20, SIPA1 L2, MSH2, RRM1, ELMO2, IQGAP3, KIAA0430, TACC3, PTPLB, NOL9, SEC22C, PBX1, UBE2C, POLQ, HK2, RFC2, TUBA4A, EXOSC3, SS18, WDHD1, CTDSPL2, HSBP1, YWHAZ, NCAPH, RBM8A, XPNPEP1, IGSF3, POLE, C11 orf82, RP11-475C16.1, SLC19A2, ADRBK2, PPAT, TWF1, FST, SGMS1, KIF22, SLC20A2, MRPL19, MKL2, TRAFD1, ALKBH5, NUCKS1, DNMT1, ACSS2, INPPL1, PRR11, RAB5B, EIF4BP7, PRTG, ARHGEF5, DESI1, TMEM135, TUBG1, EIF4BP6, LIMD1, MBNL3, PLK4, CMTM4, SLC30A9, POLR1E, BRI3BP, PITPNA, HMGB2, BOD1, NASP, SLC35D1, ELOVL2, SCAMP4, SMG6, ARHGEF12, POLR2D, FANCD2, LPHN2, SMC4, WDR48, POLA1, KIF20A, DLGAP5, RSF1, SRP54, PIP4K2A, NET1, CDCA8, SYNM, MPST, PNP, SLC18B1, IDH2, OSGIN1, NUP210, RBM10, MDC1, C11 orf24, RPL27A, CDCA2, KIF1B, DYNLL2, PTPN3, TCOF1, LBR, RPS21, KIDINS220, LGR4, KIF23, TOMM20, LAMC1, GLYR1, RPS6KB2, RCC1, TMPO, PTK2, TPX2, SPAG5, CAPRIN1, GTF3C5, SLBP, HMGB1P5, CCNB1, AFF4, ANLN, SEC31A, GSR, H2AFZ, PTTG1IP, and SQSTM1, even more preferably it is for downregulation of all of these genes. This use is preferably for treatment of liver cancer, such as for treatment of PTEN-deficient liver cancer, wherein the cancer is preferably associated with increased or exacerbating expression of said gene. These genes were found to be downregulated by miRNA-193a in HUH7 cells (Example 1.2.1).

In preferred embodiments the miRNA-193a is for use in treating a cancer associated with at least one active pathway selected from the group of active pathways as listed in tables 6, 9, 12, 15, and 18, preferably with increased activity of said pathway, more preferably with increased activity of all pathways listed in tables 6, 9, 12, 15, and 18. In preferred embodiments the miRNA-193a is for use in treating a cancer associated with at least one aberrantly expressed gene associated with a pathway selected from the group of associated genes as listed in tables 6, 9, 12, 15, and 18. Increased or active expression of a pathway or gene is preferably assessed by comparison to expression in a healthy cell or tissue sample or untreated cell or tissue sample. This use is preferably for decreasing expression of said pathway. This use is preferably for modulating expression of at least one gene associated with the pathway, wherein the associated gene is preferably selected from the group of associated genes shown in tables 6, 9, 12, 15, and 18, more preferably it is for modulating all said genes. More preferably it is for treating a lung cancer with at least one active pathway selected from the group of active pathways as listed in table 6 or 12. More preferably it is for treating a breast cancer with at least one active pathway selected from the group of active pathways as listed in table 9. More preferably it is for treating a liver cancer with at least one active pathway selected from the group of active pathways as listed in table 15 or 18.

In preferred embodiments the miRNA-193a is for use in treating a cancer associated with at least one aberrant pathway selected from the group of aberrant pathways as listed in tables 7, 10, 13, 16, and 19, more preferably with aberrant activity of all pathways listed in tables 7, 10, 13, 16, and 19. In preferred embodiments the miRNA-193a is for use in treating a cancer associated with at least one aberrantly expressed gene associated with a pathway selected from the group of associated genes as listed in tables 7, 10, 13, 16, and 19. Aberrant expression of a pathway or gene is preferably assessed by comparison to expression in a healthy cell or tissue sample or untreated cell or tissue sample. Aberrant expression is preferably an increased activity. In other preferred embodiments, aberrant expression is a decreased activity. This use is preferably for modulating expression of said pathway. This use is preferably for modulating expression of at least one gene associated with the pathway, wherein the associated gene is preferably selected from the group of associated genes shown in tables 7, 10, 13, 16, and 19, more preferably it is for modulating all said genes. More preferably it is for treating a lung cancer with at least one aberrant pathway selected from the group of aberrant pathways as listed in table 7 or 13. More preferably it is for treating a breast cancer with at least one aberrant pathway selected from the group of aberrant pathways as listed in table 10. More preferably it is for treating a liver cancer with at least one aberrant pathway selected from the group of aberrant pathways as listed in table 16 or 19.

In preferred embodiments the miRNA-193a is for use in treating a cancer associated with at least one aberrant pathway, preferably downregulated pathway, selected from the group of aberrant pathways as listed in tables 8, 11, 14, 17, and 20, preferably with decreased activity of said pathway, more preferably with decreased activity of all pathways listed in tables 8, 11, 14, 17, and 20. In preferred embodiments the miRNA-193a is for use in treating a cancer associated with at least one aberrantly expressed gene associated with a pathway selected from the group of associated genes as listed in tables 8, 11, 14, 17, and 20. Decreased or aberrant expression of a pathway or gene is preferably assessed by comparison to expression in a healthy cell or tissue sample or untreated cell or tissue sample. This use is preferably for increasing expression of said pathway. This use is preferably for modulating expression of at least one gene associated with the pathway, wherein the associated gene is preferably selected from the group of associated genes shown in tables 8, 11, 14, 17, and 20, more preferably it is for modulating all said genes. More preferably it is for treating a lung cancer with at least one aberrant pathway selected from the group of aberrant pathways as listed in table 8 or 14. More preferably it is for treating a breast cancer with at least one aberrant pathway selected from the group of aberrant pathways as listed in table 11. More preferably it is for treating a liver cancer with at least one aberrant pathway selected from the group of aberrant pathways as listed in table 17 or 20.

Compositions for use according to the invention and miRNA for use according to the invention promote cell cycle arrest in tumour cells. In preferred embodiments, the miRNA for use according to the invention or the composition for use according to the invention are for use in the treatment of cancer, wherein the use is for inducing cell cycle arrest. Cell cycle arrest profiles can be measured for example by performing either nuclei imaging or flow cytometry, preferably as demonstrated in the examples. In this context, cell cycle arrest is preferably the induction of a G2/M or a SubG1 cell cycle arrest profile. Preferably, 1%, 5%, 10%, 15%, 20%, 25%, 30%, 40%, 50%, 55%, 60%, 65%, 70% or 75%, or more tumour cells undergo cell cycle arrest. Preferably, when the miRNA for use according to the invention is for treating PTEN-deficient melanoma, liver cancer, carcinoma, lung cancer, or pancreas cancer, the miRNA for use according to the invention is for increasing cell cycle arrest profiles.

### Composition

The invention also relates to compositions comprising the miRNA for use according to the invention, wherein the composition is for that same use. Such a composition comprises a miRNA-193a or a source thereof as for use according to the invention. It is referred to hereinafter as a composition for use according to the invention. Preferably such compositions are pharmaceutical compositions. Such compositions further preferably comprise a pharmaceutically acceptable solvent, or a pharmaceutically acceptable excipient, or a pharmaceutically acceptable diluent, or a pharmaceutically acceptable carrier.

Preferred compositions for use according to the invention comprise a miRNA-193a or a source thereof, preferably wherein the miRNA-193a is a miRNA193a molecule, an isomiR, or a mimic thereof. More preferably, compositions for use according to the invention comprise a miRNA-193a or a source thereof, wherein the miRNA-193a is a miRNA-193a molecule, an isomiR, or a mimic thereof, and is an oligonucleotide with a seed sequence comprising at least 6 of the 7 nucleotides of the seed sequence represented by SEQ ID NO: 22. Highly preferred compositions comprise nanoparticles as later defined herein.

In preferred embodiments, this aspect provides the composition for use according to the invention, further comprising a further miRNA or precursor thereof, wherein the further miRNA is selected from the group consisting of miRNA-323, miRNA-342, miRNA-520f, miRNA-520f-i3, miRNA-3157, and miRNA-7, or an isomiR thereof, or a mimic thereof.

The inventors have surprisingly found that a nanoparticle formulation comprising a diamino lipid provides excellent results when used as compositions for use according to the invention. Accordingly, in preferred embodiments the composition for use according to the invention is a nanoparticle composition, the nanoparticle comprising a diamino lipid and a miRNA-193a or a source thereof as defined above, wherein the diamino lipid is of general formula (I)

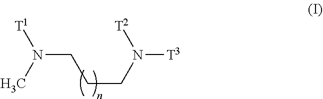

wherein
n is 0, 1, or 2, and
T¹, T², and T³ are each independently a C₁₀-C₁₈ chain with optional unsaturations and with zero, one, two, three, or four substitutions, wherein the substitutions are selected from the group consisting of C₁-C₄ alkyl, C₁-C₄ alkenyl, and C₁-C₄ alkoxy.

Such a composition is referred to hereinafter as a nanoparticle composition for use according to the invention. In the context of this application, a nanoparticle is a particle with dimensions in the nanometer range, or in some cases in the micrometer range. Preferably, a nanoparticle is as least 5, 10, 15, 20, 25, 30, 35, 40, 45, 50, 55, 60, 65, 70, 75, 80, 85, 90, 95, 100, 110, 120, 130, 140, 150, 160, 170, 180, 190, 200 or more nanometer in diameter, where a diameter is preferably an average diameter of a population of nanoparticles. Preferably, a nanoparticle is at most 100, 150, 200, 250, 300, 350, 400, 450, 500, 550, 600, 650, 700, 750, 800, 850, 900, 950, 1000, 1100, 1200, 1300, 1400, 1500, 2000, 5000, or 10000 nanometer in diameter. More preferably, nanoparticles have an average diameter of 40-300 nm, even more preferably of 50-200 nm, even more preferably of 50-150 nm, most preferably of 65-85 nm, such as about 70 nm.

Nanoparticle compositions for use according to the invention comprise lipid nanoparticles that further comprise an oligonucleotide. The oligonucleotide can be seen as the cargo or the payload of the nanoparticle. Accordingly, the nanoparticles can for example be micelles, liposomes, lipoplexes, unilamellar vesicles, multilamellar vesicles, or cross-linked variants thereof. It is preferred that the nanoparticles are micelles, liposomes, or lipoplexes. When reference is made to the composition of the nanoparticles, reference to the diamino lipid and optional further excipients is intended, and no reference to any cargo substances is intended. As a non-limiting example, when the nanoparticle is said to comprise 50 mol % of the diamino lipid and 50 mol % of other excipients, the molar percentages only relate to the diamino lipid and those other excipients; the oligonucleotide molar fraction or the molar fraction of solvents is not taken into account.

When the invention relates to a composition comprising more than one miRNA molecule, isomiR, mimic, or source thereof it is encompassed that each miRNA molecule, isomiR, mimic, or source thereof may be present each in a separate composition. Each composition can be sequentially or simultaneously administered to a subject, or mixed prior to use into a single composition. Alternatively, it is also encompassed that more than one miRNA molecules, isomiRs, mimics, or sources thereof is present in a single composition as defined herein.

The nanoparticle compositions for use according to the invention comprises a diamino lipid of general formula (I), but it may also comprise further lipids. In preferred embodiments, the diamino lipid is the most prevalent lipid in the nanoparticle by molar percent. As used herein, the term lipid refers to substances that are soluble in nonpolar solvents such as CH₂Cl₂. The diamino lipids used in the invention have three tails linked to a spacer and thus resemble naturally occurring triglyceride lipids. Several such lipids are known (U.S. Pat. No. 8,691,750).

The diamino lipid of general formula (I) comprises two tertiary amines that are separated by an aliphatic spacer of varying length. The spacer helps determine the headgroup size of the lipid. n can be 0, 1, or 2, so the spacer is in effect an 1,2-ethylene, n-1,3-propylene, or n-1,4-butylene spacer. In particular preferred embodiments, n is 0. In particular preferred embodiments, n is 1. In particular preferred embodiments, n is 2. It is most preferred that n is 1. Accordingly, in preferred embodiments the invention provides a nanoparticle composition for use according to the invention, wherein the diamino lipid is of general formula (I) wherein n is 1. Accordingly, in preferred embodiments the invention provides a nanoparticle composition for use according to the invention, wherein the diamino lipid is of general formula (I-1)

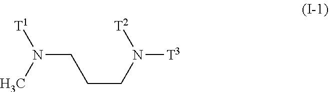

Wherein T¹, T², and T³ are each independently a C₁₀-C₁₈ chain with optional unsaturations and with zero, one, two, three, or four substitutions, wherein the substitutions are selected from the group consisting of C₁-C₄ alkyl, C₁-C₄ alkenyl, and C₁-C₄ alkoxy.

T¹, T², and T³ can be seen as the tails of the lipid, and are aliphatic C₁₀-C₁₈ with optional unsaturations and up to four optional substitutions. T¹, T², and T³ can be independently selected, or the same choice can be made for two or three of T¹, T², and T³. In preferred embodiments, this aspect provides the nanoparticle composition for use according to the invention, wherein the diamino lipid is of general formula (I) wherein T¹, T², and T³ are identical. Identical should not be so narrowly construed as to imply that the natural abundance of isotopes should be contemplated—identical should preferably only refer to the molecular structure as would be represented in a drawn structural formula.

Longer chains will generally lead to more rigid lipid membranes. In this application the number in C₁₀-C₁₈ refers to the longest continuous chain that can be determined, and not to the total C content. As a non-limiting example, an n-dodecyl chain with an n-propyl substitution at a 6-position comprises 15 C atoms but is a C₁₂ chain because the longest continuous chain has a length of 12 C atoms. Unsaturations can lead to less rigid membranes if the unsaturation is cis in the chain, bending it. A preferred unsaturation is cis. In preferred embodiments, T¹, T², and T³ contain zero, one, two, three, or four unsaturations. In more preferred embodiments, T¹, T², and T³ contain one, two, three, or four unsaturations. In even more preferred embodiments, T¹, T², and T³ contain one, two, or three unsaturations, preferably three unsaturations.

The optional substitutions are selected from the group consisting of C₁-C₄ alkyl, C₁-C₄ alkenyl, and C₁-C₄ alkoxy A preferred optional substitution is a C₁-C₄ alkyl, more preferably a C₁-C₂ alkyl, most preferably methyl (—CH₃). There are zero, one, two, three, or four of such substitutions, which means that substitutions can be absent. As such the substitutions are optional. Preferably, there are zero, one, two, or three such substitutions.

In preferred embodiments, T¹, T², and T³ are each independently a C₁₀-C₁₆ chain with optional unsaturations and with zero, one, two, three, or four substitutions, wherein the substitutions are selected from the group consisting of C₁-C₄ alkyl, C₁-C₄ alkenyl, and C₁-C₄ alkoxy. In more preferred embodiments, T¹, T², and T³ are each independently a C₁₀-C₁₄ chain with optional unsaturations and with zero, one, two, three, or four substitutions, wherein the substitutions are selected from the group consisting of C₁-C₄ alkyl, C₁-C₄ alkenyl, and C₁-C₄ alkoxy. Most preferably, T¹, T², and T³ are each independently a C₁₂ chain with optional unsaturations and with zero, one, two, three, or four substitutions, wherein the substitutions are selected from the group consisting of C₁-C₄ alkyl, C₁-C₄ alkenyl, and C₁-C₄ alkoxy.

In preferred embodiments, T¹, T², and T³ are each independently a C₁₀-C₁₈ chain with one, two, three, or four unsaturations and with zero, one, two, three, or four substitutions, wherein the substitutions are selected from the group consisting of C₁-C₄ alkyl, C₁-C₄ alkenyl, and C₁-C₄ alkoxy.

In preferred embodiments, T¹, T², and T³ are each independently a C₁₀-C₁₈ chain with one, two, or three unsaturations and with one, two, three, or four substitutions, wherein the substitutions are selected from the group consisting of C₁-C₄ alkyl, C₁-C₄ alkenyl, and C₁-C₄ alkoxy.

In preferred embodiments, T¹, T², and T³ are each independently a C₁₀-C₁₈ chain with one, two, or three unsaturations and with one, two, three, or four substitutions, wherein the substitutions are selected from the group consisting of C₁-C₄ alkyl.

In preferred embodiments, T¹, T², and T³ are each independently a C₁₀-C₁₄ chain with one, two, or three unsaturations and with one, two, or three substitutions, wherein the substitutions are selected from the group consisting of C₁-C₂ alkyl.

Preferred embodiments for T¹, T², and T³ are (with a name in systematic Cₙ numbering, wherein a number after a colon (as in C1_{2:3}) indicates the degree of unsaturation) (2E, 6E)-farnesyl (C_{12:3}), lauryl (C₁₂), tridecyl (C₁₃), myristryl (C₁₄), pentadecyl (C₁₅), cetyl (C₁₆), margaryl (C₁₇), stearyl (C₁₈), α-linolenyl (C_{18:3}), γ-linolenyl (C_{18:3}), linoleyl (C_{18:2}), stearidyl (C_{18:4}), vaccenyl (C_{18:1}), oleyl (C_{18:1}), elaidyl (C_{18:1}), palmitoleyl (C_{18:1}), (2E, 6Z)-farnesyl, (2Z, 6E)-farnesyl, (2Z, 6Z)-farnesyl, and 3,7,11-trimethyldodecyl.

Accordingly, in preferred embodiments this aspect provides the nanoparticle composition for use according to the invention, wherein the diamino lipid is of general formula (I) wherein T¹, T², and T³ are each independently selected from the group consisting of farnesyl, lauryl, tridecyl, myristryl, pentadecyl, cetyl, margaryl, stearyl, α-linolenyl, γ-linolenyl, linoleyl, stearidyl, vaccenyl, oleyl, elaidyl, palmitoleyl, and 3,7,11-trimethyldodecyl. Preferably, T¹, T², and T³ are each independently selected from the group consisting of farnesyl, lauryl, tridecyl, myristryl, pentadecyl, cetyl, α-linolenyl, γ-linolenyl, linoleyl, stearidyl, oleyl, palmitoleyl, and 3,7,11-trimethyldodecyl. More preferably, T¹, T², and T³ are each independently selected from the group consisting of farnesyl, lauryl, tridecyl, myristryl, stearidyl, palmitoleyl, and 3,7,11-trimethyldodecyl. Even more preferably, T¹, T², and T³ are each independently selected from the group consisting of farnesyl, lauryl, tridecyl, myristryl, and 3,7,11-trimethyldodecyl. Even more preferably, T¹, T², and T³ are each independently selected from the group consisting of farnesyl, lauryl, and 3,7,11-trimethyldodecyl. Most preferably, T¹, T², and T³ are each independently farnesyl, such as (2E,6E) farnesyl, (2E,6Z) farnesyl, (2Z,6E) farnesyl, or (2Z,6Z) farnesyl; preferably they are each (2E,6E) farnesyl.

Farnesyl is also known as 3,7,11-trimethyldodeca-2,6,10-trienyl and is an unsaturated linear C₁₂ chain; it can be (2E,6E), (2E,6Z), (2Z,6E), or (2Z,6Z); preferably it is (2E,6E). Lauryl is also known as dodecyl and is a saturated linear C₁₂ chain. Tridecyl is a saturated linear C₁₃ chain. Myristryl is also known as tetradecyl and is a saturated linear C₁₄ chain. Pentadecyl is a saturated linear C₁₅ chain. Cetyl is also known as palmityl and is a saturated linear C₁₆ chain. Margaryl is also known as heptadecyl and is a saturated linear C₁₇ chain. Stearyl is also known as octadecyl and is a saturated linear C₁₈ chain. α-linolenyl is also known as (9Z,12Z,15Z)-9,12,15-octadecatrienyl and is an unsaturated linear C₁₈ chain. γ-linolenyl is also known as (6Z, 9Z, 12Z)-6,9,12-octadecatrienyl and is an unsaturated linear C₁₈ chain. Linoleyl is also known as (9Z,12Z)-9,12-octadecadienyl and is an unsaturated linear C₁₈ chain. Stearidyl is also known as (6Z,9Z,12Z,15Z)-6,9,12,15-octadecatetraenyl and is an unsaturated linear C₁₈ chain. Vaccenyl is also known as (E)-octadec-11-enyl and is an unsaturated linear C₁₈ chain. Oleyl is also known as (9Z)-octadec-9-enyl and is an unsaturated linear C₁₈ chain. Elaidyl is also known as (9E)-octadec-9-enyl and is an unsaturated linear C₁₈ chain. Palmitoleyl is also known as (9Z)-hexadec-9-enyl and is an unsaturated linear C₁₆ chain. 3,7,11-trimethyldodecyl is saturated farnesyl and is a saturated linear C₁₂ chain.

The composition can further comprise solvents and/or excipients, preferably pharmaceutically acceptable excipients. Preferred solvents are aqueous solutions such as pharmaceutically acceptable buffers, for example PBS or citrate buffer. A preferred citrate buffer comprises 50 mM citrate at pH 2.5-3.5 such as pH 3, preferably set using NaOH. A preferred PBS is at pH 7-8 such as pH 7.4. PBS preferably does not comprise bivalent cations such as Ca²⁺ and Mg²⁺. Another preferred pharmaceutically acceptable excipient is ethanol. Most preferably, the composition comprises a physiological buffer such as PBS or a Good's buffer or Hepes-buffered saline or Hank's balanced salt solution or Ringer's balanced salt solution or a Tris buffer. Preferred compositions are pharmaceutical compositions. The composition can comprise further excipients. These further excipients can be comprised in the nanoparticles.

In preferred embodiments, this aspect provides the nanoparticle composition for use according to the invention, further comprising a sterol, preferably selected from the group consisting of adosterol, brassicasterol, campesterol, cholecalciferol, cholestenedione, cholestenol, cholesterol, delta-7-stigmasterol, delta-7-avenasterol, dihydrotachysterol, dimethylcolesterol, ergocalciferol, ergosterol, ergostenol, ergostatrienol, ergostadienol, ethylcholestenol, fusidic acid, lanosterol, norcholestadienol, β-sitosterol, spinasterol, stigmastanol, stigmastenol, stigmastadienol, stigmastadienone, stigmasterol, and stigmastenone, more preferably cholesterol. More particularly, in preferred embodiments, this aspect provides the nanoparticle composition for use according to the invention, wherein the nanoparticles further comprise a sterol, preferably selected from the group consisting of adosterol, brassicasterol, campesterol, cholecalciferol, cholestenedione, cholestenol, cholesterol, delta-7-stigmasterol, delta-7-avenasterol, dihydrotachysterol, dimethylcolesterol, ergocalciferol, ergosterol, ergostenol, ergostatrienol, ergostadienol, ethylcholestenol, fusidic acid, lanosterol, norcholestadienol, β-sitosterol, spinasterol, stigmastanol, stigmastenol, stigmastadienol, stigmastadienone, stigmasterol, and stigmastenone, more preferably cholesterol.

Preferably, such a further comprised sterol is not conjugated to any moiety. Conjugated sterols can also be comprised, as will be explained later herein. As such, both conjugated and unconjugated sterols can be comprised. Unless explicitly indicated otherwise, reference to a sterol is intended as reference to an unconjugated sterol.

When a sterol is comprised in the composition, it is preferably comprised in the nanoparticle, and preferably at least 5, 10, 15, 20, 25, 30, 35, 40, 45, 50, 55, 60, 65, or 70 mol % of sterol is comprised; preferably at most 80, 75, 70, 65, 60, 65, 50, 45, 40, 35, or 30 mol % of sterol is comprised. As explained above, this molar percentage only pertains to the substances making up the lipid nanoparticle, and not to solvents or cargo such as oligonucleotides. When a sterol is comprised in the composition, preferably 5 to 70 mol %, 15 to 60 mol %, 25 to 60 mol %, 35 to 60 mol %, 40 to 60 mol %, or 45 to 55 mol % is comprised; more preferably 40 to 60 mol % or 45 to 55 mol % is comprised, most preferably 45 to 55 mol % is comprised, such as 48 mol % or 54 mol %.

In preferred embodiments, this aspect provides the nanoparticle composition for use according to the invention, further comprising a phospholipid, preferably selected from the group consisting of distearoyl phosphatidylcholine (DSPC), dipalmitoyl phosphatidylcholine (DPPC), dimyristoyl phosphatidylcholine (DMPC), dilauroyl phosphatidylcholine (DLPC), dioleyl phosphatidylcholine (DOPC), 1,2-dioleoyl-sn-glycero-3-phosphoethanolamine (DOPE), egg phosphatidylcholine (EggPC), soy phosphatidylcholine (SoyPC), more preferably distearoyl phosphatidylcholine (DSPC). More particularly, in preferred embodiments, this aspect provides the nanoparticle composition for use according to the invention, wherein the nanoparticles further comprise a phospholipid, preferably selected from the group consisting of distearoyl phosphatidylcholine (DSPC), dipalmitoyl phosphatidylcholine (DPPC), dimyristoyl phosphatidylcholine (DMPC), dilauroyl phosphatidylcholine (DLPC), dioleyl phosphatidylcholine (DOPC), 1,2-dioleoyl-sn-glycero-3-phosphoethanolamine (DOPE), egg phosphatidylcholine (EggPC), soy phosphatidylcholine (SoyPC), more preferably distearoyl phosphatidylcholine (DSPC).

Preferably, such a further comprised phospholipid is not conjugated to any moiety. Conjugated phospholipids can also be comprised, as will be explained later herein. As such, both conjugated and unconjugated phospholipids can be comprised.

When a phospholipid is comprised in the composition, it is preferably comprised in the nanoparticle, and preferably at least 1, 2, 3, 4, 5, 6, 7, 8, 9, 10, 11, 12, 13, 14, 15, 16, 17, 18, 19, 20, 25, 30, 35, 40, 45, 50, 55, or 60 mol % of phospholipid is comprised; preferably at most 65, 60, 55, 50, 45, 40, 35, 30, 25, 20, 15, 14, 13, 12, 11, 10, 9, 8, 7, 6, or 5 mol % of phospholipid is comprised. As explained above, this molar percentage only pertains to the substances making up the lipid nanoparticle, and not to solvents or cargo such as oligonucleotides. When a phospholipid is comprised in the composition, preferably 0 to 40 mol %, 0 to 35 mol %, 0 to 30 mol %, 5 to 30 mol %, 5 to 25 mol %, or 5 to 20 mol % is comprised; more preferably 5 to 20 mol % or 5 to 15 mol % is comprised, most preferably 5 to 15 mol % is comprised, such as 10 mol % or 11 mol %.

In preferred embodiments, this aspect provides the nanoparticle composition for use according to the invention, further comprising a conjugate of a water soluble polymer and a lipophilic anchor, wherein:
i) the water soluble polymer is selected from the group consisting of poly(ethylene glycol) (PEG), poly(hydroxyethyl-1-asparagine) (PHEA), poly-(hydroxyethyl-L-glutamine) (PHEG), poly(glutamic acid) (PGA), polyglycerol (PG), poly(acrylamide) (PAAm), poly(vinylpyrrolidone) (PVP), poly(N-(2-hydroxypropyl)methacrylamide) (PHPMA), and poly(2-oxazoline) (POx) such as poly(2-methyl-2-oxazoline) (PMeOx) and poly(2-ethyl-2-oxazoline) (PEtOx), or copolymers thereof and wherein
ii) the lipophilic anchor is selected from the group consisting of a sterol, a lipid, and a vitamin E derivative. Preferably, the lipophilic anchor is a lipid, more preferably a diglyceride.

More particularly, in preferred embodiments, this aspect provides the nanoparticle composition for use according to the invention, wherein the nanoparticles further comprise a conjugate of a water soluble polymer and a lipophilic anchor as described above. The water soluble polymer generally increases the colloidal stability of the nanoparticles, to which is it linked via the lipophilic anchor. In general, the lipophilic anchor embeds in the lipid bilayer or in the micelle, and thus links the water soluble polymer to the surface of the nanoparticle. The use of such water soluble polymers for this purpose is known in the art (Knop et al., 2010, doi: 10.1002/anie.200902672). A preferred water soluble polymer is poly(ethylene glycol). Preferably, the water soluble polymer has a molecular weight ranging from about 750 Da to about 15000 Da, more preferably from about 1000 Da to about 6000 Da, even more preferably from about 1000 Da to about 3000 Da, most preferably from about 1500 Da to about 3000 Da, such as about 2000 Da. Accordingly, PEG-2000 is a preferred water soluble polymer for use in a conjugate as described above. The water soluble polymer is preferably a linear polymer, and is preferably conjugated at one of its two termini. The other terminus is preferably uncharged at physiological conditions, such as a hydroxyl group or a methyl or ethyl ether. Preferably, the non-conjugated terminus is a methyl ether or a hydroxyl group, most preferably a methyl ether.

The lipophilic anchor to which the water soluble polymer is conjugated generally serves to ensure a connection between the water soluble polymer and the nanoparticle. The particular conjugation between the polymer and the anchor is not important, a skilled person can select any suitable chemical bond such as an ester bond, an amide bond, an ether linkage, a triazole, or any other moiety resulting from conjugating a water soluble polymer to a lipophilic anchor. The use of small linkers is also envisaged, such as succinic acid or glutaric acid. The lipophilic anchor is selected from the group consisting of a sterol, a lipid, and a vitamin E derivative. Preferred sterols are described above. Preferred vitamin E derivatives are tocopherols and tocotrienols such as alpha-tocopherol, beta-tocopherol, gamma-tocopherol, delta-tocopherol, and corresponding tocotrienols. Preferably, the lipophilic anchor is a lipid, more preferably a diglyceride or a phospholipid. Examples of preferred lipids are described above, examples of preferred diglycerides are distearoylglycerol, preferably 1,2-distearoyl-sn-glycerol, dipalmitoylglycerol, preferably 1,2-dipalmitoyl-sn-glycerol, dioleoylglycerol, preferably 1,2-dioleoyl-sn-glycerol, and diarachidoylglycerol, preferably 1,2-diarachidoyl-sn-glycerol. A most preferred diglyceride is distearoylglycerol, preferably 1,2-distearoyl-sn-glycerol.

Suitable examples of conjugates as described above are (1,2-distearoyl-sn-glycerol)-[methoxy(polyethylene glycol-2000)] ether, (1,2-distearoyl-sn-glycerol)-[methoxy(polyethylene glycol-1500)] ether, (1,2-distearoyl-sn-glycerol)-[methoxy(polyethylene glycol-3000)]ether, (1,2-distearoyl-sn-glycerol)-[hydroxy(polyethylene glycol-2000)]ether, (1,2-distearoyl-sn-glycerol)-[hydroxy(polyethylene glycol-1500)]ether, (1,2-distearoyl-sn-glycerol)-[hydroxy(polyethylene glycol-3000)]ether, (1,2-distearoyl-sn-glyceryl)-[methoxy(polyethylene glycol-2000)carboxylate], (1,2-distearoyl-sn-glyceryl)-[methoxy(polyethylene glycol-1500) carboxylate], (1,2-distearoyl-sn-glyceryl)-[methoxy(polyethylene glycol-3000) carboxylate], (1,2-distearoyl-sn-glyceryl)-[hydroxy(polyethylene glycol-2000) carboxylate], (1,2-distearoyl-sn-glyceryl)-[hydroxy(polyethylene glycol-1500) carboxylate], (1,2-distearoyl-sn-glyceryl)-[hydroxy(polyethylene glycol-3000) carboxylate], (1,2-distearoyl-sn-glyceryl)-[methoxy(polyethylene glycol-2000) carbamate], (1,2-distearoyl-sn-glyceryl)-[methoxy(polyethylene glycol-1500) carbamate], (1,2-distearoyl-sn-glyceryl)-[methoxy(polyethylene glycol-3000) carbamate], (1,2-distearoyl-sn-glyceryl)-[hydroxy(polyethylene glycol-2000) carbamate], (1,2-distearoyl-sn-glyceryl)-[hydroxy(polyethylene glycol-1500) carbamate], and (1,2-distearoyl-sn-glyceryl)-[hydroxy(polyethylene glycol-3000) carbamate], wherein the stearoyl moieties can optionally be replaced by other fatty acids, preferably by other Coo-C₂₀ fatty acids. For carbamates and esters as described above, the parent amines and parent alcohols and parent carboxylic acids can also be switched around, for example a PEG-alcohol can be reacted with a carboxylic acid analogue of a diglyceride. Most preferred examples of conjugates are (1,2-distearoyl-sn-glycerol)-[methoxy(polyethylene glycol-2000)] ether, which is also known as DSG-PEG (CAS #: 308805-39-2), and its ester analogue (1,2-distearoyl-sn-glyceryl)-[methoxy(polyethylene glycol-2000)carboxylate], and its carbamate analogue (1,2-distearoyl-sn-glyceryl)-[methoxy(polyethylene glycol-2000) carbamate] or 1,2-distearoyloxy propylamine 3-N-methoxy(polyethylene glycol)-2000 carbamoyl which is also known as DSA-PEG, and its amide analogue.

When a conjugate as described above is comprised in the composition, it is preferably comprised in the nanoparticle, and preferably at least 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.5, 3.0, 3.5, 4.0, 4.5, or 5.0 mol % of conjugate is comprised; preferably at most 6.5, 6.0, 5.5, 5.0, 4.5, 4.0, 3.5, 3.0, 2.5, 2.0, 1.9, 1.8, 1.7, 1.6, 1.5, 1.4, 1.3, 1.2, 1.1, 1.0, 0.9, 0.8, 0.7, 0.6, or 0.5 mol % of conjugate is comprised. As explained above, this molar percentage t only pertains to the substances making up the lipid nanoparticle, and not to solvents or cargo such as oligonucleotides. When a conjugate is comprised in the composition, preferably 0 to 4 mol %, 0 to 3 mol %, 0.3 to 3 mol %, 0.5 to 3 mol %, 0.5 to 2.5 mol %, or 1 to 2.5 mol % is comprised; more preferably 0.5 to 2.5 mol % or 0.7 to 2.5 mol % is comprised, most preferably 0.8 to 2.4 mol % is comprised, such as 1 mol % or 2 mol %.

Preferred nanoparticles comprise a diamino lipid and a sterol. Further preferred nanoparticles comprise a diamino lipid and a phospholipid. Further preferred nanoparticles comprise a diamino lipid and a conjugate of a water soluble polymer and a lipophilic anchor. Preferred nanoparticles comprise a diamino lipid and a sterol and a phospholipid. Preferred nanoparticles comprise a diamino lipid and a sterol and a conjugate of a water soluble polymer and a lipophilic anchor. Preferred nanoparticles comprise a diamino lipid and a phospholipid and a conjugate of a water soluble polymer and a lipophilic anchor. Most preferred nanoparticles comprise a diamino lipid and a sterol and a phospholipid and a conjugate of a water soluble polymer and a lipophilic anchor.

In preferred embodiments, this aspect provides the nanoparticle composition for use according to the invention, wherein the nanoparticles comprise:
i) 20-60 mol % of diamino lipid, and
ii) 0-40 mol % of phospholipid, and
iii) 30-70 mol % of a sterol, preferably cholesterol, and
iv) 0-10 mol % of a conjugate of a water soluble polymer and a lipophilic anchor as defined above.
   In further preferred embodiments the nanoparticles comprise
i) 25-55 mol % of diamino lipid, and
ii) 1-30 mol % of phospholipid, and
iii) 35-65 mol % of a sterol, preferably cholesterol, and
iv) 0.1-4 mol % of a conjugate of a water soluble polymer and a lipophilic anchor.
   In further preferred embodiments the nanoparticles comprise
i) 30-50 mol % of diamino lipid, and
ii) 5-15 mol % of phospholipid, and
iii) 40-60 mol % of a sterol, preferably cholesterol, and
iv) 0.5-2.5 mol % of a conjugate of a water soluble polymer and a lipophilic anchor.
   In further preferred embodiments the nanoparticles comprise
i) about 38-42 mol % of diamino lipid, and
ii) about 8-12 mol % of phospholipid, and
iii) about 46-50 mol % of a sterol, preferably cholesterol, and
iv) about 1.8-2.2 mol % of a conjugate of a water soluble polymer and a lipophilic anchor.

The composition for use according to the invention can advantageously comprise additional therapeutically active agents. In preferred embodiments is provided the composition for use according to the invention, further comprising an additional pharmaceutically active compound, preferably selected from the group consisting of a PP2A methylating agent, an inhibitor of hepatocyte growth factor (HGF), an antibody, a PI3K inhibitor, an Akt inhibitor, an mTOR inhibitor, a binder of a T cell co-stimulatory molecule such as a binder of OX40, and a chemotherapeutic agent. Chemotherapeutic agents are defined later herein.

A PP2A methylating agent can activate PP2A, which in turn activates tumour suppressors such as p53 (see US2007280918). A particularly preferred PP2A methylating agent is betaine (betaine hydrate or also trimethylammonio-2 acetate) or one of its pharmaceutically acceptable salts, in particular betaine citrate. An HGF inhibitor can inhibit HGF, which is coexpressed, often over-expressed, on various human solid tumors including tumors derived from lung, colon, rectum, stomach, kidney, ovary, skin, multiple myeloma and thyroid tissue (see WO2009126842). Preferred HGF inhibitors are truncated HGF proteins such as NKI (N terminal domain plus kringle domain 1; Lokker et al., J. Biol. Chem. 268:17145, 1993); NK2 (N terminal domain plus kringle domains 1 and 2; Chan et al, Science 254:1382, 1991); and NK4 (N-terminal domain plus four kringle domains), which was shown to partially inhibit the primary growth and metastasis of murine lung tumor LLC in a nude mouse model (Kuba et al, Cancer Res. 60:6737, 2000), anti-HGF mAbs such as L2G7 (Kim et al, Clin Cancer Res 12:1292, 2006 and U.S. Pat. No. 7,220,410), HuL2G7 (WO 07115049 A2), the human mAbs described in WO 2005/017107 A2, and the HGF binding proteins described in WO 07143090 A2 or WO 07143098 A2. PI3K inhibitors are widely known. Preferred PI3K inhibitors are GSK2636771B, GSK2636771, idelalisib, copanlisib, duvelisib, and alpelisib. Akt inhibitors are widely known. Preferred Akt inhibitors are VQD-002, perifosine, miltefosine, MK-2206, AZD5363, and ipatasertib. mTOR inhibitors are widely known. Preferred mTOR inhibitors are sirolimus, everolimus, ridaforolimus, temsirolimus, umirolimus, and zotarolimus. Binder of a T cell co-stimulatory molecule are described in WO2019106605. A preferred such binder is a binder of OX40 such as an antibody against OX40.

### Method for Agonizing PTEN

The invention also provided a method for agonising PTEN, the method comprising the step of contacting a cell with a miRNA-193a as defined for use above, or with a composition as defined for use above. Accordingly, the cell is contacted with a miRNA-193a molecule, isomiR, mimic, or source thereof. The method van be an in vivo, in vitro, or ex vivo method, and preferably it is an in vitro or ex vivo method. Agonising PTEN is as defined elsewhere herein, and is preferably increasing expression of PTEN or increasing PTEN protein activity or increasing PTEN protein levels, more preferably it is increasing PTEN protein activity. PTEN activity of levels are preferably increased by at least 5%, more preferably by at least 25%. Ways for contacting a cell are widely known in the art; preferably the miRNA is added to the cell culture medium without further excipients, or it is transfected such as by using transfection reagents.

### General Definitions

In this document and in its claims, the verb “to comprise” and its conjugations is used in its non-limiting sense to mean that items following the word are included, but items not specifically mentioned are not excluded. In addition, reference to an element by the indefinite article “a” or “an” does not exclude the possibility that more than one of the element is present, unless the context clearly requires that there be one and only one of the elements. The indefinite article “a” or “an” thus usually means “at least one”.

The word “about” or “approximately” when used in association with a numerical value (e.g. about 10) preferably means that the value may be the given value more or less 1% of the value. When moieties or substructures of molecules are said to be identical, the natural abundance distribution of isotopes is not accounted for. The identical nature refers to a structural formula as it would be drawn.

As used herein, mol % refers to molar percentage, which is also known as a mole fraction or a molar fraction or a mole percent or an amount fraction. It relates to the amount in moles of a constituent, divided by the total amount of all constituents in a mixture, also expressed in moles.

In the context of this invention, a decrease or increase of a parameter to be assessed means a change of at least 5% of the value corresponding to that parameter. More preferably, a decrease or increase of the value means a change of at least 10%, even more preferably at least 20%, at least 30%, at least 40%, at least 50%, at least 70%, at least 90%, or 100%. In this latter case, it can be the case that there is no longer a detectable value associated with the parameter.

The use of a substance as a medicament as described in this document can also be interpreted as the use of said substance in the manufacture of a medicament. Similarly, whenever a substance is used for treatment or as a medicament, it can also be used for the manufacture of a medicament for treatment. Products for use are suitable for use in methods of treatment, for example in a method for treating a condition associated with PTEN-deficiency, preferably a PTEN-deficient cancer, the method comprising the step of administering to a subject a miRNA-193a for use according to the invention, or a composition for use according to the invention.

The present invention has been described above with reference to a number of exemplary embodiments. Modifications and alternative implementations of some parts or elements are possible, and are included in the scope of protection as defined in the appended claims. All citations of literature and patent documents are hereby incorporated by reference.

“Modulate” as used herein, for example with regard to expression of a gene, means to change any natural or existing level of function, for example it means affecting expression by increasing or reducing it. Modulation includes upregulating or agonizing, e.g., signaling, as well as downregulating, antagonizing, or blocking signaling or interactions with a ligand or compound or molecule that happen in the unchanged or unmodulated state. Thus, modulators may be agonists or antagonists. Agonist or antagonist activity can be measured in vitro by various assays know in the art such as, but not limited to, measurement of cell signalling, cell proliferation, immune cell activation markers, and cytokine production, optionally including comparison to unmodulated reference samples. Agonist or antagonist activity can also be measured in vivo by various assays that measure surrogate end points such as, but not limited to the measurement of T cell proliferation or cytokine production.

General Technologies Referred to Herein

MicroRNA molecules (“miRNAs”) are generally 21 to 22 nucleotides in length, though lengths of 17 and up to 25 nucleotides have been reported. Any length of 17, 18, 19, 20, 21, 22, 23, 24, 25 is therefore encompassed within the present invention. The miRNAs are each processed from a longer precursor RNA molecule (“precursor miRNA”). Precursor miRNAs are transcribed from non-protein-encoding genes. A precursor may have a length of at least 50, 70, 75, 80, 85, 100, 150, 200 nucleotides or more. The precursor miRNAs have two regions of complementarity that enables them to form a stem-loop- or fold-back-like structure, which is cleaved by enzymes called Dicer and Drosha in animals. Dicer and Drosha are ribonuclease III-like nucleases. The processed miRNA is typically a portion of the stem.

The processed miRNA (also referred to as “mature miRNA”) becomes part of a large complex, known as the RNA-induced Silencing Complex (RISC) complex, to (down or up)-regulate a particular target gene. Examples of animal miRNAs include those that perfectly or imperfectly basepair with the mRNA target, resulting in either mRNA degradation or inhibition of translation respectively (Olsen et al, 1999; Seggerson et al, 2002). SiRNA molecules also are processed by Dicer, but from a long, double-stranded RNA molecule. SiRNAs are not naturally found in animal cells, but they can function in such cells in a RNA-induced silencing complex (RISC) to direct the sequence-specific cleavage of an mRNA target (Denli et al, 2003).

The study of endogenous miRNA molecules is described in U.S. Patent Application 60/575,743. A miRNA is apparently active in the cell when the mature, single-stranded RNA is bound by a protein complex that regulates the translation of mRNAs that hybridize to the miRNA. Introducing exogenous RNA molecules that affect cells in the same way as endogenously expressed miRNAs requires that a single-stranded RNA molecule of the same sequence as the endogenous mature miRNA be taken up by the protein complex that facilitates translational control. A variety of RNA molecule designs have been evaluated. Three general designs that maximize uptake of the desired single-stranded miRNA by the miRNA pathway have been identified. An RNA molecule with a miRNA sequence having at least one of the three designs may be referred to as a synthetic miRNA.

miRNA molecules of the invention can replace or supplement the gene silencing activity of an endogenous miRNA. An example of such molecules, preferred characteristics and modifications of such molecules and compositions comprising such molecules is described in WO2009/091982.

miRNA molecules of the invention or isomiRs or mimics or sources thereof comprise, in some embodiments, two RNA molecules wherein one RNA is identical to a naturally occurring, mature miRNA. The RNA molecule that is identical to a mature miRNA is referred to as the active strand or the antisense strand. The second RNA molecule, referred to as the complementary strand or the sense strand, is at least partially complementary to the active strand. The active and complementary strands are hybridized to create a double-stranded RNA, that is similar to the naturally occurring miRNA precursor that is bound by the protein complex immediately prior to miRNA activation in the cell. Maximizing activity of said miRNA requires maximizing uptake of the active strand and minimizing uptake of the complementary strand by the miRNA protein complex that regulates gene expression at the level of translation. The molecular designs that provide optimal miRNA activity involve modifications of the complementary strand. Two designs incorporate chemical modifications of the complementary strand. The first modification involves creating a complementary RNA with a group other than a phosphate or hydroxyl at its 5′ terminus. The presence of the 5′ modification apparently eliminates uptake of the complementary strand and subsequently favors uptake of the active strand by the miRNA protein complex. The 5′ modification can be any of a variety of molecules including NH₂, NHCOCH₃, biotin, and others. The second chemical modification strategy that significantly reduces uptake of the complementary strand by the miRNA pathway is incorporating nucleotides with sugar modifications in the first 2-6 nucleotides of the complementary strand. It should be noted that the sugar modifications consistent with the second design strategy can be coupled with 5′ terminal modifications consistent with the first design strategy to further enhance miRNA activities. The third miRNA design involves incorporating nucleotides in the 3′ end of the complementary strand that are not complementary to the active strand. Hybrids of the resulting active and complementary RNAs are very stable at the 3′ end of the active strand but relatively unstable at the 5′ end of the active strand. Studies with siRNAs indicate that 5′ hybrid stability is a key indicator of RNA uptake by the protein complex that supports RNA interference, which is at least related to the miRNA pathway in cells. The inventors have found that the judicious use of mismatches in the complementary RNA strand significantly enhances the activity of said miRNA.

Further definitions for nucleic acids, nucleobases, nucleosides, nucleotides, nucleic acid analogues, modified nucleotides, preparation of nucleic acids, design of miRNAs, 5′ blocking agents, host cells and target cells, delivery methods, and nanoparticle functionalisation are preferably as described in WO2013/095132.

Therapeutic Applications

miRNAs that affect phenotypic traits provide intervention points for therapeutic applications as well as diagnostic applications (by screening for the presence or absence of a particular miRNA, or altered concentration of a particular miRNA). It is specifically contemplated that RNA molecules of the present invention can be used to treat any of the diseases or conditions discussed in the previous section. Moreover, any of the methods described above can also be employed with respect to therapeutic and diagnostic aspects of the invention. For example, methods with respect to detecting miRNAs or screening for them can also be employed in a diagnostic context. In therapeutic applications, an effective amount of the miRNAs of the present invention is administered to a cell, which may or may not be in an animal. In some embodiments, a therapeutically effective amount of the miRNAs of the present invention is administered to an individual for the treatment of disease or condition. The term “effective amount” as used herein is defined as the amount of the molecules of the present invention that are necessary to result in the desired physiological change in the cell or tissue to which it is administered. The term “therapeutically effective amount” as used herein is defined as the amount of the molecules of the present invention that achieves a desired effect with respect to a disease or condition associated with a disease or condition as earlier defined herein. A skilled artisan readily recognizes that in many cases the molecules may not provide a cure but may provide a partial benefit, such as alleviation or improvement of at least one symptom. In some embodiments, a physiological change having some benefit is also considered therapeutically beneficial. Thus, in some embodiments, an amount of molecules that provides a physiological change is considered an “effective amount” or a “therapeutically effective amount.”

In certain embodiments, pharmaceutical compositions may comprise, for example, at least about 0.1% of an active compound. In other embodiments, an active compound may comprise 2% to 75% of the weight of the unit, or 25% to 60%, for example, and any range derivable therein. In other non-limiting examples, a dose may also comprise less than 1 microgram/kg/body weight, or 1 microgram/kg/body weight, from 5 microgram/kg/body weight, 10 microgram/kg/body weight, 50 microgram/kg/body weight, 100 microgram/kg/body weight, 200 microgram/kg/body weight, 350 microgram/kg/body weight, 500 microgram/kg/body weight, 1 milligram/kg/body weight, 5 milligram/kg/body weight, 10 milligram/kg/body weight, 50 milligram/kg/body weight, 100 milligram/kg/body weight, 200 milligram/kg/body weight, 350 milligram/kg/body weight, or 500 milligram/kg/body weight, to 1000 mg/kg/body weight or more per administration, and any range derivable therein. In non-limiting examples of a derivable range from the numbers listed herein, a range of 5 mg/kg/body weight to 100 mg/kg/body weight, 5 microgram/kg/body weight to 500 milligram/kg/body weight, etc., can be administered, based on the numbers described above.

In any case, the composition may comprise various antioxidants to retard oxidation of one or more component. Additionally, the prevention of the action of microorganisms can be brought about by preservatives such as various antibacterial and antifungal agents, including but not limited to parabens, chlorobutanol, phenol, sorbic acid, thimerosal or combinations thereof.

The molecules may be formulated into a composition in a free base, neutral or salt form. Pharmaceutically acceptable salts, include the acid addition salts, e.g., those formed with the free amino groups of a proteinaceous composition, or which are formed with inorganic acids such as for example, hydrochloric or phosphoric acids, or such organic acids as acetic, oxalic, tartaric or mandelic acid. Salts formed with the free carboxyl groups can also be derived from inorganic bases such as for example, sodium, potassium, ammonium, calcium or ferric hydroxides; or such organic bases as isopropylamine, trimethylamine, histidine or procaine.

The composition is generally a suspension of nanoparticles in an aqueous medium. However, it can be lyophilized and provided as a powder, wherein the powder comprises the nanoparticles and optionally buffer salts or other excipients.

Effective Dosages

The molecules of the invention will generally be used in an amount effective to achieve the intended purpose. For use to treat or prevent a disease condition, the molecules of the invention, or pharmaceutical compositions thereof, are administered or applied in a therapeutically effective amount. A therapeutically effective amount is an amount effective to ameliorate or prevent the symptoms, or prolong the survival of the patient being treated. Determination of a therapeutically effective amount is well within the capabilities of those skilled in the art, especially in light of the detailed disclosure provided herein. For systemic administration, a therapeutically effective dose can be estimated initially from in vitro assays. For example, a dose can be formulated in animal models to achieve a circulating concentration range that includes the EC50 as determined in cell culture. Such information can be used to more accurately determine useful doses in humans. Initial dosages can also be estimated from in vivo data, e.g., animal models, using techniques that are well known in the art. One having ordinary skill in the art could readily optimize administration to humans based on animal data. Dosage amount and interval may be adjusted individually to provide plasma levels of the molecules which are sufficient to maintain therapeutic effect. Usual patient dosages for administration by injection range from 0.01 to 0.1 mg/kg/day, or from 0.1 to 5 mg/kg/day, preferably from 0.5 to 1 mg/kg/day or more. Therapeutically effective serum levels may be achieved by administering multiple doses each day.

In cases of local administration or selective uptake, the effective local concentration of the proteins may not be related to plasma concentration. One having skill in the art will be able to optimize therapeutically effective local dosages without undue experimentation. The amount of molecules administered will, of course, be dependent on the subject being treated, on the subject's weight, the severity of the affliction, the manner of administration and the judgment of the prescribing physician. The therapy may be repeated intermittently while symptoms detectable or even when they are not detectable. The therapy may be provided alone or in combination with other drugs or treatment (including surgery).

Sequence Identity

“Sequence identity” is herein defined as a relationship between two or more nucleic acid (nucleotide, polynucleotide, RNA, DNA) sequences, as determined by comparing the sequences. In the art, “identity” also means the degree of sequence relatedness between nucleic acid sequences, as the case may be, as determined by the match between strings of such sequences. “Identity” and “similarity” can be readily calculated by known methods, including but not limited to those described in Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heine, G., Academic Press, 1987; and Sequence Analysis Primer, Gribskov, M. and Devereux, J., eds., M Stockton Press, New York, 1991; and Carillo, H., and Lipman, D., SIAM J. Applied Math., 48:1073 (1988). In an embodiment, identity is assessed on a whole length of a given SEQ ID NO.

Preferred methods to determine identity are designed to give the largest match between the sequences tested. Methods to determine identity and similarity are codified in publicly available computer programs. Preferred computer program methods to determine identity and similarity between two sequences include e.g. the GCG program package (Devereux, J., et al., Nucleic Acids Research 12 (1): 387 (1984)), BestFit, BLASTP, BLASTN, and FASTA (Altschul, S. F. et al., J. Mol. Biol. 215:403-410 (1990). The BLAST X program is publicly available from NCBI and other sources (BLAST Manual, Altschul, S., et al., NCBI NLM NIH Bethesda, Md. 20894; Altschul, S., et al., J. Mol. Biol. 215:403-410 (1990). The well-known Smith Waterman algorithm may also be used to determine identity.

Preferred parameters for nucleic acid comparison include the following: Algorithm: Needleman and Wunsch, J. Mol. Biol. 48:443-453 (1970); Comparison matrix: matches=+10, mismatch=0; Gap Penalty: 50; Gap Length Penalty: 3. Available as the Gap program from Genetics Computer Group, located in Madison, Wis. Given above are the default parameters for nucleic acid comparisons.

Chemotherapeutic Agents

Examples of chemotherapeutic agents for use in combinations according to the invention include alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CBI-TMI); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gamma and calicheamicin omega); dynemicin, including dynemicin A; bisphosphonates, such as clodronate; an esperamicin; as well as neocarzinostatin chromophore and related chromoprotein enediyne antiobiotic chromophores, aclacinomysins, actinomycin, authrarnycin, azaserine, bleomycins, cactinomycin, carabicin, carminomycin, carzinophilin, chromomycinis, dactinomycin, daunorubicin, detorubicin, 6-diazo-5-oxo-L-norleucine, doxorubicin (including morpholmo-doxorubicm, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalarnycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK polysaccharide complex; razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., paclitaxel and doxetaxel; chlorambucil; gemcitabine; 6-thioguanine; mercaptopurine; methotrexate; platinum coordination complexes such as cisplatin, oxaliplatin and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-II); topoisomerase inhibitor RFS 2000; difluorometlhylornithine (DMFO); retinoids such as retinoic acid; capecitabine; gefitinib and pharmaceutically acceptable salts, acids or derivatives of any of the above.

Also included in this definition are anti-hormonal agents that act to regulate or inhibit hormone action on tumours such as anti-estrogens and selective estrogen receptor modulators (SERMs), including, for example, tamoxifen, raloxifene, droloxifene, 4-hydroxytamoxifen, trioxifene, keoxifene, LYI 17018, onapristone, and toremifene; aromatase inhibitors that inhibit the enzyme aromatase, which regulates estrogen production in the adrenal glands, such as, for example, 4(5)-imidazoles, aminoglutethimide, megestrol acetate, exemestane, formestanie, fadrozole, vorozole, letrozole, and anastrozole; and anti-androgens such as flutamide, nilutamide, bicalutamide, leuprolide, and goserelin; as well as troxacitabine (a 1,3-dioxolane nucleoside cytosine analog); antisense oligonucleotides, particularly those which inhibit expression of genes in signaling pathways implicated in aberrant cell proliferation, such as, for example, PKC-α, Raf and H-Ras; ribozymes such as a VEGF expression inhibitor and a HER2 expression inhibitor; vaccines such as gene therapy vaccines and pharmaceutically acceptable salts, acids or derivatives of any of the above. A list of U.S. FDA approved oncology drags with their approved indications can be found on the World Wide Web at accessdata.fda.gov/scripts/cder/onctools/druglist.cfm. A suitable RNR inhibitor is selected from the group consisting of gemcitabine, hydroxyurea, clolar, clofarabine, and triapine. A suitable AURKB inhibitor is selected from the group consisting of: AZD1152, VX-680, MLN8054, MLN8237, PHA680632, PH739358, Hesperidin, ZM447439, JNJ770621, SU6668, CCT129202, AT9283, MP529, SNS314, R763, ENMD2076, XL228, TTP687, PF03814735 and CYC116. Another suitable anticancer drug is gefitinib.

### FIGURE LEGENDS

FIG. 1—Canonical pathway analysis. (A) Top 25 canonical pathways regulated by miR-193a in at least three cell lines at 24h ranked based on P value. Dotted line indicates P<0.01. White bar: activated, black bar: inhibited, grey bar: direction not determined. (B) Treemap of genes that were identified in at least 4 significant pathways. Box size corresponds to the number of pathways in which the gene was differentially expressed.

FIG. 2—Genes downregulated by miR-193a in the PTEN pathway. Genes that were significantly downregulated by miR-193a (average relative expression compared to mock <1, P<0.05) at 24h in at least three cell lines are shown without hatching. PTEN is highlighted in black. Pointy arrows indicate stimulation whereas bar-headed arrows indicate inhibition.

FIG. 3—Biological functions affected by miRNA-193a. Biological functions relevant to (tumour) cells with z-scores <−2 and >2. All P values are smaller than 0.00001.

FIG. 4—Western blotting of miR-193a-3p targets in the PTEN pathway. Human tumor cell lines were transfected with 10 nM scrambled control or 10 nM miRNA-193a and lysed after 72h. Clarified whole cell lysates were immunoblotted for FAK, P70S6K, PIK3R1 and TGFBRIII. Vinculin and tubulin are loading controls. Boxes indicate protein downregulation.

FIG. 5—Western blotting of phosphoproteins in the PTEN pathway. Human tumor cell lines were transfected with 10 nM scrambled control or 10 nM miRNA-193a and lysed after 72h. Clarified whole cell lysates were immunoblotted for pSer473 AKT, AKT, pThr202/Tyr204 ERK1/2, ERK1/2, pSer259 c-RAF and c-RAF. Vinculin and tubulin are loading controls. Full boxes indicate protein downregulation and dashed boxes indicate protein upregulation.

FIG. 6—Transfection with miRNA-193a induces surface expression of CRT in A2058 and HEP3B cells. A-B) graphs show percentages of live (DAPI⁻) and dying (DAPI^{low}), but not dead (DAPI⁺) cells, expressing CRT on their surface, for A2058 (A) and HEP3B (B) cells transfected with 0.1, 1, 3 and 10 nM of miRNA-193a, or a mock transfection control. C-D) Panels show the cytofluorometric plots of A2058 (C) and HEP3B (D) cells, analyzed 72 hours post transfection.

FIG. 7—Co-culture with miRNA-193a transfected A2058 tumor cells enhances the proliferation of T cells. PBMCs were labeled with CFSE and kept in culture alone, or in co-culture with mock transfected or miRNA-193a transfected A2058 cells. Cytofluorometric plots show the level of CFSE of CD3′ T cells, after 2 days or 6 days of co-culture.

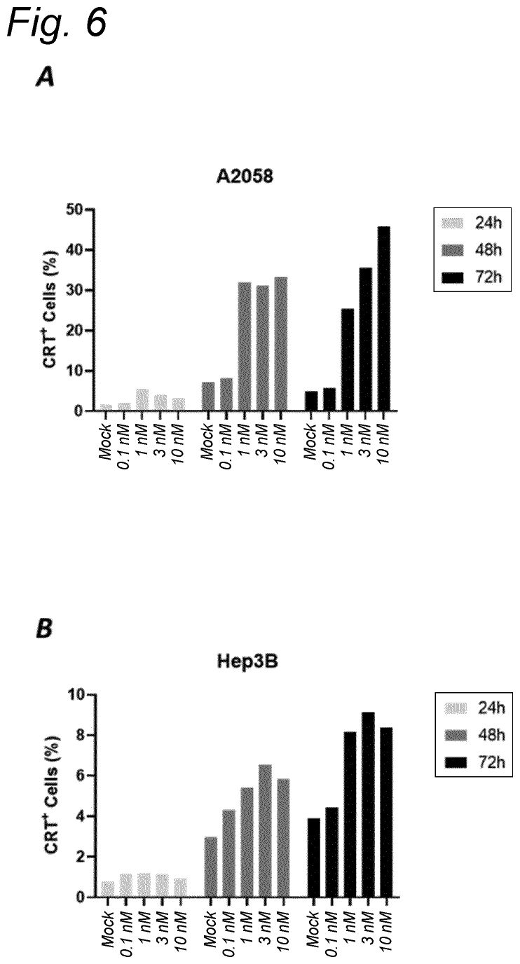

FIG. 8—Effect of human Peripheral Blood Mononuclear Cells (PBMCs) on human melanoma A2058 and NSCLC A549 tumor cells. Human melanoma A2058 (A) and NSCLC A549 (B) tumor cells were co-cultured with the indicated ratio of human PBMCs to tumor cells for 72 h either in the absence or the presence of human anti CD3/CD28 antibodies (T cell activator). Then surviving cells were fixed and stained with crystal violet. The relative percentage of surviving cells (as compared to similar experimental conditions in the absence of PBMCs) was quantified by colorimetry of the stained cells (Feoktistova et al., 2016). Error bars represent SD to the mean of 3 independent replicates.

FIG. 9—Effect of human Peripheral Blood Mononuclear Cells (PBMCs) on human melanoma A2058 (A) and NSCLC A549 (B) tumor cells upon tumor cell transfection with miRNA-193a. Human melanoma A2058 and NSCLC A549 tumor cells were transfected (RNAiMAX) with the indicated concentrations of either negative miRNA control (3A1) or miR-193a-3p, after which cells were co-cultured with the indicated ratio of human PBMCs to tumor cells for the indicated times. Then surviving cells were fixed and stained with crystal violet. The relative percentage of surviving cells (as compared to mock transfected condition)) was quantified by colorimetry of the stained cells (Feoktistova et al., 2016). N.S.: not significant, *: p <0.05 and **: p<0.01 as determined by the Student t tests (asymptotic significance [2-tailed]). Error bars represent SD to the mean of 3 independent replicates.

### EXAMPLES

### Example 1—RNA-Sequencing, Differential Gene Expression, and Pathway Analysis after Treatment of Different Cancer Cell Lines with miRNA-193a

Implementation of high-throughput RNA-sequencing (RNA-seq) has become a powerful tool for comprehensive characterization of the whole transcriptome at gene and exon levels and with a unique ability to identify differentially expressed genes, novel genes and transcripts at high resolution and efficiency. However, till date, very few miRNAs have been characterized for their specific role in cancer development. Hence, we have used the high-throughput RNA-seq after overexpressing a miRNA-193a (viz. a miRNA-193a-3p mimic) in 5 different cancer cell lines including A540 and H460 (both lung cancer), Huh7 and Hep3B (both liver cancer), and BT549 (breast cancer) at 24h post-transfection with miR-193a at 10 nM. The gene expression was compared to mock as control and differentially expressed genes and their cellular pathways were subsequently identified.

### 1.1 Materials & Methods

1.1.1 Cell Preparation for RNA-Seq

Human cancer cell lines were cultured in appropriate media (Table 1) and seeded into 6-well plates 24h before transfection with 10 nM miRNA-193a-3p mimic or mock using Lipofectamine RNAiMAX (Thermofisher). The mimic was a double stranded mimic wherein the antisense strand consisted of an RNA oligonucleotide having SEQ ID NO: 56 (the canonical miRNA-193a-3p), and wherein the sense strand consisted of an oligonucleotide represented by SEQ ID NO: 218.

Reagents were aspirated 16h after transfection and cells were passaged into new 6-well plates. Media was aspirated 24h after transfection and plates were stored at −80° C. Three independent replicates were performed for each cell line.

TABLE 1
Cell line details.

| Cell line | Cancer type | Medium |
|---|---|---|
| A549 | Lung (NSCLC) | F-12K + 10% FBS + P/S |
| BT549 | Breast (TNBC) | RPMI-1640 + 10% FBS + P/S + |
| | | 0.023 IU/mL insulin |
| H460 | Lung (NSCLC) | RPMI-1640 + 10% FBS + P/S |
| HEP3B | Liver (HCC) | EMEM + 10% FBS + P/S |
| HUH7 | Liver (HCC) | DMEM low glucose + 10% FBS + |
| | | P/S + L-glutamine |

FBS: fetal bovine serum,
P/S: penicillin streptomycin

1.1.2 RNA Isolation for RNA-Seq

RNA was isolated using the miRNeasy Mini kit (Qiagen). The procedure included on-column DNase treatment. RNA concentration was measured on NanodropOne. 150 ng of each independent replicate was pooled and 450 ng samples (having sample IDs A549 Mock_24, A549 miRNA-193a-3p_24, BT549 Mock_24, BT549 miRNA-193a-3p_24, H460 Mock 24, H460 miRNA-193a-3p_24, HEP3B Mock_24, HEP3B miRNA-193a-3p_24, HUH7 Mock_24, and HUH7 miRNA-193a-3p_24) were submitted to GenomeScan BV (Leiden, The Netherlands).

1.1.3 RNA-Seq Procedure

PolyA enrichment was performed followed by next generation RNA-Seq using Illumina NovaSeq 6000 at GenomeScan BV. The data processing workflow included raw data quality control, adapter trimming, and alignment of short reads. The reference GRCh37.75.dna.primary_assembly was used for alignment of the reads for each sample. Based on the mapped locations in the alignment file the frequency of how often a read was mapped on a transcript was determined (feature counting). The counts were saved to count files, which serve as input for downstream RNA-Seq differential expression analysis.

1.1.4 Data Analysis for RNA-Seq

Differential expression analysis was performed on the short read data set by GenomeScan BV. The read counts were loaded into the DESeq package v1.30.0, a statistical package within the R platform v3.4.4. DESeq was specifically developed to find differentially expressed genes between two conditions (mock versus miRNA-193a-3p) for RNA-seq data with small sample size and over-dispersion. The differential expression comparison grouping is provided in Table.

TABLE 2
Expression comparison setup.

| Comparison | Condition A | Condition B |
|---|---|---|
| 1 | A549_Mock_24 | A549_miRNA-193a-3p_24 |
| 2 | BT549_Mock_24 | BT549_miRNA-193a-3p_24 |
| 3 | H460_Mock_24 | H460_miRNA-193a-3p_24 |
| 4 | HEP3B_Mock_24 | HEP3B_miRNA-193a-3p_24 |
| 5 | HUH7_Mock_24 | HUH7_miRNA-193a-3p_24 |

1.1.5Pathway Analysis

Lists of genes that were significantly (P<0.05) differentially expressed in our RNA-seq dataset were uploaded and analyzed using Ingenuity Pathway Analysis (IPA) software (www.ingenuity.com).

### 1.2 Results

1.2.1 Genes Regulated by miR-193a-3p Mimic in Solid Tumor Cell Lines

Lists of significantly (P<0.05) differentially expressed genes (relative expression miRNA-193a/relative expression mock) at 24h after transfection were created for all cell lines (see description of invention). Most genes were downregulated as compared to mock (relative expression miRNA-193a/relative expression mock <1) (see Table 3).

TABLE 3
Number of genes down- and upregulated
by 193a-3p mimic per cell line.

24 h

| | Down | Up |
|---|---|---|
| A549 | 615 | 220 |
| BT549 | 620 | 168 |
| H460 | 656 | 215 |
| HEP3B | 599 | 166 |
| HUH7 | 683 | 200 |

Fout! Verwijzingsbron niet gevonden.4 shows genes with known roles in cancer that were downregulated by miRNA-193a in each cell line. Genes that were downregulated in all cell lines include: CCND1, CDK6, KRAS, MCL1, NT5E, STMN1, TGFBR3 and YWHAZ.

TABLE 4
Genes of interest downregulated by miR-193a per cell line.

| Cel lines | Downregulated genes |
|---|---|
| A549 | CAPRIN1, CCNA2, CCND1, CDK4, CDK6, |
| | CHEK1, DCAF7, DDB1, ETS1, HDAC3, HMGB1, |
| | IL17RD, KRAS, MCL, MPP2, NOTCH2, NT5E, |
| | PLAU, PSEN1, PTK2, RAB27B, SEPN1, SLC7A5, |
| | SOS2, ST3GAL4, STAT3, STMN1, TGFB2, TGFBR2, |
| | TGFBR3, TNFRSF21, YAP1, YWHAZ |
| BT549 | CCNA2, CCND1, CDC25A, CDK4, CDK6, CSF2, |
| | DCAF7, DDB1, ETS1, GRB7, HIC2, IDO1, |
| | IL17RD, KRAS, MCL1, MDM2, MPP2, NOTCH2, |
| | NT5E, PLAU, PSEN1, PTK2, RAB27B, SEPN1, |
| | SLC7A5, SOS2, ST3GAL4, STMN1, TGFBR3, |
| | TNFRSF1B, TNFRSF21, YAP1, YWHAZ |
| H460 | CAPRIN1, CCNA2, CCND1, CDK6, CDKN1A, CHEK1, |
| | CXCL1, CXCL5, DCAF7, DDB1, ETS1, HMGB1, |
| | IL17RD, KRAS, MAPK8, MCL1, MPP2, NOTCH1, |
| | NOTCH2, NOTCH3, NT5E, PLAU, PSEN1, PTK2, |
| | SEPN1, SLC7A5, ST3GAL4, STMN1, TGFBR3, |
| | TNFRSF1B, TNFRSF21 |
| HEP3B | AJUBA, CAPRIN1, CCND1, CDK6, CRYAA, DCAF7, |
| | ERBB4, ETS1, GRB7, IL17RD, KRAS, MAPK8, |
| | MCL1, MDM2, MPP2, NOTCH1, NT5E, PSEN1, |
| | PTK2, SEPN1, SLC7A5, SOS2, ST3GAL4, STMN1, |
| | TGFBR2, TGFBR3, TNFRSF1B, TNFRSF21, |
| | YAP1, YWHAZ |
| HUH7 | BRCA1, CCNA2, CCND1, CDC25A, CDK1, CDK6, |
| | CHEK1, DCAF7, E2F1, ETS1, EZH2, FEN1, |
| | FOXM1, GRB7, HMGB1, IL17RD, KRAS, MAPK8, |
| | MCL1, MDM2, MELK, MPP2, NT5E, PLAU, |
| | PLK1, RAD51, SEPN1, SLC7A5, ST3GAL4, STMN1, |
| | TGFBR3, TNFRSF21, YWHAZ |

1.2.2 Cellular Pathways Regulated by miR-193a in Solid Tumor Cell Lines

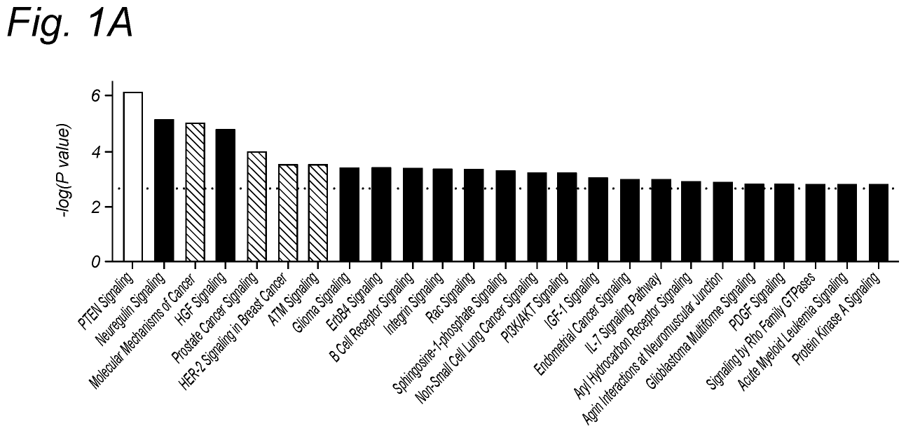

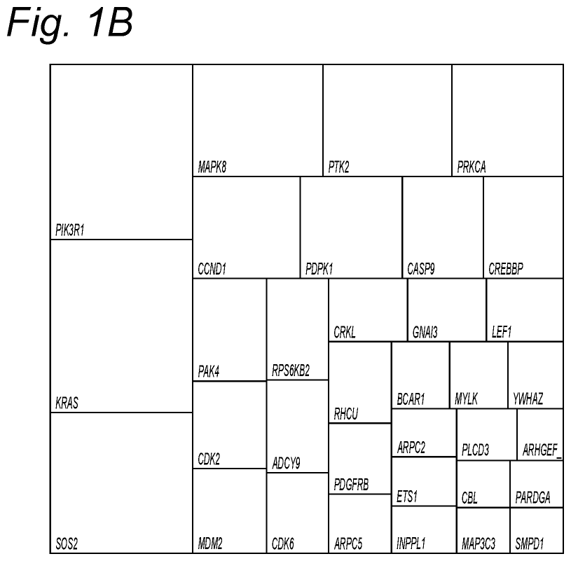

IPA was performed to identify canonical pathways that are affected in miRNA-193a treated cells compared to mock, based on the differential expression data. Tables 6-20 show all the significantly regulated pathways in each cell line. Because the objective was to develop new treatment options by more closely defining the mode of action of miR-193a across cancer types, we next analyzed the pathways that were regulated by genes differentially expressed in at least three cell lines. This analysis showed that the majority of pathways was affected or inhibited (FIG. 1A), including many growth factor signalling pathways which induce cellular proliferation and tumour progression. The most enriched canonical pathway, the tumour suppressive PTEN pathway, was indicated to be activated (z-score of 2.309). Differentially expressed genes in this pathway include RPS6KB2, KRAS, PDGFRB, SOS2, TGFBR3, CASP9, INPPL1, PIK3R1, PTK2, CBL, PDPK1, CCND1, BCAR1, and MAGI3 (FIG. 2).

Other identified pathways were significantly inhibited, including Neuregulin signalling (z-score of −2.333) and HGF signalling (z-score of −3.162). Genes from our differential expression dataset that participate in these pathways are shown in FIG. 1b and include PI3KR1, KRAS, SOS2 and PTK2. Many are important components of growth factor signalling and mitogen-activated protein kinase (MAPK) pathways, inducing nuclear signals for cellular proliferation and tumour progression.

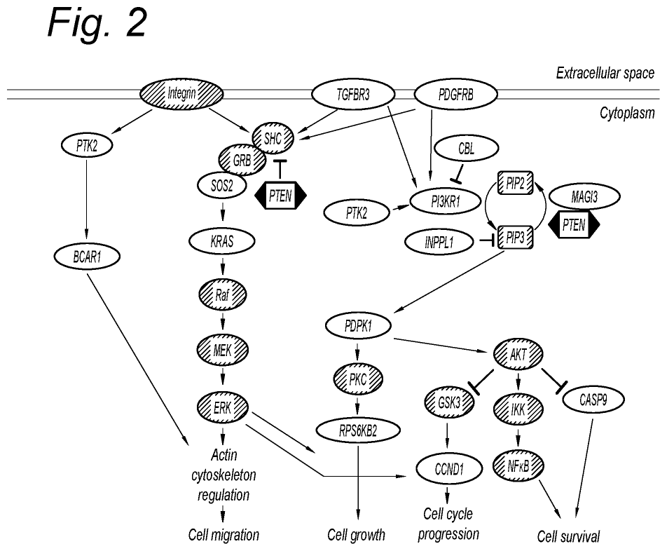

Subsequently, IPA software was used to predict downstream effects of the observed gene expression changes on biological functions and disease processes. Out of the 100 most significant biological functions that were changed by 193a at 24h, those that were inhibited (z-score <−2) were related to cell survival, proliferation, migration, or cancer, and those that were activated (z-score >2) were related to (tumour) cell death (FIG. 3). Furthermore, the majority of the affected biological functions (55% at 24h) belonged to the category Cancer (Table 5, which shows categories of top 100 biological functions ranked based on the number of miRNA-193a-regulated functions (all P<0.00001) at 24h).

TABLE 5
Biological function categories.
24 h

| Category | # functions |
|---|---|
| Cancer | 55 |
| Cell movement | 10 |
| Cell death and survival | 9 |
| Cell growth and development | 7 |
| Organismal development and survival | 6 |
| Nervous system function | 5 |
| Cardiovascular | 3 |
| Cell maintenance | 3 |
| DNA replication | 1 |
| Hematological disease | 1 |

TABLE 6
A549 lung cancer upregulated pathways and associated genes

| Pathway Name | Gene |
|---|---|
| PTEN Signaling | BCAR1, CASP9, CBL, CCND1, INPPL1, KRAS, |
| | PDPK1, PIK3R1, PTK2, RPS6KB2, SOS2, |
| | TGFBR2, TGFBR3 |
| Cell Cycle: G1/S Checkpoint Regulation | CCND1, CDK2, CDK4, CDK6, HDAC3, MAX, |
| | TGFB2 |
| RhoGDI Signaling | ACTR3, ARPC2, ARPC5, CDH1, CDH16, |
| | CDH8, GNAI3, GNAZ, GNB1, PAK4, PIP4K2C, |
| | PRKCA, RHOU |
| VDR/RXR Activation | HOXA10, IL12A, NCOA3, PRKCA, TGFB2, |
| | THBD |
| Pyrimidine Ribonucleotides Interconversion | AK7, ENTPD7, NUDT15, SLC25A42 |
| Endocannabinoid Cancer Inhibition Pathway | ADCY9, CASP9, CCND1, CDH1, GNAI3, |
| | PIK3R1, PTK2, SMPD1, TCF7L2 |
| Sumoylation Pathway | CDH1, ETS1, FOS, MYB, RHOU, SENP1, TDG |
| Pyrimidine Ribonucleotides De Novo | AK7, ENTPD7, NUDT15, SLC25A42 |
| Biosynthesis | |
| HIPPO signaling | LATS2, SFN, TP53BP2, WWTR1, YAP1, |
| | YWHAZ |
| PD-1, PD-L1 cancer immunotherapy pathway | CDK2, IL12A, LATS2, PDCD4, PIK3R1, |
| | TGFB2, YAP1 |
| PPARα/RXRα Activation | ADCY9, CHD5, KRAS, NCOA3, NOTUM, |
| | PRKCA, SOS2, TGFB2, TGFBR2, TGFBR3 |
| GPCR-Mediated Integration of Enteroendocrine | ADCY9, ADRB1, GNAI3, NOTUM |
| Signaling Exemplified by an L Cell | |
| Apoptosis Signaling | CAPN10, CASP9, KRAS, MCL1, PRKCA |
| AMPK Signaling | ADRB1, AK7, CCNA2, CCND1, PDPK1, |
| | PHLPP2, PIK3R1, PPM1F |

TABLE 7
A549 lung cancer aberrant pathways and associated genes

| Pathway Name | Gene |
|---|---|
| TR/RXR Activation | ADRB1, AKR1C1/AKR1C2, AKR1C3, ATP2A1, |
| | COL6A3, DIO2, HDAC3, KLF9, NCOA3, |
| | PIK3R1, SREBF2, TBL1XR1 |
| Molecular Mechanisms of Cancer | ADCY9, APH1B, CASP9, CBL, CCND1, CDH1, |
| | CDK2, CDK4, CDK6, CHEK1, FOS, GNAI3, |
| | GNAZ, HHAT, KRAS, MAX, NF1, PAK4, |
| | PIK3R1, PRKCA, PSEN1, PTK2, RASGRF2, |
| | RHOU, SOS2, TGFB2, TGFBR2 |
| Epithelial Adherens Junction Signaling | ACTR3, ARPC2, ARPC5, CDH1, KRAS, |
| | MYH11, NOTCH2, SSX2IP, TCF7L2, TGFB2, |
| | TGFBR2, TGFBR3, TUBA1B, TUBA4A |
| Coagulation System | F3, PLAU, PLAUR, SERPINA5, SERPINE1, |
| | THBD |
| Germ Cell-Sertoli Cell Junction Signaling | BCAR1, CDH1, KRAS, MAP3K2, MAP3K3, |
| | PAK4, PDPK1, PIK3R1, PTK2, RHOU, TGFB2, |
| | TGFBR2, TUBA1B, TUBA4A |
| Chronic Myeloid Leukemia Signaling | CCND1, CDK4, CDK6, CRKL, HDAC3, KRAS, |
| | PIK3R1, SOS2, TGFB2, TGFBR2 |
| Pancreatic Adenocarcinoma Signaling | CASP9, CCND1, CDK2, CDK4, KRAS, PIK3R1, |
| | PLD6, STAT3, TGFB2, TGFBR2 |
| Tight Junction Signaling | CDK4, CLDN2, CLDN4, FOS, GOSR2, MYH11, |
| | MYLK, NSF, NUDT21, PARD6A, TGFB2, |
| | TGFBR2, TJP3 |
| p53 Signaling | CCND1, CCNG1, CDK2, CDK4, CHEK1, |
| | PIK3R1, SFN, TP53BP2, TP53INP1 |
| Phenylalanine Degradation I (Aerobic) | PCBD1, QDPR |
| Gap Junction Signaling | ADCY9, ADRB1, GJB2, GNAI3, KRAS, |
| | MAP3K2, NOTUM, NPR1, PIK3R1, PPP3R1, |
| | PRKCA, SOS2, TUBA1B, TUBA4A |
| HER-2 Signaling in Breast Cancer | CASP9, CCND1, CDK6, KRAS, PARD6A, |
| | PIK3R1, PRKCA, SOS2 |
| Bile Acid Biosynthesis, Neutral Pathway | AKR1C1/AKR1C2, AKR1C3, SCP2 |
| Antiproliferative Role of TOB in T Cell Signaling | CCNA2, CDK2, TGFB2, TGFBR2 |
| Folate Polyglutamylation | MTHFD1, SHMT2 |
| Protein Citrullination | PADI1, PADI2 |
| Regulation of the Epithelial-Mesenchymal | APH1B, CDH1, ETS1, KRAS, NOTCH2, |
| Transition Pathway | PARD6A, PIK3R1, PSEN1, SOS2, STAT3, |
| | TCF7L2, TGFB2, TGFBR2 |
| Th1 and Th2 Activation Pathway | APH1B, BHLHE41, IL12A, IL4R, IL6R, IRF1, |
| | NOTCH2, PIK3R1, PSEN1, STAT3, TGFBR2, |
| | TGFBR3 |
| Serotonin Receptor Signaling | ADCY9, GCH1, MAOB, PCBD1, QDPR |
| Erythropoietin Signaling | CBL, FOS, KRAS, PDPK1, PIK3R1, PRKCA, |
| | SOS2 |
| FAK Signaling | BCAR1, CAPN10, KRAS, PAK4, PDPK1, |
| | PIK3R1, PTK2, SOS2 |
| Myc Mediated Apoptosis Signaling | CASP9, KRAS, PIK3R1, SFN, SOS2, YWHAZ |
| GADD45 Signaling | CCND1, CDK2, CDK4 |
| DNA Methylation and Transcriptional | DNMT3B, HIST1H4C, MTA2, SUDS3 |
| Repression Signaling | |
| Thyroid Cancer Signaling | BDNF, CCND1, CDH1, KRAS, TCF7L2 |
| Sphingomyelin Metabolism | SGMS1, SMPD1 |
| Regulation of IL-2 Expression in Activated and | FOS, KRAS, PPP3R1, SOS2, TGFB2, TGFBR2, |
| Anergic T Lymphocytes | VAV3 |
| Lipoate Salvage and Modification | LIPT1 |
| Prostate Cancer Signaling | CASP9, CCND1, CDK2, KRAS, PDPK1, |
| | PIK3R1, SOS2 |
| Folate Transformations I | MTHFD1, SHMT2 |
| Factors Promoting Cardiogenesis in Vertebrates | CDK2, NOG, PRKCA, TCF7L2, TGFB2, |
| | TGFBR2, TGFBR3 |
| Estrogen Receptor Signaling | HDAC3, KRAS, MED14, MED21, NCOA3, |
| | NRIP1, RBFOX2, SOS2, TAF7L |
| Calcium Transport I | ATP2A1, ATP2B4 |
| Hereditary Breast Cancer Signaling | CCND1, CDK4, CDK6, CHEK1, FANCE, |
| | HDAC3, KRAS, PIK3R1, SFN |
| Sertoli Cell-Sertoli Cell Junction Signaling | BCAR1, CDH1, CLDN2, CLDN4, KRAS, |
| | MAP3K2, MAP3K3, TGFBR3, TJP3, TUBA1B, |
| | TUBA4A |
| Role of JAK family kinases in IL-6-type Cytokine | IL6R, OSMR, STAT3 |
| Signaling | |
| Axonal Guidance Signaling | ACTR3, ARPC2, ARPC5, BCAR1, BDNF, |
| | CRKL, EPHB2, EPHB3, GNAI3, GNAZ, GNB1, |
| | KRAS, NOTUM, PAK4, PIK3R1, PLXNA2, |
| | PPP3R1, PRKCA, PTK2, SEMA4G, SOS2, |
| | TUBA1B, TUBA4A |
| Role of Macrophages, Fibroblasts and | CCL2, CCND1, CEBPD, F2RL1, FOS, IL6R, |
| Endothelial Cells in Rheumatoid Arthritis | KRAS, NOTUM, PIK3R1, PPP3R1, PRKCA, |
| | ROR2, STAT3, TCF7L2, TLR6, TRAF1 |
| Glucocorticoid Receptor Signaling | CCL2, FOS, HMGB1, KRAS, KRT14, MED14, |
| | NCOA3, NRIP1, PIK3R1, PLAU, PPP3R1, |
| | SERPINE1, SOS2, STAT3, TAF7L, TGFB2, |
| | TGFBR2 |
| T Cell Receptor Signaling | CBL, FOS, KRAS, PIK3R1, PPP3R1, SOS2, |
| | VAV3 |
| Glycine Biosynthesis I | SHMT2 |
| Guanine and Guanosine Salvage I | HPRT1 |
| L-cysteine Degradation III | MPST |
| Lipoate Biosynthesis and Incorporation II | LIPT1 |
| FAT10 Cancer Signaling Pathway | STAT3, TGFB2, TGFBR2, TGFBR3 |
| IL-4 Signaling | IL4R, INPPL1, KRAS, PIK3R1, RPS6KB2, |
| | SOS2 |
| Cholesterol Biosynthesis I | DHCR24, LBR |
| Cholesterol Biosynthesis II (via 24,25- | DHCR24, LBR |
| dihydrolanosterol) | |
| Cholesterol Biosynthesis III (via Desmosterol) | DHCR24, LBR |
| Breast Cancer Regulation by Stathmin1 | ADCY9, CDK2, GNAI3, GNB1, KRAS, PIK3R1, |
| | PRKCA, SOS2, STMN1, TUBA1B, TUBA4A |
| Ephrin A Signaling | BCAR1, PIK3R1, PTK2, VAV3 |
| Phenylalanine Degradation IV (Mammalian, via | HPD, MAOB |
| Side Chain) | |
| Ethanol Degradation II | ACSS2, ALDH9A1, DHRS9 |
| T Helper Cell Differentiation | IL12A, IL4R, IL6R, STAT3, TGFBR2 |
| Extrinsic Prothrombin Activation Pathway | F3, THBD |
| Granzyme B Signaling | CASP9, LMNB2 |
| Vitamin-C Transport | SLC23A2, SLC2A3 |
| L-carnitine Biosynthesis | ALDH9A1 |
| Methylglyoxal Degradation I | GLO1 |
| N-acetylglucosamine Degradation I | GNPDA1 |
| Tetrahydrobiopterin Biosynthesis I | GCH1 |
| Tetrahydrobiopterin Biosynthesis II | GCH1 |
| Thiosulfate Disproportionation III (Rhodanese) | MPST |
| Thyroid Hormone Metabolism I (via | DIO2 |
| Deiodination) | |
| Thyronamine and Iodothyronamine Metabolism | DIO2 |
| Tyrosine Biosynthesis IV | PCBD1 |
| Circadian Rhythm Signaling | BHLHE41, PER1, PER2 |
| CD27 Signaling in Lymphocytes | CASP9, FOS, MAP3K2, MAP3K3 |
| Leptin Signaling in Obesity | ADCY9, NOTUM, PDE3A, PIK3R1, STAT3 |
| Natural Killer Cell Signaling | INPPL1, KRAS, PAK4, PIK3R1, PRKCA, SOS2, |
| | VAV3 |
| Angiopoietin Signaling | CASP9, KRAS, PAK4, PIK3R1, PTK2 |
| IL-15 Production | DSTYK, EPHB2, EPHB3, IRF1, PTK2, ROR2, |
| | TWF1 |
| Dermatan Sulfate Degradation (Metazoa) | CEMIP2, IDS |
| IL-17A Signaling in Fibroblasts | CCL2, CEBPD, FOS |
| Noradrenaline and Adrenaline Degradation | ALDH9A1, DHRS9, MAOB |
| RAR Activation | ADCY9, AKR1C3, DHRS9, FOS, NR2F6, |
| | NRIP1, PDPK1, PIK3R1, PRKCA, TGFB2 |
| Dopamine Receptor Signaling | ADCY9, GCH1, MAOB, PCBD1, QDPR |
| Cell Cycle Control of Chromosomal Replication | CDK2, CDK4, CDK6, MCM2 |
| Unfolded protein response | CEBPD, DNAJC3, EDEM1, SREBF2 |
| 1D-myo-inositol Hexakisphosphate Biosynthesis | INPPL1, ITPKB |
| II (Mammalian) | |
| D-myo-inositol (1,3,4)-trisphosphate | INPPL1, ITPKB |
| Biosynthesis | |
| Differential Regulation of Cytokine Production in | CCL2, IL12A |
| Macrophages and T Helper Cells by IL-17A and | |
| IL-17F | |
| FAT10 Signaling Pathway | SQSTM1, UBE2Z |
| Methylglyoxal Degradation III | AKR1C1/AKR1C2, AKR1C3 |
| G-Protein Coupled Receptor Signaling | ADCY9, ADRB1, GNAI3, KRAS, PDE3A, |
| | PDE4D, PDE8A, PDPK1, PIK3R1, PLD6, |
| | PRKCA, SOS2, STAT3 |
| Cell Cycle Regulation by BTG Family Proteins | CCND1, CDK2, CDK4 |
| Acetate Conversion to Acetyl-CoA | ACSS2 |
| Glutathione Redox Reactions II | GSR |
| Heme Biosynthesis from Uroporphyrinogen-III I | CPOX |
| Melatonin Degradation II | MAOB |
| N-acetylglucosamine Degradation II | GNPDA1 |
| DNA damage-induced 14-3-3σ Signaling | CDK2, SFN |
| Oxidative Ethanol Degradation III | ACSS2, ALDH9A1 |
| Notch Signaling | APH1B, NOTCH2, PSEN1 |
| Nur77 Signaling in T Lymphocytes | CASP9, MAP3K2, MAP3K3, PPP3R1 |
| Docosahexaenoic Acid (DHA) Signaling | CASP9, PDPK1, PIK3R1 |
| The Visual Cycle | AKR1C3, DHRS9 |
| Endoplasmic Reticulum Stress Pathway | CASP9, DNAJC3 |
| Putrescine Degradation III | ALDH9A1, MAOB |
| IL-12 Signaling and Production in Macrophages | FOS, IL12A, IRF1, IRF8, PIK3R1, PRKCA, |
| | TGFB2 |
| Tetrahydrofolate Salvage from 5,10- | MTHFD1 |
| methenyltetrahydrofolate | |
| Tyrosine Degradation I | HPD |
| dTMP De Novo Biosynthesis | SHMT2 |
| Polyamine Regulation in Colon Cancer | KRAS, MAX |
| Human Embryonic Stem Cell Pluripotency | BDNF, NOG, PDPK1, PIK3R1, TCF7L2, |
| | TGFB2, TGFBR2 |
| Hepatic Fibrosis/Hepatic Stellate Cell | CCL2, COL20A1, COL6A3, IL4R, IL6R, MYH11, |
| Activation | SERPINE1, TGFB2, TGFBR2 |
| Role of PI3K/AKT Signaling in the Pathogenesis | CASP9, CRKL, GNAI3, PIK3R1 |
| of Influenza | |
| CD40 Signaling | FOS, PIK3R1, STAT3, TRAF1 |
| Calcium-induced T Lymphocyte Apoptosis | ATP2A1, ORAI1, PPP3R1, PRKCA |
| Apelin Cardiac Fibroblast Signaling Pathway | SERPINE1, TGFB2 |
| Differential Regulation of Cytokine Production in | CCL2, IL12A |
| Intestinal Epithelial Cells by IL-17A and IL-17F | |
| Ethanol Degradation IV | ACSS2, ALDH9A1 |
| Superpathway of D-myo-inositol (1,4,5)- | INPPL1, ITPKB |
| trisphosphate Metabolism | |
| Adenine and Adenosine Salvage III | HPRT1 |
| Glycogen Biosynthesis II (from UDP-D-Glucose) | GBE1 |
| UDP-N-acetyl-D-glucosamine Biosynthesis II | GFPT1 |
| Urea Cycle | CPS1 |
| Zymosterol Biosynthesis | LBR |
| IL-1 Signaling | ADCY9, FOS, GNAI3, GNAZ, GNB1 |
| Clathrin-mediated Endocytosis Signaling | ACTR3, AP1S2, AP2M1, ARPC2, ARPC5, CBL, |
| | EPHB2, PIK3R1, PPP3R1 |
| Tryptophan Degradation X (Mammalian, via | ALDH9A1, MAOB |
| Tryptamine) | |
| Role of JAK1 and JAK3 in γc Cytokine Signaling | IL4R, KRAS, PIK3R1, STAT3 |
| nNOS Signaling in Neurons | CAPN10, PPP3R1, PRKCA |
| Corticotropin Releasing Hormone Signaling | ADCY9, ARPC5, BDNF, FOS, GNAI3, NPR1, |
| | PRKCA |
| α-Adrenergic Signaling | ADCY9, GNAI3, GNB1, KRAS, PRKCA |
| Glycoaminoglycan-protein Linkage Region | XYLT1 |
| Biosynthesis | |
| Superpathway of Serine and Glycine | SHMT2 |
| Biosynthesis I | |
| Tryptophan Degradation to 2-amino-3- | AFMID |
| carboxymuconate Semialdehyde | |
| Role of Tissue Factor in Cancer | F2RL1, F3, KRAS, PIK3R1, PLAUR, PRKCA |
| IL-15 Signaling | KRAS, PIK3R1, PTK2, STAT3 |
| ATM Signaling | CBX1, CDK2, CHEK1, HP1BP3, RNF168 |
| Cell Cycle: G2/M DNA Damage Checkpoint | CHEK1, SFN, YWHAZ |
| Regulation | |
| T Cell Exhaustion Signaling Pathway | FOS, IL12A, IL6R, KRAS, PIK3R1, STAT3, |
| | TGFBR2, TGFBR3 |
| Phagosome Maturation | ATP6V1B2, DYNC2H1, GOSR2, NSF, TUBA1B, |
| | TUBA4A, VPS37B |
| Primary Immunodeficiency Signaling | RFX5, TAP2, UNG |
| TNFR1 Signaling | CASP9, FOS, PAK4 |
| Histidine Degradation III | MTHFD1 |
| Salvage Pathways of Pyrimidine | TK2 |
| Deoxyribonucleotides | |
| Superpathway of Cholesterol Biosynthesis | DHCR24, LBR |
| Amyloid Processing | APH1B, CAPN10, PSEN1 |
| Phagosome Formation | NOTUM, PIK3R1, PRKCA, RHOU, TLR6, VTN |
| Toll-like Receptor Signaling | FOS, IL12A, TLR6, TRAF1 |
| UVB-Induced MAPK Signaling | FOS, PIK3R1, PRKCA |
| Antiproliferative Role of Somatostatin Receptor | GNB1, KRAS, NPR1, PIK3R1 |
| 2 | |
| Dopamine Degradation | ALDH9A1, MAOB |
| TNFR2 Signaling | FOS, TRAF1 |
| Heme Biosynthesis II | CPOX |
| Leucine Degradation I | BCAT2 |
| UDP-N-acetyl-D-galactosamine Biosynthesis II | GNPDA1 |
| Transcriptional Regulatory Network in | HIST1H4C, SET, STAT3 |
| Embryonic Stem Cells | |
| Cellular Effects of Sildenafil (Viagra) | ADCY9, MYH11, MYLK, NOTUM, PDE3A, |
| | PDE4D |
| Acetone Degradation I (to Methylglyoxal) | CYP1A1 |
| Role of p14/p19ARF in Tumor Suppression | PIK3R1 |
| Sonic Hedgehog Signaling | DYRK1B |
| 4-1BB Signaling in T Lymphocytes | TNFSF9, TRAF1 |
| Dolichyl-diphosphooligosaccharide Biosynthesis | DPM3 |
| Glycine Betaine Degradation | SHMT2 |
| Virus Entry via Endocytic Pathways | AP1S2, AP2M1, KRAS, PIK3R1, PRKCA |
| Retinoic acid Mediated Apoptosis Signaling | CASP9, IRF1 |
| SPINK1 Pancreatic Cancer Pathway | F2RL1, TGFBR2 |
| Androgen Signaling | CCND1, GNAI3, GNAZ, GNB1, PRKCA, SHBG |
| Dermatan Sulfate Biosynthesis | DSEL, XYLT1 |
| Iron homeostasis signaling pathway | ABCB10, ATP6V1B2, HFE, IL6R, PCBP1, |
| | STAT3 |
| Lipid Antigen Presentation by CD1 | AP2M1 |
| NAD Salvage Pathway II | NT5E |
| Cancer Drug Resistance By Drug Efflux | KRAS, PIK3R1 |
| CTLA4 Signaling in Cytotoxic T Lymphocytes | AP1S2, AP2M1, PIK3R1 |
| MSP-RON Signaling Pathway | CCL2, IL12A, PIK3R1 |
| IL-9 Signaling | PIK3R1, STAT3 |
| Retinoate Biosynthesis I | AKR1C3, DHRS9 |
| Bupropion Degradation | CYP1A1 |
| D-myo-inositol (1,4,5)-Trisphosphate | PIP4K2C |
| Biosynthesis | |
| IL-17A Signaling in Gastric Cells | FOS |
| Role of CHK Proteins in Cell Cycle Checkpoint | CDK2, CHEK1 |
| Control | |
| Semaphorin Signaling in Neurons | PAK4, PTK2, RHOU |
| Glutathione Redox Reactions I | GSR |
| IL-22 Signaling | STAT3 |
| Role of JAK1, JAK2 and TYK2 in Interferon | STAT3 |
| Signaling | |
| Tumoricidal Function of Hepatic Natural Killer | CASP9 |
| Cells | |
| TWEAK Signaling | CASP9, TRAF1 |
| p38 MAPK Signaling | MAX, RPS6KB2, TGFB2, TGFBR2 |
| Role of IL-17A in Arthritis | CCL2, PIK3R1 |
| Cleavage and Polyadenylation of Pre-mRNA | NUDT21 |
| Guanosine Nucleotides Degradation III | NT5E |
| Interferon Signaling | IRF1, MED14 |
| Ceramide Signaling | FOS, KRAS, PIK3R1, SMPD1 |
| Phototransduction Pathway | GNB1, RGS9BP |
| Phosphatidylglycerol Biosynthesis II (Non- | AGPAT1 |
| plastidic) | |
| Pyrimidine Deoxyribonucleotides De Novo | AK7 |
| Biosynthesis I | |
| Tryptophan Degradation III (Eukaryotic) | AFMID |
| NF-κB Activation by Viruses | KRAS, PIK3R1, PRKCA |
| Acyl-CoA Hydrolysis | ACOT8 |
| Urate Biosynthesis/Inosine 5′-phosphate | NT5E |
| Degradation | |
| IL-17 Signaling | CCL2, KRAS, PIK3R1 |
| Mitochondrial Dysfunction | APH1B, ATP5PB, CASP9, GSR, MAOB, PSEN1 |
| IL-17A Signaling in Airway Cells | MUC5AC, PIK3R1, STAT3 |
| April Mediated Signaling | FOS, TRAF1 |
| Inhibition of Matrix Metalloproteases | RECK, TIMP4 |
| CDP-diacylglycerol Biosynthesis I | AGPAT1 |
| Fatty Acid α-oxidation | ALDH9A1 |
| Granzyme A Signaling | SET |
| Androgen Biosynthesis | AKR1C3 |
| Isoleucine Degradation I | BCAT2 |
| NAD biosynthesis II (from tryptophan) | AFMID |
| Hematopoiesis from Pluripotent Stem Cells | IL11, IL12A |
| CCR5 Signaling in Macrophages | FOS, GNAI3, GNB1, PRKCA |
| Role of MAPK Signaling in the Pathogenesis of | CCL2, KRAS, PRKCA |
| Influenza | |
| Serotonin Degradation | ALDH9A1, DHRS9, MAOB |
| GABA Receptor Signaling | ADCY9, ALDH9A1, AP2M1, NSF |
| Antioxidant Action of Vitamin C | NOTUM, PLD6, SLC23A2, SLC2A3 |
| Macropinocytosis Signaling | KRAS, PIK3R1, PRKCA |
| Gustation Pathway | ADCY9, GNB1, PDE3A, PDE4D, PDE8A, PLD6 |
| B Cell Activating Factor Signaling | FOS, TRAF1 |
| Estrogen Biosynthesis | AKR1C3, CYP1A1 |
| Mechanisms of Viral Exit from Host Cells | LMNB2, PRKCA |
| Role of PKR in Interferon Induction and Antiviral | CASP9, IRF1 |
| Response | |
| Adenosine Nucleotides Degradation II | NT5E |
| Choline Biosynthesis III | PLD6 |
| Superpathway of Citrulline Metabolism | CPS1 |
| FcγRIIB Signaling in B Lymphocytes | KRAS, PDPK1, PIK3R1 |
| Heparan Sulfate Biosynthesis (Late Stages) | EXTL2, EXTL3, NOTUM |
| Bladder Cancer Signaling | CCND1, CDH1, CDK4, KRAS |
| IL-10 Signaling | FOS, IL4R, STAT3 |
| Adipogenesis pathway | CEBPD, EBF1, HDAC3, PER2, TBL1XR1 |
| Purine Nucleotides Degradation II (Aerobic) | NT5E |
| Valine Degradation I | BCAT2 |
| PFKFB4 Signaling Pathway | NCOA3, TGFB2 |
| PPAR Signaling | FOS, KRAS, NRIP1, SOS2 |
| Retinol Biosynthesis | AKR1C3, DHRS9 |
| Agranulocyte Adhesion and Diapedesis | CCL2, CLDN2, CLDN4, GNAI3, MYH11, |
| | PODXL, SELPLG |
| Amyotrophic Lateral Sclerosis Signaling | CAPN10, CASP9, NEFH, PIK3R1 |
| NER Pathway | COPS3, DDB1, HIST1H4C, POLE3 |
| Chondroitin Sulfate Degradation (Metazoa) | CEMIP2 |
| Parkinson's Signaling | CASP9 |
| IL-23 Signaling Pathway | PIK3R1, STAT3 |
| Role of IL-17F in Allergic Inflammatory Airway | CCL2, IL11 |
| Diseases | |
| Stearate Biosynthesis I (Animals) | ACOT8, DHCR24 |
| iNOS Signaling | FOS, IRF1 |
| Role of Hypercytokinemia/hyperchemokinemia | CCL2, IL12A |
| in the Pathogenesis of Influenza | |
| D-myo-inositol (1,4,5)-trisphosphate | INPPL1 |
| Degradation | |
| Histamine Degradation | ALDH9A1 |
| Melatonin Signaling | GNAI3, NOTUM, PRKCA |
| Altered T Cell and B Cell Signaling in | IL12A, TLR6 |
| Rheumatoid Arthritis | |
| Antigen Presentation Pathway | TAP2 |
| Apelin Adipocyte Signaling Pathway | ADCY9, GNAI3 |
| Apelin Pancreas Signaling Pathway | PIK3R1 |
| Assembly of RNA Polymerase II Complex | TAF7L |
| Atherosclerosis Signaling | CCL2, F3, SELPLG |
| Autophagy | SQSTM1, WIPI1 |
| BMP signaling pathway | KRAS, NOG |
| Basal Cell Carcinoma Signaling | TCF7L2 |
| CDK5 Signaling | ADCY9, BDNF, KRAS |
| Caveolar-mediated Endocytosis Signaling | MAP3K2, PRKCA |
| Chondroitin Sulfate Biosynthesis | XYLT1 |
| Communication between Innate and Adaptive | IL12A, TLR6 |
| Immune Cells | |
| Complement System | C1QBP |
| Crosstalk between Dendritic Cells and Natural | IL12A |
| Killer Cells | |
| Cytotoxic T Lymphocyte-mediated Apoptosis of | CASP9 |
| Target Cells | |
| Death Receptor Signaling | CASP9, TNFRSF21 |
| Dendritic Cell Maturation | IL12A, IRF8, NOTUM, PIK3R1 |
| Dermatan Sulfate Biosynthesis (Late Stages) | DSEL |
| Eicosanoid Signaling | AKR1C3, ALOX5AP |
| FXR/RXR Activation | VTN |
| Fatty Acid β-oxidation I | SCP2 |
| G Protein Signaling Mediated by Tubby | GNB1 |
| Glutamate Receptor Signaling | GNB1 |
| Granulocyte Adhesion and Diapedesis | CCL2, CLDN2, CLDN4, GNAI3, SELPLG |
| Gαs Signaling | ADCY9, ADRB1, GNB1 |
| HIF1α Signaling | KRAS, PIK3R1, SLC2A3 |
| Hepatic Cholestasis | ADCY9, IL11, IL12A, PRKCA, TGFB2, TNFSF9 |
| Hypoxia Signaling in the Cardiovascular System | UBE2L6, UBE2Z |
| Induction of Apoptosis by HIV1 | CASP9, TRAF1 |
| Inhibition of Angiogenesis by TSP1 | TGFBR2 |
| Intrinsic Prothrombin Activation Pathway | THBD |
| LPS/IL-1 Mediated Inhibition of RXR Function | ALDH9A1, MAOB |
| LXR/RXR Activation | CCL2, VTN |
| MIF Regulation of Innate Immunity | FOS |
| Melatonin Degradation I | CYP1A1 |
| Netrin Signaling | PPP3R1 |
| Neuroprotective Role of THOP1 in Alzheimer's | TPP1 |
| Disease | |
| Nicotine Degradation II | CYP1A1 |
| Nicotine Degradation III | CYP1A1 |
| Oxidative Phosphorylation | ATP5PB |
| POP pathway | LGR4, ROR2 |
| Phospholipases | NOTUM, PLD6 |
| Protein Ubiquitination Pathway | CBL, DNAJC16, DNAJC3, HSPA13, TAP2, |
| | UBE2L6, UBE2Z, USP39, USP43 |
| Reelin Signaling in Neurons | CRKL, PIK3R1 |
| Role of BRCA1 in DNA Damage Response | CHEK1, FANCE |
| Role of Cytokines in Mediating Communication | IL12A |
| between Immune Cells | |
| Role of JAK2 in Hormone-like Cytokine | STAT3 |
| Signaling | |
| Role of Oct4 in Mammalian Embryonic Stem | NR2F6 |
| Cell Pluripotency | |
| Role of Osteoblasts, Osteoclasts and | CASP9, CBL, FOS, IL11, PIK3R1, PPP3R1, |
| Chondrocytes in Rheumatoid Arthritis | TCF7L2 |
| Role of RIG1-like Receptors in Antiviral Innate | TRIM25 |
| Immunity | |
| Role of Wnt/GSK-3β Signaling in the | NCOA3, TCF7L2 |
| Pathogenesis of Influenza | |
| Superpathway of Melatonin Degradation | CYP1A1, MAOB |
| Systemic Lupus Erythematosus Signaling | CBL, FOS, IL6R, KRAS, PIK3R1, SOS2 |
| Th17 Activation Pathway | IL12A, IL6R, STAT3 |
| Thyroid Hormone Metabolism II (via Conjugation | DIO2 |
| and/or Degradation) | |
| Triacylglycerol Biosynthesis | AGPAT1 |
| Triacylglycerol Degradation | NOTUM |
| Type I Diabetes Mellitus Signaling | CASP9, IL12A, IRF1 |
| Type II Diabetes Mellitus Signaling | PDPK1, PIK3R1, PRKCA, SMPD1 |
| Wnt/Ca⁺ pathway | NOTUM, PRKCA |
| Xenobiotic Metabolism Signaling | ALDH9A1, CYP1A1, KRAS, MAOB, MAP3K2, |
| | MAP3K3, NRIP1, PIK3R1, PRKCA |
| iCOS-iCOSL Signaling in T Helper Cells | PDPK1, PIK3R1, PPP3R1 |
| tRNA Charging | LARS2 |

TABLE 8
A549 lung cancer downregulated pathways and associated genes

| Pathway Name | Gene |
|---|---|
| HGF Signaling | CCND1, CDK2, CRKL, ELK3, ETS1, FOS, |
| | KRAS, MAP3K2, MAP3K3, PIK3R1, PRKCA, |
| | PTK2, SOS2, STAT3 |
| Signaling by Rho Family GTPases | ACTR3, ARPC2, ARPC5, CDC42EP2, CDH1, |
| | CDH16, CDH8, FOS, GNAI3, GNAZ, GNB1, |
| | MYLK, PAK4, PARD6A, PIK3R1, PIP4K2C, |
| | PTK2, RHOU, STMN1 |
| Small Cell Lung Cancer Signaling | CASP9, CCND1, CDK2, CDK4, CDK6, MAX, |
| | PIK3R1, PTK2, TRAF1 |
| IGF-1 Signaling | CASP9, FOS, KRAS, PDPK1, PIK3R1, PTK2, |
| | RPS6KB2, SFN, SOS2, STAT3, YWHAZ |
| Non-Small Cell Lung Cancer Signaling | CASP9, CCND1, CDK4, CDK6, KRAS, PDPK1, |
| | PIK3R1, PRKCA, SOS2 |
| Synaptogenesis Signaling Pathway | ACTR3, ADCY9, AP2M1, ARPC2, ARPC5, |
| | BDNF, CADM1, CDH1, CDH16, CDH8, CRKL, |
| | EIF4EBP2, EPHB2, EPHB3, GOSR2, KRAS, |
| | NSF, PIK3R1, RPS6KB2, SOS2, STX1B, SYT1 |
| Cardiac Hypertrophy Signaling (Enhanced) | ADCY9, ADRB1, ATP2A1, GNAI3, GNB1, |
| | HDAC3, IL11, IL12A, IL17RD, IL4R, IL6R, |
| | KRAS, MAP3K2, MAP3K3, NOTUM, NPR1, |
| | PDE3A, PDE4D, PDE8A, PIK3R1, PLD6, |
| | PPP3R1, PRKCA, PTK2, RPS6KB2, STAT3, |
| | TGFB2, TGFBR2, TGFBR3, TNFSF9 |
| Ephrin Receptor Signaling | ACTR3, ARPC2, ARPC5, BCAR1, CRKL, |
| | EPHB2, EPHB3, GNAI3, GNAZ, GNB1, KRAS, |
| | PAK4, PTK2, SOS2, STAT3 |
| Neuregulin Signaling | CRKL, ERBIN, ERRFI1, KRAS, PDPK1, |
| | PIK3R1, PRKCA, PSEN1, RPS6KB2, SOS2 |
| Regulation of Cellular Mechanics by Calpain | CAPN10, CCNA2, CCND1, CDK2, CDK4, |
| Protease | CDK6, KRAS, PTK2 |
| ErbB4 Signaling | APH1B, KRAS, PDPK1, PIK3R1, PRKCA, |
| | PSEN1, SOS2, YAP1 |
| Ephrin B Signaling | CBL, EPHB2, EPHB3, GNAI3, GNAZ, GNB1, |
| | PTK2, VAV3 |
| Aryl Hydrocarbon Receptor Signaling | ALDH9A1, CCNA2, CCND1, CDK2, CDK4, |
| | CDK6, CHEK1, CYP1A1, FOS, NCOA3, NRIP1, |
| | TGFB2 |
| Glioma Invasiveness Signaling | KRAS, PIK3R1, PLAU, PLAUR, PTK2, RHOU, |
| | TIMP4, VTN |
| 14-3-3-mediated Signaling | CBL, FOS, KRAS, NOTUM, PIK3R1, PRKCA, |
| | SFN, TUBA1B, TUBA4A, YAP1, YWHAZ |
| B Cell Receptor Signaling | EBF1, ETS1, INPPL1, KRAS, MAP3K2, |
| | MAP3K3, PDPK1, PIK3AP1, PIK3R1, PPP3R1, |
| | PTK2, RPS6KB2, SOS2, VAV3 |
| Fcγ Receptor-mediated Phagocytosis in | ACTR3, ARPC2, ARPC5, CBL, PIK3R1, PLD6, |
| Macrophages and Monocytes | PRKCA, RPS6KB2, VAV3 |
| Endometrial Cancer Signaling | CASP9, CCND1, CDH1, KRAS, PDPK1, |
| | PIK3R1, SOS2 |
| p70S6K Signaling | F2RL1, GNAI3, IL4R, KRAS, NOTUM, PDPK1, |
| | PIK3R1, PRKCA, SFN, SOS2, YWHAZ |
| CXCR4 Signaling | ADCY9, BCAR1, ELMO2, FOS, GNAI3, GNAZ, |
| | GNB1, KRAS, PAK4, PIK3R1, PRKCA, PTK2, |
| | RHOU |
| Rac Signaling | ABI2, ACTR3, ARPC2, ARPC5, KRAS, PAK4, |
| | PARD6A, PIK3R1, PIP4K2C, PTK2 |
| IL-7 Signaling Pathway | CCND1, CDK2, EBF1, MCL1, PDPK1, PIK3R1, |
| | PTK2, SOS2 |
| Colorectal Cancer Metastasis Signaling | ADCY9, APPL1, CASP9, CCND1, CDH1, FOS, |
| | GNB1, IL6R, KRAS, PIK3R1, RHOU, SOS2, |
| | STAT3, TCF7L2, TGFB2, TGFBR2, TLR6 |
| Prolactin Signaling | FOS, IRF1, KRAS, PDPK1, PIK3R1, PRKCA, |
| | SOS2, STAT3 |
| Renin-Angiotensin Signaling | ADCY9, CCL2, FOS, KRAS, PAK4, PIK3R1, |
| | PRKCA, PTK2, SOS2, STAT3 |
| PDGF Signaling | CRKL, FOS, INPPL1, KRAS, PIK3R1, PRKCA, |
| | SOS2, STAT3 |
| GM-CSF Signaling | CCND1, ETS1, KRAS, PIK3R1, PPP3R1, |
| | SOS2, STAT3 |
| Tec Kinase Signaling | FOS, GNAI3, GNAZ, GNB1, PAK4, PIK3R1, |
| | PRKCA, PTK2, RHOU, STAT3, TNFRSF21, |
| | VAV3 |
| ERK5 Signaling | FOS, KRAS, MAP3K2, MAP3K3, RPS6KB2, |
| | SFN, YWHAZ |
| Acute Myeloid Leukemia Signaling | CCND1, IDH2, KRAS, PIK3R1, RPS6KB2, |
| | SOS2, STAT3, TCF7L2 |
| Estrogen-mediated S-phase Entry | CCNA2, CCND1, CDK2, CDK4 |
| Relaxin Signaling | ADCY9, FOS, GNAI3, GNAZ, GNB1, NPR1, |
| | PDE3A, PDE4D, PDE8A, PIK3R1, PLD6 |
| PI3K/AKT Signaling | CCND1, INPPL1, KRAS, MCL1, PDPK1, |
| | PIK3R1, RPS6KB2, SFN, SOS2, YWHAZ |
| ERK/MAPK Signaling | BCAR1, CRKL, ELK3, ETS1, FOS, KRAS, |
| | PAK4, PIK3R1, PRKCA, PTK2, SOS2, STAT3, |
| | YWHAZ |
| Integrin Signaling | ACTR3, ARF3, ARPC2, ARPC5, BCAR1, |
| | CAPN10, CRKL, KRAS, MYLK, PAK4, PIK3R1, |
| | PTK2, RHOU, SOS2 |
| Role of NFAT in Cardiac Hypertrophy | ADCY9, GNAI3, GNB1, HDAC3, IL11, KRAS, |
| | NOTUM, PIK3R1, PPP3R1, PRKCA, RCAN3, |
| | SOS2, TGFB2, TGFBR2 |
| Cardiac Hypertrophy Signaling | ADCY9, ADRB1, GNAI3, GNAZ, GNB1, IL6R, |
| | KRAS, MAP3K2, MAP3K3, NOTUM, PIK3R1, |
| | PPP3R1, RHOU, TGFB2, TGFBR2 |
| Actin Cytoskeleton Signaling | ABI2, ACTR3, ARPC2, ARPC5, BCAR1, CRKL, |
| | KRAS, MYH11, MYLK, PAK4, PIK3R1, PTK2, |
| | SOS2, VAV3 |
| UVA-Induced MAPK Signaling | CASP9, FOS, KRAS, NOTUM, PIK3R1, |
| | PRKCA, RPS6KB2, SMPD1 |
| fMLP Signaling in Neutrophils | ACTR3, ARPC2, ARPC5, GNAI3, GNB1, KRAS, |
| | PIK3R1, PPP3R1, PRKCA |
| IL-3 Signaling | CRKL, FOS, KRAS, PIK3R1, PPP3R1, PRKCA, |
| | STAT3 |
| PI3K Signaling in B Lymphocytes | CBL, FOS, IL4R, KRAS, NOTUM, PDPK1, |
| | PIK3AP1, PIK3R1, PPP3R1, VAV3 |
| Cyclins and Cell Cycle Regulation | CCNA2, CCND1, CDK2, CDK4, CDK6, HDAC3, |
| | TGFB2 |
| SAPK/JNK Signaling | CRKL, GNB1, KRAS, MAP3K2, MAP3K3, |
| | MAP4K5, PIK3R1, SOS2 |
| ErbB2-ErbB3 Signaling | CCND1, KRAS, PDPK1, PIK3R1, SOS2, STAT3 |
| HMGB1 Signaling | CCL2, FOS, HMGB1, IL11, IL12A, KRAS, |
| | PIK3R1, RHOU, SERPINE1, TGFB2, TNFSF9 |
| Protein Kinase A Signaling | ADCY9, AKAP12, GNAI3, GNB1, HHAT, MYLK, |
| | NOTUM, PDE3A, PDE4D, PDE8A, PLD6, |
| | PPP3R1, PRKCA, PTK2, PTPN9, PTPRA, SFN, |
| | TCF7L2, TGFB2, TGFBR2, YWHAZ |
| Remodeling of Epithelial Adherens Junctions | ACTR3, ARPC2, ARPC5, CDH1, TUBA1B, |
| | TUBA4A |
| Melanoma Signaling | CCND1, CDH1, CDK4, KRAS, PIK3R1 |
| Telomerase Signaling | ELK3, ETS1, HDAC3, KRAS, PDPK1, PIK3R1, |
| | SOS2, TPP1 |
| GNRH Signaling | ADCY9, FOS, GNAI3, GNB1, KRAS, MAP3K2, |
| | MAP3K3, PAK4, PRKCA, PTK2, SOS2 |
| Glioma Signaling | CCND1, CDK4, CDK6, IDH2, KRAS, PIK3R1, |
| | PRKCA, SOS2 |
| Actin Nucleation by ARP-WASP Complex | ACTR3, ARPC2, ARPC5, KRAS, RHOU, SOS2 |
| Growth Hormone Signaling | FOS, PDPK1, PIK3R1, PRKCA, RPS6KB2, |
| | STAT3 |
| Regulation of Actin-based Motility by Rho | ACTR3, ARPC2, ARPC5, MYLK, PAK4, |
| | PIP4K2C, RHOU |
| EGF Signaling | FOS, PIK3R1, PRKCA, SOS2, STAT3 |
| NGF Signaling | KRAS, MAP3K2, MAP3K3, PDPK1, PIK3R1, |
| | RPS6KB2, SMPD1, SOS2 |
| ErbB Signaling | FOS, KRAS, PAK4, PDPK1, PIK3R1, PRKCA, |
| | SOS2 |
| Aldosterone Signaling in Epithelial Cells | DNAJC16, DNAJC3, HSPA13, KRAS, NOTUM, |
| | PDPK1, PIK3R1, PIP4K2C, PRKCA, SOS2 |
| Apelin Endothelial Signaling Pathway | ADCY9, CCL2, FOS, GNAI3, KRAS, PIK3R1, |
| | PRKCA, RPS6KB2 |
| Th2 Pathway | APH1B, BHLHE41, IL12A, IL4R, NOTCH2, |
| | PIK3R1, PSEN1, TGFBR2, TGFBR3 |
| Neurotrophin/TRK Signaling | BDNF, FOS, KRAS, PDPK1, PIK3R1, SOS2 |
| Sphingosine-1-phosphate Signaling | ADCY9, CASP9, GNAI3, NOTUM, PIK3R1, |
| | PTK2, RHOU, SMPD1 |
| PAK Signaling | EPHB3, KRAS, MYLK, PAK4, PIK3R1, PTK2, |
| | SOS2 |
| Cardiac β-adrenergic Signaling | ADCY9, ADRB1, AKAP12, ATP2A1, GNB1, |
| | PDE3A, PDE4D, PDE8A, PLD6 |
| Agrin Interactions at Neuromuscular Junction | GABPA, GABPB1, KRAS, PAK4, PTK2, RAPSN |
| Thrombin Signaling | ADCY9, GNAI3, GNAZ, GNB1, KRAS, MYLK, |
| | NOTUM, PDPK1, PIK3R1, PRKCA, PTK2, |
| | RHOU |
| tRNA Splicing | PDE3A, PDE4D, PDE8A, PLD6 |
| Chemokine Signaling | CCL2, FOS, GNAI3, KRAS, PRKCA, PTK2 |
| Glioblastoma Multiforme Signaling | CCND1, CDK2, CDK4, CDK6, KRAS, NF1, |
| | NOTUM, PIK3R1, RHOU, SOS2 |
| Th1 Pathway | APH1B, IL12A, IL6R, IRF1, NOTCH2, PIK3R1, |
| | PSEN1, STAT3 |
| Renal Cell Carcinoma Signaling | ETS1, FOS, KRAS, PAK4, PIK3R1, SOS2 |
| Oncostatin M Signaling | KRAS, OSMR, PLAU, STAT3 |
| Thrombopoietin Signaling | FOS, KRAS, PIK3R1, PRKCA, STAT3 |
| FLT3 Signaling in Hematopoietic Progenitor | CBL, KRAS, PDPK1, PIK3R1, SOS2, STAT3 |
| Cells | |
| P2Y Purigenic Receptor Signaling Pathway | ADCY9, FOS, GNAI3, GNB1, KRAS, NOTUM, |
| | PIK3R1, PRKCA |
| Adrenomedullin signaling pathway | ADCY9, ADM, FOS, KRAS, MAX, MYLK, |
| | NOTUM, NPR1, PIK3R1, PTK2, SOS2 |
| Leukocyte Extravasation Signaling | BCAR1, CLDN2, CLDN4, CRKL, GNAI3, |
| | PIK3R1, PRKCA, PTK2, SELPLG, TIMP4, VAV3 |
| Gα12/13 Signaling | CDH1, CDH16, CDH8, F2RL1, KRAS, PIK3R1, |
| | PTK2, VAV3 |
| IL-8 Signaling | CCND1, CDH1, FOS, GNAI3, GNB1, KRAS, |
| | PIK3R1, PLD6, PRKCA, PTK2, RHOU |
| RANK Signaling in Osteoclasts | CBL, FOS, MAP3K2, MAP3K3, PIK3R1, |
| | PPP3R1 |
| STAT3 Pathway | IL17RD, IL4R, IL6R, KRAS, STAT3, TGFB2, |
| | TGFBR2, TGFBR3 |
| Role of NFAT in Regulation of the Immune | FOS, GNAI3, GNAZ, GNB1, KRAS, ORAI1, |
| Response | PIK3R1, PPP3R1, RCAN3, SOS2 |
| UVC-Induced MAPK Signaling | FOS, KRAS, PRKCA, SMPD1 |
| Endocannabinoid Developing Neuron Pathway | ADCY9, CCND1, GNAI3, GNB1, KRAS, |
| | PIK3R1, STAT3 |
| Insulin Receptor Signaling | CBL, CRKL, INPPL1, KRAS, PDPK1, PIK3R1, |
| | RPS6KB2, SOS2 |
| Endothelin-1 Signaling | ADCY9, CASP9, FOS, GNAI3, GNAZ, KRAS, |
| | NOTUM, PIK3R1, PLD6, PRKCA |
| TGF-β Signaling | FOS, KRAS, SERPINE1, SOS2, TGFB2, |
| | TGFBR2 |
| Huntington's Disease Signaling | ATP5PB, BDNF, CAPN10, CASP9, GNB1, |
| | GOSR2, HDAC3, NSF, PDPK1, PIK3R1, |
| | PRKCA, SOS2 |
| Fc Epsilon RI Signaling | INPPL1, KRAS, PDPK1, PIK3R1, PRKCA, |
| | SOS2, VAV3 |
| Cholecystokinin/Gastrin-mediated Signaling | BCAR1, FOS, KRAS, PRKCA, PTK2, RHOU, |
| | SOS2 |
| Lymphotoxin β Receptor Signaling | CASP9, PDPK1, PIK3R1, TRAF1 |
| GP6 Signaling Pathway | COL20A1, COL6A3, LAMC2, PDPK1, PIK3R1, |
| | PRKCA, PTK2 |
| CD28 Signaling in T Helper Cells | ACTR3, ARPC2, ARPC5, FOS, PDPK1, |
| | PIK3R1, PPP3R1 |
| G Beta Gamma Signaling | GNAI3, GNAZ, GNB1, KRAS, PDPK1, PRKCA, |
| | SOS2 |
| RhoA Signaling | ACTR3, ARPC2, ARPC5, CDC42EP2, MYLK, |
| | PIP4K2C, PTK2 |
| Apelin Cardiomyocyte Signaling Pathway | ATP2A1, GNAI3, MYLK, NOTUM, PIK3R1, |
| | PRKCA |
| VEGF Signaling | KRAS, PIK3R1, PRKCA, PTK2, SFN, SOS2 |
| CCR3 Signaling in Eosinophils | GNAI3, GNB1, KRAS, MYLK, PAK4, PIK3R1, |
| | PRKCA |
| IL-6 Signaling | FOS, IL6R, KRAS, MCL1, PIK3R1, SOS2, |
| | STAT3 |
| Neuroinflammation Signaling Pathway | APH1B, BDNF, CCL2, FOS, HMGB1, IL12A, |
| | IL6R, PIK3R1, PPP3R1, PSEN1, TGFB2, |
| | TGFBR2, TGFBR3, TLR6 |
| JAK/Stat Signaling | FOS, KRAS, PIK3R1, SOS2, STAT3 |
| Acute Phase Response Signaling | FOS, IL6R, KRAS, OSMR, PDPK1, PIK3R1, |
| | SERPINE1, SOS2, STAT3 |
| CNTF Signaling | KRAS, PIK3R1, RPS6KB2, STAT3 |
| Role of Pattern Recognition Receptors in | IL11, IL12A, PIK3R1, PRKCA, RIPK2, TGFB2, |
| Recognition of Bacteria and Viruses | TLR6, TNFSF9 |
| PKCθ Signaling in T Lymphocytes | FOS, KRAS, MAP3K2, MAP3K3, PIK3R1, |
| | PPP3R1, SOS2, VAV3 |
| IL-2 Signaling | FOS, KRAS, PIK3R1, SOS2 |
| FGF Signaling | CRKL, PIK3R1, PRKCA, SOS2, STAT3 |
| VEGF Family Ligand-Receptor Interactions | FOS, KRAS, PIK3R1, PRKCA, SOS2 |
| Paxillin Signaling | BCAR1, KRAS, PAK4, PIK3R1, PTK2, SOS2 |
| mTOR Signaling | EIF4B, KRAS, PDPK1, PIK3R1, PLD6, PRKCA, |
| | PRR5L, RHOU, RPS17, RPS6KB2 |
| Pyridoxal 5′-phosphate Salvage Pathway | CDK2, CDK4, CDK6, FAM20B |
| Dopamine-DARPP32 Feedback in cAMP | ADCY9, ATP2A1, CAMKK1, GNAI3, KCNJ16, |
| Signaling | NOTUM, PPP3R1, PRKCA |
| Production of Nitric Oxide and Reactive Oxygen | FOS, HOXA10, IRF1, IRF8, MAP3K2, MAP3K3, |
| Species in Macrophages | PIK3R1, PRKCA, RHOU |
| Cdc42 Signaling | ACTR3, ARPC2, ARPC5, CDC42EP2, FOS, |
| | MYLK, PAK4, PARD6A |
| Sperm Motility | DSTYK, EPHB2, EPHB3, NOTUM, NPR1, |
| | PDE4D, PRKCA, PTK2, ROR2, TWF1 |
| SPINK1 General Cancer Pathway | IL6R, KRAS, PIK3R1, STAT3 |
| Melanocyte Development and Pigmentation | ADCY9, KRAS, PIK3R1, RPS6KB2, SOS2 |
| Signaling | |
| Superpathway of Inositol Phosphate | HACD2, INPPL1, ITPKB, NUDT15, NUDT3, |
| Compounds | PIK3R1, PIP4K2C, PPTC7, SET |
| Salvage Pathways of Pyrimidine | AK7, CDK2, CDK4, CDK6, FAM20B |
| Ribonucleotides | |
| cAMP-mediated signaling | ADCY9, ADRB1, AKAP12, GNAI3, PDE3A, |
| | PDE4D, PDE8A, PLD6, PPP3R1, STAT3 |
| Systemic Lupus Erythematosus In T Cell | CASP9, CBL, FOS, GNAI3, KRAS, ORAI1, |
| Signaling Pathway | PIK3R1, PPP3R1, PTK2, RHOU, RPS6KB2, |
| | SELPLG, SOS2, STAT3 |
| Gαi Signaling | ADCY9, GNAI3, GNB1, KRAS, SOS2, STAT3 |
| Estrogen-Dependent Breast Cancer Signaling | CCND1, FOS, KRAS, PIK3R1 |
| TREM1 Signaling | CCL2, LAT2, STAT3, TLR6 |
| Neuropathic Pain Signaling In Dorsal Horn | BDNF, FOS, NOTUM, PIK3R1, PRKCA |
| Neurons | |
| GDNF Family Ligand-Receptor Interactions | FOS, KRAS, PIK3R1, SOS2 |
| CREB Signaling in Neurons | ADCY9, GNAI3, GNAZ, GNB1, KRAS, NOTUM, |
| | PIK3R1, PRKCA, SOS2 |
| Mouse Embryonic Stem Cell Pluripotency | KRAS, PIK3R1, SOS2, STAT3, TCF7L2 |
| Sirtuin Signaling Pathway | ACSS2, ATP5PB, CDH1, CPS1, GABPA, |
| | GABPB1, IDH2, STAT3, TOMM20, TUBA1B, |
| | TUBA4A, ZBTB14 |
| Heparan Sulfate Biosynthesis | EXTL2, EXTL3, NOTUM, XYLT1 |
| LPS-stimulated MAPK Signaling | FOS, KRAS, PIK3R1, PRKCA |
| PEDF Signaling | BDNF, KRAS, PIK3R1, TCF7L2 |
| NRF2-mediated Oxidative Stress Response | DNAJC16, DNAJC3, FOS, GSR, KRAS, |
| | PIK3R1, PRKCA, SQSTM1 |
| ILK Signaling | CCND1, CDH1, FOS, MYH11, PDPK1, PIK3R1, |
| | PTK2, RHOU |
| 3-phosphoinositide Biosynthesis | HACD2, NUDT15, NUDT3, PIK3R1, PIP4K2C, |
| | PPTC7, SET |
| Role of NANOG in Mammalian Embryonic Stem | KRAS, PIK3R1, SOS2, STAT3 |
| Cell Pluripotency | |
| Opioid Signaling Pathway | ADCY9, AP2M1, FOS, GNAI3, GNB1, KRAS, |
| | PPP3R1, PRKCA, RPS6KB2, SOS2 |
| Ovarian Cancer Signaling | CCND1, CDK4, KRAS, PIK3R1, RPS6KB2, |
| | TCF7L2 |
| NF-κB Signaling | KRAS, MAP3K3, PIK3R1, TGFBR2, TGFBR3, |
| | TLR6 |
| Calcium Signaling | ATP2A1, ATP2B4, CAMKK1, HDAC3, MYH11, |
| | PPP3R1, RCAN3 |
| Wnt/β-catenin Signaling | APPL1, CCND1, CDH1, TCF7L2, TGFB2, |
| | TGFBR2, TGFBR3 |
| GPCR-Mediated Nutrient Sensing in | ADCY9, GNAI3, NOTUM, PRKCA |
| Enteroendocrine Cells | |
| D-myo-inositol (1,4,5,6)-Tetrakisphosphate | HACD2, NUDT15, NUDT3, PPTC7, SET |
| Biosynthesis | |
| D-myo-inositol (3,4,5,6)-tetrakisphosphate | HACD2, NUDT15, NUDT3, PPTC7, SET |
| Biosynthesis | |
| Phospholipase C Signaling | ADCY9, GNB1, HDAC3, KRAS, PLD6, PPP3R1, |
| | PRKCA, RHOU, SOS2 |
| EIF2 Signaling | CCND1, EIF2AK1, KRAS, PDPK1, PIK3R1, |
| | RPL26, RPS17, SOS2 |
| 3-phosphoinositide Degradation | HACD2, INPPL1, NUDT15, NUDT3, PPTC7, |
| | SET |
| D-myo-inositol-5-phosphate Metabolism | HACD2, NUDT15, NUDT3, PIP4K2C, PPTC7, |
| | SET |
| Endocannabinoid Neuronal Synapse Pathway | ADCY9, GNAI3, GNB1, NOTUM, PPP3R1 |
| eNOS Signaling | ADCY9, CASP9, CCNA2, PDPK1, PIK3R1, |
| | PRKCA |
| Nitric Oxide Signaling in the Cardiovascular | ADRB1, ATP2A1, PIK3R1, PRKCA |
| System | |
| Gαq Signaling | GNB1, PIK3R1, PLD6, PPP3R1, PRKCA, |
| | RHOU |
| Regulation of eIF4 and p70S6K Signaling | EIF4EBP2, KRAS, PDPK1, PIK3R1, RPS17, |
| | SOS2 |
| Osteoarthritis Pathway | CASP9, HDAC3, HMGB1, TCF7L2, TGFBR2 |
| Synaptic Long Term Depression | GNAI3, GNAZ, KRAS, NOTUM, NPR1, PRKCA |
| Synaptic Long Term Potentiation | KRAS, NOTUM, PPP3R1, PRKCA |

TABLE 9
BT549 breast cancer upregulated pathways and associated genes

| Pathway Name | Gene |
|---|---|
| PTEN Signaling | BCAR1, BMPR2, CBL, CCND1, INPPL1, ITGA2, |
| | KRAS, MAGI3, NGFR, PDGFRB, PDPK1, |
| | PIK3R1, PTK2, RPS6KB2, SOS2, SYNJ2, |
| | TGFBR3 |
| RhoGDI Signaling | ACTG2, ARHGDIB, ARHGEF1, ARHGEF9, |
| | ARPC2, ARPC5, CREBBP, GNAI1, GNAI3, |
| | GNB1L, ITGA2, PAK4, PIP4K2C, PRKCA, |
| | RHOU, RND1, RND3 |
| Cell Cycle: G1/S Checkpoint Regulation | ABL1, CCND1, CDC25A, CDK4, CDK6, E2F8, |
| | MAX, MDM2, SKP2 |
| Sumoylation Pathway | ARHGDIB, CREBBP, ETS1, MDM2, MYB, |
| | RFC4, RHOU, RND1, RND3, SENP1 |
| PPARα/RXRα Activation | ADCY7, BMPR2, CREBBP, HELZ2, IL1B, JAK2, |
| | KRAS, NCOA3, PLCB2, PLCD1, PLCD3, |
| | PRKCA, SOS2, TGFBR3 |
| Endocannabinoid Cancer Inhibition Pathway | ADCY7, CCND1, CREBBP, GNAI1, GNAI3, |
| | GNB1L, PIK3R1, PTK2, SMPD1, TCF4 |
| PPAR Signaling | CREBBP, IL1B, KRAS, NGFR, PDGFD, |
| | PDGFRB, SOS2, TNFRSF1B |
| HIPPO signaling | FAT4, FRMD6, LATS2, LLGL1, SKP2, YAP1, |
| | YWHAZ |
| Antioxidant Action of Vitamin C | CSF2, JAK2, PLCB2, PLCD1, PLCD3, PLD6, |
| | SLC23A2, SLC2A3 |
| GPCR-Mediated Integration of Enteroendocrine | ADCY7, GNAI1, GNAI3, PLCB2, PLCD1, |
| Signaling Exemplified by an L Cell | PLCD3 |
| Apoptosis Signaling | APAF1, KRAS, MCL1, PRKCA, TNFRSF1B |

TABLE 10
BT549 breast cancer aberrant pathways and associated genes

| Pathway Name | Gene |
|---|---|
| Molecular Mechanisms of Cancer | ABL1, ADCY7, APAF1, ARHGEF1, ARHGEF9, |
| | BMPR2, CBL, CCND1, CDC25A, CDK4, CDK6, |
| | CREBBP, E2F8, GAB2, GNAI1, GNAI3, ITGA2, |
| | JAK2, KRAS, LRP5, MAX, MDM2, PAK4, |
| | PIK3R1, PLCB2, PRKCA, PSEN1, PTCH1, |
| | PTK2, RHOU, RND1, RND3, SOS2, TCF4 |
| Axonal Guidance Signaling | ABL1, ADAM22, ADAMTS1, ADAMTS15, |
| | ADAMTS18, ARPC2, ARPC5, BCAR1, CRKL, |
| | ERAP2, ERBB2, GIT1, GNAI1, GNAI3, GNB1L, |
| | ITGA2, KRAS, LNPEP, MMP1, MMP14, NGFR, |
| | PAK4, PDGFD, PIK3R1, PLCB2, PLCD1, |
| | PLCD3, PRKCA, PTCH1, PTK2, RND1, SOS2 |
| Estrogen-mediated S-phase Entry | CCNA2, CCND1, CDC25A, CDK4, E2F8, SKP2 |
| HER-2 Signaling in Breast Cancer | CCND1, CDK6, ERBB2, ITGB3, KRAS, MDM2, |
| | PARD6A, PIK3R1, PRKCA, SOS2 |
| Chronic Myeloid Leukemia Signaling | ABL1, CCND1, CDK4, CDK6, CRKL, E2F8, |
| | GAB2, KRAS, MDM2, PIK3R1, SOS2 |
| Bladder Cancer Signaling | ABL1, CCND1, CDK4, CXCL8, DAPK1, ERBB2, |
| | KRAS, MDM2, MMP1, MMP14 |
| Role of Macrophages, Fibroblasts and | CCND1, CEBPD, CREBBP, CSF2, CXCL8, |
| Endothelial Cells in Rheumatoid Arthritis | F2RL1, IL1B, JAK2, KRAS, LRP5, MMP1, |
| | NGFR, PDGFD, PIK3R1, PLCB2, PLCD1, |
| | PLCD3, PRKCA, TCF4, TNFRSF1B, TRAF1 |
| Clathrin-mediated Endocytosis Signaling | ACTG2, AP2M1, ARPC2, ARPC5, CBL, DAB2, |
| | DNM3, ITGB3, MDM2, PDGFD, PIK3R1, |
| | RAB5B, SNAP91, SNX9, STAM |
| Role of Tissue Factor in Cancer | CSF2, CXCL8, F2RL1, IL1B, ITGB3, JAK2, |
| | KRAS, MMP1, PIK3R1, PLAUR, PRKCA |
| G-Protein Coupled Receptor Signaling | ADCY7, CREBBP, DUSP4, GNAI1, GNAI3, |
| | HTR7, KRAS, LPAR1, PDE3A, PDE4D, PDE5A, |
| | PDPK1, PIK3R1, PLCB2, PLD6, PRKCA, |
| | RGS2, SOS2 |
| FAK Signaling | ACTG2, BCAR1, ITGA2, KRAS, PAK4, PDPK1, |
| | PIK3R1, PTK2, SOS2 |
| Breast Cancer Regulation by Stathmin1 | ADCY7, ARHGEF1, ARHGEF9, E2F8, GNAI1, |
| | GNAI3, GNB1L, KRAS, PIK3R1, PLCB2, |
| | PRKCA, SOS2, STMN1, UHMK1 |
| TR/RXR Activation | AKR1C3, DIO2, GPS2, MDM2, NCOA3, |
| | PIK3R1, SREBF2, THRB |
| Phenylalanine Degradation I (Aerobic) | PCBD1, QDPR |
| Prostate Cancer Signaling | ABL1, CCND1, CREBBP, KRAS, MDM2, |
| | PDPK1, PIK3R1, SOS2 |
| Regulation of the Epithelial-Mesenchymal | ETS1, HMGA2, JAK2, KRAS, LOX, NOTCH2, |
| Transition Pathway | PARD6A, PDGFD, PDGFRB, PIK3R1, PSEN1, |
| | SOS2, TCF4 |
| NAD biosynthesis II (from tryptophan) | ABL1, IDO1, TDO2 |
| Germ Cell-Sertoli Cell Junction Signaling | ACTG2, BCAR1, ITGA2, KRAS, MAP3K3, |
| | PAK4, PDPK1, PIK3R1, PTK2, RHOU, RND1, |
| | RND3 |
| Leptin Signaling in Obesity | ADCY7, JAK2, PDE3A, PIK3R1, PLCB2, |
| | PLCD1, PLCD3 |
| Erythropoietin Signaling | CBL, JAK2, KRAS, PDPK1, PIK3R1, PRKCA, |
| | SOS2 |
| Granulocyte Adhesion and Diapedesis | CCL20, CXCL2, CXCL8, GNAI1, GNAI3, IL1B, |
| | ITGA2, ITGB3, MMP1, MMP14, NGFR, |
| | TNFRSF1B |
| Reelin Signaling in Neurons | ARHGEF1, ARHGEF9, CDK5R1, CRKL, ITGA2, |
| | ITGB3, PIK3R1 |
| G Protein Signaling Mediated by Tubby | ABL1, GNB1L, JAK2, PLCB2 |
| 1D-myo-inositol Hexakisphosphate Biosynthesis | INPPL1, ITPKA, SYNJ2 |
| II (Mammalian) | |
| D-myo-inositol (1,3,4)-trisphosphate | INPPL1, ITPKA, SYNJ2 |
| Biosynthesis | |
| Tryptophan Degradation to 2-amino-3- | IDO1, TDO2 |
| carboxymuconate Semialdehyde | |
| Phagosome Formation | ITGA2, PIK3R1, PLCB2, PLCD1, PLCD3, |
| | PRKCA, RHOU, RND1, RND3 |
| IL-4 Signaling | INPPL1, JAK2, KRAS, PIK3R1, RPS6KB2, |
| | SOS2, SYNJ2 |
| Virus Entry via Endocytic Pathways | ABL1, ACTG2, AP2M1, ITGA2, ITGB3, KRAS, |
| | PIK3R1, PRKCA |
| Cellular Effects of Sildenafil (Viagra) | ACTG2, ADCY7, CACNG4, PDE3A, PDE4D, |
| | PDE5A, PLCB2, PLCD1, PLCD3 |
| Role of Osteoblasts, Osteoclasts and | BMPR2, CBL, CSF2, IL1B, ITGA2, ITGB3, |
| Chondrocytes in Rheumatoid Arthritis | LRP5, MMP1, MMP14, NGFR, PIK3R1, TCF4, |
| | TNFRSF1B |
| Airway Pathology in Chronic Obstructive | CXCL8, MMP1 |
| Pulmonary Disease | |
| Salvage Pathways of Pyrimidine | APOBEC3G, TK2 |
| Deoxyribonucleotides | |
| Gap Junction Signaling | ACTG2, ADCY7, GNAI1, GNAI3, KRAS, |
| | LPAR1, PIK3R1, PLCB2, PLCD1, PLCD3, |
| | PRKCA, SOS2 |
| IL-15 Signaling | CSF2, CXCL8, JAK2, KRAS, PIK3R1, PTK2 |
| Caveolar-mediated Endocytosis Signaling | ABL1, ACTG2, ITGA2, ITGB3, PRKCA, RAB5B |
| HIF1α Signaling | CREBBP, KRAS, MAPK15, MDM2, MMP1, |
| | MMP14, PIK3R1, SLC2A3 |
| Leucine Degradation I | BCAT2, HMGCLL1 |
| Polyamine Regulation in Colon Cancer | KRAS, MAX, TCF4 |
| Docosahexaenoic Acid (DHA) Signaling | APAF1, IL1B, PDPK1, PIK3R1 |
| Hereditary Breast Cancer Signaling | CCND1, CDK4, CDK6, CREBBP, DPF1, |
| | H2AFX, KRAS, PIK3R1, RFC4 |
| Superpathway of D-myo-inositol (1,4,5)- | INPPL1, ITPKA, SYNJ2 |
| trisphosphate Metabolism | |
| Glucocorticoid Receptor Signaling | CREBBP, CSF2, CXCL8, DPF1, IL1B, JAK2, |
| | KRAS, KRT10, KRT15, KRT79, MMP1, NCOA3, |
| | PIK3R1, PLAU, POU2F2, SOS2, TAF9B |
| Semaphorin Signaling in Neurons | PAK4, PTK2, RHOU, RND1, RND3 |
| Agranulocyte Adhesion and Diapedesis | ACTG2, AOC3, CCL20, CXCL2, CXCL8, |
| | GNAI1, GNAI3, IL1B, ITGA2, MMP1, MMP14 |
| RAR Activation | ADCY7, AKR1C3, CREBBP, DPF1, JAK2, |
| | MMP1, NR2F2, NSD1, PDPK1, PIK3R1, |
| | PRKCA |
| Serotonin Receptor Signaling | ADCY7, HTR7, PCBD1, QDPR |
| Relaxin Signaling | ADCY7, GNAI1, GNAI3, GNB1L, PDE3A, |
| | PDE4D, PDE5A, PIK3R1, PLD6 |
| Epithelial Adherens Junction Signaling | ACTG2, ARPC2, ARPC5, BMPR2, DLL1, |
| | KRAS, NOTCH2, TCF4, TGFBR3 |
| Gustation Pathway | ADCY7, CACNG4, PDE3A, PDE4D, PDE5A, |
| | PLCB2, PLD6, SCNN1A, TAS2R14 |
| Hematopoiesis from Multipotent Stem Cells | CSF2, KITLG |
| Myc Mediated Apoptosis Signaling | APAF1, KRAS, PIK3R1, SOS2, YWHAZ |
| Glycine Biosynthesis I | SHMT2 |
| Guanine and Guanosine Salvage I | HPRT1 |
| L-cysteine Degradation III | MPST |
| Bile Acid Biosynthesis, Neutral Pathway | AKR1C3, SCP2 |
| Role of IL-17A in Psoriasis | CCL20, CXCL8 |
| Ephrin A Signaling | BCAR1, NGFR, PIK3R1, PTK2 |
| Remodeling of Epithelial Adherens Junctions | ACTG2, ARPC2, ARPC5, DNM3, RAB5B |
| Hematopoiesis from Pluripotent Stem Cells | CSF2, CXCL8, KITLG, LIF |
| Iron homeostasis signaling pathway | ATP6V1B2, BMPR2, ERFE, GDF15, HFE, |
| | JAK2, PDGFRB, SKP2 |
| Factors Promoting Cardiogenesis in Vertebrates | BMPR2, LRP5, NKX2-5, PRKCA, TCF4, |
| | TGFBR3 |
| Thyroid Cancer Signaling | CCND1, CXCL8, KRAS, TCF4 |
| Ephrin B Signaling | CBL, GNAI1, GNAI3, GNB1L, PTK2 |
| Natural Killer Cell Signaling | INPPL1, KRAS, PAK4, PIK3R1, PRKCA, SOS2, |
| | SYNJ2 |
| ATM Signaling | ABL1, CDC25A, CREBBP, H2AFX, MDM2, |
| | RAD17 |
| p53 Signaling | APAF1, CCND1, CDK4, MDM2, PIK3R1, |
| | TP53INP1 |
| Granzyme B Signaling | APAF1, LMNB1 |
| Vitamin-C Transport | SLC23A2, SLC2A3 |
| L-carnitine Biosynthesis | ALDH9A1 |
| N-acetylglucosamine Degradation I | GNPDA1 |
| Thiosulfate Disproportionation III (Rhodanese) | MPST |
| Thyroid Hormone Metabolism I (via | DIO2 |
| Deiodination) | |
| Thyronamine and Iodothyronamine Metabolism | DIO2 |
| Tyrosine Biosynthesis IV | PCBD1 |
| Protein Ubiquitination Pathway | CBL, DNAJB9, DNAJC3, HSPA13, HSPB7, |
| | MDM2, PSMB8, SKP2, UBE2L6, USP18, |
| | USP27X, USP39, USP40 |
| Macropinocytosis Signaling | ITGB3, KRAS, PDGFD, PIK3R1, PRKCA |
| Role of IL-17A in Arthritis | CCL20, CXCL8, MMP1, PIK3R1 |
| Unfolded protein response | CEBPD, DNAJB9, DNAJC3, SREBF2 |
| D-myo-inositol (1,4,5)-trisphosphate | INPPL1, SYNJ2 |
| Degradation | |
| Dermatan Sulfate Degradation (Metazoa) | HYAL2, IDS |
| Role of CHK Proteins in Cell Cycle Checkpoint | CDC25A, E2F8, RAD17, RFC4 |
| Control | |
| Differential Regulation of Cytokine Production in | CSF2, IL1B |
| Macrophages and T Helper Cells by IL-17A and | |
| IL-17F | |
| Cell Cycle Regulation by BTG Family Proteins | CCND1, CDK4, E2F8 |
| Notch Signaling | DLL1, NOTCH2, PSEN1 |
| GADD45 Signaling | CCND1, CDK4 |
| Oxidative Ethanol Degradation III | ACSS2, ALDH9A1 |
| Acetate Conversion to Acetyl-CoA | ACSS2 |
| N-acetylglucosamine Degradation II | GNPDA1 |
| Phenylethylamine Degradation I | AOC3 |
| Cardiomyocyte Differentiation via BMP | BMPR2, NKX2-5 |
| Receptors | |
| Hepatic Fibrosis/Hepatic Stellate Cell | COL5A1, CXCL8, EDNRA, IL1B, MMP1, NGFR, |
| Activation | PDGFD, PDGFRB, TNFRSF1B |
| Mechanisms of Viral Exit from Host Cells | ACTG2, LMNB1, PRKCA |
| Pyrimidine Ribonucleotides Interconversion | AK4, NUDT18, SMARCA1 |
| Folate Polyglutamylation | SHMT2 |
| Protein Citrullination | PADI2 |
| dTMP De Novo Biosynthesis | SHMT2 |
| Role of PI3K/AKT Signaling in the Pathogenesis | CRKL, GNAI1, GNAI3, PIK3R1 |
| of Influenza | |
| Pyrimidine Deoxyribonucleotides De Novo | AK4, RRM2 |
| Biosynthesis I | |
| Tryptophan Degradation III (Eukaryotic) | IDO1, TDO2 |
| Pyrimidine Ribonucleotides De Novo | AK4, NUDT18, SMARCA1 |
| Biosynthesis | |
| Tight Junction Signaling | ACTG2, CDK4, LLGL1, NGFR, NSF, NUDT21, |
| | PARD6A, TNFRSF1B |
| Differential Regulation of Cytokine Production in | CSF2, IL1B |
| Intestinal Epithelial Cells by IL-17A and IL-17F | |
| Ethanol Degradation IV | ACSS2, ALDH9A1 |
| Stearate Biosynthesis I (Animals) | ACOT8, ACSL5, DHCR24 |
| iNOS Signaling | CREBBP, IRF1, JAK2 |
| Adenine and Adenosine Salvage III | HPRT1 |
| Glycogen Biosynthesis II (from UDP-D-Glucose) | GBE1 |
| UDP-N-acetyl-D-glucosamine Biosynthesis II | GFPT1 |
| CCR5 Signaling in Macrophages | CACNG4, GNAI1, GNAI3, GNB1L, PRKCA |
| PFKFB4 Signaling Pathway | CREBBP, HK2, NCOA3 |
| Role of JAK1 and JAK3 in γc Cytokine Signaling | IL7R, JAK2, KRAS, PIK3R1 |
| GABA Receptor Signaling | ADCY7, ALDH9A1, AP2M1, CACNG4, NSF |
| Th1 and Th2 Activation Pathway | BMPR2, DLL1, IRF1, JAK2, NOTCH2, PIK3R1, |
| | PSEN1, TGFBR3 |
| IL-15 Production | ABL1, ERBB2, IRF1, JAK2, PDGFRB, PTK2 |
| IL-17A Signaling in Gastric Cells | CCL20, CXCL8 |
| Role of JAK family kinases in IL-6-type Cytokine | JAK2, OSMR |
| Signaling | |
| Antiproliferative Role of TOB in T Cell Signaling | CCNA2, SKP2 |
| NAD Salvage Pathway II | NT5E, PXYLP1 |
| GDP-glucose Biosynthesis | PGM5 |
| NAD Biosynthesis from 2-amino-3- | ABL1 |
| carboxymuconate Semialdehyde | |
| Superpathway of Serine and Glycine | SHMT2 |
| Biosynthesis I | |
| UVC-Induced MAPK Signaling | KRAS, PRKCA, SMPD1 |
| FcγRIIB Signaling in B Lymphocytes | CACNG4, KRAS, PDPK1, PIK3R1 |
| Hepatic Cholestasis | ADCY7, CSF2, CXCL8, IL1B, LIF, NGFR, |
| | PRKCA, TNFRSF1B |
| Glucose and Glucose-1-phosphate Degradation | PGM5 |
| Sphingomyelin Metabolism | SMPD1 |
| VDR/RXR Activation | CSF2, LRP5, NCOA3, PRKCA |
| Role of Cytokines in Mediating Communication | CSF2, CXCL8, IL1B |
| between Immune Cells | |
| T Cell Receptor Signaling | CBL, KRAS, PAG1, PIK3R1, SOS2 |
| Role of p14/p19ARF in Tumor Suppression | MDM2, PIK3R1 |
| TNFR2 Signaling | TNFRSF1B, TRAF1 |
| Folate Transformations I | SHMT2 |
| Sucrose Degradation V (Mammalian) | TKFC |
| UDP-N-acetyl-D-galactosamine Biosynthesis II | GNPDA1 |
| IL-17 Signaling | CXCL8, JAK2, KRAS, PIK3R1 |
| Human Embryonic Stem Cell Pluripotency | BMPR2, PDGFD, PDGFRB, PDPK1, PIK3R1, |
| | TCF4 |
| EGF Signaling | PIK3R1, PRKCA, SOS2 |
| Estrogen Receptor Signaling | CREBBP, KRAS, MED21, NCOA3, SOS2, |
| | TAF9B |
| Ethanol Degradation II | ACSS2, ALDH9A1 |
| Fatty Acid β-oxidation I | ACSL5, SCP2 |
| Dolichyl-diphosphooligosaccharide Biosynthesis | DPM3 |
| Embryonic Stem Cell Differentiation into Cardiac | NKX2-5 |
| Lineages | |
| Glycine Betaine Degradation | SHMT2 |
| Ketogenesis | HMGCLL1 |
| Cancer Drug Resistance By Drug Efflux | KRAS, MDM2, PIK3R1 |
| Dopamine Degradation | ALDH9A1 |
| Sonic Hedgehog Signaling | PTCH1 |
| MSP-RON Signaling Pathway | ACTG2, JAK2, PIK3R1 |
| Retinoic acid Mediated Apoptosis Signaling | APAF1, IRF1, PARP14 |
| Superpathway of Cholesterol Biosynthesis | DHCR24 |
| Coagulation System | PLAU, PLAUR |
| IL-17A Signaling in Fibroblasts | CEBPD, MMP1 |
| TWEAK Signaling | APAF1, TRAF1 |
| Autophagy | CTSO, SQSTM1 |
| IL-2 Signaling | KRAS, PIK3R1, SOS2 |
| SPINK1 Pancreatic Cancer Pathway | CPA4, F2RL1 |
| Apelin Liver Signaling Pathway | PDGFRB |
| Lipid Antigen Presentation by CD1 | AP2M1 |
| Nur77 Signaling in T Lymphocytes | APAF1, MAP3K3 |
| Cleavage and Polyadenylation of Pre-mRNA | NUDT21 |
| Glycogen Degradation II | PGM5 |
| Guanosine Nucleotides Degradation III | NT5E |
| IL-1 Signaling | ADCY7, GNAI1, GNAI3, GNB1L |
| Atherosclerosis Signaling | CXCL8, IL1B, MMP1, PDGFD |
| Tryptophan Degradation X (Mammalian, via | ALDH9A1 |
| Tryptamine) | |
| CTLA4 Signaling in Cytotoxic T Lymphocytes | AP2M1, JAK2, PIK3R1 |
| Role of JAK1, JAK2 and TYK2 in Interferon | JAK2 |
| Signaling | |
| Tumoricidal Function of Hepatic Natural Killer | APAF1 |
| Cells | |
| Crosstalk between Dendritic Cells and Natural | ACTG2, CSF2, TNFRSF1B |
| Killer Cells | |
| RANK Signaling in Osteoclasts | CBL, MAP3K3, PIK3R1 |
| Sertoli Cell-Sertoli Cell Junction Signaling | ACTG2, BCAR1, ITGA2, KRAS, MAP3K3, |
| | TGFBR3 |
| IL-17A Signaling in Airway Cells | CCL20, JAK2, PIK3R1 |
| Cell Cycle Control of Chromosomal Replication | CDK4, CDK6 |
| Acyl-CoA Hydrolysis | ACOT8 |
| Cholesterol Biosynthesis I | DHCR24 |
| Cholesterol Biosynthesis II (via 24,25- | DHCR24 |
| dihydrolanosterol) | |
| Cholesterol Biosynthesis III (via Desmosterol) | DHCR24 |
| Fatty Acid Activation | ACSL5 |
| NAD Phosphorylation and Dephosphorylation | PXYLP1 |
| Urate Biosynthesis/Inosine 5′-phosphate | NT5E |
| Degradation | |
| Inhibition of Matrix Metalloproteases | MMP1, MMP14 |
| Phosphatidylglycerol Biosynthesis II (Non- | AGPAT1 |
| plastidic) | |
| Androgen Biosynthesis | AKR1C3 |
| DNA Double-Strand Break Repair by | ABL1 |
| Homologous Recombination | |
| Glycogen Degradation III | PGM5 |
| Isoleucine Degradation I | BCAT2 |
| CD27 Signaling in Lymphocytes | APAF1, MAP3K3 |
| UVB-Induced MAPK Signaling | PIK3R1, PRKCA |
| Role of PKR in Interferon Induction and Antiviral | APAF1, IRF1 |
| Response | |
| Endoplasmic Reticulum Stress Pathway | DNAJC3 |
| Putrescine Degradation III | ALDH9A1 |
| LPS-stimulated MAPK Signaling | KRAS, PIK3R1, PRKCA |
| PEDF Signaling | KRAS, PIK3R1, TCF4 |
| Amyloid Processing | CDK5R1, PSEN1 |
| SPINK1 General Cancer Pathway | JAK2, KRAS, PIK3R1 |
| Adenosine Nucleotides Degradation II | NT5E |
| Choline Biosynthesis III | PLD6 |
| Retinol Biosynthesis | AKR1C3, LIPE |
| CDP-diacylglycerol Biosynthesis I | AGPAT1 |
| Fatty Acid α-oxidation | ALDH9A1 |
| Granzyme A Signaling | CREBBP |
| Inflammasome pathway | IL1B |
| The Visual Cycle | AKR1C3 |
| iCOS-iCOSL Signaling in T Helper Cells | GAB2, PDPK1, PIK3R1, PLEKHA2 |
| TNFR1 Signaling | APAF1, PAK4 |
| Role of BRCA1 in DNA Damage Response | DPF1, E2F8, RFC4 |
| Role of Hypercytokinemia/hyperchemokinemia | CXCL8, IL1B |
| in the Pathogenesis of Influenza | |
| Chondroitin Sulfate Degradation (Metazoa) | HYAL2 |
| Mismatch Repair in Eukaryotes | RFC4 |
| DNA damage-induced 14-3-3σ Signaling | RAD17 |
| NER Pathway | COPS3, DDB1, POLE3, RFC4 |
| Antiproliferative Role of Somatostatin Receptor | GNB1L, KRAS, PIK3R1 |
| 2 | |
| Hypoxia Signaling in the Cardiovascular System | CREBBP, MDM2, UBE2L6 |
| Adipogenesis pathway | BMPR2, CEBPD, EBF1, KLF3, NR2F2 |
| Dopamine Receptor Signaling | ADCY7, PCBD1, QDPR |
| FAT10 Signaling Pathway | SQSTM1 |
| Methylglyoxal Degradation III | AKR1C3 |
| Purine Nucleotides Degradation II (Aerobic) | NT5E |
| Valine Degradation I | BCAT2 |
| Histamine Degradation | ALDH9A1 |
| Mitochondrial L-carnitine Shuttle Pathway | ACSL5 |
| RAN Signaling | KPNA5 |
| γ-linolenate Biosynthesis II (Animals) | ACSL5 |
| Role of Oct4 in Mammalian Embryonic Stem | NR2F2, NR6A1 |
| Cell Pluripotency | |
| 4-1BB Signaling in T Lymphocytes | TRAF1 |
| Activation of IRF by Cytosolic Pattern | CREBBP |
| Recognition Receptors | |
| Altered T Cell and B Cell Signaling in | CSF2, IL1B |
| Rheumatoid Arthritis | |
| Antigen Presentation Pathway | PSMB8 |
| Apelin Pancreas Signaling Pathway | PIK3R1 |
| April Mediated Signaling | TRAF1 |
| Assembly of RNA Polymerase II Complex | TAF9B |
| B Cell Activating Factor Signaling | TRAF1 |
| B Cell Development | IL7R |
| BAG2 Signaling Pathway | MDM2 |
| Basal Cell Carcinoma Signaling | PTCH1, TCF4 |
| CD40 Signaling | PIK3R1, TRAF1 |
| Calcium Signaling | CACNG4, CREBBP, RCAN1 |
| Calcium-induced T Lymphocyte Apoptosis | PRKCA |
| Circadian Rhythm Signaling | CREBBP |
| Communication between Innate and Adaptive | CSF2, CXCL8, IL1B |
| Immune Cells | |
| Complement System | C1QBP |
| Cytotoxic T Lymphocyte-mediated Apoptosis of | APAF1 |
| Target Cells | |
| DNA Methylation and Transcriptional | MTA2 |
| Repression Signaling | |
| Eicosanoid Signaling | AKR1C3 |
| Estrogen Biosynthesis | AKR1C3 |
| FXR/RXR Activation | CREBBP, IL1B |
| Graft-versus-Host Disease Signaling | IL1B |
| Heparan Sulfate Biosynthesis | EXTL2, LIPE |
| Heparan Sulfate Biosynthesis (Late Stages) | EXTL2, LIPE |
| IL-10 Signaling | IL1B |
| IL-12 Signaling and Production in Macrophages | IRF1, PIK3R1, PRKCA |
| IL-9 Signaling | PIK3R1 |
| LPS/IL-1 Mediated Inhibition of RXR Function | ACSL5, ALDH9A1, IL1B, NGFR, TNFRSF1B |
| LXR/RXR Activation | IL1B, NGFR, TNFRSF1B |
| Mitochondrial Dysfunction | ATP5PB, PSEN1 |
| Mitotic Roles of Polo-Like Kinase | CDC25A |
| Netrin Signaling | CACNG4 |
| Neuroprotective Role of THOP1 in Alzheimer's | TPP1 |
| Disease | |
| Nitric Oxide Signaling in the Cardiovascular | PDE5A, PIK3R1, PRKCA |
| System | |
| Noradrenaline and Adrenaline Degradation | ALDH9A1 |
| Oxidative Phosphorylation | ATP5PB |
| PCP pathway | LGR4 |
| Phagosome Maturation | ATP6V1B2, CTSO, NSF, RAB5B |
| Primary Immunodeficiency Signaling | IL7R |
| Regulation of IL-2 Expression in Activated and | KRAS, SOS2 |
| Anergic T Lymphocytes | |
| Retinoate Biosynthesis I | AKR1C3 |
| Role of JAK2 in Hormone-like Cytokine | JAK2 |
| Signaling | |
| Role of MAPK Signaling in the Pathogenesis of | KRAS, PRKCA |
| Influenza | |
| Role of RIG1-like Receptors in Antiviral Innate | CREBBP |
| Immunity | |
| Role of Wnt/GSK-3β Signaling in the | NCOA3, TCF4 |
| Pathogenesis of Influenza | |
| Serotonin Degradation | ALDH9A1 |
| Systemic Lupus Erythematosus Signaling | CBL, IL1B, KRAS, PIK3R1, PRPF40B, SOS2 |
| T Helper Cell Differentiation | NGFR, TNFRSF1B |
| Thyroid Hormone Metabolism II (via Conjugation | DIO2 |
| and/or Degradation) | |
| Toll-like Receptor Signaling | IL1B, TRAF1 |
| Transcriptional Regulatory Network in | PAX6 |
| Embryonic Stem Cells | |
| Triacylglycerol Biosynthesis | AGPAT1 |
| Triacylglycerol Degradation | LIPE |
| Xenobiotic Metabolism Signaling | ALDH9A1, CREBBP, IL1B, KRAS, MAP3K3, |
| | PIK3R1, PRKCA |
| nNOS Signaling in Neurons | PRKCA |
| nNOS Signaling in Skeletal Muscle Cells | CACNG4 |
| tRNA Charging | LARS2 |

TABLE 11
BT549 breast cancer downregulated pathways and associated genes

| Pathway Name | Gene |
|---|---|
| Protein Kinase A Signaling | ADCY7, AKAP12, CDC14B, CDC25A, |
| | CREBBP, DUSP16, DUSP2, DUSP4, EPM2A, |
| | GNAI1, GNAI3, GNB1L, LIPE, NGFR, PDE3A, |
| | PDE4D, PDE5A, PLCB2, PLCD1, PLCD3, |
| | PLD6, PRKCA, PTCH1, PTK2, PTPN21, |
| | PTPN3, PTPN9, PTPRA, PTPRE, PTPRZ1, |
| | TCF4, YWHAZ |
| Neuregulin Signaling | CDK5R1, CRKL, ERBB2, ERBIN, ERRFI1, |
| | GRB7, ITGA2, KRAS, PDPK1, PIK3R1, PRKCA, |
| | PSEN1, RPS6KB2, SOS2 |
| PI3K/AKT Signaling | CCND1, GAB2, GDF15, INPPL1, ITGA2, JAK2, |
| | KRAS, MCL1, MDM2, PDPK1, PIK3R1, |
| | RPS6KB2, SOS2, SYNJ2, YWHAZ |
| Sphingosine-1-phosphate Signaling | ADCY7, GNAI1, GNAI3, PDGFD, PDGFRB, |
| | PIK3R1, PLCB2, PLCD1, PLCD3, PTK2, RHOU, |
| | RND1, RND3, SMPD1 |
| Glioma Signaling | ABL1, CCND1, CDK4, CDK6, E2F8, IDH1, |
| | KRAS, MDM2, PDGFD, PDGFRB, PIK3R1, |
| | PRKCA, SOS2 |
| Integrin Signaling | ABL1, ACTG2, ARF3, ARPC2, ARPC5, BCAR1, |
| | CRKL, GIT1, GRB7, ITGA2, ITGB3, KRAS, |
| | PAK4, PIK3R1, PTK2, RHOU, RND1, RND3, |
| | SOS2 |
| Glioblastoma Multiforme Signaling | CCND1, CDK4, CDK6, E2F8, KRAS, MDM2, |
| | PDGFD, PDGFRB, PIK3R1, PLCB2, PLCD1, |
| | PLCD3, RHOU, RND1, RND3, SOS2 |
| Small Cell Lung Cancer Signaling | ABL1, APAF1, CCND1, CDK4, CDK6, MAX, |
| | PIK3R1, PTK2, SKP2, TRAF1 |
| Cardiac Hypertrophy Signaling (Enhanced) | ADCY7, BMPR2, CSF2, CXCL8, EDNRA, |
| | GNAI1, GNAI3, HSPB7, IL17RD, IL1B, IL7R, |
| | ITGA2, JAK2, KRAS, LIF, MAP3K3, NGFR, |
| | NKX2-5, PDE3A, PDE4D, PDE5A, PIK3R1, |
| | PLCB2, PLCD1, PLCD3, PLD6, PRKCA, PTK2, |
| | RCAN1, RPS6KB2, TGFBR3, TNFRSF1B |
| CXCR4 Signaling | ADCY7, BCAR1, ELMO2, ELMO3, GNAI1, |
| | GNAI3, GNB1L, KRAS, PAK4, PIK3R1, PLCB2, |
| | PRKCA, PTK2, RHOU, RND1, RND3 |
| B Cell Receptor Signaling | ABL1, CREBBP, EBF1, ETS1, GAB2, INPPL1, |
| | KRAS, MAP3K3, PAG1, PAX5, PDPK1, |
| | PIK3R1, POU2F2, PTK2, RPS6KB2, SOS2, |
| | SYNJ2 |
| PDGF Signaling | ABL1, CRKL, INPPL1, JAK2, KRAS, PDGFD, |
| | PDGFRB, PIK3R1, PRKCA, SOS2, SYNJ2 |
| Signaling by Rho Family GTPases | ACTG2, ARHGEF1, ARHGEF9, ARPC2, |
| | ARPC5, CDC42EP2, CDC42EP3, GNAI1, |
| | GNAI3, GNB1L, ITGA2, PAK4, PARD6A, |
| | PIK3R1, PIP4K2C, PTK2, RHOU, RND1, RND3, |
| | STMN1 |
| Non-Small Cell Lung Cancer Signaling | ABL1, CCND1, CDK4, CDK6, ERBB2, KRAS, |
| | PDPK1, PIK3R1, PRKCA, SOS2 |
| Fcγ Receptor-mediated Phagocytosis in | ACTG2, ARPC2, ARPC5, CBL, CSF2, DGKB, |
| Macrophages and Monocytes | GAB2, PIK3R1, PLD6, PRKCA, RPS6KB2 |
| IL-7 Signaling Pathway | CCND1, CDC25A, EBF1, IL7R, MCL1, PAX5, |
| | PDPK1, PIK3R1, PTK2, SOS2 |
| Ephrin Receptor Signaling | ABL1, ARPC2, ARPC5, BCAR1, CREBBP, |
| | CRKL, GNAI1, GNAI3, GNB1L, ITGA2, JAK2, |
| | KRAS, PAK4, PDGFD, PTK2, SOS2 |
| Superpathway of Inositol Phosphate | CDC25A, DUSP16, DUSP2, HACD2, INPPL1, |
| Compounds | ITPKA, NUDT3, PIK3R1, PIP4K2A, PIP4K2C, |
| | PLCB2, PLCD1, PLCD3, PPM1H, PPTC7, |
| | PXYLP1, SYNJ2 |
| Thrombin Signaling | ADCY7, ARHGEF1, ARHGEF9, GNAI1, GNAI3, |
| | GNB1L, KRAS, PDPK1, PIK3R1, PLCB2, |
| | PLCD1, PLCD3, PRKCA, PTK2, RHOU, RND1, |
| | RND3 |
| ERK/MAPK Signaling | BCAR1, CREBBP, CRKL, DUSP2, DUSP4, |
| | ELK3, ETS1, HSPB7, ITGA2, KRAS, PAK4, |
| | PIK3R1, PRKCA, PTK2, SOS2, YWHAZ |
| Aldosterone Signaling in Epithelial Cells | DNAJB9, DNAJC3, HSPA13, HSPB7, KRAS, |
| | PDPK1, PIK3R1, PIP4K2C, PLCB2, PLCD1, |
| | PLCD3, PRKCA, SCNN1A, SOS2 |
| Glioma Invasiveness Signaling | ITGB3, KRAS, PIK3R1, PLAU, PLAUR, PTK2, |
| | RHOU, RND1, RND3 |
| P2Y Purigenic Receptor Signaling Pathway | ADCY7, CREBBP, GNAI1, GNAI3, GNB1L, |
| | ITGB3, KRAS, PIK3R1, PLCB2, PLCD1, |
| | PLCD3, PRKCA |
| Tec Kinase Signaling | ACTG2, GNAI1, GNAI3, GNB1L, ITGA2, JAK2, |
| | PAK4, PIK3R1, PRKCA, PTK2, RHOU, RND1, |
| | RND3, TNFRSF21 |
| Agrin Interactions at Neuromuscular Junction | ACTG2, ERBB2, GABPA, ITGA2, ITGB3, |
| | KRAS, LAMC1, PAK4, PTK2 |
| HGF Signaling | CCND1, CRKL, ELK3, ETS1, ITGA2, KRAS, |
| | MAP3K3, PIK3R1, PRKCA, PTK2, SOS2 |
| p70S6K Signaling | F2RL1, GNAI1, GNAI3, KRAS, PDPK1, PIK3R1, |
| | PLCB2, PLCD1, PLCD3, PRKCA, SOS2, |
| | YWHAZ |
| D-myo-inositol (1,4,5)-Trisphosphate | PIP4K2A, PIP4K2C, PLCB2, PLCD1, PLCD3 |
| Biosynthesis | |
| D-myo-inositol-5-phosphate Metabolism | CDC25A, DUSP16, DUSP2, HACD2, NUDT3, |
| | PIP4K2A, PIP4K2C, PLCB2, PLCD1, PLCD3, |
| | PPM1H, PPTC7, PXYLP1 |
| Insulin Receptor Signaling | CBL, CRKL, INPPL1, JAK2, KRAS, LIPE, |
| | PDPK1, PIK3R1, RPS6KB2, SCNN1A, SOS2, |
| | SYNJ2 |
| RhoA Signaling | ACTG2, ARHGEF1, ARPC2, ARPC5, |
| | CDC42EP2, CDC42EP3, LPAR1, LPAR2, |
| | PIP4K2C, PTK2, RND3 |
| IL-8 Signaling | CCND1, CXCL8, GNAI1, GNAI3, GNB1L, |
| | ITGB3, KRAS, PIK3R1, PLCB2, PLD6, PRKCA, |
| | PTK2, RHOU, RND1, RND3 |
| Actin Nucleation by ARP-WASP Complex | ARPC2, ARPC5, ITGA2, KRAS, RHOU, RND1, |
| | RND3, SOS2 |
| Pancreatic Adenocarcinoma Signaling | ABL1, CCND1, CDK4, E2F8, ERBB2, JAK2, |
| | KRAS, MDM2, PIK3R1, PLD6 |
| Rac Signaling | ARPC2, ARPC5, CDK5R1, ITGA2, KRAS, |
| | PAK4, PARD6A, PIK3R1, PIP4K2C, PTK2 |
| Regulation of Actin-based Motility by Rho | ACTG2, ARPC2, ARPC5, ITGA2, PAK4, |
| | PIP4K2C, RHOU, RND1, RND3 |
| Phospholipase C Signaling | ADCY7, ARHGEF1, ARHGEF9, CREBBP, |
| | GNB1L, ITGA2, KRAS, PLCB2, PLCD1, PLCD3, |
| | PLD6, PRKCA, RHOU, RND1, RND3, SOS2, |
| | TGM2 |
| Role of NFAT in Cardiac Hypertrophy | ADCY7, CACNG4, GNAI1, GNAI3, GNB1L, |
| | KRAS, LIF, NKX2-5, PIK3R1, PLCB2, PLCD1, |
| | PLCD3, PRKCA, RCAN1, SOS2 |
| Endocannabinoid Developing Neuron Pathway | ADCY7, CCND1, CREBBP, GNAI1, GNAI3, |
| | GNB1L, KRAS, MAPK15, PAX6, PIK3R1 |
| PAK Signaling | GIT1, ITGA2, KRAS, PAK4, PDGFD, PDGFRB, |
| | PIK3R1, PTK2, SOS2 |
| UVA-Induced MAPK Signaling | KRAS, PARP14, PIK3R1, PLCB2, PLCD1, |
| | PLCD3, PRKCA, RPS6KB2, SMPD1 |
| Cyclins and Cell Cycle Regulation | ABL1, CCNA2, CCND1, CDC25A, CDK4, |
| | CDK6, E2F8, SKP2 |
| Prolactin Signaling | CREBBP, IRF1, JAK2, KRAS, PDPK1, PIK3R1, |
| | PRKCA, SOS2 |
| Apelin Cardiomyocyte Signaling Pathway | GNAI1, GNAI3, MAPK15, PIK3R1, PLCB2, |
| | PLCD1, PLCD3, PRKCA, SLC9A3 |
| Cholecystokinin/Gastrin-mediated Signaling | BCAR1, IL1B, KRAS, PLCB2, PRKCA, PTK2, |
| | RHOU, RND1, RND3, SOS2 |
| Regulation of Cellular Mechanics by Calpain | CCNA2, CCND1, CDK4, CDK6, ITGA2, KRAS, |
| Protease | PTK2 |
| Interferon Signaling | IFIT3, IRF1, JAK2, MX1, PSMB8 |
| Aryl Hydrocarbon Receptor Signaling | ALDH9A1, APAF1, CCNA2, CCND1, CDK4, |
| | CDK6, HSPB7, IL1B, MDM2, NCOA3, TGM2 |
| ErbB4 Signaling | KRAS, PDPK1, PIK3R1, PRKCA, PSEN1, |
| | SOS2, YAP1 |
| IL-6 Signaling | CXCL8, HSPB7, IL1B, JAK2, KRAS, MCL1, |
| | NGFR, PIK3R1, SOS2, TNFRSF1B |
| GM-CSF Signaling | CCND1, CSF2, ETS1, JAK2, KRAS, PIK3R1, |
| | SOS2 |
| Acute Myeloid Leukemia Signaling | CCND1, IDH1, KITLG, KRAS, PIK3R1, |
| | RPS6KB2, SOS2, TCF4 |
| Melatonin Signaling | GNAI1, GNAI3, PLCB2, PLCD1, PLCD3, |
| | PRKCA, RORA |
| Paxillin Signaling | ACTG2, BCAR1, ITGA2, ITGB3, KRAS, PAK4, |
| | PIK3R1, PTK2, SOS2 |
| GNRH Signaling | ADCY7, CACNG4, CREBBP, GNAI1, GNAI3, |
| | KRAS, MAP3K3, PAK4, PLCB2, PRKCA, PTK2, |
| | SOS2 |
| Cardiac Hypertrophy Signaling | ADCY7, CREBBP, GNAI1, GNAI3, GNB1L, |
| | KRAS, MAP3K3, NKX2-5, PIK3R1, PLCB2, |
| | PLCD1, PLCD3, RHOU, RND1, RND3 |
| Actin Cytoskeleton Signaling | ACTG2, ARHGEF1, ARPC2, ARPC5, BCAR1, |
| | CRKL, GIT1, ITGA2, KRAS, PAK4, PDGFD, |
| | PIK3R1, PTK2, SOS2 |
| STAT3 Pathway | BMPR2, CDC25A, IL17RD, IL1B, IL7R, JAK2, |
| | KRAS, NGFR, PDGFRB, TGFBR3 |
| Leukocyte Extravasation Signaling | ABL1, ACTG2, BCAR1, CRKL, GNAI1, GNAI3, |
| | ITGA2, ITGB3, MMP1, MMP14, PIK3R1, |
| | PRKCA, PTK2 |
| NGF Signaling | CREBBP, KRAS, MAP3K3, NGFR, PDPK1, |
| | PIK3R1, RPS6KB2, SMPD1, SOS2 |
| Oncostatin M Signaling | JAK2, KRAS, MMP1, OSMR, PLAU |
| fMLP Signaling in Neutrophils | ARPC2, ARPC5, GNAI1, GNAI3, GNB1L, |
| | KRAS, PIK3R1, PLCB2, PRKCA |
| Salvage Pathways of Pyrimidine | AK4, APOBEC3F, APOBEC3G, CDK4, CDK6, |
| Ribonucleotides | DAPK1, FAM20B, UPRT |
| Endometrial Cancer Signaling | CCND1, ERBB2, KRAS, PDPK1, PIK3R1, |
| | SOS2 |
| IL-23 Signaling Pathway | CSF2, IL1B, JAK2, PIK3R1, RORA |
| FAT10 Cancer Signaling Pathway | BMPR2, NGFR, TCF4, TGFBR3, TNFRSF1B |
| CREB Signaling in Neurons | ADCY7, CACNG4, CREBBP, GNAI1, GNAI3, |
| | GNB1L, KRAS, PIK3R1, PLCB2, PLCD1, |
| | PLCD3, PRKCA, SOS2 |
| Colorectal Cancer Metastasis Signaling | ADCY7, APPL1, CCND1, GNB1L, JAK2, KRAS, |
| | LRP5, MMP1, MMP14, PIK3R1, RHOU, RND1, |
| | RND3, SOS2, TCF4 |
| Endothelin-1 Signaling | ADCY7, EDNRA, GNAI1, GNAI3, KRAS, |
| | MAPK15, PIK3R1, PLCB2, PLCD1, PLCD3, |
| | PLD6, PRKCA |
| HMGB1 Signaling | CSF2, CXCL8, IL1B, KRAS, LIF, NGFR, |
| | PIK3R1, RHOU, RND1, RND3, TNFRSF1B |
| Mouse Embryonic Stem Cell Pluripotency | BMPR2, CREBBP, JAK2, KRAS, LIF, PIK3R1, |
| | SOS2, TCF4 |
| FLT3 Signaling in Hematopoietic Progenitor | CBL, CREBBP, GAB2, KRAS, PDPK1, PIK3R1, |
| Cells | SOS2 |
| ErbB2-ErbB3 Signaling | CCND1, ERBB2, KRAS, PDPK1, PIK3R1, |
| | SOS2 |
| IGF-1 Signaling | JAK2, KRAS, PDPK1, PIK3R1, PTK2, |
| | RPS6KB2, SOS2, YWHAZ |
| Huntington's Disease Signaling | APAF1, ATP5PB, CDK5R1, CREBBP, DNM3, |
| | GNB1L, NSF, PDPK1, PIK3R1, PLCB2, |
| | PRKCA, SOS2, TAF9B, TGM2 |
| 14-3-3-mediated Signaling | CBL, KRAS, PIK3R1, PLCB2, PLCD1, PLCD3, |
| | PRKCA, YAP1, YWHAZ |
| CDK5 Signaling | ABL1, ADCY7, CDK5R1, ITGA2, KRAS, |
| | LAMC1, MAPK15, NGFR |
| Telomerase Signaling | ABL1, ELK3, ETS1, KRAS, PDPK1, PIK3R1, |
| | SOS2, TPP1 |
| Melanoma Signaling | CCND1, CDK4, KRAS, MDM2, PIK3R1 |
| 3-phosphoinositide Degradation | CDC25A, DUSP16, DUSP2, HACD2, INPPL1, |
| | NUDT3, PPM1H, PPTC7, PXYLP1, SYNJ2 |
| Death Receptor Signaling | ACTG2, APAF1, ARHGDIB, HSPB7, PARP14, |
| | TNFRSF1B, TNFRSF21 |
| ERK5 Signaling | CREBBP, KRAS, LIF, MAP3K3, RPS6KB2, |
| | YWHAZ |
| GPCR-Mediated Nutrient Sensing in | ADCY7, CACNG4, GNAI1, GNAI3, PLCB2, |
| Enteroendocrine Cells | PLCD1, PLCD3, PRKCA |
| Lymphotoxin β Receptor Signaling | APAF1, CREBBP, PDPK1, PIK3R1, TRAF1 |
| cAMP-mediated signaling | ADCY7, AKAP12, CREBBP, DUSP4, GNAI1, |
| | GNAI3, HTR7, LPAR1, PDE3A, PDE4D, |
| | PDE5A, PLD6, RGS2 |
| PI3K Signaling in B Lymphocytes | ABL1, CBL, KRAS, PDPK1, PIK3R1, PLCB2, |
| | PLCD1, PLCD3, PLEKHA2 |
| ErbB Signaling | ERBB2, KRAS, PAK4, PDPK1, PIK3R1, |
| | PRKCA, SOS2 |
| Melanocyte Development and Pigmentation | ADCY7, CREBBP, KITLG, KRAS, PIK3R1, |
| Signaling | RPS6KB2, SOS2 |
| Apelin Endothelial Signaling Pathway | ADCY7, GNAI1, GNAI3, KRAS, PIK3R1, |
| | PLCB2, PRKCA, RPS6KB2 |
| Fc Epsilon RI Signaling | CSF2, INPPL1, KRAS, PDPK1, PIK3R1, |
| | PRKCA, SOS2, SYNJ2 |
| Renin-Angiotensin Signaling | ADCY7, JAK2, KRAS, PAK4, PIK3R1, PRKCA, |
| | PTK2, SOS2 |
| Neurotrophin/TRK Signaling | CREBBP, KRAS, NGFR, PDPK1, PIK3R1, |
| | SOS2 |
| GP6 Signaling Pathway | COL5A1, ITGB3, LAMC1, LAMC2, PDPK1, |
| | PIK3R1, PRKCA, PTK2 |
| mTOR Signaling | EIF4A1, EIF4B, GNB1L, KRAS, PDPK1, |
| | PIK3R1, PLD6, PRKCA, RHOU, RND1, RND3, |
| | RPS6KB2 |
| 3-phosphoinositide Biosynthesis | CDC25A, DUSP16, DUSP2, HACD2, NUDT3, |
| | PIK3R1, PIP4K2C, PPM1H, PPTC7, PXYLP1 |
| Production of Nitric Oxide and Reactive Oxygen | CREBBP, IRF1, JAK2, MAP3K3, NGFR, |
| Species in Macrophages | PIK3R1, PRKCA, RHOU, RND1, RND3, |
| | TNFRSF1B |
| G Beta Gamma Signaling | CACNG4, GNAI1, GNAI3, GNB1L, KRAS, |
| | PDPK1, PRKCA, SOS2 |
| Chemokine Signaling | GNAI1, GNAI3, KRAS, PLCB2, PRKCA, PTK2 |
| IL-3 Signaling | CRKL, GAB2, JAK2, KRAS, PIK3R1, PRKCA |
| Renal Cell Carcinoma Signaling | CREBBP, ETS1, KRAS, PAK4, PIK3R1, SOS2 |
| tRNA Splicing | PDE3A, PDE4D, PDE5A, PLD6 |
| CCR3 Signaling in Eosinophils | GNAI1, GNAI3, GNB1L, KRAS, PAK4, PIK3R1, |
| | PLCB2, PRKCA |
| SAPK/JNK Signaling | CRKL, DUSP4, KRAS, MAP3K3, MAP4K5, |
| | PIK3R1, SOS2 |
| Wnt/Ca+ pathway | CREBBP, PLCB2, PLCD1, PLCD3, PRKCA |
| Endocannabinoid Neuronal Synapse Pathway | ADCY7, CACNG4, GNAI1, GNAI3, MAPK15, |
| | PLCB2, PLCD1, PLCD3 |
| Adrenomedullin signaling pathway | ADCY7, IL1B, KRAS, MAPK15, MAX, PIK3R1, |
| | PLCB2, PLCD1, PLCD3, PTK2, SOS2 |
| Thrombopoietin Signaling | GAB2, JAK2, KRAS, PIK3R1, PRKCA |
| Role of IL-17F in Allergic Inflammatory Airway | CREBBP, CSF2, CXCL8, IL1B |
| Diseases | |
| PD-1, PD-L1 cancer immunotherapy pathway | JAK2, LATS2, NGFR, PIK3R1, SKP2, |
| | TNFRSF1B, YAP1 |
| Acute Phase Response Signaling | IL1B, JAK2, KRAS, NGFR, OSMR, PDPK1, |
| | PIK3R1, SOS2, TCF4, TNFRSF1B |
| NF-κB Signaling | BMPR2, CREBBP, IL1B, KRAS, MAP3K3, |
| | NGFR, PDGFRB, PIK3R1, TGFBR3, |
| | TNFRSF1B |
| Gαq Signaling | GNB1L, PIK3R1, PLCB2, PLD6, PRKCA, |
| | RGS2, RHOU, RND1, RND3 |
| Cell Cycle: G2/M DNA Damage Checkpoint | ABL1, MDM2, SKP2, YWHAZ |
| Regulation | |
| Dendritic Cell Maturation | CREBBP, CSF2, IL1B, JAK2, NGFR, PIK3R1, |
| | PLCB2, PLCD1, PLCD3, TNFRSF1B |
| Ovarian Cancer Signaling | ABL1, CCND1, CDK4, EDNRA, KRAS, PIK3R1, |
| | RPS6KB2, TCF4 |
| AMPK Signaling | AK4, CCNA2, CCND1, CREBBP, DPF1, |
| | GNB1L, LIPE, PDPK1, PIK3R1, PPM1F, |
| | PPM1H |
| D-myo-inositol (1,4,5,6)-Tetrakisphosphate | CDC25A, DUSP16, DUSP2, HACD2, NUDT3, |
| Biosynthesis | PPM1H, PPTC7, PXYLP1 |
| D-myo-inositol (3,4,5,6)-tetrakisphosphate | CDC25A, DUSP16, DUSP2, HACD2, NUDT3, |
| Biosynthesis | PPM1H, PPTC7, PXYLP1 |
| Growth Hormone Signaling | JAK2, PDPK1, PIK3R1, PRKCA, RPS6KB2 |
| α-Adrenergic Signaling | ADCY7, GNAI1, GNAI3, GNB1L, KRAS, PRKCA |
| ILK Signaling | ACTG2, CCND1, CREBBP, ITGB3, PDPK1, |
| | PIK3R1, PTK2, RHOU, RND1, RND3 |
| Type II Diabetes Mellitus Signaling | ACSL5, CACNG4, NGFR, PDPK1, PIK3R1, |
| | PRKCA, SMPD1, TNFRSF1B |
| Corticotropin Releasing Hormone Signaling | ADCY7, ARPC5, CACNG4, CREBBP, GNAI1, |
| | GNAI3, PRKCA, PTCH1 |
| Angiopoietin Signaling | GRB7, KRAS, PAK4, PIK3R1, PTK2 |
| VEGF Signaling | ACTG2, KRAS, PIK3R1, PRKCA, PTK2, SOS2 |
| Gαi Signaling | ADCY7, GNAI1, GNAI3, GNB1L, KRAS, LPAR1, |
| | SOS2 |
| Apelin Adipocyte Signaling Pathway | ADCY7, GNAI1, GNAI3, LIPE, MAPK15 |
| Role of Pattern Recognition Receptors in | CSF2, CXCL8, IL1B, LIF, OAS2, PIK3R1, |
| Recognition of Bacteria and Viruses | PRKCA, RIPK2 |
| Gα12/13 Signaling | ARHGEF1, F2RL1, KRAS, LPAR1, LPAR2, |
| | PIK3R1, PTK2 |
| NF-κB Activation by Viruses | ITGA2, ITGB3, KRAS, PIK3R1, PRKCA |
| CNTF Signaling | JAK2, KRAS, PIK3R1, RPS6KB2 |
| Induction of Apoptosis by HIV1 | APAF1, NGFR, TNFRSF1B, TRAF1 |
| Phospholipases | PLCB2, PLCD1, PLCD3, PLD6 |
| FGF Signaling | CREBBP, CRKL, PIK3R1, PRKCA, SOS2 |
| Androgen Signaling | CACNG4, CCND1, CREBBP, GNAI1, GNAI3, |
| | GNB1L, PRKCA |
| Type I Diabetes Mellitus Signaling | APAF1, IL1B, IRF1, JAK2, NGFR, TNFRSF1B |
| Dopamine-DARPP32 Feedback in cAMP | ADCY7, CREBBP, GNAI1, GNAI3, PLCB2, |
| Signaling | PLCD1, PLCD3, PRKCA |
| Th2 Pathway | BMPR2, DLL1, JAK2, NOTCH2, PIK3R1, |
| | PSEN1, TGFBR3 |
| Pyridoxal 5′-phosphate Salvage Pathway | CDK4, CDK6, DAPK1, FAM20B |
| Ceramide Signaling | KRAS, NGFR, PIK3R1, SMPD1, TNFRSF1B |
| NRF2-mediated Oxidative Stress Response | ACTG2, BACH1, CREBBP, DNAJB9, DNAJC3, |
| | KRAS, PIK3R1, PRKCA, SQSTM1 |
| Cardiac β-adrenergic Signaling | ADCY7, AKAP12, GNB1L, PDE3A, PDE4D, |
| | PDE5A, PLD6 |
| Th17 Activation Pathway | CCL20, CSF2, IL1B, JAK2, RORA |
| Sperm Motility | ABL1, ERBB2, JAK2, PDE4D, PDGFRB, |
| | PLCB2, PLCD1, PLCD3, PRKCA, PTK2 |
| Opioid Signaling Pathway | ADCY7, AP2M1, CACNG4, CREBBP, GNAI1, |
| | GNAI3, KRAS, MAPK15, PRKCA, RPS6KB2, |
| | SOS2 |
| p38 MAPK Signaling | CREBBP, HSPB7, IL1B, MAX, RPS6KB2, |
| | TNFRSF1B |
| Role of NANOG in Mammalian Embryonic Stem | BMPR2, JAK2, KRAS, LIF, PIK3R1, SOS2 |
| Cell Pluripotency | |
| Wnt/β-catenin Signaling | APPL1, BMPR2, CCND1, CREBBP, LRP5, |
| | MDM2, TCF4, TGFBR3 |
| TGF-β Signaling | BMPR2, CREBBP, KRAS, NKX2-5, SOS2 |
| Synaptogenesis Signaling Pathway | ADCY7, AP2M1, ARPC2, ARPC5, CREBBP, |
| | CRKL, EIF4EBP2, KRAS, NSF, PIK3R1, |
| | RAB5B, RPS6KB2, SOS2 |
| Th1 Pathway | DLL1, IRF1, JAK2, NOTCH2, PIK3R1, PSEN1 |
| Estrogen-Dependent Breast Cancer Signaling | CCND1, CREBBP, KRAS, PIK3R1 |
| TREM1 Signaling | CSF2, CXCL8, IL1B, JAK2 |
| Role of NFAT in Regulation of the Immune | GNAI1, GNAI3, GNB1L, KRAS, PIK3R1, |
| Response | PLCB2, RCAN1, SOS2 |
| Neuropathic Pain Signaling In Dorsal Horn | PIK3R1, PLCB2, PLCD1, PLCD3, PRKCA |
| Neurons | |
| Synaptic Long Term Potentiation | CREBBP, KRAS, PLCB2, PLCD1, PLCD3, |
| | PRKCA |
| GDNF Family Ligand-Receptor Interactions | DOK3, KRAS, PIK3R1, SOS2 |
| Osteoarthritis Pathway | BMPR2, CREBBP, CXCL8, IL1B, ITGA2, |
| | MMP1, PTCH1, TCF4, TNFRSF1B |
| Regulation of eIF4 and p70S6K Signaling | EIF4A1, EIF4EBP2, ITGA2, KRAS, PDPK1, |
| | PIK3R1, SOS2 |
| Synaptic Long Term Depression | CACNG4, GNAI1, GNAI3, KRAS, PLCB2, |
| | PLCD1, PLCD3, PRKCA |
| eNOS Signaling | ADCY7, CCNA2, LPAR1, LPAR2, PDPK1, |
| | PIK3R1, PRKCA |
| Gαs Signaling | ADCY7, CREBBP, GNB1L, HTR7, RGS2 |
| JAK/Stat Signaling | JAK2, KRAS, PIK3R1, SOS2 |
| Systemic Lupus Erythematosus In T Cell | CBL, CREBBP, GNAI1, GNAI3, GNB1L, KRAS, |
| Signaling Pathway | PIK3R1, PTK2, RHOU, RND1, RND3, |
| | RPS6KB2, SOS2 |
| Cdc42 Signaling | ARPC2, ARPC5, CDC42EP2, ITGA2, LLGL1, |
| | PAK4, PARD6A |
| BMP signaling pathway | BMPR2, CREBBP, KRAS, NKX2-5 |
| VEGF Family Ligand-Receptor Interactions | KRAS, PIK3R1, PRKCA, SOS2 |
| PKCθ Signaling in T Lymphocytes | CACNG4, KRAS, MAP3K3, PIK3R1, SOS2 |
| CD28 Signaling in T Helper Cells | ARPC2, ARPC5, PDPK1, PIK3R1 |
| Amyotrophic Lateral Sclerosis Signaling | APAF1, HECW1, PIK3R1, RAB5B |
| EIF2 Signaling | ACTG2, CCND1, EIF4A1, KRAS, PDPK1, |
| | PIK3R1, SOS2 |
| Neuroinflammation Signaling Pathway | BMPR2, CREBBP, CXCL8, IL1B, JAK2, |
| | MAPK15, PIK3R1, PSEN1, TGFBR3 |
| Sirtuin Signaling Pathway | ACSS2, ATP5PB, CXCL8, GABPA, MAPK15, |
| | SCNN1A |
| T Cell Exhaustion Signaling Pathway | BMPR2, JAK2, KRAS, PIK3R1, TGFBR3 |

TABLE 12
H460 lung cancer upregulated pathways and associated genes

| Pathway Name | Gene |
|---|---|
| PTEN Signaling | CASP9, CBL, CCND1, CDKN1A, FGFR2, |
| | INPPL1, KRAS, PDGFRB, PDPK1, PIK3R1, |
| | PIK3R3, PTK2, SYNJ2, TGFBR3 |
| Endocannabinoid Cancer Inhibition Pathway | ADCY1, ADCY9, CASP2, CASP7, CASP9, |
| | CCND1, CDKN1A, GNAI3, LEF1, PIK3R1, |
| | PIK3R3, PRKAA2, PTK2, SMPD1, TRIB3 |
| Pancreatic Adenocarcinoma Signaling | CASP9, CCND1, CDK2, CDKN1A, HMOX1, |
| | KRAS, MAPK8, NOTCH1, PIK3R1, PIK3R3, |
| | PLD1 |
| PPARα/RXRα Activation | ABCA1, ADCY1, ADCY9, FASN, GNA15, |
| | HELZ2, IL1RAP, IL1RL1, KRAS, MAPK8, |
| | PLCD3, PRKAA2, TGFBR3 |
| AMPK Signaling | ACTB, AK8, CCNA2, CCND1, CDKN1A, |
| | CPT1C, FASN, MAPK13, PDPK1, PIK3R1, |
| | PIK3R3, PPAT, PPM1F, PRKAA2 |
| RhoGDI Signaling | ACTB, ARPC2, ARPC4, ARPC5, GNA15, |
| | GNAI3, GNB1, PAK4, PIP4K2C, RHOB, RHOV |
| Antioxidant Action of Vitamin C | HMOX1, MAPK13, MAPK8, PLCD3, PLD1, |
| | SLC23A2, SLC2A3 |
| Cell Cycle: G1/S Checkpoint Regulation | CCND1, CDK2, CDK6, CDKN1A, MAX |
| GPCR-Mediated Integration of Enteroendocrine | ADCY1, ADCY9, GNA15, GNAI3, PLCD3 |
| Signaling Exemplified by an L Cell | |
| PPAR Signaling | FOS, IL1RAP, IL1RL1, KRAS, PDGFRB, |
| | TNFRSF1B |
| HIPPO signaling | LATS2, TEAD3, TEAD4, WWTR1, YWHAZ |
| Systemic Lupus Erythematosus In T Cell | CASP2, CASP7, CASP9, CBL, FOS, GNAI3, |
| Signaling Pathway | HLA-DMA, KRAS, ORAI1, PIK3R1, PIK3R3, |
| | PPP3R1, PTK2, RHOB, RHOV |
| LXR/RXR Activation | ABCA1, FASN, IL1 RAP, IL1RL1, TNFRSF1B |

TABLE 13
H460 lung cancer aberrant pathways and associated genes

| Pathway Name | Gene |
|---|---|
| Molecular Mechanisms of Cancer | ADCY1, ADCY9, APH1B, BMP6, CAMK2B, |
| | CASP7, CASP9, CBL, CCND1, CDK2, CDK6, |
| | CDKN1A, CHEK1, FOS, FZD8, GNA15, GNAI3, |
| | HHAT, KRAS, LEF1, MAPK13, MAPK8, MAX, |
| | NOTCH1, PAK4, PIK3R1, PIK3R3, PMAIP1, |
| | PRKCH, PSEN1, PTK2, RHOB, RHOV, |
| | WNT5A, WNT9A |
| Regulation of the Epithelial-Mesenchymal | APH1B, EGR1, ETS1, FGFR2, FGFRL1, FZD8, |
| Transition Pathway | JAG1, JAG2, KRAS, LEF1, NOTCH1, NOTCH2, |
| | NOTCH3, PARD6A, PDGFRB, PIK3R1, |
| | PIK3R3, PSEN1, WNT5A, WNT9A |
| Role of IL-17A in Arthritis | CXCL1, CXCL3, CXCL5, CXCL8, MAPK13, |
| | MAPK8, MMP1, PIK3R1, PIK3R3 |
| Th1 and Th2 Activation Pathway | APH1B, HLA-DMA, ICAM1, IL18R1, IL1RL1, |
| | IL4R, IL6R, IRF1, JAG1, JAG2, NOTCH1, |
| | NOTCH2, NOTCH3, PIK3R1, PIK3R3, PSEN1, |
| | TGFBR3 |
| Axonal Guidance Signaling | ADAMTS16, ARPC2, ARPC4, ARPC5, BMP6, |
| | CRKL, EFNB1, EFNB2, FZD8, GLI2, GNA15, |
| | GNAI3, GNB1, KALRN, KRAS, MMP1, PAK4, |
| | PAPPA2, PIK3R1, PIK3R3, PLCD3, PLXNA1, |
| | PLXNA2, PPP3R1, PRKCH, PTK2, PXN, |
| | RASSF5, TUBB, TUBG1, WNT5A, WNT9A |
| Role of Macrophages, Fibroblasts and | C5, CAMK2B, CCND1, CXCL8, F2RL1, FOS, |
| Endothelial Cells in Rheumatoid Arthritis | FZD8, ICAM1, IL18R1, IL1RAP, IL1RL1, IL6R, |
| | KRAS, LEF1, MMP1, PIK3R1, PIK3R3, PLCD3, |
| | PPP3R1, PRKCH, TNFRSF1B, WNT5A, |
| | WNT9A |
| Role of IL-17A in Psoriasis | CXCL1, CXCL3, CXCL5, CXCL8 |
| Role of Osteoblasts, Osteoclasts and | BMP6, CASP9, CBL, FOS, FZD8, IL18R1, |
| Chondrocytes in Rheumatoid Arthritis | IL1RAP, IL1RL1, LEF1, MAPK8, MMP1, |
| | PIK3R1, PIK3R3, PPP3R1, TNFRSF1B, |
| | WNT5A, WNT9A |
| Airway Pathology in Chronic Obstructive | CXCL3, CXCL8, MMP1 |
| Pulmonary Disease | |
| HER-2 Signaling in Breast Cancer | CASP9, CCND1, CDK6, CDKN1A, KRAS, |
| | PARD6A, PIK3R1, PIK3R3, PRKCH |
| TR/RXR Activation | AKR1C3, FASN, KLF9, PIK3R1, PIK3R3, |
| | SREBF2, TBL1XR1, THRA, THRB |
| Germ Cell-Sertoli Cell Junction Signaling | ACTB, KRAS, MAP3K3, MAPK8, PAK4, |
| | PDPK1, PIK3R1, PIK3R3, PTK2, PXN, RHOB, |
| | RHOV, TUBB, TUBG1 |
| Role of Tissue Factor in Cancer | CXCL1, CXCL8, EGR1, F2RL1, GNA15, KRAS, |
| | MAPK13, MMP1, PIK3R1, PIK3R3, PLAUR |
| Serotonin Receptor Signaling | ADCY1, ADCY9, GCH1, PCBD1, QDPR, SMOX |
| Granulocyte Adhesion and Diapedesis | C5, CCL26, CXCL1, CXCL2, CXCL3, CXCL5, |
| | CXCL8, GNAI3, ICAM1, IL1RAP, IL1RL1, |
| | MMP1, SDC1, TNFRSF1B |
| Breast Cancer Regulation by Stathmin1 | ADCY1, ADCY9, CAMK2B, CDK2, CDKN1A, |
| | GNAI3, GNB1, KRAS, PIK3R1, PIK3R3, |
| | PRKCH, STMN1, TUBB, TUBG1, UHMK1 |
| Prostate Cancer Signaling | CASP9, CCND1, CDK2, CDKN1A, KRAS, |
| | LEF1, PDPK1, PIK3R1, PIK3R3 |
| Hepatic Fibrosis/Hepatic Stellate Cell | COL5A2, COL5A3, CXCL3, CXCL8, FGFR2, |
| Activation | ICAM1, IL1RAP, IL1RL1, IL4R, IL6R, MMP1, |
| | PDGFRB, SERPINE1, TNFRSF1B |
| FAK Signaling | ACTB, ARHGAP26, KRAS, PAK4, PDPK1, |
| | PIK3R1, PIK3R3, PTK2, PXN |
| Acetate Conversion to Acetyl-CoA | ACSS2, ACSS3 |
| Phenylalanine Degradation I (Aerobic) | PCBD1, QDPR |
| IL-17 Signaling | CXCL1, CXCL5, CXCL8, KRAS, MAPK13, |
| | MAPK8, PIK3R1, PIK3R3 |
| Epithelial Adherens Junction Signaling | ACTB, ARPC2, ARPC4, ARPC5, KRAS, LEF1, |
| | NOTCH1, NOTCH2, NOTCH3, TGFBR3, TUBB, |
| | TUBG1 |
| RAR Activation | ACTB, ADCY1, ADCY9, AKR1C3, ALDH1A3, |
| | FOS, MAPK13, MAPK8, MMP1, NR2F6, |
| | PDPK1, PIK3R1, PIK3R3, PRKCH |
| Human Embryonic Stem Cell Pluripotency | BMP6, FGFR2, FGFRL1, FZD8, LEF1, |
| | PDGFRB, PDPK1, PIK3R1, PIK3R3, WNT5A, |
| | WNT9A |
| Iron homeostasis signaling pathway | ABCB10, ATP6V0A2, ATP6V1B2, BMP6, |
| | CYBRD1, ERFE, HFE, HMOX1, IL6R, PCBP1, |
| | PDGFRB |
| Gαq Signaling | GNA15, GNB1, GRK2, HMOX1, PIK3R1, |
| | PIK3R3, PLD1, PPP3R1, PRKCH, RGS2, |
| | RHOB, RHOV |
| IL-10 Signaling | FOS, HMOX1, IL1RAP, IL1RL1, IL4R, MAPK13, |
| | MAPK8 |
| Role of JAK family kinases in IL-6-type Cytokine | IL6R, MAPK13, MAPK8, OSMR |
| Signaling | |
| Cell Cycle Control of Chromosomal Replication | CDC45, CDC6, CDK2, CDK6, CDT1, MCM2 |
| Glucocorticoid Receptor Signaling | ACTB, CDKN1A, CXCL3, CXCL8, FOS, |
| | HMGB1, ICAM1, KRAS, KRT80, MAPK13, |
| | MAPK8, MED14, MMP1, PIK3R1, PIK3R3, |
| | PLAU, PPP3R1, PRKAA2, SERPINE1, TAF4B |
| Parkinson's Signaling | CASP9, MAPK13, MAPK8 |
| Erythropoietin Signaling | CBL, FOS, KRAS, PDPK1, PIK3R1, PIK3R3, |
| | PRKCH |
| Gap Junction Signaling | ACTB, ADCY1, ADCY9, GJB2, GNAI3, KRAS, |
| | PIK3R1, PIK3R3, PLCD3, PPP3R1, PRKCH, |
| | TUBB, TUBG1 |
| Hereditary Breast Cancer Signaling | ACTB, CCND1, CDK6, CDKN1A, CHEK1, |
| | KRAS, PIK3R1, PIK3R3, RFC3, TUBG1 |
| GADD45 Signaling | CCND1, CDK2, CDKN1A |
| Myc Mediated Apoptosis Signaling | CASP9, KRAS, MAPK8, PIK3R1, PIK3R3, |
| | YWHAZ |
| Sphingomyelin Metabolism | SGMS1, SMPD1 |
| IL-4 Signaling | HLA-DMA, IL4R, INPPL1, KRAS, PIK3R1, |
| | PIK3R3, SYNJ2 |
| T Cell Receptor Signaling | CBL, FOS, KRAS, MAPK8, PAG1, PIK3R1, |
| | PIK3R3, PPP3R1 |
| IL-17A Signaling in Fibroblasts | CXCL5, FOS, MAPK13, MMP1 |
| Methylglyoxal Degradation VI | LDHD |
| Endoplasmic Reticulum Stress Pathway | CASP7, CASP9, MAPK8 |
| Putrescine Degradation III | ALDH1A3, ALDH9A1, SMOX |
| Agranulocyte Adhesion and Diapedesis | ACTB, C5, CCL26, CXCL1, CXCL2, CXCL3, |
| | CXCL5, CXCL8, GNAI3, ICAM1, MMP1, PODXL |
| Clathrin-mediated Endocytosis Signaling | ACTB, AP2M1, ARPC2, ARPC4, ARPC5, CBL, |
| | HIP1, PIK3R1, PIK3R3, PPP3R1, RAB5B, |
| | STON2 |
| IL-15 Signaling | CXCL8, KRAS, MAPK13, PIK3R1, PIK3R3, |
| | PTK2 |
| Apelin Cardiac Fibroblast Signaling Pathway | APLN, PRKAA2, SERPINE1 |
| Factors Promoting Cardiogenesis in Vertebrates | BMP6, CDC6, CDK2, FZD8, LEF1, PRKCH, |
| | TGFBR3 |
| Leptin Signaling in Obesity | ADCY1, ADCY9, PDE3A, PIK3R1, PIK3R3, |
| | PLCD3 |
| Docosahexaenoic Acid (DHA) Signaling | CASP9, PDPK1, PIK3R1, PIK3R3 |
| Role of CHK Proteins in Cell Cycle Checkpoint | CDK2, CDKN1A, CHEK1, MDC1, RFC3 |
| Control | |
| Tumoricidal Function of Hepatic Natural Killer | CASP7, CASP9, ICAM1 |
| Cells | |
| Angiopoietin Signaling | CASP9, KRAS, PAK4, PIK3R1, PIK3R3, PTK2 |
| Dopamine Receptor Signaling | ADCY1, ADCY9, GCH1, PCBD1, QDPR, SMOX |
| G-Protein Coupled Receptor Signaling | ADCY1, ADCY9, CAMK2B, GNA15, GNAI3, |
| | GRK2, KRAS, PDE3A, PDE4D, PDE9A, |
| | PDPK1, PIK3R1, PIK3R3, PTGER2, RGS2 |
| Tryptophan Degradation X (Mammalian, via | ALDH1A3, ALDH9A1, SMOX |
| Tryptamine) | |
| Semaphorin Signaling in Neurons | PAK4, PLXNA1, PTK2, RHOB, RHOV |
| Cleavage and Polyadenylation of Pre-mRNA | NUDT21, PAPOLA |
| 4-hydroxyproline Degradation I | HOGA1 |
| Fatty Acid Biosynthesis Initiation II | FASN |
| Glycine Biosynthesis I | SHMT2 |
| Guanine and Guanosine Salvage I | HPRT1 |
| L-cysteine Degradation III | MPST |
| Palmitate Biosynthesis I (Animals) | FASN |
| Chronic Myeloid Leukemia Signaling | CCND1, CDK6, CDKN1A, CRKL, KRAS, |
| | PIK3R1, PIK3R3 |
| Bile Acid Biosynthesis, Neutral Pathway | AKR1C3, SCP2 |
| Role of PI3K/AKT Signaling in the Pathogenesis | CASP9, CRKL, GNAI3, PIK3R1, PIK3R3 |
| of Influenza | |
| Role of Oct4 in Mammalian Embryonic Stem | ETS2, FBXO15, KDM5B, NR2F6 |
| Cell Pluripotency | |
| PD-1, PD-L1 cancer immunotherapy pathway | CDK2, HLA-DMA, LATS2, PDCD4, PIK3R1, |
| | PIK3R3, TNFRSF1B |
| Dopamine Degradation | ALDH1A3, ALDH9A1, SMOX |
| TNFR2 Signaling | FOS, MAPK8, TNFRSF1B |
| Choline Biosynthesis III | HMOX1, PLD1 |
| 5-aminoimidazole Ribonucleotide Biosynthesis I | PPAT |
| L-carnitine Biosynthesis | ALDH9A1 |
| Methylglyoxal Degradation I | GLO1 |
| N-acetylglucosamine Degradation I | GNPDA1 |
| Tetrahydrobiopterin Biosynthesis I | GCH1 |
| Tetrahydrobiopterin Biosynthesis II | GCH1 |
| Thiosulfate Disproportionation III (Rhodanese) | MPST |
| Tyrosine Biosynthesis IV | PCBD1 |
| Thyroid Cancer Signaling | CCND1, CXCL8, KRAS, LEF1 |
| HIF1α Signaling | KRAS, MAPK13, MAPK8, MMP1, PIK3R1, |
| | PIK3R3, SLC2A3 |
| Granzyme B Signaling | CASP9, LMNB2 |
| Vitamin-C Transport | SLC23A2, SLC2A3 |
| T Helper Cell Differentiation | HLA-DMA, IL18R1, IL4R, IL6R, TNFRSF1B |
| Inhibition of Angiogenesis by TSP1 | MAPK13, MAPK8, SDC1 |
| GABA Receptor Signaling | ADCY1, ADCY9, ALDH9A1, AP2M1, CACNG6, |
| | NSF |
| α-Adrenergic Signaling | ADCY1, ADCY9, GNAI3, GNB1, KRAS, PRKCH |
| D-myo-inositol (1, 4, 5)-trisphosphate | INPPL1, SYNJ2 |
| Degradation | |
| Dermatan Sulfate Degradation (Metazoa) | FGFRL1, IDS |
| Histamine Degradation | ALDH1A3, ALDH9A1 |
| Ovarian Cancer Signaling | CCND1, FZD8, KRAS, LEF1, PIK3R1, PIK3R3, |
| | WNT5A, WNT9A |
| Coagulation System | PLAU, PLAUR, SERPINE1 |
| Noradrenaline and Adrenaline Degradation | ALDH1A3, ALDH9A1, SMOX |
| Natural Killer Cell Signaling | INPPL1, KRAS, PAK4, PIK3R1, PIK3R3, |
| | PRKCH, SYNJ2 |
| 1D-myo-inositol Hexakisphosphate Biosynthesis | INPPL1, SYNJ2 |
| II (Mammalian) | |
| D-myo-inositol (1, 3, 4)-trisphosphate | INPPL1, SYNJ2 |
| Biosynthesis | |
| FAT10 Signaling Pathway | SQSTM1, UBE2Z |
| Interferon Signaling | IRF1, MED14, PSMB8 |
| Heme Degradation | HMOX1 |
| Melatonin Degradation II | SMOX |
| N-acetylglucosamine Degradation II | GNPDA1 |
| Spermine and Spermidine Degradation I | SMOX |
| Cell Cycle Regulation by BTG Family Proteins | BTG2, CCND1, CDK2 |
| Apelin Muscle Signaling Pathway | APLN, PRKAA2 |
| Role of BRCA1 in DNA Damage Response | ACTB, CDKN1A, CHEK1, MDC1, RFC3 |
| Nur77 Signaling in T Lymphocytes | CASP9, HLA-DMA, MAP3K3, PPP3R1 |
| CDP-diacylglycerol Biosynthesis I | AGPAT1, CDS1 |
| Fatty Acid α-oxidation | ALDH1A3, ALDH9A1 |
| April Mediated Signaling | FOS, MAPK13, MAPK8 |
| Inhibition of Matrix Metalloproteases | MMP1, SDC1, TFPI2 |
| Folate Polyglutamylation | SHMT2 |
| dTMP De Novo Biosynthesis | SHMT2 |
| Gustation Pathway | ADCY1, ADCY9, CACNG6, GNB1, P2RX5, |
| | PDE3A, PDE4D, PDE9A |
| B Cell Activating Factor Signaling | FOS, MAPK13, MAPK8 |
| Mechanisms of Viral Exit from Host Cells | ACTB, LMNB2, PRKCH |
| Cellular Effects of Sildenafil (Viagra) | ACTB, ADCY1, ADCY9, CACNG6, PDE3A, |
| | PDE4D, PLCD3 |
| Virus Entry via Endocytic Pathways | ACTB, AP2M1, KRAS, PIK3R1, PIK3R3, |
| | PRKCH |
| Phosphatidylglycerol Biosynthesis II (Non- | AGPAT1, CDS1 |
| plastidic) | |
| Polyamine Regulation in Colon Cancer | KRAS, MAX |
| Pyrimidine Deoxyribonucleotides De Novo | AK8, NME5 |
| Biosynthesis | |
| Pyrimidine Ribonucleotides Interconversion | AK8, NME5, NUDT15 |
| tRNA Splicing | PDE3A, PDE4D, PDE9A |
| IL-12 Signaling and Production in Macrophages | FOS, IRF1, MAPK13, MAPK8, PIK3R1, PIK3R3, |
| | PRKCH |
| Superpathway of D-myo-inositol (1, 4, 5)- | INPPL1, SYNJ2 |
| trisphosphate Metabolism | |
| Adenine and Adenosine Salvage III | HPRT1 |
| UDP-N-acetyl-D-glucosamine Biosynthesis II | GFPT1 |
| Urea Cycle | CPS1 |
| Hepatic Cholestasis | ADCY1, ADCY9, CXCL8, IL1RAP, IL1RL1, LIF, |
| | MAPK8, PRKCH, TNFRSF1B |
| Pyrimidine Ribonucleotides De Novo | AK8, NME5, NUDT15 |
| Biosynthesis | |
| Calcium-induced T Lymphocyte Apoptosis | HLA-DMA, ORAI1, PPP3R1, PRKCH |
| IL-22 Signaling | MAPK13, MAPK8 |
| TCA Cycle II (Eukaryotic) | IDH3A, MDH1B |
| Role of IL-17F in Allergic Inflammatory Airway | CXCL1, CXCL5, CXCL8 |
| Diseases | |
| iNOS Signaling | FOS, IRF1, MAPK13 |
| D-myo-inositol (1, 4, 5)-Trisphosphate | PIP4K2C, PLCD3 |
| Biosynthesis | |
| Aspartate Degradation II | MDH1B |
| Superpathway of Serine and Glycine | SHMT2 |
| Biosynthesis I | |
| Role of JAK1 and JAK3 in γc Cytokine Signaling | IL4R, KRAS, PIK3R1, PIK3R3 |
| Antiproliferative Role of TOB in T Cell Signaling | CCNA2, CDK2 |
| Gluconeogenesis I | ENO4, MDH1B |
| NAD Salvage Pathway II | NT5E, PXYLP1 |
| Ephrin A Signaling | PIK3R1, PIK3R3, PTK2 |
| Cell Cycle: G2/M DNA Damage Checkpoint | CDKN1A, CHEK1, YWHAZ |
| Regulation | |
| Histidine Degradation III | AMDHD1 |
| Salvage Pathways of Pyrimidine | TK2 |
| Deoxyribonucleotides | |
| IL-15 Production | FGFR2, IRF1, PDGFRB, PTK2, PTK7, ROR1 |
| Bladder Cancer Signaling | CCND1, CDKN1A, CXCL8, KRAS, MMP1 |
| Amyloid Processing | APH1B, MAPK13, PSEN1 |
| Superpathway of Cholesterol Biosynthesis | DHCR24 |
| Toll-like Receptor Signaling | FOS, IL1RL1, MAPK13, MAPK8 |
| Folate Transformations I | SHMT2 |
| Leucine Degradation I | BCAT2 |
| UDP-N-acetyl-D-galactosamine Biosynthesis II | GNPDA1 |
| Phagosome Formation | PIK3R1, PIK3R3, PLCD3, PRKCH, RHOB, |
| | RHOV |
| Role of p14/p19ARF in Tumor Suppression | PIK3R1, PIK3R3 |
| Sonic Hedgehog Signaling | GLI2, GRK2 |
| Glycolysis I | ENO4 |
| Lipid Antigen Presentation by CD1 | AP2M1 |
| Role of Wnt/GSK-3β Signaling in the | FZD8, LEF1, WNT5A, WNT9A |
| Pathogenesis of Influenza | |
| Reelin Signaling in Neurons | CRKL, MAPK8, PIK3R1, PIK3R3 |
| Unfolded protein response | MAPK8, SREBF2 |
| Dolichyl-diphosphooligosaccharide Biosynthesis | DPM3 |
| Glycine Betaine Degradation | SHMT2 |
| Xenobiotic Metabolism Signaling | ALDH1A3, ALDH9A1, CAMK2B, HMOX1, |
| | KRAS, MAP3K3, MAPK13, MAPK8, PIK3R1, |
| | PIK3R3, PRKCH, SMOX |
| 4-1BB Signaling in T Lymphocytes | MAPK13, MAPK8 |
| Transcriptional Regulatory Network in | HIST1H4H, SET |
| Embryonic Stem Cells | |
| Differential Regulation of Cytokine Production in | CXCL1 |
| Intestinal Epithelial Cells by IL-17A and IL-17F | |
| Phototransduction Pathway | GNB1, OPN3 |
| Mitochondrial Dysfunction | APH1B, ATP5PB, CASP9, CPT1C, MAPK8, |
| | PSEN1 |
| Purine Nucleotides De Novo Biosynthesis II | PPAT |
| Sertoli Cell-Sertoli Cell Junction Signaling | ACTB, KRAS, MAP3K3, MAPK13, MAPK8, |
| | TGFBR3, TUBB, TUBG1 |
| Cancer Drug Resistance By Drug Efflux | KRAS, PIK3R1, PIK3R3 |
| Cytotoxic T Lymphocyte-mediated Apoptosis of | CASP7, CASP9 |
| Target Cells | |
| DNA Methylation and Transcriptional | HIST1H4H, MTA2 |
| Repression Signaling | |
| IL-9 Signaling | PIK3R1, PIK3R3 |
| Retinoate Biosynthesis I | AKR1C3, ALDH1A3 |
| Role of JAK2 in Hormone-like Cytokine | SH2B2, SH2B3 |
| Signaling | |
| Androgen Signaling | CACNG6, CCND1, GNA15, GNAI3, GNB1, |
| | PRKCH |
| MSP-RON Signaling Pathway | ACTB, PIK3R1, PIK3R3 |
| Primary Immunodeficiency Signaling | RFX5, UNG |
| TWEAK Signaling | CASP7, CASP9 |
| Guanosine Nucleotides Degradation III | NT5E |
| Role of MAPK Signaling in the Pathogenesis of | KRAS, MAPK13, MAPK8 |
| Influenza | |
| VDR/RXR Activation | CDKN1A, IL1RL1, PRKCH |
| Hematopoiesis from Pluripotent Stem Cells | CXCL8, LIF |
| Granzyme A Signaling | SET |
| The Visual Cycle | AKR1C3 |
| CNTF Signaling | KRAS, PIK3R1, PIK3R3 |
| Induction of Apoptosis by HIV1 | CASP9, MAPK8, TNFRSF1B |
| Phospholipases | HMOX1, PLCD3, PLD1 |
| Adipogenesis pathway | FGFR2, FGFRL1, FZD8, TBL1XR1, WNT5A |
| Cholesterol Biosynthesis I | DHCR24 |
| Cholesterol Biosynthesis II (via 24, 25- | DHCR24 |
| dihydrolanosterol) | |
| Cholesterol Biosynthesis III (via Desmosterol) | DHCR24 |
| NAD Phosphorylation and Dephosphorylation | PXYLP1 |
| Urate Biosynthesis/Inosine 5′-phosphate | NT5E |
| Degradation | |
| nNOS Signaling in Neurons | PPP3R1, PRKCH |
| DNA damage-induced 14-3-3σ Signaling | CDK2 |
| Complement System | C1QBP, C5 |
| Tight Junction Signaling | ACTB, FOS, HSF1, NSF, NUDT21, PARD6A, |
| | TNFRSF1B |
| Regulation of IL-2 Expression in Activated and | FOS, KRAS, MAPK8, PPP3R1 |
| Anergic T Lymphocytes | |
| FAT10 Cancer Signaling Pathway | TGFBR3, TNFRSF1B |
| Differential Regulation of Cytokine Production in | CXCL1 |
| Macrophages and T Helper Cells by IL-17A and | |
| IL-17F | |
| Methylglyoxal Degradation III | AKR1C3 |
| Purine Nucleotides Degradation II (Aerobic) | NT5E |
| Valine Degradation I | BCAT2 |
| IL-23 Signaling Pathway | PIK3R1, PIK3R3 |
| Stearate Biosynthesis I (Animals) | DHCR24, FASN |
| Androgen Biosynthesis | AKR1C3 |
| Isoleucine Degradation I | BCAT2 |
| Phenylalanine Degradation IV (Mammalian, via | SMOX |
| Side Chain) | |
| Antigen Presentation Pathway | HLA-DMA, PSMB8 |
| tRNA Charging | FARSA, LARS2 |
| Mitochondrial L-carnitine Shuttle Pathway | CPT1C |
| RAN Signaling | TNPO1 |
| Adenosine Nucleotides Degradation II | NT5E |
| Histidine Degradation VI | AMDHD1 |
| Superpathway of Citrulline Metabolism | CPS1 |
| Serotonin Degradation | ALDH1A3, ALDH9A1, SMOX |
| Role of PKR in Interferon Induction and Antiviral | CASP9, IRF1 |
| Response | |
| MIF Regulation of Innate Immunity | FOS, MAPK8 |
| Mismatch Repair in Eukaryotes | RFC3 |
| Phagosome Maturation | ATP6V0A2, ATP6V1B2, NSF, RAB5B, TUBB, |
| | TUBG1 |
| Activation of IRF by Cytosolic Pattern | MAPK8 |
| Recognition Receptors | |
| Allograft Rejection Signaling | HLA-DMA |
| Altered T Cell and B Cell Signaling in | HLA-DMA |
| Rheumatoid Arthritis | |
| Assembly of RNA Polymerase II Complex | TAF4B |
| Atherosclerosis Signaling | COL5A3, CXCL8, ICAM1, MMP1 |
| Autoimmune Thyroid Disease Signaling | HLA-DMA |
| Autophagy | SQSTM1 |
| B Cell Development | HLA-DMA |
| BAG2 Signaling Pathway | CDKN1A |
| CTLA4 Signaling in Cytotoxic T Lymphocytes | AP2M1, PIK3R1, PIK3R3 |
| Calcium Signaling | CACNG6, CAMK2B, PPP3R1 |
| Caveolar-mediated Endocytosis Signaling | ACTB, RAB5B |
| Circadian Rhythm Signaling | BHLHE40 |
| Communication between Innate and Adaptive | CXCL8 |
| Immune Cells | |
| Crosstalk between Dendritic Cells and Natural | ACTB, CAMK2B, TNFRSF1B |
| Killer Cells | |
| Dermatan Sulfate Biosynthesis | CHST14 |
| Dermatan Sulfate Biosynthesis (Late Stages) | CHST14 |
| Eicosanoid Signaling | AKR1C3, PTGER2 |
| Estrogen Biosynthesis | AKR1C3 |
| Estrogen Receptor Signaling | KRAS, MED14, MED21, TAF4B |
| FXR/RXR Activation | FASN, MAPK8, SDC1 |
| Fatty Acid β-oxidation I | SCP2 |
| G Protein Signaling Mediated by Tubby | GNB1 |
| Glutamate Receptor Signaling | GNB1 |
| Graft-versus-Host Disease Signaling | HLA-DMA |
| Hypoxia Signaling in the Cardiovascular System | UBE2L6, UBE2Z |
| Intrinsic Prothrombin Activation Pathway | COL5A3 |
| Netrin Signaling | CACNG6, PPP3R1 |
| Nitric Oxide Signaling in the Cardiovascular | PIK3R1, PIK3R3, PRKCH |
| System | |
| OX40 Signaling Pathway | HLA-DMA, MAPK8 |
| Oxidative Phosphorylation | ATP5PB |
| PFKFB4 Signaling Pathway | HK2 |
| Protein Ubiquitination Pathway | CBL, DNAJB5, HSPA13, HSPB8, PSMB8, |
| | UBE2L6, UBE2Z, USP39, USP49 |
| Retinoic acid Mediated Apoptosis Signaling | CASP9, IRF1 |
| Retinol Biosynthesis | AKR1C3 |
| Role of Cytokines in Mediating Communication | CXCL8 |
| between Immune Cells | |
| Role of Hypercytokinemia/hyperchemokinemia | CXCL8 |
| in the Pathogenesis of Influenza | |
| Role of RIG1-like Receptors in Antiviral Innate | TRIM25 |
| Immunity | |
| SPINK1 Pancreatic Cancer Pathway | F2RL1 |
| Superpathway of Melatonin Degradation | SMOX |
| Systemic Lupus Erythematosus Signaling | C5, CBL, FOS, IL6R, KRAS, LSM14B, PIK3R1, |
| | PIK3R3 |
| Th17 Activation Pathway | IL6R, PTGER2 |
| Triacylglycerol Biosynthesis | AGPAT1 |
| nNOS Signaling in Skeletal Muscle Cells | CACNG6 |

TABLE 14
H460 lung cancer downregulated pathways and associated genes

| Pathway Name | Gene |
|---|---|
| HGF Signaling | CCND1, CDK2, CDKN1A, CRKL, ELF4, ELK3, |
| | ETS1, ETS2, FOS, KRAS, MAP3K3, MAPK8, |
| | PIK3R1, PIK3R3, PRKCH, PTK2, PXN |
| CXCR4 Signaling | ADCY1, ADCY9, EGR1, ELMO2, FOS, GNA15, |
| | GNAI3, GNB1, KRAS, MAPK8, PAK4, PIK3R1, |
| | PIK3R3, PRKCH, PTK2, PXN, RHOB, RHOV |
| Sphingosine-1-phosphate Signaling | ADCY1, ADCY9, CASP2, CASP7, CASP9, |
| | GNAI3, PDGFRB, PIK3R1, PIK3R3, PLCD3, |
| | PTK2, RHOB, RHOV, SMPD1 |
| Endothelin-1 Signaling | ADCY1, ADCY9, CASP2, CASP7, CASP9, |
| | FOS, GNA15, GNAI3, HMOX1, KRAS, |
| | MAPK13, MAPK8, PIK3R1, PIK3R3, PLCD3, |
| | PLD1, PRKCH, PTGER2 |
| GNRH Signaling | ADCY1, ADCY9, CACNG6, CAMK2B, EGR1, |
| | FOS, GNA15, GNAI3, GNB1, KRAS, MAP3K3, |
| | MAPK13, MAPK8, PAK4, PRKCH, PTK2, PXN |
| Rac Signaling | ABI2, ARPC2, ARPC4, ARPC5, KRAS, MAPK8, |
| | PAK4, PARD6A, PIK3R1, PIK3R3, PIP4K2C, |
| | PLD1, PTK2 |
| IL-8 Signaling | CCND1, CXCL1, CXCL8, FOS, GNAI3, GNB1, |
| | HMOX1, ICAM1, KRAS, LASP1, MAPK8, |
| | PIK3R1, PIK3R3, PLD1, PRKCH, PTK2, RHOB, |
| | RHOV |
| Colorectal Cancer Metastasis Signaling | ADCY1, ADCY9, APPL1, CASP9, CCND1, |
| | FOS, FZD8, GNB1, GRK2, IL6R, KRAS, LEF1, |
| | MAPK8, MMP1, PIK3R1, PIK3R3, PTGER2, |
| | RHOB, RHOV, WNT5A, WNT9A |
| Notch Signaling | APH1B, JAG1, JAG2, NOTCH1, NOTCH2, |
| | NOTCH3, PSEN1 |
| Th2 Pathway | APH1B, HLA-DMA, ICAM1, IL1RL1, IL4R, |
| | JAG1, JAG2, NOTCH1, NOTCH2, NOTCH3, |
| | PIK3R1, PIK3R3, PSEN1, TGFBR3 |
| Signaling by Rho Family GTPases | ACTB, ARPC2, ARPC4, ARPC5, CDC42EP2, |
| | FOS, GNA15, GNAI3, GNB1, MAPK8, PAK4, |
| | PARD6A, PIK3R1, PIK3R3, PIP4K2C, PLD1, |
| | PTK2, RHOB, RHOV, STMN1 |
| Fcγ Receptor-mediated Phagocytosis in | ACTB, ARPC2, ARPC4, ARPC5, CBL, HMOX1, |
| Macrophages and Monocytes | PIK3R1, PIK3R3, PLD1, PRKCH, PXN |
| Cardiac Hypertrophy Signaling (Enhanced) | ADCY1, ADCY9, CAMK2B, CXCL8, FGFR2, |
| | FGFRL1, FZD8, GNA15, GNAI3, GNB1, |
| | IL18R1, IL1RL1, IL4R, IL6R, KRAS, LIF, |
| | MAP3K3, MAPK13, MAPK8, PDE3A, PDE4D, |
| | PDE9A, PIK3R1, PIK3R3, PLCD3, PPP3R1, |
| | PRKCH, PTK2, TGFBR3, TNFRSF1B, WNT5A, |
| | WNT9A |
| Glioblastoma Multiforme Signaling | CCND1, CDK2, CDK6, CDKN1A, FZD8, KRAS, |
| | LEF1, PDGFRB, PIK3R1, PIK3R3, PLCD3, |
| | RHOB, RHOV, WNT5A, WNT9A |
| Apelin Endothelial Signaling Pathway | ADCY1, ADCY9, APLN, FOS, GNAI3, ICAM1, |
| | KRAS, MAPK8, PIK3R1, PIK3R3, PRKAA2, |
| | PRKCH |
| B Cell Receptor Signaling | CAMK2B, EGR1, ETS1, INPPL1, KRAS, |
| | MAP3K3, MAPK13, MAPK8, PAG1, PDPK1, |
| | PIK3R1, PIK3R3, PPP3R1, PTK2, RASSF5, |
| | SYNJ2 |
| Ephrin B Signaling | CBL, EFNB1, EFNB2, GNA15, GNAI3, GNB1, |
| | KALRN, PTK2, PXN |
| Renin-Angiotensin Signaling | ADCY1, ADCY9, FOS, KRAS, MAPK13, |
| | MAPK8, PAK4, PIK3R1, PIK3R3, PRKCH, |
| | PTGER2, PTK2 |
| Thrombin Signaling | ADCY1, ADCY9, CAMK2B, GATA2, GNA15, |
| | GNAI3, GNB1, KRAS, MAPK13, PDPK1, |
| | PIK3R1, PIK3R3, PLCD3, PRKCH, PTK2, |
| | RHOB, RHOV |
| Th1 Pathway | APH1B, HLA-DMA, ICAM1, IL18R1, IL6R, IRF1, |
| | NOTCH1, NOTCH2, NOTCH3, PIK3R1, |
| | PIK3R3, PSEN1 |
| IL-17A Signaling in Gastric Cells | CXCL1, CXCL8, FOS, MAPK13, MAPK8 |
| IL-6 Signaling | CXCL8, FOS, IL1RAP, IL1RL1, IL6R, KRAS, |
| | MAPK13, MAPK8, MCL1, PIK3R1, PIK3R3, |
| | TNFRSF1B |
| Glioma Signaling | CAMK2B, CCND1, CDK6, CDKN1A, IDH1, |
| | IDH2, KRAS, PDGFRB, PIK3R1, PIK3R3, |
| | PRKCH |
| Superpathway of Inositol Phosphate | DUSP5, HACD2, INPPL1, NUDT15, NUDT3, |
| Compounds | NUDT4, PIK3R1, PIK3R3, PIP4K2C, PLCD3, |
| | PPP4R1, PPTC7, PXYLP1, SET, SSH3, SYNJ2 |
| 14-3-3-mediated Signaling | CBL, FOS, KRAS, MAPK8, PIK3R1, PIK3R3, |
| | PLCD3, PRKCH, TP73, TUBB, TUBG1, YWHAZ |
| Tec Kinase Signaling | ACTB, FOS, GNA15, GNAI3, GNB1, MAPK8, |
| | PAK4, PIK3R1, PIK3R3, PRKCH, PTK2, RHOB, |
| | RHOV, TNFRSF21 |
| ATM Signaling | CBX1, CDK2, CDKN1A, CHEK1, HP1BP3, |
| | MAPK13, MAPK8, MDC1, RNF168, TP73 |
| Salvage Pathways of Pyrimidine | AK8, CDK2, CDK6, FAM20B, GRK5, MAPK8, |
| Ribonucleotides | NME5, PRKAA2, PRKCH, UPP1 |
| HMGB1 Signaling | CXCL8, FOS, HMGB1, ICAM1, KRAS, LIF, |
| | MAPK13, MAPK8, PIK3R1, PIK3R3, RHOB, |
| | RHOV, SERPINE1, TNFRSF1B |
| STAT3 Pathway | BMP6, CDKN1A, FGFR2, IL18R1, IL1RL1, |
| | IL4R, IL6R, KRAS, MAPK13, MAPK8, PDGFRB, |
| | TGFBR3 |
| PDGF Signaling | CRKL, FOS, INPPL1, KRAS, MAPK8, PDGFRB, |
| | PIK3R1, PIK3R3, SYNJ2 |
| Small Cell Lung Cancer Signaling | CASP9, CCND1, CDK2, CDK6, MAX, PIK3R1, |
| | PIK3R3, PTK2 |
| IGF-1 Signaling | CASP9, FOS, KRAS, MAPK8, PDPK1, PIK3R1, |
| | PIK3R3, PTK2, PXN, YWHAZ |
| Oxidative Ethanol Degradation III | ACSS2, ACSS3, ALDH1A3, ALDH9A1 |
| Integrin Signaling | ACTB, ARF3, ARHGAP26, ARPC2, ARPC4, |
| | ARPC5, CRKL, KRAS, MAPK8, PAK4, PIK3R1, |
| | PIK3R3, PTK2, PXN, RHOB, RHOV |
| Acute Phase Response Signaling | C5, FOS, HMOX1, IL1RAP, IL6R, KRAS, |
| | MAPK13, MAPK8, OSMR, PDPK1, PIK3R1, |
| | PIK3R3, SERPINE1, TNFRSF1B |
| Non-Small Cell Lung Cancer Signaling | CASP9, CCND1, CDK6, KRAS, PDPK1, |
| | PIK3R1, PIK3R3, RASSF5 |
| Ephrin Receptor Signaling | ARPC2, ARPC4, ARPC5, CRKL, EFNB1, |
| | EFNB2, GNA15, GNAI3, GNB1, KALRN, KRAS, |
| | PAK4, PTK2, PXN |
| IL-1 Signaling | ADCY1, ADCY9, FOS, GNA15, GNAI3, GNB1, |
| | IL1RAP, MAPK13, MAPK8 |
| Aryl Hydrocarbon Receptor Signaling | ALDH1A3, ALDH9A1, CCNA2, CCND1, CDK2, |
| | CDK6, CDKN1A, CHEK1, FOS, MAPK8, TGM2, |
| | TP73 |
| Glioma Invasiveness Signaling | KRAS, PIK3R1, PIK3R3, PLAU, PLAUR, PTK2, |
| | RHOB, RHOV |
| Telomerase Signaling | CDKN1A, ELF4, ELK3, ETS1, ETS2, KRAS, |
| | PDPK1, PIK3R1, PIK3R3, TERT |
| Endometrial Cancer Signaling | CASP9, CCND1, KRAS, LEF1, PDPK1, |
| | PIK3R1, PIK3R3 |
| 3-phosphoinositide Biosynthesis | DUSP5, HACD2, NUDT15, NUDT3, NUDT4, |
| | PIK3R1, PIK3R3, PIP4K2C, PPP4R1, PPTC7, |
| | PXYLP1, SET, SSH3 |
| Agrin Interactions at Neuromuscular Junction | ACTB, GABPA, GABPB1, KRAS, MAPK8, |
| | PAK4, PTK2, PXN |
| p70S6K Signaling | F2RL1, GNAI3, IL4R, KRAS, PDPK1, PIK3R1, |
| | PIK3R3, PLCD3, PLD1, PRKCH, YWHAZ |
| ErbB Signaling | FOS, KRAS, MAPK13, MAPK8, PAK4, PDPK1, |
| | PIK3R1, PIK3R3, PRKCH |
| IL-7 Signaling Pathway | CCND1, CDK2, MAPK13, MCL1, PDPK1, |
| | PIK3R1, PIK3R3, PTK2 |
| Neuregulin Signaling | CRKL, ERBIN, ERRFI1, KRAS, PDPK1, |
| | PIK3R1, PIK3R3, PRKCH, PSEN1 |
| UVA-Induced MAPK Signaling | CASP9, FOS, KRAS, MAPK13, MAPK8, |
| | PIK3R1, PIK3R3, PLCD3, SMPD1 |
| p53 Signaling | CCND1, CDK2, CDKN1A, CHEK1, MAPK8, |
| | PIK3R1, PIK3R3, PMAIP1, TP73 |
| Endocannabinoid Developing Neuron Pathway | ADCY1, ADCY9, CCND1, GNAI3, GNB1, |
| | KRAS, MAPK13, MAPK8, PIK3R1, PIK3R3 |
| IL-17A Signaling in Airway Cells | CXCL1, CXCL3, CXCL5, MAPK13, MAPK8, |
| | PIK3R1, PIK3R3 |
| Pyridoxal 5′-phosphate Salvage Pathway | CDK2, CDK6, FAM20B, GRK5, MAPK8, |
| | PRKAA2, PRKCH |
| TNFR1 Signaling | CASP2, CASP7, CASP9, FOS, MAPK8, PAK4 |
| Ethanol Degradation IV | ACSS2, ACSS3, ALDH1A3, ALDH9A1 |
| fMLP Signaling in Neutrophils | ARPC2, ARPC4, ARPC5, GNAI3, GNB1, KRAS, |
| | PIK3R1, PIK3R3, PPP3R1, PRKCH |
| Osteoarthritis Pathway | CASP2, CASP7, CASP9, CXCL8, FZD8, GLI2, |
| | HMGB1, IL1 RAP, IL1RL1, JAG1, LEF1, MMP1, |
| | NOTCH1, PRKAA2, TNFRSF1B |
| Regulation of Cellular Mechanics by Calpain | CCNA2, CCND1, CDK2, CDK6, KRAS, PTK2, |
| Protease | PXN |
| 3-phosphoinositide Degradation | DUSP5, HACD2, INPPL1, NUDT15, NUDT3, |
| | NUDT4, PPP4R1, PPTC7, PXYLP1, SET, |
| | SSH3, SYNJ2 |
| UVC-Induced MAPK Signaling | FOS, KRAS, MAPK13, MAPK8, PRKCH, |
| | SMPD1 |
| Role of NFAT in Cardiac Hypertrophy | ADCY1, ADCY9, CACNG6, CAMK2B, GNAI3, |
| | GNB1, KRAS, LIF, MAPK13, MAPK8, PIK3R1, |
| | PIK3R3, PLCD3, PPP3R1, PRKCH |
| D-myo-inositol-5-phosphate Metabolism | DUSP5, HACD2, NUDT15, NUDT3, NUDT4, |
| | PIP4K2C, PLCD3, PPP4R1, PPTC7, PXYLP1, |
| | SET, SSH3 |
| CD28 Signaling in T Helper Cells | ARPC2, ARPC4, ARPC5, FOS, HLA-DMA, |
| | MAPK8, PDPK1, PIK3R1, PIK3R3, PPP3R1 |
| ErbB4 Signaling | APH1B, KRAS, PDPK1, PIK3R1, PIK3R3, |
| | PRKCH, PSEN1 |
| Remodeling of Epithelial Adherens Junctions | ACTB, ARPC2, ARPC4, ARPC5, RAB5B, |
| | TUBB, TUBG1 |
| UVB-Induced MAPK Signaling | FOS, MAPK13, MAPK8, PIK3R1, PIK3R3, |
| | PRKCH |
| Mouse Embryonic Stem Cell Pluripotency | FZD8, ID1, ID3, KRAS, LEF1, LIF, MAPK13, |
| | PIK3R1, PIK3R3 |
| Cardiac Hypertrophy Signaling | ADCY1, ADCY9, GNA15, GNAI3, GNB1, IL6R, |
| | KRAS, MAP3K3, MAPK13, MAPK8, PIK3R1, |
| | PIK3R3, PLCD3, PPP3R1, RHOB, RHOV |
| GM-CSF Signaling | CAMK2B, CCND1, ETS1, KRAS, PIK3R1, |
| | PIK3R3, PPP3R1 |
| Apelin Liver Signaling Pathway | APLN, COL5A3, MAPK8, PDGFRB |
| Estrogen-mediated S-phase Entry | CCNA2, CCND1, CDK2, CDKN1A |
| RANK Signaling in Osteoclasts | CBL, FOS, MAP3K3, MAPK13, MAPK8, |
| | PIK3R1, PIK3R3, PPP3R1 |
| Endocannabinoid Neuronal Synapse Pathway | ADCY1, ADCY9, CACNG6, GNA15, GNAI3, |
| | GNB1, MAPK13, MAPK8, PLCD3, PPP3R1 |
| P2Y Purigenic Receptor Signaling Pathway | ADCY1, ADCY9, FOS, GNAI3, GNB1, KRAS, |
| | PIK3R1, PIK3R3, PLCD3, PRKCH |
| Paxillin Signaling | ACTB, KRAS, MAPK13, MAPK8, PAK4, |
| | PIK3R1, PIK3R3, PTK2, PXN |
| Relaxin Signaling | ADCY1, ADCY9, FOS, GNA15, GNAI3, GNB1, |
| | PDE3A, PDE4D, PDE9A, PIK3R1, PIK3R3 |
| Regulation of Actin-based Motility by Rho | ACTB, ARPC2, ARPC4, ARPC5, PAK4, |
| | PIP4K2C, RHOB, RHOV |
| NRF2-mediated Oxidative Stress Response | ACTB, DNAJB5, FOS, FOSL1, HMOX1, |
| | HSPB8, KRAS, MAFF, MAPK8, PIK3R1, |
| | PIK3R3, PRKCH, SQSTM1 |
| PI3K/AKT Signaling | CCND1, CDKN1A, INPPL1, KRAS, MCL1, |
| | PDPK1, PIK3R1, PIK3R3, SYNJ2, YWHAZ |
| ERK/MAPK Signaling | CRKL, ELF4, ELK3, ETS1, ETS2, FOS, KRAS, |
| | PAK4, PIK3R1, PIK3R3, PTK2, PXN, YWHAZ |
| Apelin Pancreas Signaling Pathway | APLN, MAPK8, PIK3R1, PIK3R3, PRKAA2 |
| PAK Signaling | KRAS, MAPK8, PAK4, PDGFRB, PIK3R1, |
| | PIK3R3, PTK2, PXN |
| Chemokine Signaling | CAMK2B, FOS, GNAI3, KRAS, MAPK13, |
| | MAPK8, PTK2 |
| IL-3 Signaling | CRKL, FOS, KRAS, PIK3R1, PIK3R3, PPP3R1, |
| | PRKCH |
| Fc Epsilon RI Signaling | INPPL1, KRAS, MAPK13, MAPK8, PDPK1, |
| | PIK3R1, PIK3R3, PRKCH, SYNJ2 |
| Apelin Adipocyte Signaling Pathway | ADCY1, ADCY9, APLN, GNAI3, MAPK13, |
| | MAPK8, PRKAA2 |
| Leukocyte Extravasation Signaling | ACTB, CRKL, GNAI3, ICAM1, MAPK13, |
| | MAPK8, MMP1, PIK3R1, PIK3R3, PRKCH, |
| | PTK2, PXN, RASSF5 |
| Apelin Cardiomyocyte Signaling Pathway | APLN, GNAI3, MAPK13, MAPK8, PIK3R1, |
| | PIK3R3, PLCD3, PRKCH |
| Prolactin Signaling | FOS, IRF1, KRAS, PDPK1, PIK3R1, PIK3R3, |
| | PRKCH |
| Ethanol Degradation II | ACSS2, ACSS3, ALDH1A3, ALDH9A1 |
| LPS-stimulated MAPK Signaling | FOS, KRAS, MAPK13, MAPK8, PIK3R1, |
| | PIK3R3, PRKCH |
| D-myo-inositol (1, 4, 5, 6)-Tetrakisphosphate | DUSP5, HACD2, NUDT15, NUDT3, NUDT4, |
| Biosynthesis | PPP4R1, PPTC7, PXYLP1, SET, SSH3 |
| D-myo-inositol (3, 4, 5, 6)-tetrakisphosphate | DUSP5, HACD2, NUDT15, NUDT3, NUDT4, |
| Biosynthesis | PPP4R1, PPTC7, PXYLP1, SET, SSH3 |
| SAPK/JNK Signaling | CRKL, GNB1, KRAS, MAP3K3, MAP4K5, |
| | MAPK8, PIK3R1, PIK3R3 |
| CD40 Signaling | FOS, ICAM1, MAPK13, MAPK8, PIK3R1, |
| | PIK3R3 |
| FGF Signaling | CRKL, FGFR2, FGFRL1, MAPK13, MAPK8, |
| | PIK3R1, PIK3R3 |
| RhoA Signaling | ACTB, ARPC2, ARPC4, ARPC5, CDC42EP2, |
| | PIP4K2C, PLD1, PLXNA1, PTK2 |
| Opioid Signaling Pathway | ADCY1, ADCY9, AP2M1, CACNG6, CAMK2B, |
| | FOS, FOSB, GNAI3, GNB1, GRK2, GRK5, |
| | KRAS, OGFR, PPP3R1, PRKCH |
| CCR3 Signaling in Eosinophils | CCL26, GNAI3, GNB1, KRAS, MAPK13, PAK4, |
| | PIK3R1, PIK3R3, PRKCH |
| Melanoma Signaling | CCND1, CDKN1A, KRAS, PIK3R1, PIK3R3 |
| mTOR Signaling | EIF4B, HMOX1, KRAS, PDPK1, PIK3R1, |
| | PIK3R3, PLD1, PRKAA2, PRKCH, RHOB, |
| | RHOV, RPS17, RPS21 |
| Ceramide Signaling | FOS, KRAS, MAPK8, PIK3R1, PIK3R3, SMPD1, |
| | TNFRSF1B |
| Acute Myeloid Leukemia Signaling | CCND1, IDH1, IDH2, KRAS, LEF1, PIK3R1, |
| | PIK3R3 |
| ILK Signaling | ACTB, CCND1, FOS, LEF1, MAPK8, PDPK1, |
| | PIK3R1, PIK3R3, PTK2, PXN, RHOB, RHOV |
| Death Receptor Signaling | ACTB, CASP2, CASP7, CASP9, MAPK8, |
| | TNFRSF1B, TNFRSF21 |
| Actin Nucleation by ARP-WASP Complex | ARPC2, ARPC4, ARPC5, KRAS, RHOB, RHOV |
| ERK5 Signaling | FOS, FOSL1, KRAS, LIF, MAP3K3, YWHAZ |
| Lymphotoxin β Receptor Signaling | CASP9, CXCL1, PDPK1, PIK3R1, PIK3R3 |
| Huntington's Disease Signaling | ATP5PB, CASP2, CASP7, CASP9, GNA15, |
| | GNB1, HIP1, MAPK8, NSF, PDPK1, PIK3R1, |
| | PIK3R3, PRKCH, TGM2 |
| Basal Cell Carcinoma Signaling | BMP6, FZD8, GLI2, LEF1, WNT5A, WNT9A |
| Protein Kinase A Signaling | ADCY1, ADCY9, AKAP12, AKAP6, CAMK2B, |
| | DUSP18, DUSP5, GNAI3, GNB1, HHAT, LEF1, |
| | PDE3A, PDE4D, PDE9A, PLCD3, PPP3R1, |
| | PRKCH, PTK2, PTPRA, PXN, YWHAZ |
| Actin Cytoskeleton Signaling | ABI2, ACTB, ARPC2, ARPC4, ARPC5, CRKL, |
| | KRAS, PAK4, PIK3R1, PIK3R3, PTK2, PXN, |
| | SSH3 |
| Adrenomedullin signaling pathway | ADCY1, ADCY9, FOS, GNA15, KRAS, |
| | MAPK13, MAPK8, MAX, PIK3R1, PIK3R3, |
| | PLCD3, PTK2 |
| EGF Signaling | FOS, MAPK13, MAPK8, PIK3R1, PIK3R3 |
| Estrogen-Dependent Breast Cancer Signaling | CCND1, FOS, KRAS, PIK3R1, PIK3R3, TERT |
| CCR5 Signaling in Macrophages | CACNG6, FOS, GNAI3, GNB1, MAPK13, |
| | MAPK8, PRKCH |
| PKCθ Signaling in T Lymphocytes | CACNG6, CAMK2B, FOS, HLA-DMA, KRAS, |
| | MAP3K3, MAPK8, PIK3R1, PIK3R3, PPP3R1 |
| Synaptogenesis Signaling Pathway | ADCY1, ADCY9, AP2M1, ARPC2, ARPC4, |
| | ARPC5, CAMK2B, CRKL, EFNB1, EFNB2, |
| | KALRN, KRAS, NSF, PIK3R1, PIK3R3, RAB5B, |
| | THBS4 |
| FcγRIIB Signaling in B Lymphocytes | CACNG6, KRAS, MAPK8, PDPK1, PIK3R1, |
| | PIK3R3 |
| Aldosterone Signaling in Epithelial Cells | DNAJB5, HSPA13, HSPB8, KRAS, PDPK1, |
| | PIK3R1, PIK3R3, PIP4K2C, PLCD3, PRKCH |
| Neurotrophin/TRK Signaling | FOS, KRAS, MAPK8, PDPK1, PIK3R1, PIK3R3 |
| PI3K Signaling in B Lymphocytes | CAMK2B, CBL, FOS, IL4R, KRAS, PDPK1, |
| | PIK3R1, PLCD3, PPP3R1 |
| cAMP-mediated signaling | ADCY1, ADCY9, AKAP12, AKAP6, CAMK2B, |
| | GNAI3, GRK2, PDE3A, PDE4D, PDE9A, |
| | PPP3R1, PTGER2, RGS2 |
| Cardiac β-adrenergic Signaling | ADCY1, ADCY9, AKAP12, AKAP6, GNB1, |
| | GRK2, PDE3A, PDE4D, PDE9A |
| Insulin Receptor Signaling | CBL, CRKL, INPPL1, KRAS, MAPK8, PDPK1, |
| | PIK3R1, PIK3R3, SYNJ2 |
| Cholecystokinin/Gastrin-mediated Signaling | FOS, KRAS, MAPK8, PRKCH, PTK2, PXN, |
| | RHOB, RHOV |
| Role of NANOG in Mammalian Embryonic Stem | BMP6, FZD8, KRAS, LIF, PIK3R1, PIK3R3, |
| Cell Pluripotency | WNT5A, WNT9A |
| Apoptosis Signaling | CASP2, CASP7, CASP9, KRAS, MAPK8, |
| | MCL1, TNFRSF1B |
| Oncostatin M Signaling | KRAS, MMP1, OSMR, PLAU |
| CREB Signaling in Neurons | ADCY1, ADCY9, CACNG6, CAMK2B, GNA15, |
| | GNAI3, GNB1, KRAS, PIK3R1, PIK3R3, |
| | PLCD3, PRKCH |
| PCP pathway | EFNB1, FZD8, MAPK8, WNT5A, WNT9A |
| Type II Diabetes Mellitus Signaling | CACNG6, MAPK8, PDPK1, PIK3R1, PIK3R3, |
| | PRKAA2, PRKCH, SMPD1, TNFRSF1B |
| IL-2 Signaling | FOS, KRAS, MAPK8, PIK3R1, PIK3R3 |
| G Beta Gamma Signaling | ADCY1, CACNG6, GNA15, GNAI3, GNB1, |
| | KRAS, PDPK1, PRKCH |
| Renal Cell Carcinoma Signaling | ETS1, FOS, KRAS, PAK4, PIK3R1, PIK3R3 |
| Production of Nitric Oxide and Reactive Oxygen | FOS, IRF1, MAP3K3, MAPK13, MAPK8, |
| Species in Macrophages | PIK3R1, PIK3R3, PRKCH, RHOB, RHOV, |
| | TNFRSF1B |
| Corticotropin Releasing Hormone Signaling | ADCY1, ADCY9, ARPC5, CACNG6, FOS, GLI2, |
| | GNAI3, MAPK13, PRKCH |
| Sumoylation Pathway | ETS1, FOS, MAPK8, RFC3, RHOB, RHOV, |
| | SENP1 |
| Cdc42 Signaling | ARPC2, ARPC4, ARPC5, CDC42EP2, FOS, |
| | HLA-DMA, MAPK13, MAPK8, PAK4, PARD6A |
| Thrombopoietin Signaling | FOS, KRAS, PIK3R1, PIK3R3, PRKCH |
| FLT3 Signaling in Hematopoietic Progenitor | CBL, KRAS, MAPK13, PDPK1, PIK3R1, PIK3R3 |
| Cells | |
| ErbB2-ErbB3 Signaling | CCND1, KRAS, PDPK1, PIK3R1, PIK3R3 |
| Wnt/β-catenin Signaling | APPL1, CCND1, FZD8, LEF1, SOX18, SOX6, |
| | SOX7, TGFBR3, WNT5A, WNT9A |
| CDK5 Signaling | ADCY1, ADCY9, EGR1, FOSB, KRAS, |
| | MAPK13, MAPK8 |
| Sirtuin Signaling Pathway | ABCA1, ACSS2, ATP5PB, CPS1, CPT1C, |
| | CXCL8, GABPA, GABPB1, HSF1, IDH2, LDHD, |
| | PRKAA2, TOMM20, TOMM34, TP73 |
| Type I Diabetes Mellitus Signaling | CASP9, HLA-DMA, IL1RAP, IRF1, MAPK13, |
| | MAPK8, TNFRSF1B |
| GPCR-Mediated Nutrient Sensing in | ADCY1, ADCY9, CACNG6, GNA15, GNAI3, |
| Enteroendocrine Cells | PLCD3, PRKCH |
| Role of NFAT in Regulation of the Immune | FOS, GNA15, GNAI3, GNB1, HLA-DMA, KRAS, |
| Response | ORAI1, PIK3R1, PIK3R3, PPP3R1 |
| Growth Hormone Signaling | FOS, PDPK1, PIK3R1, PIK3R3, PRKCH |
| Neuroinflammation Signaling Pathway | APH1B, CXCL8, FOS, HLA-DMA, HMGB1, |
| | HMOX1, ICAM1, IL6R, MAPK13, MAPK8, |
| | PIK3R1, PIK3R3, PPP3R1, PSEN1, TGFBR3 |
| NGF Signaling | KRAS, MAP3K3, MAPK8, PDPK1, PIK3R1, |
| | PIK3R3, SMPD1 |
| eNOS Signaling | ADCY1, ADCY9, CASP9, CCNA2, PDPK1, |
| | PIK3R1, PIK3R3, PRKAA2, PRKCH |
| CD27 Signaling in Lymphocytes | CASP9, FOS, MAP3K3, MAPK8 |
| Melanocyte Development and Pigmentation | ADCY1, ADCY9, KRAS, PIK3R1, PIK3R3, |
| Signaling | SH2B2 |
| GP6 Signaling Pathway | COL5A2, COL5A3, PDPK1, PIK3R1, PIK3R3, |
| | PRKCH, PTK2 |
| Amyotrophic Lateral Sclerosis Signaling | CASP7, CASP9, NEFH, PIK3R1, PIK3R3, |
| | RAB5B |
| GDNF Family Ligand-Receptor Interactions | FOS, KRAS, MAPK8, PIK3R1, PIK3R3 |
| Antiproliferative Role of Somatostatin | CDKN1A, GNB1, KRAS, PIK3R1, PIK3R3 |
| Receptor 2 | |
| VEGF Signaling | ACTB, KRAS, PIK3R1, PIK3R3, PTK2, PXN |
| Neuropathic Pain Signaling In Dorsal Horn | CAMK2B, FOS, PIK3R1, PIK3R3, PLCD3, |
| Neurons | PRKCH |
| Cyclins and Cell Cycle Regulation | CCNA2, CCND1, CDK2, CDK6, CDKN1A |
| JAK/Stat Signaling | CDKN1A, FOS, KRAS, PIK3R1, PIK3R3 |
| PEDF Signaling | CASP7, KRAS, MAPK13, PIK3R1, PIK3R3 |
| Synaptic Long Term Potentiation | ADCY1, CAMK2B, GNA15, KRAS, PLCD3, |
| | PPP3R1, PRKCH |
| Wnt/Ca+ pathway | FZD8, PLCD3, ROR1, WNT5A |
| Gα12/13 Signaling | F2RL1, KRAS, MAPK8, PIK3R1, PIK3R3, PTK2, |
| | PXN |
| Role of Pattern Recognition Receptors in | C5, CXCL8, LIF, MAPK8, PIK3R1, PIK3R3, |
| Recognition of Bacteria and Viruses | PRKCH, PTX3 |
| VEGF Family Ligand-Receptor Interactions | FOS, KRAS, PIK3R1, PIK3R3, PRKCH |
| NF-κB Signaling | FGFR2, KRAS, MAP3K3, MAPK8, PDGFRB, |
| | PIK3R1, PIK3R3, TGFBR3, TNFRSF1B |
| Phospholipase C Signaling | ADCY1, ADCY9, GNB1, HMOX1, KRAS, |
| | PLCD3, PLD1, PPP3R1, PRKCH, RHOB, |
| | RHOV, TGM2 |
| Dendritic Cell Maturation | COL5A3, HLA-DMA, ICAM1, MAPK13, MAPK8, |
| | PIK3R1, PIK3R3, PLCD3, TNFRSF1B |
| iCOS-iCOSL Signaling in T Helper Cells | CAMK2B, HLA-DMA, PDPK1, PIK3R1, PIK3R3, |
| | PPP3R1 |
| SPINK1 General Cancer Pathway | IL6R, KRAS, PIK3R1, PIK3R3 |
| Melatonin Signaling | CAMK2B, GNAI3, PLCD3, PRKCH |
| TGF-β Signaling | FOS, KRAS, MAPK13, MAPK8, SERPINE1 |
| TREM1 Signaling | CXCL3, CXCL8, ICAM1, IL1RL1 |
| T Cell Exhaustion Signaling Pathway | FOS, HLA-DMA, IL6R, KRAS, MAPK8, PIK3R1, |
| | PIK3R3, TGFBR3 |
| Macropinocytosis Signaling | KRAS, PIK3R1, PIK3R3, PRKCH |
| NER Pathway | COPS3, DDB1, HIST1H4H, POLE3, RFC3 |
| Regulation of elF4 and p70S6K Signaling | KRAS, MAPK13, PDPK1, PIK3R1, PIK3R3, |
| | RPS17, RPS21 |
| Gαs Signaling | ADCY1, ADCY9, GNB1, PTGER2, RGS2 |
| NF-κB Activation by Viruses | KRAS, PIK3R1, PIK3R3, PRKCH |
| BMP signaling pathway | BMP6, KRAS, MAPK13, MAPK8 |
| Sperm Motility | FGFR2, PDE4D, PDGFRB, PLCD3, PRKCH, |
| | PTK2, PTK7, ROR1 |
| Dopamine-DARPP32 Feedback in cAMP | ADCY1, ADCY9, GNAI3, PLCD3, PPP3R1, |
| Signaling | PRKCH |
| LPS/IL-1 Mediated Inhibition of RXR Function | ABCA1, ALDH1A3, ALDH9A1, CPT1C, IL1 RAP, |
| | IL1RL1, MAPK8, SMOX, TNFRSF1B |
| EIF2 Signaling | ACTB, CCND1, KRAS, PDPK1, PIK3R1, |
| | PIK3R3, RPS17, RPS21, TRIB3 |
| p38 MAPK Signaling | IL1RAP, IL1RL1, MAPK13, MAX, TNFRSF1B |
| Gαi Signaling | ADCY1, ADCY9, GNAI3, GNB1, KRAS |
| Synaptic Long Term Depression | CACNG6, GNA15, GNAI3, KRAS, PLCD3, |
| | PRKCH |

TABLE 15
HEP3B liver cancer upregulated pathways and associated genes

| Pathway Name | Gene |
|---|---|
| PTEN Signaling | BCAR1, CBL, CCND1, INPP5B, INPPL1, KRAS, |
| | MAGI3, PDGFRB, PDPK1, PIK3R1, PTK2, |
| | RPS6KB2, RRAS, SOS2, TGFBR2, TGFBR3 |
| PPARα/RXRα Activation | ABCA1, ADCY9, CREBBP, CYP2C8, EP300, |
| | KRAS, MAPK8, NOTUM, PLCD3, PPARGC1A, |
| | PRKAA2, RRAS, SMAD3, SOS2, TGFB2, |
| | TGFBR2, TGFBR3 |
| Sumoylation Pathway | CREBBP, EP300, ETS1, MAPK8, MDM2, MYB, |
| | RHOH, RND3, RNF4, SENP5 |
| RhoGDI Signaling | ACTB, ARHGEF12, ARPC2, ARPC5, CREBBP, |
| | DLC1, EP300, GNAI3, PAK4, PIP4K2C, |
| | PIP5K1A, RHOH, RND3 |
| Endocannabinoid Cancer Inhibition Pathway | ADCY9, CASP2, CASP7, CCND1, CREBBP, |
| | GNAI3, LEF1, PIK3R1, PRKAA2, PTK2, SMPD1 |
| Apelin Cardiac Fibroblast Signaling Pathway | CCN2, PRKAA2, SERPINE1, TGFB2 |
| HIPPO signaling | AJUBA, DLG1, FAT4, SMAD3, TEAD4, YAP1, |
| | YWHAZ |
| Regulation of Cellular Mechanics by Calpain | CCNA2, CCND1, CDK6, KRAS, PTK2, RRAS |
| Protease | |
| Cell Cycle: G1/S Checkpoint Regulation | CCND1, CDK6, MAX, MDM2, SMAD3, TGFB2 |
| GPCR-Mediated Integration of Enteroendocrine | ADCY9, GNAI3, NOTUM, PLCD3 |
| Signaling Exemplified by an L Cell | |

TABLE 16
HEP3B liver cancer aberrant pathways and associated genes

| Pathway Name | Gene |
|---|---|
| Molecular Mechanisms of Cancer | ADCY9, ARHGEF12, CASP7, CBL, CCND1, |
| | CDK6, CREBBP, EP300, FZD5, GNAI3, HHAT, |
| | KRAS, LEF1, MAPK8, MAX, MDM2, NF1, |
| | NOTCH1, PAK4, PIK3R1, PSEN1, PTK2, |
| | RHOH, RND3, RRAS, SMAD3, SOS2, TGFB2, |
| | TGFBR2 |
| Chronic Myeloid Leukemia Signaling | CCND1, CDK6, CRKL, KRAS, MDM2, PIK3R1, |
| | RRAS, SMAD3, SOS2, TGFB2, TGFBR2 |
| Germ Cell-Sertoli Cell Junction Signaling | ACTB, BCAR1, KRAS, MAPK8, PAK4, PDPK1, |
| | PIK3R1, PTK2, RHOH, RND3, RRAS, TGFB2, |
| | TGFBR2, TUBB4A |
| Antiproliferative Role of TOB in T Cell Signaling | CCNA2, SMAD3, TGFB2, TGFBR2, TWSG1 |
| Glucocorticoid Receptor Signaling | ACTB, CREBBP, DUSP1, EP300, KRAS, |
| | KRT17, KRT80, MAPK8, PBX1, PHF10, |
| | PIK3R1, PRKAA2, RRAS, SERPINE1, SMAD3, |
| | SMARCA2, SOS2, TAF4B, TGFB2, TGFBR2, |
| | TSC22D3 |
| Prostate Cancer Signaling | CCND1, CREBBP, KRAS, LEF1, MDM2, |
| | PDPK1, PIK3R1, RRAS, SOS2 |
| RAR Activation | ACTB, ADCY9, CREBBP, DUSP1, EP300, |
| | MAPK8, NR2F6, PDPK1, PHF10, PIK3R1, |
| | PPARGC1A, SMAD3, SMARCA2, TGFB2 |
| Regulation of the Epithelial-Mesenchymal | ETS1, FZD5, KRAS, LEF1, LOX, NOTCH1, |
| Transition Pathway | PDGFRB, PIK3R1, PSEN1, RRAS, SMAD3, |
| | SOS2, TGFB2, TGFBR2 |
| FAK Signaling | ACTB, BCAR1, KRAS, PAK4, PDPK1, PIK3R1, |
| | PTK2, RRAS, SOS2 |
| Hereditary Breast Cancer Signaling | ACTB, CCND1, CDK6, CREBBP, EP300, |
| | GADD45B, KRAS, PHF10, PIK3R1, RRAS, |
| | SMARCA2 |
| Acetate Conversion to Acetyl-CoA | ACSS2, ACSS3 |
| Epithelial Adherens Junction Signaling | ACTB, ARPC2, ARPC5, KRAS, LEF1, |
| | NOTCH1, RRAS, TGFB2, TGFBR2, TGFBR3, |
| | TUBB4A |
| Hypoxia Signaling in the Cardiovascular System | CREBBP, EDN1, EP300, MDM2, UBE2F, |
| | UBE2J1, UBE2L6 |
| Erythropoietin Signaling | CBL, KRAS, PDPK1, PIK3R1, RRAS, SOCS3, |
| | SOS2 |
| p53 Signaling | CCND1, EP300, GADD45B, MAPK8, MDM2, |
| | PIK3R1, TNFRSF10B, TP53INP1 |
| Axonal Guidance Signaling | ADAMTS1, ARHGEF12, ARPC2, ARPC5, |
| | BCAR1, CRKL, CXCR4, EFNA4, EFNB1, |
| | EFNB2, EPHA2, FZD5, GLIS2, GNAI3, KRAS, |
| | NOTUM, NRP2, PAK4, PIK3R1, PLCD3, PTK2, |
| | RRAS, SOS2, TUBB4A |
| HER-2 Signaling in Breast Cancer | CCND1, CDK6, KRAS, MDM2, PIK3R1, RRAS, |
| | SOS2 |
| Ephrin A Signaling | BCAR1, EFNA4, EPHA2, PIK3R1, PTK2 |
| Myc Mediated Apoptosis Signaling | KRAS, MAPK8, PIK3R1, RRAS, SOS2, YWHAZ |
| IL-4 Signaling | INPP5B, INPPL1, KRAS, PIK3R1, RPS6KB2, |
| | RRAS, SOS2 |
| Superpathway of Serine and Glycine | PHGDH, SHMT2 |
| Biosynthesis I | |
| Regulation of IL-2 Expression in Activated and | KRAS, MAPK8, RRAS, SMAD3, SOS2, TGFB2, |
| Anergic T Lymphocytes | TGFBR2 |
| Oxidative Ethanol Degradation III | ACSS2, ACSS3, ALDH9A1 |
| Factors Promoting Cardiogenesis in Vertebrates | CER1, FZD5, LEF1, NPPB, TGFB2, TGFBR2, |
| | TGFBR3 |
| HIF1α Signaling | CREBBP, EDN1, EP300, KRAS, MAPK8, |
| | MDM2, PIK3R1, RRAS |
| Estrogen Receptor Signaling | CREBBP, EP300, KRAS, MED21, PPARGC1A, |
| | RBFOX2, RRAS, SOS2, TAF4B |
| Sphingomyelin Metabolism | SGMS1, SMPD1 |
| Leptin Signaling in Obesity | ADCY9, NOTUM, PDE3A, PIK3R1, PLCD3, |
| | SOCS3 |
| Thio-molybdenum Cofactor Biosynthesis | MOCOS |
| Ethanol Degradation IV | ACSS2, ACSS3, ALDH9A1 |
| Semaphorin Signaling in Neurons | ARHGEF12, PAK4, PTK2, RHOH, RND3 |
| D-myo-inositol (1, 4, 5)-Trisphosphate | PIP4K2C, PIP5K1A, PLCD3 |
| Biosynthesis | |
| Role of JAK family kinases in IL-6-type Cytokine | IL6R, MAPK8, SOCS3 |
| Signaling | |
| T Cell Receptor Signaling | CBL, KRAS, MAPK8, PAG1, PIK3R1, RRAS, |
| | SOS2 |
| Apelin Liver Signaling Pathway | EDN1, MAPK8, PDGFRB |
| Role of Oct4 in Mammalian Embryonic Stem | IGF2BP1, NR2F6, NR6A1, SPP1 |
| Cell Pluripotency | |
| Human Embryonic Stem Cell Pluripotency | FZD5, LEF1, PDGFRB, PDPK1, PIK3R1, |
| | SMAD3, TGFB2, TGFBR2 |
| Iron homeostasis signaling pathway | ATP6V1B2, GDF15, HFE, HJV, IL6R, PDGFRB, |
| | SMAD3, TWSG1 |
| Remodeling of Epithelial Adherens Junctions | ACTB, ARPC2, ARPC5, RAB5B, TUBB4A |
| Glycine Biosynthesis I | SHMT2 |
| Guanine and Guanosine Salvage I | HPRT1 |
| L-cysteine Degradation III | MPST |
| Thyroid Cancer Signaling | CCND1, KRAS, LEF1, RRAS |
| Natural Killer Cell Signaling | INPP5B, INPPL1, KRAS, PAK4, PIK3R1, RRAS, |
| | SOS2 |
| Gap Junction Signaling | ACTB, ADCY9, GNAI3, KRAS, NOTUM, |
| | PIK3R1, PLCD3, RRAS, SOS2, TUBB4A |
| Th1 and Th2 Activation Pathway | CXCR4, IL6R, IRF1, NOTCH1, PIK3R1, PSEN1, |
| | SOCS3, TGFBR2, TGFBR3 |
| Apoptosis Signaling | CASP2, CASP7, KRAS, MAPK8, MCL1, RRAS |
| Lymphotoxin β Receptor Signaling | CREBBP, EP300, PDPK1, PIK3R1 |
| Reelin Signaling in Neurons | ARHGEF12, CDK5R1, CRKL, MAPK8, PIK3R1 |
| L-carnitine Biosynthesis | ALDH9A1 |
| Methylglyoxal Degradation I | GLO1 |
| N-acetylglucosamine Degradation I | GNPDA1 |
| S-adenosyl-L-methionine Biosynthesis | MAT1A |
| Tetrahydrobiopterin Biosynthesis I | GCH1 |
| Tetrahydrobiopterin Biosynthesis II | GCH1 |
| Thiosulfate Disproportionation III (Rhodanese) | MPST |
| Tyrosine Biosynthesis IV | PCBD1 |
| D-myo-inositol (1, 4, 5)-trisphosphate | INPP5B, INPPL1 |
| Degradation | |
| RAN Signaling | KPNA2, KPNA5 |
| Cancer Drug Resistance By Drug Efflux | KRAS, MDM2, PIK3R1, RRAS |
| Cellular Effects of Sildenafil (Viagra) | ACTB, ADCY9, MYLK, NOTUM, PDE3A, |
| | PDE4D, PLCD3 |
| 1D-myo-inositol Hexakisphosphate Biosynthesis | INPP5B, INPPL1 |
| II (Mammalian) | |
| D-myo-inositol (1, 3, 4)-trisphosphate | INPP5B, INPPL1 |
| Biosynthesis | |
| Virus Entry via Endocytic Pathways | ACTB, AP2M1, FLNC, KRAS, PIK3R1, RRAS |
| Hepatic Fibrosis/Hepatic Stellate Cell | CCN2, EDN1, IGFBP4, IL6R, PDGFRB, |
| Activation | SERPINE1, SMAD3, TGFB2, TGFBR2 |
| Apelin Muscle Signaling Pathway | PPARGC1A, PRKAA2 |
| GADD45 Signaling | CCND1, GADD45B |
| TR/RXR Activation | EP300, MDM2, PIK3R1, PPARGC1A, TBL1XR1 |
| G-Protein Coupled Receptor Signaling | ADCY9, CREBBP, DUSP1, GNAI3, KRAS, |
| | PDE3A, PDE4D, PDPK1, PIK3R1, RGS2, |
| | RRAS, SOS2 |
| Glutathione Redox Reactions II | GSR |
| N-acetylglucosamine Degradation II | GNPDA1 |
| Phenylalanine Degradation I (Aerobic) | PCBD1 |
| CDP-diacylglycerol Biosynthesis I | AGPAT1, CDS1 |
| Fatty Acid α-oxidation | ALDH9A1, BCO2 |
| Granzyme A Signaling | CREBBP, EP300 |
| Clathrin-mediated Endocytosis Signaling | ACTB, AP2M1, ARPC2, ARPC5, CBL, CD2AP, |
| | MDM2, PIK3R1, RAB5B |
| Ceramide Signaling | KRAS, MAPK8, PIK3R1, RRAS, SMPD1 |
| Endoplasmic Reticulum Stress Pathway | CASP7, MAPK8 |
| tRNA Splicing | PDE3A, PDE4D, TSEN2 |
| Serotonin Receptor Signaling | ADCY9, GCH1, PCBD1 |
| Phosphatidylglycerol Biosynthesis II (Non- | AGPAT1, CDS1 |
| plastidic) | |
| Polyamine Regulation in Colon Cancer | KRAS, MAX |
| Folate Polyglutamylation | SHMT2 |
| Serine Biosynthesis | PHGDH |
| dTMP De Novo Biosynthesis | SHMT2 |
| Breast Cancer Regulation by Stathmin1 | ADCY9, ARHGEF12, GNAI3, KRAS, PIK3R1, |
| | RRAS, SOS2, STMN1, TUBB4A |
| Superpathway of D-myo-inositol (1, 4, 5)- | INPP5B, INPPL1 |
| trisphosphate Metabolism | |
| Apelin Pancreas Signaling Pathway | MAPK8, PIK3R1, PRKAA2 |
| Stearate Biosynthesis I (Animals) | ACOT8, ACSL4, DHCR24 |
| GABA Receptor Signaling | ADCY9, ALDH9A1, AP2M1, NSF, SLC6A12 |
| Role of JAK1 and JAK3 in γc Cytokine Signaling | KRAS, PIK3R1, RRAS, SOCS3 |
| Glutathione Redox Reactions I | GPX2, GSR |
| IL-22 Signaling | MAPK8, SOCS3 |
| Tumoricidal Function of Hepatic Natural Killer | CASP7, M6PR |
| Cells | |
| ATM Signaling | CBX1, CREBBP, GADD45B, MAPK8, MDM2 |
| Adenine and Adenosine Salvage III | HPRT1 |
| Chondroitin and Dermatan Biosynthesis | CHSY1 |
| UDP-N-acetyl-D-glucosamine Biosynthesis II | GFPT1 |
| IL-15 Signaling | KRAS, PIK3R1, PTK2, RRAS |
| Cell Cycle: G2/M DNA Damage Checkpoint | EP300, MDM2, YWHAZ |
| Regulation | |
| Estrogen-mediated S-phase Entry | CCNA2, CCND1 |
| NAD Salvage Pathway II | NT5E, PXYLP1 |
| Sertoli Cell-Sertoli Cell Junction Signaling | ACTB, BCAR1, DLG1, KRAS, MAPK8, RRAS, |
| | TGFBR3, TUBB4A |
| Protein Ubiquitination Pathway | CBL, CRYAA/CRYAA2, DNAJA1, HSPA13, |
| | HSPB8, MDM2, UBE2F, UBE2J1, UBE2L6, |
| | USP39, USP53 |
| Superpathway of Cholesterol Biosynthesis | DHCR24, MVK |
| VDR/RXR Activation | EP300, HR, SPP1, TGFB2 |
| Salvage Pathways of Pyrimidine | TK2 |
| Deoxyribonucleotides | |
| IL-17 Signaling | KRAS, MAPK8, PIK3R1, RRAS |
| Role of Macrophages, Fibroblasts and | CCND1, CREBBP, FRZB, FZD5, IL6R, KRAS, |
| Endothelial Cells in Rheumatoid Arthritis | LEF1, NOTUM, PIK3R1, PLCD3, RRAS, |
| | SOCS3 |
| Role of p14/p19ARF in Tumor Suppression | MDM2, PIK3R1 |
| EGF Signaling | MAPK8, PIK3R1, SOS2 |
| PEDF Signaling | CASP7, KRAS, PIK3R1, RRAS |
| Folate Transformations I | SHMT2 |
| Leucine Degradation I | BCAT2 |
| Phosphatidylethanolamine Biosynthesis II | ETNK1 |
| UDP-N-acetyl-D-galactosamine Biosynthesis II | GNPDA1 |
| 4-1BB Signaling in T Lymphocytes | MAPK8, TNFSF9 |
| Fatty Acid β-oxidation I | ACSL4, SCP2 |
| Circadian Rhythm Signaling | CREBBP, GRIN2D |
| Glycine Betaine Degradation | SHMT2 |
| DNA Methylation and Transcriptional | MECP2, MTA2 |
| Repression Signaling | |
| IL-9 Signaling | PIK3R1, SOCS3 |
| Inhibition of Angiogenesis by TSP1 | MAPK8, TGFBR2 |
| Noradrenaline and Adrenaline Degradation | ADH1A, ALDH9A1 |
| Phagosome Maturation | ATP6V1B2, M6PR, NCF2, NSF, RAB5B, |
| | TUBB4A |
| IL-15 Production | EPHA2, ERBB4, IRF1, PDGFRB, PTK2 |
| Acetone Degradation I (to Methylglyoxal) | CYP2C8 |
| Dopamine Degradation | ALDH9A1 |
| Sonic Hedgehog Signaling | GLIS2 |
| TNFR2 Signaling | MAPK8 |
| Thrombopoietin Signaling | KRAS, PIK3R1, RRAS |
| CD40 Signaling | MAPK8, PIK3R1 |
| Complement System | C1QBP, C8A |
| Role of PI3K/AKT Signaling in the Pathogenesis | CRKL, GNAI3, PIK3R1 |
| of Influenza | |
| Cleavage and Polyadenylation of Pre-mRNA | NUDT21 |
| Guanosine Nucleotides Degradation III | NT5E |
| IL-17A Signaling in Airway Cells | MAPK8, PIK3R1 |
| Nicotine Degradation II | CYP2C8, UGT2B7 |
| PXR/RXR Activation | CYP2C8, PPARGC1A |
| Superpathway of Melatonin Degradation | CYP2C8, UGT2B7 |
| Notch Signaling | NOTCH1, PSEN1 |
| α-Adrenergic Signaling | ADCY9, GNAI3, KRAS, RRAS |
| Role of Pattern Recognition Receptors in | IL11, MAPK8, PIK3R1, PTX3, TGFB2, TNFSF9 |
| Recognition of Bacteria and Viruses | |
| Activation of IRF by Cytosolic Pattern | CREBBP, MAPK8 |
| Recognition Receptors | |
| Phagosome Formation | NOTUM, PIK3R1, PLCD3, RHOH, RND3 |
| Serotonin Degradation | ADH1A, ALDH9A1, UGT2B7 |
| Docosahexaenoic Acid (DHA) Signaling | PDPK1, PIK3R1 |
| Acyl-CoA Hydrolysis | ACOT8 |
| Bile Acid Biosynthesis, Neutral Pathway | SCP2 |
| Cholesterol Biosynthesis I | DHCR24 |
| Cholesterol Biosynthesis II (via 24, 25- | DHCR24 |
| dihydrolanosterol) | |
| Cholesterol Biosynthesis III (via Desmosterol) | DHCR24 |
| Fatty Acid Activation | ACSL4 |
| Mevalonate Pathway I | MVK |
| NAD Phosphorylation and Dephosphorylation | PXYLP1 |
| Urate Biosynthesis/Inosine 5′-phosphate | NT5E |
| Degradation | |
| Bladder Cancer Signaling | CCND1, KRAS, MDM2, RRAS |
| Induction of Apoptosis by HIV1 | CXCR4, MAPK8 |
| Phospholipases | NOTUM, PLCD3 |
| Lipid Antigen Presentation by CD1 | AP2M1 |
| Role of Osteoblasts, Osteoclasts and | CBL, FRZB, FZD5, IL11, LEF1, MAPK8, |
| Chondrocytes in Rheumatoid Arthritis | PIK3R1, SPP1 |
| Melatonin Degradation I | CYP2C8, UGT2B7 |
| Retinoic acid Mediated Apoptosis Signaling | IRF1, TNFRSF10B |
| SPINK1 Pancreatic Cancer Pathway | SMAD3, TGFBR2 |
| Bupropion Degradation | CYP2C8 |
| IL-17A Signaling in Gastric Cells | MAPK8 |
| Tryptophan Degradation X (Mammalian, via | ALDH9A1 |
| Tryptamine) | |
| MSP-RON Signaling Pathway | ACTB, PIK3R1 |
| Isoleucine Degradation I | BCAT2 |
| FXR/RXR Activation | APOH, CREBBP, MAPK8, PPARGC1A |
| eNOS Signaling | ADCY9, CCNA2, PDPK1, PIK3R1, PRKAA2 |
| Xenobiotic Metabolism Signaling | ALDH9A1, CREBBP, CYP2C8, EP300, KRAS, |
| | MAPK8, PIK3R1, PPARGC1A, RRAS, UGT2B7 |
| Cysteine Biosynthesis III (mammalia) | MAT1A |
| Pyrimidine Ribonucleotides Interconversion | ENTPD7, NUDT15 |
| IL-1 Signaling | ADCY9, GNAI3, MAPK8 |
| IL-12 Signaling and Production in Macrophages | EP300, IRF1, MAPK8, PIK3R1, TGFB2 |
| Melatonin Signaling | GNAI3, NOTUM, PLCD3 |
| Adenosine Nucleotides Degradation II | NT5E |
| NER Pathway | COPS3, EP300, POLD3, POLE3 |
| Oncostatin M Signaling | KRAS, RRAS |
| Nicotine Degradation III | CYP2C8, UGT2B7 |
| Basal Cell Carcinoma Signaling | FZD5, GLIS2, LEF1 |
| Caveolar-mediated Endocytosis Signaling | ACTB, FLNC, RAB5B |
| RANK Signaling in Osteoclasts | CBL, MAPK8, PIK3R1 |
| Role of IL-17A in Arthritis | MAPK8, PIK3R1 |
| Pyrimidine Ribonucleotides De Novo | ENTPD7, NUDT15 |
| Biosynthesis | |
| Role of RIG1-like Receptors in Antiviral Innate | CREBBP, EP300 |
| Immunity | |
| Methionine Degradation I (to Homocysteine) | MAT1A |
| Role of Tissue Factor in Cancer | CCN2, KRAS, PIK3R1, RRAS |
| Androgen Signaling | CCND1, CREBBP, EP300, GNAI3, SMAD3 |
| Systemic Lupus Erythematosus Signaling | C8A, CBL, IL6R, KRAS, LSM14B, PIK3R1, |
| | RRAS, SOS2 |
| Tight Junction Signaling | ACTB, MYLK, NSF, NUDT21, TGFB2, TGFBR2 |
| Parkinson's Signaling | MAPK8 |
| Vitamin-C Transport | SLC23A2 |
| Gustation Pathway | ADCY9, P2RY1, PDE3A, PDE4D, SCNN1A |
| IL-23 Signaling Pathway | PIK3R1, SOCS3 |
| Role of IL-17F in Allergic Inflammatory Airway | CREBBP, IL11 |
| Diseases | |
| iNOS Signaling | CREBBP, IRF1 |
| UVB-Induced MAPK Signaling | MAPK8, PIK3R1 |
| Putrescine Degradation III | ALDH9A1 |
| Antioxidant Action of Vitamin C | MAPK8, NOTUM, PLCD3, SLC23A2 |
| PD-1, PD-L1 cancer immunotherapy pathway | PIK3R1, SMAD3, TGFB2, YAP1 |
| Hepatic Cholestasis | ADCY9, ATP8B1, IL11, MAPK8, TGFB2, |
| | TNFSF9 |
| Relaxin Signaling | ADCY9, GNAI3, PDE3A, PDE4D, PIK3R1 |
| Dopamine Receptor Signaling | ADCY9, GCH1, PCBD1 |
| Macropinocytosis Signaling | KRAS, PIK3R1, RRAS |
| Dermatan Sulfate Degradation (Metazoa) | IDS |
| Histamine Degradation | ALDH9A1 |
| Mitochondrial L-carnitine Shuttle Pathway | ACSL4 |
| Superpathway of Geranylgeranyldiphosphate | MVK |
| Biosynthesis I (via Mevalonate) | |
| γ-linolenate Biosynthesis II (Animals) | ACSL4 |
| Cardiomyocyte Differentiation via BMP | NPPB |
| Receptors | |
| Antiproliferative Role of Somatostatin Receptor | KRAS, PIK3R1, RRAS |
| 2 | |
| Role of MAPK Signaling in the Pathogenesis of | KRAS, MAPK8, RRAS |
| Influenza | |
| Amyloid Processing | CDK5R1, PSEN1 |
| NF-κB Activation by Viruses | KRAS, PIK3R1, RRAS |
| FAT10 Signaling Pathway | SQSTM1 |
| Purine Nucleotides Degradation II (Aerobic) | NT5E |
| Valine Degradation I | BCAT2 |
| Role of BRCA1 in DNA Damage Response | ACTB, PHF10, SMARCA2 |
| Adipogenesis pathway | FZD5, SMAD3, TBL1XR1 |
| Agranulocyte Adhesion and Diapedesis | ACTB, CXCR4, GNAI3 |
| Altered T Cell and B Cell Signaling in | SPP1 |
| Rheumatoid Arthritis | |
| April Mediated Signaling | MAPK8 |
| Assembly of RNA Polymerase II Complex | TAF4B |
| Atherosclerosis Signaling | CXCR4 |
| Autophagy | SQSTM1 |
| B Cell Activating Factor Signaling | MAPK8 |
| BAG2 Signaling Pathway | MDM2 |
| CCR5 Signaling in Macrophages | GNAI3, MAPK8 |
| CD27 Signaling in Lymphocytes | MAPK8 |
| CTLA4 Signaling in Cytotoxic T Lymphocytes | AP2M1, PIK3R1 |
| Calcium Signaling | CREBBP, EP300, GRIN2D |
| Calcium-induced T Lymphocyte Apoptosis | EP300 |
| Cardiac β-adrenergic Signaling | ADCY9, AKAP12, PDE3A, PDE4D |
| Cell Cycle Control of Chromosomal Replication | CDK6 |
| Cell Cycle Regulation by BTG Family Proteins | CCND1 |
| Chondroitin Sulfate Biosynthesis | CHSY1 |
| Chondroitin Sulfate Biosynthesis (Late Stages) | CHSY1 |
| Coagulation System | SERPINE1 |
| Corticotropin Releasing Hormone Signaling | ADCY9, ARPC5, CREBBP, GNAI3 |
| Crosstalk between Dendritic Cells and Natural | ACTB |
| Killer Cells | |
| Cytotoxic T Lymphocyte-mediated Apoptosis of | CASP7 |
| Target Cells | |
| Dermatan Sulfate Biosynthesis | CHSY1 |
| Estrogen Biosynthesis | CYP2C8 |
| Glutamate Receptor Signaling | GRIN2D |
| Granulocyte Adhesion and Diapedesis | CXCR4, GNAI3 |
| Gαq Signaling | PIK3R1, RGS2, RHOH, RND3 |
| Gαs Signaling | ADCY9, CREBBP, RGS2 |
| Hematopoiesis from Pluripotent Stem Cells | IL11 |
| Heparan Sulfate Biosynthesis | NOTUM |
| Heparan Sulfate Biosynthesis (Late Stages) | NOTUM |
| IL-10 Signaling | MAPK8, SOCS3 |
| Inhibition of Matrix Metalloproteases | TIMP4 |
| Interferon Signaling | IRF1 |
| LXR/RXR Activation | ABCA1, APOH |
| MIF Regulation of Innate Immunity | MAPK8 |
| Mechanisms of Viral Exit from Host Cells | ACTB |
| Mitochondrial Dysfunction | ATP5PB, GSR, MAPK8, MT-CO3, PSEN1 |
| Neuroprotective Role of THOP1 in Alzheimer's | TPP1 |
| Disease | |
| Nitric Oxide Signaling in the Cardiovascular | PIK3R1 |
| System | |
| Nur77 Signaling in T Lymphocytes | EP300 |
| OX40 Signaling Pathway | MAPK8 |
| Oxidative Phosphorylation | ATP5PB, MT-CO3 |
| Primary Immunodeficiency Signaling | UNG |
| Role of JAK2 in Hormone-like Cytokine | SOCS3 |
| Signaling | |
| Role of PKR in Interferon Induction and Antiviral | IRF1 |
| Response | |
| Role of Wnt/GSK-3β Signaling in the | FZD5, LEF1 |
| Pathogenesis of Influenza | |
| Superpathway of Methionine Degradation | MAT1A |
| T Helper Cell Differentiation | IL6R, TGFBR2 |
| TWEAK Signaling | CASP7 |
| Th17 Activation Pathway | IL6R, SOCS3 |
| Thyroid Hormone Metabolism II (via Conjugation | UGT2B7 |
| and/or Degradation) | |
| Toll-like Receptor Signaling | MAPK8 |
| Transcriptional Regulatory Network in | FOXC1 |
| Embryonic Stem Cells | |
| Triacylglycerol Biosynthesis | AGPAT1 |
| Triacylglycerol Degradation | NOTUM |
| Type I Diabetes Mellitus Signaling | IRF1, MAPK8, SOCS3 |
| Unfolded protein response | MAPK8 |
| iCOS-iCOSL Signaling in T Helper Cells | PDPK1, PIK3R1 |
| nNOS Signaling in Neurons | GRIN2D |
| tRNA Charging | LARS2 |

TABLE 17
HEP3B liver cancer downregulated pathways and associated genes

| Pathway Name | Gene |
|---|---|
| Rac Signaling | ABI2, ARPC2, ARPC5, CDK5R1, ELK4, KRAS, |
| | MAPK8, NCF2, PAK4, PIK3R1, PIP4K2C, |
| | PIP5K1A, PTK2, RRAS |
| RhoA Signaling | ACTB, ARHGEF12, ARPC2, ARPC5, |
| | CDC42EP2, CDC42EP3, CDC42EP4, DLC1, |
| | MYLK, NRP2, PIP4K2C, PIP5K1A, PTK2, RND3 |
| Neuregulin Signaling | CDK5R1, CRKL, ERBB4, ERBIN, GRB7, KRAS, |
| | PDPK1, PIK3R1, PSEN1, RPS6KB2, RRAS, |
| | SOS2 |
| Ephrin Receptor Signaling | ARPC2, ARPC5, BCAR1, CREBBP, CRKL, |
| | CXCR4, EFNA4, EFNB1, EFNB2, EPHA2, |
| | GNAI3, GRIN2D, KRAS, PAK4, PTK2, RRAS, |
| | SOS2 |
| IGF-1 Signaling | CCN2, IGFBP4, KRAS, MAPK8, PDPK1, |
| | PIK3R1, PTK2, RPS6KB2, RRAS, SOCS3, |
| | SOS2, YWHAZ |
| Integrin Signaling | ACTB, ARF3, ARPC2, ARPC5, BCAR1, CRKL, |
| | GRB7, KRAS, MAPK8, MYLK, NEDD9, PAK4, |
| | PIK3R1, PTK2, RHOH, RND3, RRAS, SOS2 |
| Agrin Interactions at Neuromuscular Junction | ACTB, ERBB4, GABPA, GABPB1, KRAS, |
| | LAMC1, MAPK8, PAK4, PTK2, RRAS |
| Signaling by Rho Family GTPases | ACTB, ARHGEF12, ARPC2, ARPC5, |
| | CDC42EP2, CDC42EP3, CDC42EP4, GNAI3, |
| | MAPK8, MYLK, NCF2, PAK4, PIK3R1, |
| | PIP4K2C, PIP5K1A, PTK2, RHOH, RND3, |
| | STMN1 |
| Glioblastoma Multiforme Signaling | CCND1, CDK6, FZD5, KRAS, LEF1, MDM2, |
| | NF1, NOTUM, PDGFRB, PIK3R1, PLCD3, |
| | RHOH, RND3, RRAS, SOS2 |
| PI3K/AKT Signaling | CCND1, GDF15, INPP5B, INPPL1, KRAS, |
| | MCL1, MDM2, PDPK1, PIK3R1, RPS6KB2, |
| | RRAS, SOS2, YWHAZ |
| Sphingosine-1-phosphate Signaling | ADCY9, CASP2, CASP7, GNAI3, NOTUM, |
| | PDGFRB, PIK3R1, PLCD3, PTK2, RHOH, |
| | RND3, SMPD1 |
| Insulin Receptor Signaling | CBL, CRKL, INPP5B, INPPL1, KRAS, MAPK8, |
| | PDPK1, PIK3R1, RPS6KB2, RRAS, SCNN1A, |
| | SOCS3, SOS2 |
| Aldosterone Signaling in Epithelial Cells | CRYAA/CRYAA2, DNAJA1, DUSP1, HSPA13, |
| | HSPB8, KRAS, NOTUM, PDPK1, PIK3R1, |
| | PIP4K2C, PIP5K1A, PLCD3, SCNN1A, SOS2 |
| TGF-β Signaling | CREBBP, EP300, KRAS, MAPK8, RRAS, |
| | SERPINE1, SMAD3, SOS2, TGFB2, TGFBR2 |
| Prolactin Signaling | CREBBP, EP300, IRF1, KRAS, PDPK1, |
| | PIK3R1, RRAS, SOCS3, SOS2 |
| Superpathway of Inositol Phosphate | DUSP1, DUSP16, HACD2, INPP5B, INPPL1, |
| Compounds | NUDT15, NUDT4, PIK3R1, PIP4K2C, PIP5K1A, |
| | PLCD3, PPM1K, PPTC7, PXYLP1, SOCS3 |
| ErbB4 Signaling | ERBB4, KRAS, PDPK1, PIK3R1, PSEN1, |
| | RRAS, SOS2, YAP1 |
| PDGF Signaling | CRKL, INPP5B, INPPL1, KRAS, MAPK8, |
| | PDGFRB, PIK3R1, RRAS, SOS2 |
| 3-phosphoinositide Biosynthesis | DUSP1, DUSP16, HACD2, INPP5B, NUDT15, |
| | NUDT4, PIK3R1, PIP4K2C, PIP5K1A, PPM1K, |
| | PPTC7, PXYLP1, SOCS3 |
| Acute Myeloid Leukemia Signaling | CCND1, IDH1, IDH2, KRAS, LEF1, PIK3R1, |
| | RPS6KB2, RRAS, SOS2 |
| CXCR4 Signaling | ADCY9, BCAR1, CXCR4, ELMO2, GNAI3, |
| | KRAS, MAPK8, PAK4, PIK3R1, PTK2, RHOH, |
| | RND3, RRAS |
| Glioma Signaling | CCND1, CDK6, IDH1, IDH2, KRAS, MDM2, |
| | PDGFRB, PIK3R1, RRAS, SOS2 |
| HGF Signaling | CCND1, CRKL, ELF4, ETS1, KRAS, MAPK8, |
| | PIK3R1, PTK2, RRAS, SOS2 |
| Regulation of Actin-based Motility by Rho | ACTB, ARPC2, ARPC5, MYLK, PAK4, |
| | PIP4K2C, PIP5K1A, RHOH, RND3 |
| ERK/MAPK Signaling | BCAR1, CREBBP, CRKL, DUSP1, ELF4, ETS1, |
| | KRAS, MYCN, PAK4, PIK3R1, PTK2, RRAS, |
| | SOS2, YWHAZ |
| Wnt/β-catenin Signaling | APPL1, CCND1, CREBBP, EP300, FRZB, |
| | FZD5, LEF1, MDM2, SOX5, TGFB2, TGFBR2, |
| | TGFBR3, TLE4 |
| Endometrial Cancer Signaling | CCND1, KRAS, LEF1, PDPK1, PIK3R1, RRAS, |
| | SOS2 |
| Actin Cytoskeleton Signaling | ABI2, ACTB, ARHGEF12, ARPC2, ARPC5, |
| | BCAR1, CRKL, KRAS, MYLK, PAK4, PIK3R1, |
| | PIP5K1A, PTK2, RRAS, SOS2 |
| D-myo-inositol-5-phosphate Metabolism | DUSP1, DUSP16, HACD2, INPP5B, NUDT15, |
| | NUDT4, PIP4K2C, PLCD3, PPM1K, PPTC7, |
| | PXYLP1, SOCS3 |
| PAK Signaling | KRAS, MAPK8, MYLK, PAK4, PDGFRB, |
| | PIK3R1, PTK2, RRAS, SOS2 |
| Renal Cell Carcinoma Signaling | CREBBP, EP300, ETS1, KRAS, PAK4, PIK3R1, |
| | RRAS, SOS2 |
| B Cell Receptor Signaling | CREBBP, ETS1, INPP5B, INPPL1, KRAS, |
| | MAPK8, PAG1, PDPK1, PIK3R1, PTK2, |
| | RPS6KB2, RRAS, SOS2 |
| Colorectal Cancer Metastasis Signaling | ADCY9, APPL1, CCND1, FZD5, IL6R, KRAS, |
| | LEF1, MAPK8, PIK3R1, RHOH, RND3, RRAS, |
| | SMAD3, SOS2, TGFB2, TGFBR2 |
| Protein Kinase A Signaling | ADCY9, AKAP12, CDC14B, CREBBP, DUSP1, |
| | DUSP16, FLNC, GNAI3, HHAT, LEF1, MYLK, |
| | NOTUM, PDE3A, PDE4D, PLCD3, PTK2, |
| | PTPN9, PTPRA, SMAD3, TGFB2, TGFBR2, |
| | YWHAZ |
| AMPK Signaling | ACTB, CAB39, CCNA2, CCND1, CREBBP, |
| | EP300, PDPK1, PFKFB4, PHF10, PIK3R1, |
| | PPARGC1A, PPM1K, PRKAA2, SMARCA2 |
| 14-3-3-mediated Signaling | CBL, KRAS, MAPK8, NOTUM, PIK3R1, PLCD3, |
| | RRAS, TUBB4A, YAP1, YWHAZ |
| Actin Nucleation by ARP-WASP Complex | ARPC2, ARPC5, KRAS, RHOH, RND3, RRAS, |
| | SOS2 |
| Pancreatic Adenocarcinoma Signaling | CCND1, KRAS, MAPK8, MDM2, NOTCH1, |
| | PIK3R1, SMAD3, TGFB2, TGFBR2 |
| Paxillin Signaling | ACTB, BCAR1, KRAS, MAPK8, PAK4, PIK3R1, |
| | PTK2, RRAS, SOS2 |
| Cardiac Hypertrophy Signaling | ADCY9, CREBBP, EP300, GNAI3, IL6R, KRAS, |
| | MAPK8, NOTUM, PIK3R1, PLCD3, RHOH, |
| | RND3, RRAS, TGFB2, TGFBR2 |
| Synaptogenesis Signaling Pathway | ADCY9, AP2M1, ARPC2, ARPC5, CREBBP, |
| | CRKL, EFNA4, EFNB1, EFNB2, EPHA2, |
| | GRIN2D, KRAS, NSF, PIK3R1, RAB5B, |
| | RPS6KB2, RRAS, SOS2 |
| Non-Small Cell Lung Cancer Signaling | CCND1, CDK6, KRAS, PDPK1, PIK3R1, RRAS, |
| | SOS2 |
| 3-phosphoinositide Degradation | DUSP1, DUSP16, HACD2, INPP5B, INPPL1, |
| | NUDT15, NUDT4, PPM1K, PPTC7, PXYLP1, |
| | SOCS3 |
| Glioma Invasiveness Signaling | KRAS, PIK3R1, PTK2, RHOH, RND3, RRAS, |
| | TIMP4 |
| STAT3 Pathway | IL17RD, IL6R, KRAS, MAPK8, PDGFRB, |
| | RRAS, SOCS3, TGFB2, TGFBR2, TGFBR3 |
| NGF Signaling | CREBBP, KRAS, MAPK8, PDPK1, PIK3R1, |
| | RPS6KB2, RRAS, SMPD1, SOS2 |
| ErbB Signaling | ERBB4, KRAS, MAPK8, PAK4, PDPK1, |
| | PIK3R1, RRAS, SOS2 |
| Melanocyte Development and Pigmentation | ADCY9, CREBBP, EP300, KRAS, PIK3R1, |
| Signaling | RPS6KB2, RRAS, SOS2 |
| GDNF Family Ligand-Receptor Interactions | DOK7, GFRA3, KRAS, MAPK8, PIK3R1, RRAS, |
| | SOS2 |
| Neurotrophin/TRK Signaling | CREBBP, KRAS, MAPK8, PDPK1, PIK3R1, |
| | RRAS, SOS2 |
| Apelin Endothelial Signaling Pathway | ADCY9, GNAI3, KRAS, MAPK8, PIK3R1, |
| | PRKAA2, RPS6KB2, RRAS, SMAD3 |
| UVA-lnduced MAPK Signaling | KRAS, MAPK8, NOTUM, PIK3R1, PLCD3, |
| | RPS6KB2, RRAS, SMPD1 |
| D-myo-inositol (1,4,5,6)-Tetrakisphosphate | DUSP1, DUSP16, HACD2, INPP5B, NUDT15, |
| Biosynthesis | NUDT4, PPM1K, PPTC7, PXYLP1, SOCS3 |
| D-myo-inositol (3,4,5,6)-tetrakisphosphate | DUSP1, DUSP16, HACD2, INPP5B, NUDT15, |
| Biosynthesis | NUDT4, PPM1K, PPTC7, PXYLP1, SOCS3 |
| Thrombin Signaling | ADCY9, ARHGEF12, GNAI3, KRAS, MYLK, |
| | NOTUM, PDPK1, PIK3R1, PLCD3, PTK2, |
| | RHOH, RND3, RRAS |
| Apelin Adipocyte Signaling Pathway | ADCY9, GNAI3, GPX2, MAPK8, NCF2, |
| | PPARGC1A, PRKAA2 |
| Cardiac Hypertrophy Signaling (Enhanced) | ADCY9, DLG1, EDN1, EP300, FZD5, GNAI3, |
| | IL11, IL17RD, IL6R, KRAS, MAPK8, NOTUM, |
| | NPPB, PDE3A, PDE4D, PIK3R1, PLCD3, PTK2, |
| | RPS6KB2, RRAS, TGFB2, TGFBR2, TGFBR3, |
| | TNFSF9 |
| FAT10 Cancer Signaling Pathway | CXCR4, SMAD3, TGFB2, TGFBR2, TGFBR3 |
| Osteoarthritis Pathway | CASP2, CASP7, CREBBP, FRZB, FZD5, |
| | GLIS2, LEF1, NOTCH1, PPARGC1A, PRKAA2, |
| | SMAD3, SPP1, TGFBR2 |
| SAPK/JNK Signaling | CRKL, KRAS, MAP4K5, MAPK8, MINK1, |
| | PIK3R1, RRAS, SOS2 |
| NRF2-mediated Oxidative Stress Response | ACTB, CREBBP, DNAJA1, EP300, GPX2, GSR, |
| | HSPB8, KRAS, MAPK8, PIK3R1, RRAS, |
| | SQSTM1 |
| FLT3 Signaling in Hematopoietic Progenitor | CBL, CREBBP, KRAS, PDPK1, PIK3R1, RRAS, |
| Cells | SOS2 |
| Mouse Embryonic Stem Cell Pluripotency | CREBBP, FZD5, ID3, KRAS, LEF1, PIK3R1, |
| | RRAS, SOS2 |
| Role of NFAT in Cardiac Hypertrophy | ADCY9, EP300, GNAI3, IL11, KRAS, MAPK8, |
| | NOTUM, PIK3R1, PLCD3, RRAS, SOS2, |
| | TGFB2, TGFBR2 |
| ErbB2-ErbB3 Signaling | CCND1, KRAS, PDPK1, PIK3R1, RRAS, SOS2 |
| Ethanol Degradation II | ACSS2, ACSS3, ADH1A, ALDH9A1 |
| P2Y Purigenic Receptor Signaling Pathway | ADCY9, CREBBP, GNAI3, KRAS, NOTUM, |
| | P2RY1, PIK3R1, PLCD3, RRAS |
| Telomerase Signaling | ELF4, ETS1, KRAS, PDPK1, PIK3R1, RRAS, |
| | SOS2, TPP1 |
| p70S6K Signaling | GNAI3, KRAS, NOTUM, PDPK1, PIK3R1, |
| | PLCD3, RRAS, SOS2, YWHAZ |
| Leukocyte Extravasation Signaling | ACTB, BCAR1, CRKL, CXCR4, DLC1, GNAI3, |
| | MAPK8, NCF2, PIK3R1, PTK2, RHOH, TIMP4 |
| Melanoma Signaling | CCND1, KRAS, MDM2, PIK3R1, RRAS |
| GM-CSF Signaling | CCND1, ETS1, KRAS, PIK3R1, RRAS, SOS2 |
| ERK5 Signaling | CREBBP, ELK4, KRAS, RPS6KB2, RRAS, |
| | YWHAZ |
| Ephrin B Signaling | CBL, CXCR4, EFNB1, EFNB2, GNAI3, PTK2 |
| Fcγ Receptor-mediated Phagocytosis in | ACTB, ARPC2, ARPC5, CBL, PIK3R1, |
| Macrophages and Monocytes | PIP5K1A, RPS6KB2 |
| Endocannabinoid Developing Neuron Pathway | ADCY9, CCND1, CREBBP, GNAI3, KRAS, |
| | MAPK8, PIK3R1, RRAS |
| Endothelin-1 Signaling | ADCY9, CASP2, CASP7, EDN1, GNAI3, KRAS, |
| | MAPK8, NOTUM, PIK3R1, PLCD3, RRAS |
| Fc Epsilon Rl Signaling | INPP5B, INPPL1, KRAS, MAPK8, PDPK1, |
| | PIK3R1, RRAS, SOS2 |
| Angiopoietin Signaling | GRB7, KRAS, PAK4, PIK3R1, PTK2, RRAS |
| Tec Kinase Signaling | ACTB, GNAI3, MAPK8, PAK4, PIK3R1, PTK2, |
| | RHOH, RND3, TNFRSF10B, TNFRSF21 |
| Cholecystokinin/Gastrin-mediated Signaling | BCAR1, KRAS, MAPK8, PTK2, RHOH, RND3, |
| | RRAS, SOS2 |
| Renin-Angiotensin Signaling | ADCY9, KRAS, MAPK8, PAK4, PIK3R1, PTK2, |
| | RRAS, SOS2 |
| HMGB1 Signaling | IL11, KRAS, MAPK8, PIK3R1, RHOH, RND3, |
| | RRAS, SERPINE1, TGFB2, TNFSF9 |
| Aryl Hydrocarbon Receptor Signaling | ALDH9A1, CCNA2, CCND1, CDK6, EP300, |
| | MAPK8, MDM2, TGFB2, TGM2 |
| ILK Signaling | ACTB, CCND1, CREBBP, FLNC, LEF1, |
| | MAPK8, PDPK1, PIK3R1, PTK2, RHOH, RND3 |
| IL-7 Signaling Pathway | CCND1, MCL1, PDPK1, PIK3R1, PTK2, SOS2 |
| Chemokine Signaling | CXCR4, GNAI3, KRAS, MAPK8, PTK2, RRAS |
| IL-6 Signaling | IL6R, KRAS, MAPK8, MCL1, PIK3R1, RRAS, |
| | SOCS3, SOS2 |
| Adrenomedullin signaling pathway | ADCY9, KRAS, MAPK8, MAX, MYLK, NOTUM, |
| | PIK3R1, PLCD3, PTK2, RRAS, SOS2 |
| PPAR Signaling | CREBBP, EP300, KRAS, PDGFRB, |
| | PPARGC1A, RRAS, SOS2 |
| IL-2 Signaling | KRAS, MAPK8, PIK3R1, RRAS, SOS2 |
| Acute Phase Response Signaling | APOH, IL6R, KRAS, MAPK8, PDPK1, PIK3R1, |
| | RRAS, SERPINE1, SOCS3, SOS2 |
| CDK5 Signaling | ADCY9, CABLES1, CDK5R1, KRAS, LAMC1, |
| | MAPK8, RRAS |
| Pyridoxal 5′-phosphate Salvage Pathway | CDK6, FAM20B, MAPK8, PDXK, PRKAA2 |
| PFKFB4 Signaling Pathway | CREBBP, HK2, PFKFB4, TGFB2 |
| CREB Signaling in Neurons | ADCY9, CREBBP, EP300, GNAI3, GRIN2D, |
| | KRAS, NOTUM, PIK3R1, PLCD3, RRAS, SOS2 |
| Huntington's Disease Signaling | ATP5PB, CASP2, CASP7, CDK5R1, CREBBP, |
| | EP300, MAPK8, NSF, PDPK1, PIK3R1, SOS2, |
| | TGM2 |
| Death Receptor Signaling | ACTB, CASP2, CASP7, MAPK8, TNFRSF10B, |
| | TNFRSF21 |
| Ovarian Cancer Signaling | CCND1, EDN1, FZD5, KRAS, LEF1, PIK3R1, |
| | RPS6KB2, RRAS |
| fMLP Signaling in Neutrophils | ARPC2, ARPC5, GNAI3, KRAS, NCF2, PIK3R1, |
| | RRAS |
| TNFR1 Signaling | CASP2, CASP7, MAPK8, PAK4 |
| Small Cell Lung Cancer Signaling | CCND1, CDK6, MAX, PIK3R1, PTK2 |
| UVC-Induced MAPK Signaling | KRAS, MAPK8, RRAS, SMPD1 |
| GNRH Signaling | ADCY9, CREBBP, GNAI3, KRAS, MAPK8, |
| | PAK4, PTK2, RRAS, SOS2 |
| Estrogen-Dependent Breast Cancer Signaling | CCND1, CREBBP, KRAS, PIK3R1, RRAS |
| IL-8 Signaling | CCND1, GNAI3, KRAS, MAPK8, NCF2, |
| | PIK3R1, PTK2, RHOH, RND3, RRAS |
| FcγRIIB Signaling in B Lymphocytes | KRAS, MAPK8, PDPK1, PIK3R1, RRAS |
| Apelin Cardiomyocyte Signaling Pathway | GNAI3, MAPK8, MYLK, NOTUM, PIK3R1, |
| | PLCD3 |
| VEGF Signaling | ACTB, KRAS, PIK3R1, PTK2, RRAS, SOS2 |
| Synaptic Long Term Potentiation | CREBBP, EP300, GRIN2D, KRAS, NOTUM, |
| | PLCD3, RRAS |
| NF-κB Signaling | CREBBP, EP300, KRAS, MAPK8, PDGFRB, |
| | PIK3R1, RRAS, TGFBR2, TGFBR3 |
| JAK/Stat Signaling | KRAS, PIK3R1, RRAS, SOCS3, SOS2 |
| Sirtuin Signaling Pathway | ABCA1, ACSS2, ATP5PB, GABARAPL1, |
| | GABPA, GABPB1, GADD45B, IDH2, MYCN, |
| | PPARGC1A, PRKAA2, SCNN1A, TOMM20 |
| mTOR Signaling | EIF4B, KRAS, PDPK1, PIK3R1, PRKAA2, |
| | RHOH, RND3, RPS17, RPS6KB2, RRAS |
| CNTF Signaling | KRAS, PIK3R1, RPS6KB2, RRAS |
| PCP pathway | EFNB1, FZD5, LGR4, MAPK8 |
| FGF Signaling | CREBBP, CRKL, MAPK8, PIK3R1, SOS2 |
| VEGF Family Ligand-Receptor Interactions | KRAS, NRP2, PIK3R1, RRAS, SOS2 |
| Wnt/Ca+ pathway | CREBBP, FZD5, NOTUM, PLCD3 |
| PI3K Signaling in B Lymphocytes | CBL, KRAS, NOTUM, PDPK1, PIK3R1, PLCD3, |
| | RRAS |
| Th2 Pathway | CXCR4, NOTCH1, PIK3R1, PSEN1, SOCS3, |
| | TGFBR2, TGFBR3 |
| EIF2 Signaling | ACTB, CCND1, KRAS, MYCN, PDPK1, PIK3R1, |
| | RPL27A, RPS17, RRAS, SOS2 |
| Type II Diabetes Mellitus Signaling | ACSL4, MAPK8, PDPK1, PIK3R1, PRKAA2, |
| | SMPD1, SOCS3 |
| p38 MAPK Signaling | CREBBP, DUSP1, MAX, RPS6KB2, TGFB2, |
| | TGFBR2 |
| Phospholipase C Signaling | ADCY9, ARHGEF12, CREBBP, EP300, KRAS, |
| | PLCD3, RHOH, RND3, RRAS, SOS2, TGM2 |
| T Cell Exhaustion Signaling Pathway | IL6R, KRAS, MAPK8, PIK3R1, RRAS, SMAD3, |
| | TGFBR2, TGFBR3 |
| SPINK1 General Cancer Pathway | IL6R, KRAS, PIK3R1, RRAS |
| Th1 Pathway | IL6R, IRF1, NOTCH1, PIK3R1, PSEN1, SOCS3 |
| Growth Hormone Signaling | PDPK1, PIK3R1, RPS6KB2, SOCS3 |
| CCR3 Signaling in Eosinophils | GNAI3, KRAS, MYLK, PAK4, PIK3R1, RRAS |
| Endocannabinoid Neuronal Synapse Pathway | ADCY9, GNAI3, GRIN2D, MAPK8, NOTUM, |
| | PLCD3 |
| Systemic Lupus Erythematosus In T Cell | CASP2, CASP7, CBL, CREBBP, GNAI3, KRAS, |
| Signaling Pathway | PIK3R1, PTK2, RHOH, RND3, RPS6KB2, |
| | RRAS, SOS2 |
| Opioid Signaling Pathway | ADCY9, AP2M1, CREBBP, EP300, GNAI3, |
| | GRIN2D, KRAS, RPS6KB2, RRAS, SOS2 |
| IL-3 Signaling | CRKL, KRAS, PIK3R1, RRAS |
| Cyclins and Cell Cycle Regulation | CCNA2, CCND1, CDK6, TGFB2 |
| LPS-stimulated MAPK Signaling | KRAS, MAPK8, PIK3R1, RRAS |
| BMP signaling pathway | CREBBP, KRAS, MAPK8, RRAS |
| Neuroinflammation Signaling Pathway | CREBBP, GRIN2D, IL6R, MAPK8, NCF2, |
| | PIK3R1, PSEN1, SLC6A12, TGFB2, TGFBR2, |
| | TGFBR3 |
| GP6 Signaling Pathway | LAMC1, LAMC2, PDPK1, PIK3R1, PTK2 |
| Role of NANOG in Mammalian Embryonic Stem | FZD5, KRAS, PIK3R1, RRAS, SOS2 |
| Cell Pluripotency | |
| CD28 Signaling in T Helper Cells | ARPC2, ARPC5, MAPK8, PDPK1, PIK3R1 |
| G Beta Gamma Signaling | GNAI3, KRAS, PDPK1, RRAS, SOS2 |
| Gai Signaling | ADCY9, GNAI3, KRAS, RRAS, SOS2 |
| Salvage Pathways of Pyrimidine | CDK6, FAM20B, MAPK8, PRKAA2 |
| Ribonucleotides | |
| Regulation of eIF4 and p70S6K Signaling | KRAS, PDPK1, PIK3R1, RPS17, RRAS, SOS2 |
| Production of Nitric Oxide and Reactive Oxygen | CREBBP, IRF1, MAPK8, NCF2, PIK3R1, |
| Species in Macrophages | RHOH, RND3 |
| Amyotrophic Lateral Sclerosis Signaling | CASP7, GRIN2D, PIK3R1, RAB5B |
| Sperm Motility | EPHA2, ERBB4, NOTUM, NPPB, PDE4D, |
| | PDGFRB, PLCD3, PTK2 |
| Gα12/13 Signaling | KRAS, MAPK8, PIK3R1, PTK2, RRAS |
| Dopamine-DARPP32 Feedback in cAMP | ADCY9, CREBBP, GNAI3, GRIN2D, NOTUM, |
| Signaling | PLCD3 |
| Neuropathic Pain Signaling In Dorsal Horn | GRIN2D, NOTUM, PIK3R1, PLCD3 |
| Neurons | |
| cAMP-mediated signaling | ADCY9, AKAP12, CREBBP, DUSP1, GNAI3, |
| | PDE3A, PDE4D, RGS2 |
| PKCθ Signaling in T Lymphocytes | KRAS, MAPK8, PIK3R1, RRAS, SOS2 |
| Cdc42 Signaling | ARPC2, ARPC5, CDC42EP2, MAPK8, MYLK, |
| | PAK4 |
| GPCR-Mediated Nutrient Sensing in | ADCY9, GNAI3, NOTUM, PLCD3 |
| Enteroendocrine Cells | |
| Dendritic Cell Maturation | CREBBP, MAPK8, NOTUM, PIK3R1, PLCD3 |
| LPS/IL-1 Mediated Inhibition of RXR Function | ABCA1, ACSL4, ALDH9A1, CYP2C8, MAPK8, |
| | PPARGC1A |
| Role of NFAT in Regulation of the Immune | GNAI3, KRAS, PIK3R1, RRAS, SOS2 |
| Response | |
| Synaptic Long Term Depression | GNAI3, KRAS, NOTUM, PLCD3, RRAS |

TABLE 18
HUH7 liver cancer upregulated pathways and associated genes

| Pathway Name | Gene |
|---|---|
| Role of CHK Proteins in Cell Cycle Checkpoint | BRCA1, CDC25A, CDK1, CDK2, CHEK1, |
| Control | CLSPN, E2F1, E2F2, E2F7, E2F8, MDC1, |
| | PCNA, PLK1, PPP2R2B, RAD17, RFC2, RFC3 |
| Cell Cycle: G1/S Checkpoint Regulation | CCND1, CCNE2, CDC25A, CDK2, CDK6, |
| | CDKN2C, E2F1, E2F2, E2F7, E2F8, MAX, |
| | MDM2, RBL1, TFDP1 |
| Cell Cycle: G2/M DNA Damage Checkpoint | BRCA1, CCNB1, CDK1, CHEK1, MDM2, |
| Regulation | PKMYT1, PLK1, TOP2A, YWHAZ |
| PTEN Signaling | CASP9, CCND1, INPPL1, KRAS, MAGI3, |
| | PDGFRB, PDPK1, PTK2, RAC2, RPS6KB2, |
| | SOS2, TGFBR3 |
| Sumoylation Pathway | ETS1, MAPK8, MDM2, MYB, PCNA, RAC2, |
| | RCC1, RFC2, RFC3 |
| Endocannabinoid Cancer Inhibition Pathway | ADCY9, ATF3, CASP2, CASP9, CCND1, |
| | CCNE2, LEF1, PTK2, SMPD1, TCF4 |
| PPAR Signaling | KRAS, NR0B2, PDGFRB, SOS2 |
| AMPK Signaling | CCNA2, CCND1, CHRNA5, PDPK1, PFKP, |
| | PHLPP2, PPAT, PPP2R2B |
| RhoGDI Signaling | ARHGEF12, ARHGEF5, ARPC2, ARPC5, |
| | PAK4, PIP4K2C, RAC2 |
| Huntington's Disease Signaling | ATP5PB, CASP2, CASP9, MAPK8, NSF, |
| | PDPK1, POLR2D, SOS2 |
| PPARα/RXRα Activation | ADCY9, KRAS, MAPK8, NR0B2, SOS2, |
| | TGFBR3 |

TABLE 19
HUH7 liver cancer aberrant pathways and associated genes

| Pathway Name | Gene |
|---|---|
| Cell Cycle Control of Chromosomal Replication | CDC45, CDC6, CDK1, CDK2, CDK6, CDT1, |
| | DNA2, LIG1, MCM2, MCM3, MCM4, MCM5, |
| | MCM6, MCM7, ORC1, PCNA, POLA1, POLA2, |
| | POLE, PRIM1, TOP2A |
| Hereditary Breast Cancer Signaling | BLM, BRCA1, BRCA2, CCNB1, CCND1, CDK1, |
| | CDK6, CHEK1, E2F1, FANCD2, FANCE, |
| | FANCG, H2AFX, KRAS, MSH2, POLR2D, |
| | RAD51, RFC2, RFC3, TUBG1 |
| GADD45 Signaling | BRCA1, CCNB1, CCND1, CCNE2, CDK1, |
| | CDK2, PCNA |
| Mismatch Repair in Eukaryotes | EXO1, FEN1, MSH2, PCNA, RFC2, RFC3 |
| Molecular Mechanisms of Cancer | ADCY9, APH1B, ARHGEF12, ARHGEF5, |
| | BRCA1, CASP9, CCND1, CCNE2, CDC25A, |
| | CDK1, CDK2, CDK6, CDKN2C, CHEK1, E2F1, |
| | E2F2, E2F7, E2F8, FANCD2, KRAS, LEF1, |
| | MAPK8, MAX, MDM2, PAK4, PTK2, RAC2, |
| | RBL1, SOS2, TCF4, TFDP1 |
| DNA damage-induced 14-3-3σ Signaling | BRCA1, CCNB1, CCNE2, CDK1, CDK2, RAD17 |
| Glioma Signaling | CCND1, CDK6, CDKN2C, E2F1, E2F2, E2F7, |
| | E2F8, IDH2, KRAS, MDM2, PDGFRB, RBL1, |
| | SOS2, TFDP1 |
| DNA Double-Strand Break Repair by | BRCA1, BRCA2, LIG1, POLA1, RAD51 |
| Homologous Recombination | |
| Breast Cancer Regulation by Stathmin1 | ADCY9, ARHGEF12, ARHGEF5, CCNE2, |
| | CDK1, CDK2, E2F1, E2F2, E2F7, E2F8, KRAS, |
| | PPP2R2B, SOS2, STMN1, TUBA1A, TUBA1B, |
| | TUBA4A, TUBB4B, TUBG1 |
| Chronic Myeloid Leukemia Signaling | CCND1, CDK6, CRKL, E2F1, E2F2, E2F7, |
| | E2F8, KRAS, MDM2, RBL1, SOS2, TFDP1 |
| Prostate Cancer Signaling | CASP9, CCND1, CCNE2, CDK2, E2F1, KRAS, |
| | LEF1, MDM2, PDPK1, SOS2, TFDP1 |
| dTMP De Novo Biosynthesis | DHFR, SHMT2, TYMS |
| BER pathway | FEN1, LIG1, PCNA, POLE |
| Guanine and Guanosine Salvage I | HPRT1, PNP |
| Remodeling of Epithelial Adherens Junctions | ARPC2, ARPC5, RAB5B, TUBA1A, TUBA1B, |
| | TUBA4A, TUBB4B, TUBG1 |
| Role of Oct4 in Mammalian Embryonic Stem | BRCA1, CCNF, IGF2BP1, NR2F6, NR6A1, |
| Cell Pluripotency | SPP1 |
| Acetate Conversion to Acetyl-CoA | ACSS2, ACSS3 |
| Phenylalanine Degradation I (Aerobic) | PCBD1, QDPR |
| Regulation of Cellular Mechanics by Calpain | CCNA2, CCND1, CDK1, CDK2, CDK6, KRAS, |
| Protease | PTK2 |
| Bile Acid Biosynthesis, Neutral Pathway | AKR1C1/AKR1C2, BAAT, SCP2 |
| HER-2 Signaling in Breast Cancer | CASP9, CCND1, CCNE2, CDK6, ITGB8, KRAS, |
| | MDM2, SOS2 |
| Ovarian Cancer Signaling | BRCA1, BRCA2, CCND1, E2F1, KRAS, LEF1, |
| | MSH2, RAD51, RPS6KB2, TCF4, TFDP1 |
| Folate Polyglutamylation | MTHFD1, SHMT2 |
| Adenine and Adenosine Salvage III | HPRT1, PNP |
| Granzyme B Signaling | CASP9, LMNB1, LMNB2 |
| Epithelial Adherens Junction Signaling | ARPC2, ARPC5, KRAS, LEF1, TCF4, TGFBR3, |
| | TUBA1A, TUBA1B, TUBA4A, TUBB4B, TUBG1 |
| Germ Cell-Sertoli Cell Junction Signaling | KRAS, MAP3K3, MAPK8, PAK4, PDPK1, PTK2, |
| | RAC2, TUBA1A, TUBA1B, TUBA4A, TUBB4B, |
| | TUBG1 |
| Oxidative Ethanol Degradation III | ACSS2, ACSS3, ALDH9A1 |
| Sphingomyelin Metabolism | SGMS1, SMPD1 |
| Thyroid Cancer Signaling | CCND1, CXCL12, KRAS, LEF1, TCF4 |
| Thio-molybdenum Cofactor Biosynthesis | MOCOS |
| Xanthine and Xanthosine Salvage | PNP |
| Folate Transformations I | MTHFD1, SHMT2 |
| Polyamine Regulation in Colon Cancer | KRAS, MAX, TCF4 |
| Factors Promoting Cardiogenesis in Vertebrates | CCNE2, CDC6, CDK2, DKK1, LEF1, TCF4, |
| | TGFBR3 |
| Ethanol Degradation IV | ACSS2, ACSS3, ALDH9A1 |
| Glycine Betaine Degradation | BHMT, SHMT2 |
| Role of JAK family kinases in IL-6-type Cytokine | IL6R, MAPK8, OSMR |
| Signaling | |
| Antiproliferative Role of TOB in T Cell Signaling | CCNA2, CCNE2, CDK2 |
| Guanosine Nucleotides Degradation III | NT5E, PNP |
| Adenine and Adenosine Salvage I | PNP |
| Glycine Biosynthesis I | SHMT2 |
| L-cysteine Degradation III | MPST |
| Stearate Biosynthesis I (Animals) | ACOT11, ACOT8, DHCR24, ELOVL2 |
| Acyl-CoA Hydrolysis | ACOT11, ACOT8 |
| Cholesterol Biosynthesis I | DHCR24, LBR |
| Cholesterol Biosynthesis II (via 24,25- | DHCR24, LBR |
| dihydrolanosterol) | |
| Cholesterol Biosynthesis III (via Desmosterol) | DHCR24, LBR |
| Urate Biosynthesis/Inosine 5′-phosphate | NT5E, PNP |
| Degradation | |
| Myc Mediated Apoptosis Signaling | CASP9, KRAS, MAPK8, SOS2, YWHAZ |
| 14-3-3-mediated Signaling | KRAS, MAPK8, TUBA1A, TUBA1B, TUBA4A, |
| | TUBB4B, TUBG1, YWHAZ |
| Androgen Biosynthesis | HSD17B3, SRD5A2 |
| Sonic Hedgehog Signaling | CCNB1, CDK1, DYRK1B |
| Adenosine Nucleotides Degradation II | NT5E, PNP |
| Ethanol Degradation II | ACSS2, ACSS3, ALDH9A1 |
| 5-aminoimidazole Ribonucleotide Biosynthesis I | PPAT |
| L-carnitine Biosynthesis | ALDH9A1 |
| Methionine Salvage II (Mammalian) | BHMT |
| Methylglyoxal Degradation I | GLO1 |
| N-acetylglucosamine Degradation I | GNPDA1 |
| Thiosulfate Disproportionation III (Rhodanese) | MPST |
| Tyrosine Biosynthesis IV | PCBD1 |
| Parkinson's Signaling | CASP9, MAPK8 |
| DNA Methylation and Transcriptional | DNMT1, HIST4H4, MTA2 |
| Repression Signaling | |
| Sertoli Cell-Sertoli Cell Junction Signaling | KRAS, MAP3K3, MAPK8, PRKG2, TGFBR3, |
| | TUBA1A, TUBA1B, TUBA4A, TUBB4B, TUBG1 |
| Sphingosine-1-phosphate Signaling | ADCY9, CASP2, CASP9, PDGFRB, PTK2, |
| | RAC2, SMPD1 |
| Bladder Cancer Signaling | CCND1, E2F1, FGF19, KRAS, MDM2, TFDP1 |
| UVA-Induced MAPK Signaling | CASP9, KRAS, MAPK8, PARP14, RPS6KB2, |
| | SMPD1 |
| 1D-myo-inositol Hexakisphosphate Biosynthesis | INPPL1, ITPKA |
| II (Mammalian) | |
| D-myo-inositol (1,3,4)-trisphosphate | INPPL1, ITPKA |
| Biosynthesis | |
| Purine Nucleotides Degradation II (Aerobic) | NT5E, PNP |
| Dopamine Receptor Signaling | ADCY9, PCBD1, PPP2R2B, PRL, QDPR |
| Arsenate Detoxification I (Glutaredoxin) | PNP |
| Glutathione Redox Reactions II | GSR |
| N-acetylglucosamine Degradation II | GNPDA1 |
| PRPP Biosynthesis I | PRPS2 |
| Granzyme A Signaling | HIST1H1E, HMGB2 |
| Phagosome Maturation | CTSK, NSF, RAB5B, TUBA1A, TUBA1B, |
| | TUBA4A, TUBB4B, TUBG1 |
| Semaphorin Signaling in Neurons | ARHGEF12, PAK4, PTK2, RAC2 |
| Tetrahydrofolate Salvage from 5,10- | MTHFD1 |
| methenyltetrahydrofolate | |
| PCP pathway | CTHRC1, JUND, LGR4, MAPK8 |
| Endoplasmic Reticulum Stress Pathway | CASP9, MAPK8 |
| TR/RXR Activation | AKR1C1/AKR1C2, MDM2, NRGN, PFKP, |
| | TBL1XR1 |
| tRNA Splicing | PDE3A, PDE4D, PLD6 |
| Oncostatin M Signaling | KRAS, OSMR, PLAU |
| Serotonin Receptor Signaling | ADCY9, PCBD1, QDPR |
| Superpathway of D-myo-inositol (1,4,5)- | INPPL1, ITPKA |
| trisphosphate Metabolism | |
| Glycine Cleavage Complex | OCA2 |
| Zymosterol Biosynthesis | LBR |
| IL-22 Signaling | IL22RA1, MAPK8 |
| D-myo-inositol (1,4,5)-Trisphosphate | PIP4K2A, PIP4K2C |
| Biosynthesis | |
| Purine Ribonucleosides Degradation to Ribose- | PNP |
| 1-phosphate | |
| Superpathway of Serine and Glycine | SHMT2 |
| Biosynthesis I | |
| Apelin Liver Signaling Pathway | MAPK8, PDGFRB |
| NAD Salvage Pathway II | NT5E, PXYLP1 |
| GABA Receptor Signaling | ADCY9, ALDH9A1, AP2M1, KCNH2, NSF |
| FAK Signaling | KRAS, PAK4, PDPK1, PTK2, SOS2 |
| Histidine Degradation III | MTHFD1 |
| Salvage Pathways of Pyrimidine | TK1 |
| Deoxyribonucleotides | |
| Superpathway of Cholesterol Biosynthesis | DHCR24, LBR |
| Axonal Guidance Signaling | ARHGEF12, ARPC2, ARPC5, CRKL, CXCL12, |
| | CXCR4, EFNA4, KRAS, PAK4, PLXNA3, |
| | PLXNC1, PTK2, RAC2, SEMA4B, SOS2, |
| | TUBA1A, TUBA1B, TUBA4A, TUBB4B, TUBG1 |
| Hypoxia Signaling in the Cardiovascular System | MDM2, UBE2C, UBE2L6, UBE2T |
| Gap Junction Signaling | ADCY9, KRAS, PRKG2, SOS2, TUBA1A, |
| | TUBA1B, TUBA4A, TUBB4B, TUBG1 |
| UVC-Induced MAPK Signaling | KRAS, MAPK8, SMPD1 |
| Leucine Degradation I | BCAT2 |
| UDP-N-acetyl-D-galactosamine Biosynthesis II | GNPDA1 |
| CD27 Signaling in Lymphocytes | CASP9, MAP3K3, MAPK8 |
| Role of p14/p19ARF in Tumor Suppression | E2F1, MDM2 |
| Glycolysis I | PFKP |
| Lipid Antigen Presentation by CD1 | AP2M1 |
| Reelin Signaling in Neurons | ARHGEF12, ARHGEF5, CRKL, MAPK8 |
| IL-17A Signaling in Gastric Cells | MAPK8 |
| Tryptophan Degradation X (Mammalian, via | ALDH9A1 |
| Tryptamine) | |
| Embryonic Stem Cell Differentiation into Cardiac | SP4 |
| Lineages | |
| EGF Signaling | MAPK8, SOS2 |
| Cellular Effects of Sildenafil (Viagra) | ADCY9, KCNH2, MYLK, PDE3A, PDE4D, |
| | PRKG2 |
| Glutathione Redox Reactions I | GSR |
| TCA Cycle II (Eukaryotic) | IDH3A |
| Tumoricidal Function of Hepatic Natural Killer | CASP9 |
| Cells | |
| BMP signaling pathway | FST, KRAS, MAPK8 |
| Lymphotoxin β Receptor Signaling | CASP9, PDPK1 |
| Transcriptional Regulatory Network in | HIST4H4, PAX6 |
| Embryonic Stem Cells | |
| HIF1α Signaling | KRAS, MAPK8, MDM2, SLC2A14 |
| FLT3 Signaling in Hematopoietic Progenitor | KRAS, PDPK1, SOS2 |
| Cells | |
| PEDF Signaling | ARHGAP22, KRAS, TCF4 |
| Purine Nucleotides De Novo Biosynthesis II | PPAT |
| Phosphatidylglycerol Biosynthesis II (Non- | AGPAT1 |
| plastidic) | |
| JAK/Stat Signaling | KRAS, SOCS2, SOS2 |
| Role of JAK2 in Hormone-like Cytokine | PRL, SOCS2 |
| Signaling | |
| Putrescine Degradation III | ALDH9A1 |
| Virus Entry via Endocytic Pathways | AP2M1, ITGB8, KRAS, RAC2 |
| Estrogen Receptor Signaling | KRAS, MED21, NR0B2, POLR2D, SOS2 |
| Assembly of RNA Polymerase I Complex | POLR1E |
| Cleavage and Polyadenylation of Pre-mRNA | NUDT21 |
| Coagulation System | PLAU, PLAUR |
| Clathrin-mediated Endocytosis Signaling | AP2M1, ARPC2, ARPC5, FGF19, ITGB8, |
| | MDM2, RAB5B |
| Antioxidant Action of Vitamin C | MAPK8, PLD6, SLC23A2, SLC2A14 |
| IL-4 Signaling | INPPL1, KRAS, RPS6KB2, SOS2 |
| CDP-diacylglycerol Biosynthesis I | AGPAT1 |
| Fatty Acid α-oxidation | ALDH9A1 |
| The Visual Cycle | RDH5 |
| T Cell Receptor Signaling | KRAS, MAPK8, PAG1, SOS2 |
| Induction of Apoptosis by HIV1 | CASP9, CXCR4, MAPK8 |
| Interferon Signaling | IFIT3, PSMB8 |
| Erythropoietin Signaling | KRAS, PDPK1, SOS2 |
| GDNF Family Ligand-Receptor Interactions | KRAS, MAPK8, SOS2 |
| Ephrin A Signaling | EFNA4, PTK2 |
| Regulation of the Epithelial-Mesenchymal | APH1B, ETS1, FGF19, KRAS, LEF1, PDGFRB, |
| Transition Pathway | SOS2, TCF4 |
| FcγRIIB Signaling in B Lymphocytes | KRAS, MAPK8, PDPK1 |
| IL-2 Signaling | KRAS, MAPK8, SOS2 |
| Assembly of RNA Polymerase III Complex | GTF3C5 |
| NAD Phosphorylation and Dephosphorylation | PXYLP1 |
| Complement System | C1QBP, MBL2 |
| Estrogen-Dependent Breast Cancer Signaling | CCND1, HSD17B3, KRAS |
| Ceramide Signaling | KRAS, MAPK8, PPP2R2B, SMPD1 |
| Protein Ubiquitination Pathway | BRCA1, CDC20, DNAJB5, MDM2, PSMB8, |
| | UBE2C, UBE2L6, UBE2T, USP1, USP39 |
| G-Protein Coupled Receptor Signaling | ADCY9, KRAS, PDE3A, PDE4D, PDPK1, PLD6, |
| | RGS10, RGS16, RGS2, SOS2 |
| FAT10 Signaling Pathway | SQSTM1 |
| Methylglyoxal Degradation III | AKR1C1/AKR1C2 |
| Valine Degradation I | BCAT2 |
| Isoleucine Degradation I | BCAT2 |
| Role of PI3K/AKT Signaling in the Pathogenesis | CASP9, CRKL, ZNF346 |
| of Influenza | |
| Docosahexaenoic Acid (DHA) Signaling | CASP9, PDPK1 |
| tRNA Charging | FARSA, LARS2 |
| Wnt/β-catenin Signaling | CCND1, DKK1, LEF1, MDM2, PPP2R2B, TCF4, |
| | TGFBR3 |
| D-myo-inositol (1,4,5)-trisphosphate | INPPL1 |
| Degradation | |
| Dermatan Sulfate Degradation (Metazoa) | IDS |
| Histamine Degradation | ALDH9A1 |
| RAN Signaling | RCC1 |
| Natural Killer Cell Signaling | INPPL1, KRAS, PAK4, RAC2, SOS2 |
| Choline Biosynthesis III | PLD6 |
| IL-15 Production | MST1R, PDGFRB, PTK2, ROR1, TWF1 |
| Mechanisms of Viral Exit from Host Cells | LMNB1, LMNB2 |
| Triacylglycerol Biosynthesis | AGPAT1, ELOVL2 |
| Vitamin-C Transport | SLC23A2 |
| 4-1BB Signaling in T Lymphocytes | MAPK8 |
| Activation of IRF by Cytosolic Pattern | MAPK8 |
| Recognition Receptors | |
| Adipogenesis pathway | EZH2, TBL1XR1 |
| Agranulocyte Adhesion and Diapedesis | CXCL12, CXCR4, PODXL |
| Altered T Cell and B Cell Signaling in | SPP1 |
| Rheumatoid Arthritis | |
| Amyloid Processing | APH1B |
| Amyotrophic Lateral Sclerosis Signaling | CASP9, NEFH, RAB5B |
| Androgen Signaling | CCND1, POLR2D |
| Antigen Presentation Pathway | PSMB8 |
| Antiproliferative Role of Somatostatin Receptor | KRAS |
| 2 | |
| Apelin Adipocyte Signaling Pathway | ADCY9, MAPK8 |
| Apelin Cardiomyocyte Signaling Pathway | MAPK8, MYLK |
| Apelin Pancreas Signaling Pathway | MAPK8 |
| April Mediated Signaling | MAPK8 |
| Assembly of RNA Polymerase II Complex | POLR2D |
| Atherosclerosis Signaling | CXCL12, CXCR4 |
| Autophagy | CTSK, SQSTM1 |
| B Cell Activating Factor Signaling | MAPK8 |
| BAG2 Signaling Pathway | MDM2 |
| Basal Cell Carcinoma Signaling | LEF1, TCF4 |
| CCR3 Signaling in Eosinophils | KRAS, MYLK, PAK4 |
| CCR5 Signaling in Macrophages | MAPK8 |
| CD40 Signaling | MAPK8 |
| CNTF Signaling | KRAS, RPS6KB2 |
| CREB Signaling in Neurons | ADCY9, KRAS, POLR2D, SOS2 |
| CTLA4 Signaling in Cytotoxic T Lymphocytes | AP2M1, PPP2R2B |
| Calcium Signaling | CHRNA5, RCAN3 |
| Calcium-induced T Lymphocyte Apoptosis | ORAI1 |
| Cancer Drug Resistance By Drug Efflux | KRAS, MDM2 |
| Caveolar-mediated Endocytosis Signaling | ITGB8, RAB5B |
| Corticotropin Releasing Hormone Signaling | ADCY9, ARPC5, JUND |
| Cytotoxic T Lymphocyte-mediated Apoptosis of | CASP9 |
| Target Cells | |
| Dendritic Cell Maturation | MAPK8 |
| Dermatan Sulfate Biosynthesis | CHST14 |
| Dermatan Sulfate Biosynthesis (Late Stages) | CHST14 |
| Dopamine Degradation | ALDH9A1 |
| Dopamine-DARPP32 Feedback in cAMP | ADCY9, PPP2R2B, PRKG2 |
| Signaling | |
| Endocannabinoid Neuronal Synapse Pathway | ADCY9, MAPK8 |
| Estrogen Biosynthesis | HSD17B3 |
| FXR/RXR Activation | BAAT, FGF19, MAPK8, NR0B2 |
| Fatty Acid β-oxidation I | SCP2 |
| G Beta Gamma Signaling | KRAS, PDPK1, SOS2 |
| GP6 Signaling Pathway | LAMC1, PDPK1, PTK2 |
| GPCR-Mediated Integration of Enteroendocrine | ADCY9 |
| Signaling Exemplified by an L Cell | |
| GPCR-Mediated Nutrient Sensing in | ADCY9 |
| Enteroendocrine Cells | |
| Glucocorticoid Receptor Signaling | HMGB1, KRAS, KRT23, MAPK8, PBX1, PLAU, |
| | POLR2D, PRL, SOS2 |
| Granulocyte Adhesion and Diapedesis | CXCL12, CXCR4 |
| Gustation Pathway | ADCY9, ASIC3, PDE3A, PDE4D, PLD6 |
| Gai Signaling | ADCY9, KRAS, RGS10, SOS2 |
| Gaq Signaling | PLD6, RAC2, RGS16, RGS2 |
| Gas Signaling | ADCY9, RGS2 |
| HIPPO signaling | PPP2R2B, YWHAZ |
| HMGB1 Signaling | HMGB1, KRAS, MAPK8, RAC2 |
| Heparan Sulfate Biosynthesis | PNPLA7 |
| Heparan Sulfate Biosynthesis (Late Stages) | PNPLA7 |
| Hepatic Cholestasis | ADCY9, FGF19, MAPK8, NR0B2 |
| Hepatic Fibrosis/Hepatic Stellate Cell | IL6R, PDGFRB |
| Activation | |
| Human Embryonic Stem Cell Pluripotency | LEF1, PDGFRB, PDPK1, TCF4 |
| IL-1 Signaling | ADCY9, MAPK8 |
| IL-10 Signaling | MAPK8 |
| IL-12 Signaling and Production in Macrophages | MAPK8, MST1R |
| IL-15 Signaling | KRAS, PTK2 |
| IL-17 Signaling | KRAS, MAPK8 |
| IL-17A Signaling in Airway Cells | MAPK8 |
| IL-3 Signaling | CRKL, KRAS |
| IL-9 Signaling | SOCS2 |
| Inhibition of Angiogenesis by TSP1 | MAPK8 |
| Inhibition of Matrix Metalloproteases | TIMP4 |
| Iron homeostasis signaling pathway | ABCB10, IL6R, PDGFRB |
| LPS-stimulated MAPK Signaling | KRAS, MAPK8 |
| LPS/IL-1 Mediated Inhibition of RXR Function | ALDH9A1, MAPK8, NR0B2 |
| Leptin Signaling in Obesity | ADCY9, PDE3A |
| MIF Regulation of Innate Immunity | MAPK8 |
| MSP-RON Signaling Pathway | MST1R |
| Macropinocytosis Signaling | ITGB8, KRAS |
| Mitochondrial Dysfunction | APH1B, ATP5PB, CASP9, GSR, MAPK8 |
| NF-κB Activation by Viruses | KRAS |
| Netrin Signaling | RAC2 |
| Neuropathic Pain Signaling In Dorsal Horn | KCNH2 |
| Neurons | |
| Neuroprotective Role of THOP1 in Alzheimer's | TPP1 |
| Disease | |
| Nitric Oxide Signaling in the Cardiovascular | PRKG2 |
| System | |
| Noradrenaline and Adrenaline Degradation | ALDH9A1 |
| Notch Signaling | APH1B |
| Nucleotide Excision Repair Pathway | POLR2D |
| Nur77 Signaling in T Lymphocytes | CASP9, MAP3K3 |
| OX40 Signaling Pathway | MAPK8 |
| Oxidative Phosphorylation | ATP5PB |
| P2Y Purigenic Receptor Signaling Pathway | ADCY9, KRAS |
| PD-1, PD-L1 cancer immunotherapy pathway | CDK2, PDCD4 |
| PFKFB4 Signaling Pathway | HK2 |
| PI3K Signaling in B Lymphocytes | ATF3, KRAS, PDPK1 |
| PXR/RXR Activation | NR0B2 |
| Phagosome Formation | MBL2, RAC2 |
| Phospholipases | PLD6 |
| Primary Immunodeficiency Signaling | UNG |
| Production of Nitric Oxide and Reactive Oxygen | MAP3K3, MAPK8, PPP2R2B, RAC2 |
| Species in Macrophages | |
| RANK Signaling in Osteoclasts | MAP3K3, MAPK8 |
| RAR Activation | ADCY9, MAPK8, NR2F6, PDPK1, PNRC1, |
| | RDH5 |
| Regulation of IL-2 Expression in Activated and | KRAS, MAPK8, SOS2 |
| Anergic T Lymphocytes | |
| Relaxin Signaling | ADCY9, PDE3A, PDE4D, PLD6 |
| Retinoate Biosynthesis I | RDH5 |
| Retinoic acid Mediated Apoptosis Signaling | CASP9, PARP14 |
| Role of IL-17A in Arthritis | MAPK8 |
| Role of JAK1 and JAK3 in γc Cytokine Signaling | KRAS |
| Role of MAPK Signaling in the Pathogenesis of | KRAS, MAPK8 |
| Influenza | |
| Role of Macrophages, Fibroblasts and | CCND1, CXCL12, DKK1, IL6R, KRAS, LEF1, |
| Endothelial Cells in Rheumatoid Arthritis | TCF4 |
| Role of NANOG in Mammalian Embryonic Stem | KRAS, SOS2 |
| Cell Pluripotency | |
| Role of NFAT in Regulation of the Immune | KRAS, ORAI1, RCAN3, SOS2 |
| Response | |
| Role of Osteoblasts, Osteoclasts and | CASP9, CTSK, DKK1, LEF1, MAPK8, SPP1, |
| Chondrocytes in Rheumatoid Arthritis | TCF4 |
| Role of PKR in Interferon Induction and Antiviral | CASP9 |
| Response | |
| Role of Pattern Recognition Receptors in | MAPK8, MBL2, OAS3 |
| Recognition of Bacteria and Viruses | |
| Role of Tissue Factor in Cancer | KRAS, PLAUR |
| Role of Wnt/GSK-3β Signaling in the | LEF1, TCF4 |
| Pathogenesis of Influenza | |
| Serotonin Degradation | ALDH9A1 |
| Superpathway of Methionine Degradation | BHMT |
| Synaptic Long Term Depression | KRAS, PPP2R2B, PRKG2 |
| Synaptic Long Term Potentiation | KRAS |
| Systemic Lupus Erythematosus In T Cell | CASP2, CASP9, DNMT1, KRAS, ORAI1, |
| Signaling Pathway | PPP2R2B, PTK2, RAC2, RPS6KB2, SOS2 |
| Systemic Lupus Erythematosus Signaling | IL6R, KRAS, LSM14B, SOS2 |
| T Cell Exhaustion Signaling Pathway | IL6R, KRAS, MAPK8, PPP2R2B, TGFBR3 |
| T Helper Cell Differentiation | IL6R |
| TGF-β Signaling | KRAS, MAPK8, SOS2 |
| TNFR2 Signaling | MAPK8 |
| TWEAK Signaling | CASP9 |
| Th1 Pathway | APH1B, IL6R |
| Th1 and Th2 Activation Pathway | APH1B, CXCR4, IL6R, PTGDR2, TGFBR3 |
| Th17 Activation Pathway | IL6R |
| Th2 Pathway | APH1B, CXCR4, PTGDR2, TGFBR3 |
| Thrombopoietin Signaling | KRAS |
| Tight Junction Signaling | MYLK, NSF, NUDT21, PPP2R2B, STX4 |
| Toll-like Receptor Signaling | MAPK8 |
| Triacylglycerol Degradation | PNPLA7 |
| Type I Diabetes Mellitus Signaling | CASP9, MAPK8, SOCS2 |
| Type II Diabetes Mellitus Signaling | MAPK8, PDPK1, SMPD1, SOCS2 |
| UVB-Induced MAPK Signaling | MAPK8 |
| Unfolded protein response | MAPK8 |
| VDR/RXR Activation | SPP1 |
| VEGF Family Ligand-Receptor Interactions | KRAS, SOS2 |
| VEGF Signaling | KRAS, PTK2, SOS2 |
| Wnt/Ca+ pathway | ROR1 |
| Xenobiotic Metabolism Signaling | ALDH9A1, KRAS, MAP3K3, MAPK8, PPP2R2B |
| fMLP Signaling in Neutrophils | ARPC2, ARPC5, KRAS |
| iCOS-iCOSL Signaling in T Helper Cells | PDPK1 |
| p38 MAPK Signaling | MAX, RPS6KB2 |
| α-Adrenergic Signaling | ADCY9, KRAS |

TABLE 20
HUH7 liver cancer downregulated pathways and associated genes

| Pathway Name | Gene |
|---|---|
| Estrogen-mediated S-phase Entry | CCNA2, CCND1, CCNE2, CDC25A, CDK1, |
| | CDK2, E2F1, E2F2, E2F7, E2F8, RBL1, TFDP1 |
| Role of BRCA1 in DNA Damage Response | BLM, BRCA1, BRCA2, BRIP1, CHEK1, E2F1, |
| | E2F2, E2F7, E2F8, FANCD2, FANCE, FANCG, |
| | MDC1, MSH2, PLK1, RAD51, RBL1, RFC2, |
| | RFC3 |
| ATM Signaling | BLM, BRCA1, CBX1, CCNB1, CDC25A, CDK1, |
| | CDK2, CHEK1, FANCD2, H2AFX, HP1BP3, |
| | MAPK8, MDC1, MDM2, PPP2R2B, RAD17, |
| | RAD51, RNF168 |
| Cyclins and Cell Cycle Regulation | CCNA2, CCNB1, CCND1, CCNE2, CDC25A, |
| | CDK1, CDK2, CDK6, CDKN2C, E2F1, E2F2, |
| | E2F7, E2F8, PPP2R2B, TFDP1 |
| NER Pathway | CHAF1A, CHAF1B, COPS3, DNA2, HIST4H4, |
| | LIG1, PCNA, POLA1, POLA2, POLE, POLE3, |
| | POLR2D, PRIM1, RFC2, RFC3, TOP2A |
| Mitotic Roles of Polo-Like Kinase | CCNB1, CDC20, CDC25A, CDK1, ESPL1, |
| | FBXO5, KIF11, KIF23, PKMYT1, PLK1, PLK4, |
| | PPP2R2B |
| Cell Cycle Regulation by BTG Family Proteins | BTG2, CCND1, CCNE2, CDK2, E2F1, E2F2, |
| | E2F7, E2F8, PPP2R2B |
| Pancreatic Adenocarcinoma Signaling | BRCA2, CASP9, CCND1, CDK2, E2F1, E2F2, |
| | E2F7, E2F8, KRAS, MAPK8, MDM2, PLD6, |
| | RAD51, TFDP1 |
| Aryl Hydrocarbon Receptor Signaling | ALDH9A1, CCNA2, CCND1, CCNE2, CDK2, |
| | CDK6, CHEK1, DHFR, E2F1, MAPK8, MCM7, |
| | MDM2, NR0B2, POLA1, RBL1, TFDP1 |
| Small Cell Lung Cancer Signaling | CASP9, CCND1, CCNE2, CDK2, CDK6, E2F1, |
| | MAX, PTK2, TFDP1 |
| Agrin Interactions at Neuromuscular Junction | GABPA, KRAS, LAMC1, MAPK8, NRG3, NRG4, |
| | PAK4, PTK2, RAC2 |
| Neuregulin Signaling | CRKL, ERBIN, GRB7, KRAS, NRG3, NRG4, |
| | PDPK1, RPS6KB2, SOS2, TMEFF2 |
| p53 Signaling | BRCA1, CCND1, CDK2, CHEK1, E2F1, |
| | MAPK8, MDM2, PCNA, TP53INP1, TRIM29 |
| Non-Small Cell Lung Cancer Signaling | CASP9, CCND1, CDK6, E2F1, KRAS, PDPK1, |
| | SOS2, TFDP1 |
| Glioblastoma Multiforme Signaling | CCND1, CDK2, CDK6, E2F1, E2F2, E2F7, |
| | E2F8, KRAS, LEF1, MDM2, PDGFRB, RAC2, |
| | SOS2 |
| Pyrimidine Deoxyribonucleotides De Novo | DUT, RRM1, RRM2, TYMS |
| Biosynthesis | |
| IGF-1 Signaling | CASP9, KRAS, MAPK8, PDPK1, PTK2, |
| | RPS6KB2, SOCS2, SOS2, YWHAZ |
| HGF Signaling | CCND1, CDK2, CRKL, ETS1, KRAS, MAP3K3, |
| | MAPK8, PTK2, SOS2 |
| Rac Signaling | ABI2, ARPC2, ARPC5, IQGAP3, KRAS, |
| | MAPK8, PAK4, PIP4K2C, PTK2 |
| PI3K/AKT Signaling | CCND1, INPPL1, KRAS, MCL1, MDM2, PDPK1, |
| | PPP2R2B, RPS6KB2, SOS2, YWHAZ |
| Integrin Signaling | ARF3, ARPC2, ARPC5, CRKL, GRB7, ITGB8, |
| | KRAS, MAPK8, MYLK, MYLK2, PAK4, PTK2, |
| | RAC2, SOS2 |
| Endometrial Cancer Signaling | CASP9, CCND1, KRAS, LEF1, PDPK1, SOS2 |
| IL-7 Signaling Pathway | CCND1, CDC25A, CDK2, MCL1, PDPK1, PTK2, |
| | SOS2 |
| FAT10 Cancer Signaling Pathway | CXCR4, MAD2L1, PCNA, TCF4, TGFBR3 |
| Actin Cytoskeleton Signaling | ABI2, ARHGEF12, ARPC2, ARPC5, CRKL, |
| | FGF19, IQGAP3, KRAS, MYLK, MYLK2, PAK4, |
| | PTK2, RAC2, SOS2 |
| Pyridoxal 5′-phosphate Salvage Pathway | CDK1, CDK2, CDK6, FAM20B, MAPK8, PLK1 |
| ErbB2-ErbB3 Signaling | CCND1, KRAS, NRG3, NRG4, PDPK1, SOS2 |
| RhoA Signaling | ANLN, ARHGEF12, ARPC2, ARPC5, LPAR3, |
| | MYLK, MYLK2, PIP4K2C, PTK2 |
| ErbB4 Signaling | APH1B, KRAS, NRG3, NRG4, PDPK1, SOS2 |
| Acute Myeloid Leukemia Signaling | CCND1, IDH2, KRAS, LEF1, RPS6KB2, SOS2, |
| | TCF4 |
| STAT3 Pathway | CDC25A, IL17RD, IL22RA1, IL6R, KRAS, |
| | MAPK8, PDGFRB, SOCS2, TGFBR3 |
| D-myo-inositol-5-phosphate Metabolism | CDC25A, DUSP8, HACD2, MTMR9, NUDT15, |
| | PIP4K2A, PIP4K2C, PPFIA3, PPTC7, PXYLP1 |
| Superpathway of Inositol Phosphate | CDC25A, DUSP8, HACD2, INPPL1, ITPKA, |
| Compounds | MTMR9, NUDT15, PIP4K2A, PIP4K2C, PPFIA3, |
| | PPTC7, PXYLP1 |
| Glioma Invasiveness Signaling | KRAS, PLAU, PLAUR, PTK2, RAC2, TIMP4 |
| ErbB Signaling | KRAS, MAPK8, NRG3, NRG4, PAK4, PDPK1, |
| | SOS2 |
| Ephrin Receptor Signaling | ARPC2, ARPC5, CRKL, CXCL12, CXCR4, |
| | EFNA4, KRAS, PAK4, PTK2, RAC2, SOS2 |
| PAK Signaling | KRAS, MAPK8, MYLK, PAK4, PDGFRB, PTK2, |
| | SOS2 |
| Insulin Receptor Signaling | ASIC3, CRKL, INPPL1, KRAS, MAPK8, PDPK1, |
| | RPS6KB2, SOS2, STX4 |
| Apoptosis Signaling | CASP2, CASP9, CDK1, GAS2, KRAS, MAPK8, |
| | MCL1 |
| Pyrimidine Ribonucleotides Interconversion | BLM, NUDT15, RAD54L, RECQL4 |
| B Cell Receptor Signaling | ETS1, INPPL1, KRAS, MAP3K3, MAPK8, |
| | PAG1, PDPK1, PTK2, RAC2, RPS6KB2, SOS2 |
| Pyrimidine Ribonucleotides De Novo | BLM, NUDT15, RAD54L, RECQL4 |
| Biosynthesis | |
| SAPK/JNK Signaling | CRKL, DUSP8, KRAS, MAP3K3, MAPK8, |
| | RAC2, SOS2 |
| Telomerase Signaling | E2F1, ETS1, KRAS, PDPK1, PPP2R2B, SOS2, |
| | TPP1 |
| PDGF Signaling | CRKL, INPPL1, KRAS, MAPK8, PDGFRB, |
| | SOS2 |
| 3-phosphoinositide Degradation | CDC25A, DUSP8, HACD2, INPPL1, MTMR9, |
| | NUDT15, PPFIA3, PPTC7, PXYLP1 |
| TNFR1 Signaling | CASP2, CASP9, MAPK8, PAK4 |
| Death Receptor Signaling | CASP2, CASP9, GAS2, MAPK8, PARP14, |
| | TNFRSF21 |
| SPINK1 General Cancer Pathway | IL6R, KRAS, MT1F, MT1M, MT1X |
| Melanoma Signaling | CCND1, E2F1, KRAS, MDM2 |
| Actin Nucleation by ARP-WASP Complex | ARPC2, ARPC5, KRAS, RAC2, SOS2 |
| NGF Signaling | KRAS, MAP3K3, MAPK8, PDPK1, RPS6KB2, |
| | SMPD1, SOS2 |
| Regulation of Actin-based Motility by Rho | ARPC2, ARPC5, MYLK, PAK4, PIP4K2C, RAC2 |
| Endocannabinoid Developing Neuron Pathway | ADCY9, BRCA1, CCND1, KRAS, MAPK8, |
| | PAX6, RAC2 |
| D-myo-inositol (1,4,5,6)-Tetrakisphosphate | CDC25A, DUSP8, HACD2, MTMR9, NUDT15, |
| Biosynthesis | PPFIA3, PPTC7, PXYLP1 |
| D-myo-inositol (3,4,5,6)-tetrakisphosphate | CDC25A, DUSP8, HACD2, MTMR9, NUDT15, |
| Biosynthesis | PPFIA3, PPTC7, PXYLP1 |
| Salvage Pathways of Pyrimidine | CDK1, CDK2, CDK6, FAM20B, MAPK8, PLK1 |
| Ribonucleotides | |
| Angiopoietin Signaling | CASP9, GRB7, KRAS, PAK4, PTK2 |
| 3-phosphoinositide Biosynthesis | CDC25A, DUSP8, HACD2, MTMR9, NUDT15, |
| | PIP4K2C, PPFIA3, PPTC7, PXYLP1 |
| CXCR4 Signaling | ADCY9, CXCL12, CXCR4, ELMO2, KRAS, |
| | MAPK8, PAK4, PTK2, RAC2 |
| ERK/MAPK Signaling | CRKL, ETS1, KRAS, MYCN, PAK4, PPP2R2B, |
| | PTK2, RAC2, SOS2, YWHAZ |
| Chemokine Signaling | CXCL12, CXCR4, KRAS, MAPK8, PTK2 |
| Adrenomedullin signaling pathway | ADCY9, KCNH2, KRAS, MAPK8, MAX, MYLK, |
| | MYLK2, PRKG2, PTK2, SOS2 |
| Prolactin Signaling | KRAS, PDPK1, PRL, SOCS2, SOS2 |
| Acute Phase Response Signaling | IL6R, KRAS, MAPK8, MBL2, OSMR, PDPK1, |
| | SOCS2, SOS2, TCF4 |
| Colorectal Cancer Metastasis Signaling | ADCY9, CASP9, CCND1, GRK3, IL6R, KRAS, |
| | LEF1, MAPK8, MSH2, RAC2, SOS2, TCF4 |
| Paxillin Signaling | ITGB8, KRAS, MAPK8, PAK4, PTK2, SOS2 |
| Fc Epsilon RI Signaling | INPPL1, KRAS, MAPK8, PDPK1, RAC2, SOS2 |
| Fcγ Receptor-mediated Phagocytosis in | ARPC2, ARPC5, PLD6, RAC2, RPS6KB2 |
| Macrophages and Monocytes | |
| Signaling by Rho Family GTPases | ARHGEF12, ARHGEF5, ARPC2, ARPC5, |
| | MAPK8, MYLK, PAK4, PIP4K2C, PTK2, RAC2, |
| | STMN1 |
| GM-CSF Signaling | CCND1, ETS1, KRAS, SOS2 |
| Renin-Angiotensin Signaling | ADCY9, KRAS, MAPK8, PAK4, PTK2, SOS2 |
| ERK5 Signaling | KRAS, MAP3K3, RPS6KB2, YWHAZ |
| Ephrin B Signaling | CXCL12, CXCR4, PTK2, RAC2 |
| Growth Hormone Signaling | PDPK1, PRL, RPS6KB2, SOCS2 |
| Neurotrophin/TRK Signaling | KRAS, MAPK8, PDPK1, SOS2 |
| Synaptogenesis Signaling Pathway | ADCY9, AP2M1, ARPC2, ARPC5, CADM1, |
| | CRKL, EFNA4, KRAS, NSF, RAB5B, RPS6KB2, |
| | SOS2, THBS3 |
| Renal Cell Carcinoma Signaling | ETS1, KRAS, PAK4, SOS2 |
| mTOR Signaling | EIF4B, KRAS, PDPK1, PLD6, PPP2R2B, RAC2, |
| | RPS17, RPS21, RPS6KB2 |
| Sirtuin Signaling Pathway | ACSS2, ATP5PB, E2F1, GABPA, HIST1H1E, |
| | IDH2, MYCN, POLR1E, TOMM20, TUBA1A, |
| | TUBA1B, TUBA4A |
| CDK5 Signaling | ADCY9, KRAS, LAMC1, MAPK8, PPP2R2B |
| Leukocyte Extravasation Signaling | CRKL, CXCL12, CXCR4, MAPK8, PTK2, RAC2, |
| | TIMP4 |
| Cdc42 Signaling | ARPC2, ARPC5, IQGAP3, MAPK8, MYLK, |
| | PAK4 |
| FGF Signaling | CRKL, FGF19, MAPK8, SOS2 |
| ILK Signaling | CCND1, ITGB8, LEF1, MAPK8, PDPK1, |
| | PPP2R2B, PTK2, RAC2 |
| Opioid Signaling Pathway | ADCY9, AP2M1, GRK3, KRAS, RAC2, RGS10, |
| | RGS16, RPS6KB2, SOS2 |
| NRF2-mediated Oxidative Stress Response | DNAJB5, GSR, HERPUD1, JUND, KRAS, |
| | MAPK8, SQSTM1 |
| Cardiac β-adrenergic Signaling | ADCY9, GRK3, PDE3A, PDE4D, PLD6, |
| | PPP2R2B |
| Mouse Embryonic Stem Cell Pluripotency | KRAS, LEF1, SOS2, TCF4 |
| eNOS Signaling | ADCY9, CASP9, CCNA2, CHRNA5, LPAR3, |
| | PDPK1 |
| Aldosterone Signaling in Epithelial Cells | ASIC3, DNAJB5, KRAS, PDPK1, PIP4K2C, |
| | SOS2 |
| p70S6K Signaling | KRAS, PDPK1, PPP2R2B, SOS2, YWHAZ |
| Regulation of eIF4 and p70S6K Signaling | KRAS, PDPK1, PPP2R2B, RPS17, RPS21, |
| | SOS2 |
| EIF2 Signaling | ATF3, CCND1, KRAS, MYCN, PDPK1, |
| | RPL27A, RPS17, RPS21, SOS2 |
| GNRH Signaling | ADCY9, KRAS, MAP3K3, MAPK8, PAK4, PTK2, |
| | SOS2 |
| Cholecystokinin/Gastrin-mediated Signaling | KRAS, MAPK8, PTK2, RAC2, SOS2 |
| IL-6 Signaling | IL6R, KRAS, MAPK8, MCL1, SOS2 |
| Thrombin Signaling | ADCY9, ARHGEF12, ARHGEF5, KRAS, MYLK, |
| | PDPK1, PTK2, RAC2 |
| Protein Kinase A Signaling | ADCY9, CDC25A, DUSP8, HIST1H1E, LEF1, |
| | MYLK, MYLK2, PDE3A, PDE4D, PLD6, PTK2, |
| | PTPN3, PTPN9, TCF4, YWHAZ |
| Melanocyte Development and Pigmentation | ADCY9, KRAS, RPS6KB2, SOS2 |
| Signaling | |
| Apelin Endothelial Signaling Pathway | ADCY9, KRAS, MAPK8, RPS6KB2 |
| CD28 Signaling in T Helper Cells | ARPC2, ARPC5, MAPK8, PDPK1 |
| Cardiac Hypertrophy Signaling | ADCY9, IL6R, KRAS, MAP3K3, MAPK8, RAC2 |
| Cardiac Hypertrophy Signaling (Enhanced) | ADCY9, FGF19, IL17RD, IL22RA1, IL6R, |
| | KRAS, MAP3K3, MAPK8, PDE3A, PDE4D, |
| | PLD6, PTK2, RPS6KB2, TGFBR3 |
| Endothelin-1 Signaling | ADCY9, CASP2, CASP9, KRAS, MAPK8, PLD6 |
| Gα12/13 Signaling | KRAS, LPAR3, MAPK8, PTK2 |
| IL-8 Signaling | CCND1, KRAS, MAPK8, PLD6, PTK2, RAC2 |
| NF-κB Signaling | KRAS, MAP3K3, MAPK8, PDGFRB, TGFBR3 |
| Neuroinflammation Signaling Pathway | APH1B, CXCL12, HMGB1, IL6R, MAPK8, |
| | TGFBR3 |
| Osteoarthritis Pathway | CASP2, CASP9, DKK1, HMGB1, LEF1, SPP1, |
| | TCF4 |
| PKCθ Signaling in T Lymphocytes | KRAS, MAP3K3, MAPK8, RAC2, SOS2 |
| Phospholipase C Signaling | ADCY9, ARHGEF12, ARHGEF5, KRAS, PLD6, |
| | RAC2, SOS2 |
| Role of NFAT in Cardiac Hypertrophy | ADCY9, KRAS, MAPK8, RCAN3, SOS2 |
| Sperm Motility | MST1R, PDE4D, PDGFRB, PRKG2, PTK2, |
| | ROR1, TWF1 |
| Tec Kinase Signaling | MAPK8, PAK4, PTK2, RAC2, TNFRSF21 |
| cAMP-mediated signaling | ADCY9, PDE3A, PDE4D, PLD6, RGS10, RGS2 |

### Example 2—RNA-Sequencing, Differential Gene Expression, and Pathway Analysis after Treatment of Different Cancer Cell Lines with miRNA-193a

miRNA-193a was tested in different cancer cell lines (see Table 2.1). The cells were treated with miRNA-193a as described for example 1 at different concentrations (1, 3, 10 nM). Controls (mock, untreated, and scrambled) were measured for all cell types. Assays were performed after 24h, 48h and 72h. Table 2.1 shows results at 10 nM concentration at indicated time points. The results were quantified and normalized to the mock control. 10 nM was a suitable concentration, because the cells showed no signs of a toxic effect at that concentration.

TABLE 2.1
effect of miRN-193a on various tumours

| | | | | Cell cycle | |
|---|---|---|---|---|---|
| Cancer | Cell | Viability | Apoptosis | arrest | Motility |
| type | line | (96 h) | (48/72 h) | (72 h) | (18 to 24 h) |
| Liver | HEP3B | <50% | <2x | G2/M | >50% |
| | HUH7 | <50% | <2x | — | n.a. |
| Lung | A549 | <50% | >2x | SubG1 | >50% |
| | H460 | <50% | >2x | SubG1 | n.a |

miRNA-193a treatment in the cancer cell lines decreased cell viability over time as measured by either an MTS assay or by cell count. Apoptosis induction was enhanced overtime as measured by a caspase 3/7 apoptosis assay. Cell cycle arrest profiles were measured performing either nuclei imaging or flow cytometry. miRNA-193a treatment induced either a G2/M or a SubG1 cell cycle arrest profile in a manner depending on the cell line. While in HUH7 no obvious cell cycle arrest profile was observed following the indicated methods, an increased apoptosis was observed indicated by Caspase 3/7 activation and enhanced cleaved-parp protein on western blot (data not shown) following miRNA-193a treatment in this cell line. This result indicates that miRNA-193a treatment affects the viability of the cells. Cell motility of two cell lines was significantly decreased after treatment as assessed via a Boyden chamber assay.

Conclusion

miRNA-193a treatment decreased cell viability partly by inducing apoptosis and by an increase in the cell cycle arrest profile. miRNA-193a treatment also decreases cell motility of cancer cells, indicating its role in the inhibition of cancer cell migration.

### Example 3—Further Study of the PTEN Pathway Activation

Example 1 shows that the IPA analysis identified the tumor suppressive PTEN pathway as the most enriched canonical pathway which was activated by miRNA-193a. Here regulation of selected miRNA-193a targets is analysed at the protein level by western blotting, including: FAK (PTK2), P70S6 (RPS6KB2), PI3KR1, TGFBRIII and other important signaling molecules including P-AKT, AKT, p-ERK1/2, ERK1/2, p-c-RAF and c-RAF, all factors in the PTEN pathway.

Materials and Methods

Cell Preparation

Human cancer cell lines were cultured in appropriate media (see table below) and seeded into 6-well plates before transfection with 10 nM miRNA-193a-3p mimic as described in example 1, 10 nM scrambled random control, or mock using Lipofectamine RNAiMAX (Thermofisher). Media was aspirated 72h after transfection and plates were stored at −80° C.

### 3. 1. Cell Lines Details

| Cell line | Cancer type | Medium |
|---|---|---|
| A549 | Lung (NSCLC) | F-12K + 10% FBS + P/S |
| BT549 | Breast (TNBC) | RPMI-1640 + 10% FBS + P/S + |
| | | 0.023 IU/mL insulin |
| H460 | Lung (NSCLC) | RPMI-1640 + 10% FBS + P/S |
| HEP3B | Liver (HCC) | EMEM + 10% FBS + P/S |
| HUH7 | Liver (HCC) | DMEM low glucose + 10% FBS + |
| | | P/S + L-glutamine |
| PANC-1 | Pancreas | DMEM + 10% FBS + P/S |
| SNU449 | Liver (HCC) | RPMI-1640 + 10% FBS + P/S |

FBS: fetal bovine serum,
P/S: penicillin streptomycin

Protein Isolation and Quantification

RIPA buffer (50 mM Tris-HCl pH 8, 150 mM NaCl, 1% NP40, 0.5% sodium deoxycholate, 0.1% SDS, 0.5 mM EDTA), supplemented with protease and phosphatase inhibitor cocktails, was added to harvested cells. Lysates were centrifugated at 15,000 g for 1 h at 4° C. and clarified by removing the cell debris pellet. Protein concentration was determined using the Pierce BCA Protein Assay Kit (Thermo Fisher).

Electrophoresis and Immunoblotting

Samples were separated at 120 V by SDS-PAGE on Mini-PROTEAN TGX Stain-Free precast gels (Bio-Rad). Proteins were transferred at 200 mA for 2 h to PVDF membranes in transfer buffer (25 mM Tris, 192 mM glycine, 20% methanol). The membranes were blocked using 5% milk or 5% BSA in Tris-buffered saline with Tween (20 mM Tris pH 7.6, 137 mM NaCl, 0.1% Tween). Blots were probed with primary and horseradish peroxidase-conjugated secondary antibodies. Proteins were detected using ECL reagents. Membranes were stripped in stripping buffer (62.5 mM Tris pH 6.8, 2% SDS, 100 mM 2-mercaptoethanol) for 30 min at 50° C. and reprobed as appropriate.

Results

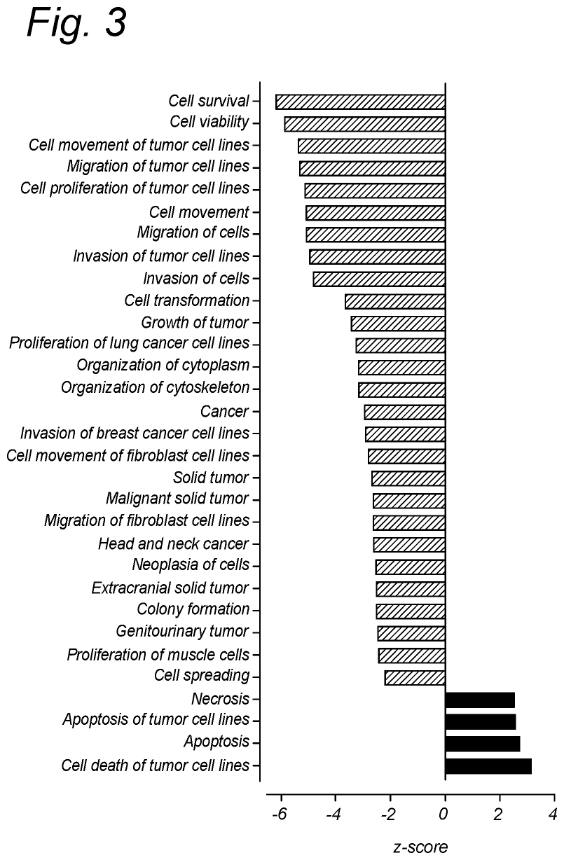

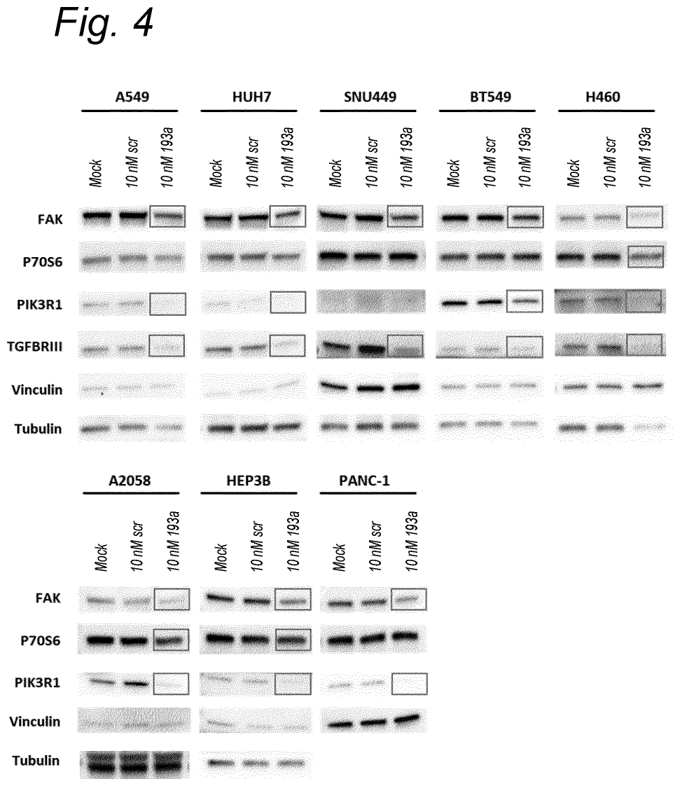

Lysates from cells transfected with 10 nM scrambled control or 10 nM miRNA-193a-3p mimic as described in example 1 were immunoblotted to assess the protein level of selected predicted miR-193a-3p target genes as well as phosphorylation status of key signalling proteins in the PTEN pathway. In all tested cell lines (A549, HUH7, SNU449, BT549, H460, A2058, HEP3B and PANC-1) downregulation of FAK, also called PTK2, was observed in the miRNA-193a sample compared to mock and scrambled control (FIG. 4). TGFBRIII was also downregulated by miRNA-193a in cell lines where a constitutive expression level could be observed (A549, HUH7, SNU449, BT549 and H460). Protein level of PIK3R1, the regulatory subunit of PI3K, was decreased in all cell lines except SNU449. P70S6, also called RPS6KB2, was downregulated in H460, A2058 and HEP3B. Vinculin and tubulin were used as loading controls. In A549 and H460, tubulin was affected by miRNA-193a, whereas vinculin was stable, indicating that miRNA-193a does not reduce general protein levels. Additionally, we observed downregulation of AKT phosphorylation by miRNA-193a in most cell lines (A549, A2058, SNU449, HUH7, H460 and HEP3B (FIG. 5). Interestingly, miRNA-193a increased phosphorylation of ERK in at least two cell lines (A549 and A2058). In SNU449, both ERK phosphorylation and ERK total protein level were upregulated. Phosphorylation of c-RAF was only downregulated in PANC-1.

Conclusion

These results are in line with the RNA-sequencing data obtained previously. miRNA-193a-3p mimic miRNA-193a decreased protein expression of FAK, P70S6K, PIK3R1 and TGFBRIII in multiple human tumor cell lines. In addition, treatment of cells with miRNA-193a-3p mimic miRNA-193a lead to reduced phosphorylation of AKT, which could be due to downregulation of upstream signaling proteins such as PIK3R1 and FAK. Furthermore, we observed increased phosphorylation of ERK, which could be a consequence of decreased AKT activity via effects on RAF, although phosphorylation of c-RAF was decreased in only one cell line (PANC-1). Increased phosphorylation of ERK may also be the result of other upstream events, including decreased phosphatase activity or increased activity of upstream kinases.

### Example 4—miRNA-193a is an Immunogenic Cell Death (ICD) Inducer

Introduction: The concept of Immunogenic Cell Death (ICD) has been defined as a unique class of regulated cell death capable of eliciting antigen-specific adaptive immune responses through the emission of a spatiotemporally defined set of danger signals known as damage associated molecular patterns (DAMPs) (Krysko et al., Nat. Rev. Cancer, 2012; Casares et al., J. Exp. Med., 2005; Kroemer et al., Annu. Rev. Immunol., 2013). The most notable DAMPs are: release of HGMB1, release of ATP and surface expression of calreticulin (CRT), as a sign of ER stress. Induction of ICD by some (specific) anticancer agents upon induction of cancer cell death leads to release of DAMPs into the tumor microenvironment (TME), which operate on receptors expressed by dendritic cells, and in turn stimulate presentation of tumor-associated antigens to T cells, leading to T cell activation and proliferation eventually culminating in enhanced cytotoxicity against the tumor cells, and formation of an immunological memory against the tumor antigens.

Materials and Methods

Transfection: A2058 melanoma and HEP3B hepatocyte tumor cells were transfected with different concentrations of miRNA-193a as described in example 1, or a mock (“fake transfection”) control. In brief, 5×10⁵ A2058 or HEP3B cells were seeded in 1.5 mL complete media in 6-well cell culture plates. Both cell lines were transfected 4 h later. A 500 μL transfection mix containing 7.5 μL Lipofectamine RNAiMAX (Thermo Fisher) and the appropriate concentration miRNA-193a-3p was added to each well. Transfection conditions included were 0.1, 1, 3 or 10 nM miRNA-193a and the mock-transfected negative control. Both cell lines were passaged into 24-well plates 16 h after transfection by aspirating and retaining media in 5-mL tubes, washing 1× with TrypLE (Gibco), and incubating for 10 to 12 min until detached. Cells were collected with 1 mL fresh media and added to the retained media. Tubes were centrifuged for 5 min at 1,500 RPM and supernatant removed. Cells were resuspended in 500 μL fresh media and counted using a 1:1 dilution with trypan blue using the Luna-II cell counter (Westburg). 5×10⁴ cells in 1 mL fresh media were seeded per well.

Flow cytometry: For flow cytometric analysis at mentioned time post transfection, cells were harvested afterwashing 1× with TrypLE (Gibco), and incubating for 10 to 12 min until detached. For each condition, 200 μL of single cell suspensions containing 5×10⁴ cells were prepared in 4-mL polypropylene tubes. Cells were stained with fluorescently labeled antibodies in a 1:200 dilution. The expression of CRT was measured using a DyLight™ 488 conjugate anti-human Calreticulin (CRT) antibody (Clone FMC 75, Enzo Life science). Also, DAPI (BioLegend) was added at a final concentration of 2 μM, to detect live/dead cells, and dead cells were excluded from further analyses. Flow cytometry was performed using a FACSCanto II cytometer (BD Biosciences), data was analyzed with FlowJo software (Tree Star inc.).

Co-culture with CFSE labeled PBMCs: PBMCs were isolated from fresh blood buffy coat (Sanquin), using SepMate™-50 tubes (STEMCELL), following manufacturer's protocol. Ficoll® Paque Plus (SigmaAldrich) was used as the density gradient medium. PBMCs were then labeled with CFSE using CFSE Cell Division Tracker Kit (BioLegend), following the manufacturer's protocol. A2058 cells were transfected and 16 h after transfection, cells were passaged to a 24 well plate as explained before. 3×10⁴ A2058 cells were seeded in 0.5 mL of fresh medium into each well. Also, 0.5 mL of CFSE labeled PBMC suspensions containing 1.2×10⁵PBMCs was added into each well. Same amount of PBMCs, without any A2058 cells, was cultured as “PBMC only” control condition. The co-culture was incubated at 370 C for mentioned time. For detection of T cells, cells were stained with Brilliant Violet 510™ anti-human CD3 Antibody (Clone UCHT1, BioLegend) in a 1:200 dilution.

Results

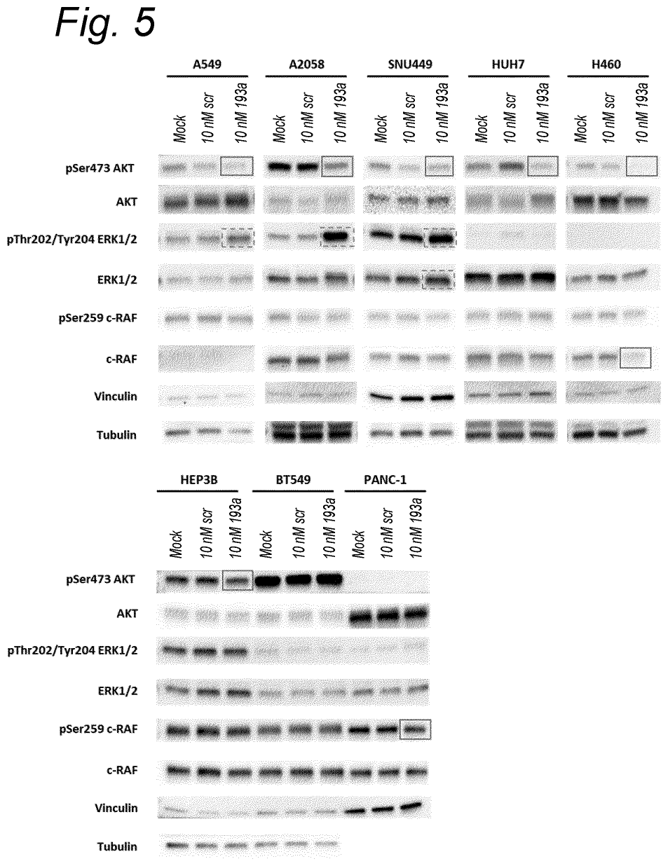

To investigate the effect of miRNA-193a on tumor cells, the expression of CRT on the surface of miRNA-193a transfected tumor cells was assessed by flow cytometry. As shown in FIGS. 6A and 6B, miRNA-193a induced expression of the CRT marker on the cell surface in A2058 cells (up to 46% after 72h) and to a lesser extent in Hep3B cells (up to 8% after 72h), compared with mock transfected cells containing only 5% and 4% surface-CRT⁺ cells, respectively. Moreover, via targeting two major ectonucleotidases CD39 and CD73, miRNA-193a can prevent the conversion of extracellular ATP to ADP, AMP and adenosine, and thereby retains the ATP content of the TME.

Next, we addressed the effect of miRNA-193a on proliferation of T cells in co-culture with miRNA-193a transfected tumor cells. PBMCs were labeled with CFSE, a fluorescent non-toxic marker that can be retained within the cells and gets diluted with each cell division. Levels of CFSE measured by flow cytometry were compared between three conditions: 1) PBMCs in culture alone, 2) PBMCs in culture with mock transfected A2058 cells, and 3) PBMCs in culture with miRNA-193a 1 nM transfected A2058 cells. The results show that keeping PBMCs in co-culture with miRNA-193a-transfected A2058 cells enhanced the proliferation of T cells.

Furthermore, miRNA-193a increased the vulnerability of tumor cells to PBMC-mediated cytotoxicity, as showed by fixation, staining and colorimetric quantification of survived tumor cells following co-culture with PBMCs. Interestingly, in vivo experiments in a syngeneic murine 4T1 orthotopic breast cancer model confirmed the formation of a long-term T cell mediated anti-tumor immunity in miRNA-193a treated animals, or in naïve mice that had received an adoptive T cell transfer from miRNA-193a treated mice.

Taken together, these results strongly suggest that miRNA-193a is a bona fide ICD inducer which kills the tumor cells in a way that not only stimulates PBMC-mediated cytotoxicity to enhance overall anti-tumor efficacy, but also activates the formation of an adaptive anti-tumor immunity.

### Example 5 Effect of miRNA-193a on Human PBMC-Mediated Tumor Cell Killing Following Transfection in Human Tumor Cells

One of the most recent developments in the understanding of cancer biology is the field of immuno-oncology (10). Often tumors present the ability to evade cancer immunosurveillance, which represents one of the hallmarks of cancer (Hanahan et al., 2011) Accordingly, the main goals of cancer immunotherapy are to strengthen the patient's immune response to the tumor by improving its capacity for tumor recognition and the disruption of immunosuppressive mechanisms (Chen et al., 2017). As part of the induction mechanisms supporting pronounced immune suppression of the tumors, adenosine levels in the tumor microenvironment (TME) have recently attracted significant attention to develop novel therapeutic intervention in oncology. Adenosine in the tumor microenvironment (TME) is generated mainly by ectonucleotidases CD39 (ENTPD1; which converts extracellular adenosine triphosphate (ATP) to adenosine diphosphate (ADP) and then to adenosine monophosphate (AMP)) and CD73 (NT5E; which is responsible for the generation of adenosine from AMP) (Stagg et al., 2010). NT5E can act as inhibitory immune checkpoint molecule, since free adenosine generated by NT5E inhibits cellular immune responses, thereby promoting immune escape of tumor cells. Indeed, adenosine is a potent immunosuppressive metabolite that is generated in response to pro-inflammatory stimuli, such as cellular stress initiated by hypoxia or ischemia. Landmark studies by Ohta and colleagues have highlighted the importance of adenosine for tumor immune escape (Ohta et al., 2006). Extracellular adenosine concentrations in solid tumors are reported to be higher than under normal physiological conditions (Blay et al., 1997).

Our transcriptome analysis identified a pool of immune related genes among the genes whose expression was affected by a mimic of miR-193a-3p as described in example 1. Among them were modifiers of TME, such as CD73. Moreover, our in vivo studies in murine models strongly suggested that miR-193a-3p, on top of its other effects, modifies the interaction between tumor cells and the immune system in a way that immune cells become more active in killing tumor cells. To assess the 10 related effect of miR-193a-3p in human cells, and also to investigate the mechanism of the miR-193a-3p mediated 10 effect, we established an in vitro assay in which, tumor cells were co-cultured together with human peripheral blood mononuclear cells (hPBMCs) isolated from healthy donor's peripheral blood, and the cytotoxic effect of hPBMCs on tumor cells was assessed with or without transfection with miR-193a-3p (see example 4).

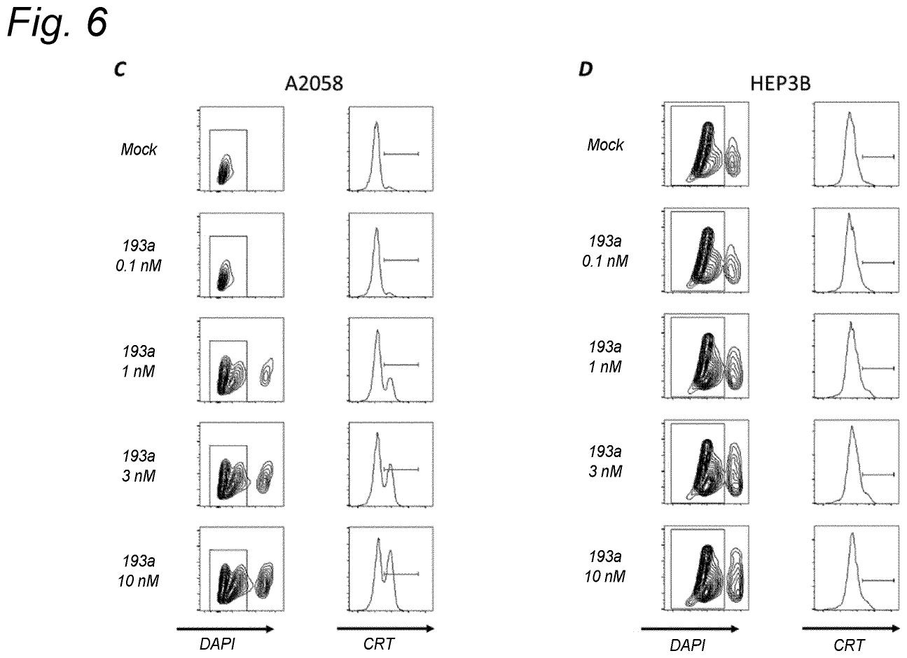

As a first step and to establish the technical validity of such a cell-based assay, human anti CD3/CD28 T cell activator antibodies (positive control) was added to the tumor cells and PBMCs co-culture. The used activator comprises a soluble tetrameric antibody complex that binds CD3 and CD28 immune cell surface ligands. This binding results in cross-linking of CD3 and CD28, thereby providing the required primary and co-stimulatory signals for an effective T cell activation (Riddell et al., 1990; Bashour et al., 2014). As illustrated in FIG. 8, although unstimulated human PBMCs showed limited effect on tumor cell survival (co-culture), addition of anti CD3/CD28 antibodies in the co-culture led to a pronounced decrease in tumor cell survival, most likely consequent to an efficient T cell activation and subsequent T cell-mediated tumor cell killing. Interestingly, in similar study performed with primary human dermal fibroblasts, no effect of anti CD3/CD28 on fibroblast viability was observed (data not shown), strongly suggesting that experimental T cell activation does not lead to T cell-mediated normal fibroblast killing.

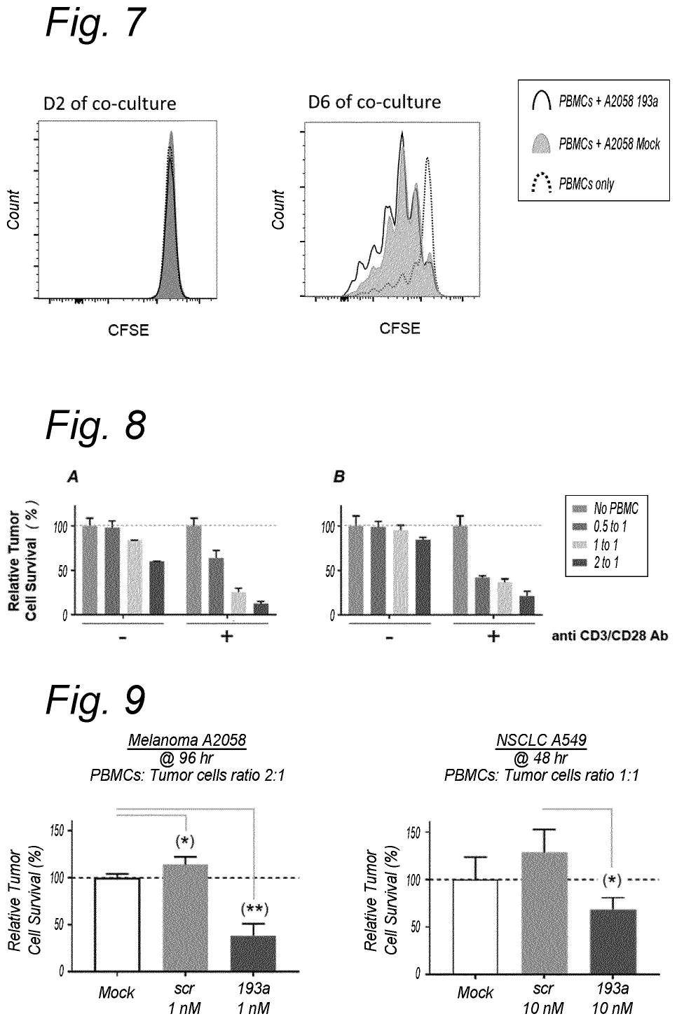

Next, human melanoma A2058 and NSCLC A549 tumor cells were transfected with increasing concentrations of miR-193a-3p after which they were co-cultured with human PBMCs (at different PBMCs:Tumor cells ratio) for different times. Human PBMCs from independent donors were able to induce time-dependent marked tumor cell killing upon transfection of tumor cells with miRNA-193a as described in example 1, but not the (negative) miRNA control (scramble), validating sequence-specificity of miRNA-193a activity (FIG. 9).

Taken together, our results demonstrate that transfection of tumor cells with miR-193a-3p clearly increases the vulnerability of tumor cells (e.g., A2058 and A549 tumor cells) to human PBMC cytotoxicity, by sensitizing tumor cells to PBMCs, and/or by releasing signals from transfected tumor cells to activate T cell-containing PBMCs.

1-14. (canceled)

15. A method for treating a condition associated with PTEN deficiency, the method comprising the step of administering to a subject a miRNA-193a or a source thereof, or a composition comprising a miRNA-193a or a source thereof.

16. The method according to claim 15, wherein the miRNA-193a is a PTEN agonist.

17. The method according to claim 15, wherein the miRNA-193a is a miRNA-193a molecule, an isomiR, or a mimic thereof.

18. The method according to claim 17, wherein the miRNA-193a is an oligonucleotide with a seed sequence comprising at least 6 of the 7 nucleotides of the seed sequence represented by SEQ ID NO: 22.

19. The method according to claim 15, wherein the source of the miRNA-193a is a precursor of the miRNA and is a nucleic acid of at least 50 nucleotides in length.

20. The method according to claim 15, wherein the miRNA-193a shares at least 70% sequence identity with any one of SEQ ID NOs: 56, 121, or 122.

21. The method according to claim 15, wherein the miRNA-193a is from 15-30 nucleotides in length.

22. The method according to claim 15, wherein the source of the miRNA-193a is a precursor of said miRNA-193a and shares at least 70% sequence identity with any one of SEQ ID NOs: 5 or 13.

23. The method according to claim 15, wherein the condition associated with PTEN deficiency is a PTEN-deficient cancer.

24. The method according to claim 23, wherein the PTEN-deficient cancer is a PTEN-deficient sarcoma, brain cancer, head and neck cancer, breast cancer, lung cancer, kidney cancer, liver cancer, colon cancer, ovarian cancer, melanoma, pancreatic cancer, thyroid cancer, hamartoma, tumour of the haematopoietic and lymphoid malignancy, or prostate cancer.

25. The method according to claim 15, wherein the miRNA-193a modulates expression of a gene selected from the group consisting of RPS6KB2, KRAS, PDGFRB, SOS2, TGFBR3, CASP9, INPPL1, PIK3R1, PTK2, CBL, PDPK1, CCND1, BCAR1, MAGI3, MDM2, YWHAZ, and MCL1.

26. The method according to claim 25, wherein the miRNA-193a modulates expression of a gene selected from the group consisting of RPS6KB2, KRAS, PDGFRB, CASP9, INPPL1, PIK3R1, PTK2, CBL, PDPK1, CCND1, BCAR1, MAGI3, MDM2, YWHAZ, and MCL1.

27. The method according to claim 25, wherein the miRNA-193a modulates expression of PDPK1 or INPPL1.

28. The method according to claim 15, wherein the composition comprising the miRNA-193a or a source thereof is administered to the subject.

29. The method according to claim 28, wherein the composition further comprises a further miRNA or precursor thereof, wherein the further miRNA is selected from the group consisting of miRNA-323, miRNA-342, miRNA-520f, miRNA-520f-i3, miRNA-3157, and miRNA-7, or an isomiR thereof, or a mimic thereof.

30. The method according to claim 28, wherein the composition further comprises an additional pharmaceutically active compound.

31. The method according to claim 30, wherein the additional pharmaceutically active compound is selected from the group consisting of a PP2A methylating agent, an inhibitor of hepatocyte growth factor (HGF), an antibody, a PI3K inhibitor, an Akt inhibitor, an mTOR inhibitor, a binder of a T cell co-stimulatory molecule such as a binder of OX40, and a chemotherapeutic agent.

32. The method according to claim 28, wherein the composition is a nanoparticle comprising a diamino lipid and the miRNA-193a or a source thereof, wherein the diamino lipid is of general formula (I): 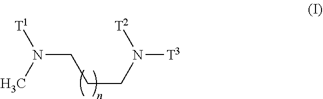
wherein
n is 0, 1, or 2, and
T¹, T², and T³ are each independently a C₁₀-C₁₈ chain with optional unsaturations and with zero, one, two, three, or four substitutions, wherein the substitutions are selected from the group consisting of C₁-C₄ alkyl, C₁-C₄ alkenyl, and C₁-C₄ alkoxy.

33. The method according to claim 32, wherein the nanoparticle comprises:
i) 20-60 mol % of the diamino lipid, and
ii) 0-40 mol % of a phospholipid, and
iii) 30-70 mol % of a sterol, and
iv) 0-10 mol % of a conjugate of a water soluble polymer and a lipophilic anchor.

34. An in vivo, in vitro, or ex vivo method for agonising PTEN, the method comprising the step of contacting a cell with a miRNA-193a or a source thereof, or with a composition comprising a miRNA-193a or a source thereof.
